(19) United States
(12) Patent Application Publication
(10) Pub. No.: US 2023/0002790 A1
(43) Pub. Date: Jan. 5, 2023

(54) POLY(Beta-AMINO ESTER) NANOPARTICLES FOR THE NON-VIRAL DELIVERY OF PLASMID DNA FOR GENE EDITING AND RETINAL GENE THERAPY

(71) Applicant: The Johns Hopkins University, Baltimore, MD (US)

(72) Inventors: Jordan J. Green, Nottingham, MD (US); David Wilson, Smithsburg, MD (US); Yuan Rui, Baltimore, MD (US); Donald J. Zack, Baltimore, MD (US); Srinivasa Rao Sripathi, Baltimore, MD (US); Cynthia A. Berlinicke, Baltimore, MD (US)

(21) Appl. No.: 17/282,939

(22) PCT Filed: Oct. 10, 2019
(86) PCT No.: PCT/US2019/055741

§ 371 Date: Apr. 5, 2021

Related U.S. Application Data
(60) Provisional application No. 62/743,883, filed on Oct. 10, 2018.

(51) Int. Cl. C12N 15/88; C12N 15/11; C08G 69/26

(52) U.S. Cl. CPC C12N 15/88; C12N 15/111; C08G 69/26

(57) ABSTRACT

Biodegradable particles for delivering a nucleic acid encoding gene-editing factors or a nucleic acid associated with a therapeutic protein to a cell, and compositions, methods, systems, and kits for gene editing in vivo or ex vivo or gene therapy for treating retinal eye diseases are disclosed.

### FEDERALLY SPONSORED RESEARCH OR DEVELOPMENT

This invention was made with government support under grant numbers EB016721, EB022148, and EY001765 awarded by the National Institutes of Health. The government has certain rights in the invention.

### BACKGROUND

In gene editing, DNA is inserted, deleted, modified, or replaced in the genome of a living cell in vivo or ex vivo. Gene editing can be used to correct for genetic mutations that lead to human disease. For example, the CRISPR/Cas9 system can direct site-specific gene disruption. The Cas9 endonuclease introduces double stranded breaks at sites specified by a single guide RNA (sgRNA), and gene disruption occurs by the introduction of indels that cause frame-shift mutations or by the removal of large segments of the gene. While gene editing platforms (including the CRISPR/Cas9) hold great promise, effective and/or efficient delivery of gene editing factors to cells in vivo or ex vivo remains challenging.

Further, gene therapy holds potential promise for treating both acquired and inherited blinding disorders as most of the identified disease to date is associated with retinal pigment epithelial (RPE) cells. See Bainbridge et al., 2006. Modulating specific gene targets simply by turning off or turning on its function has become a standard tool to enhance stem cell differentiation or to reprogram induced pluripotent stem cells (iPSCs) from somatic cells. See Jia et al., 2010; Nauta et al., 2013. Routinely approached gene therapy utilizes viral vectors to deliver pDNA. This approach, however, is limited by several factors. To overcome these challenges and to follow an alternative safer approach, significant attempts have been made to formulate and develop biodegradable non-viral vehicles agents to facilitate delivery of the gene of interest to the target sites. Such approaches, however, have been limited by poor transfection efficacy.

### SUMMARY

In aspects, the presently disclosed subject matter provides a composition comprising a poly(beta-amino ester) (PBAE) of formula (I) or formula (II):

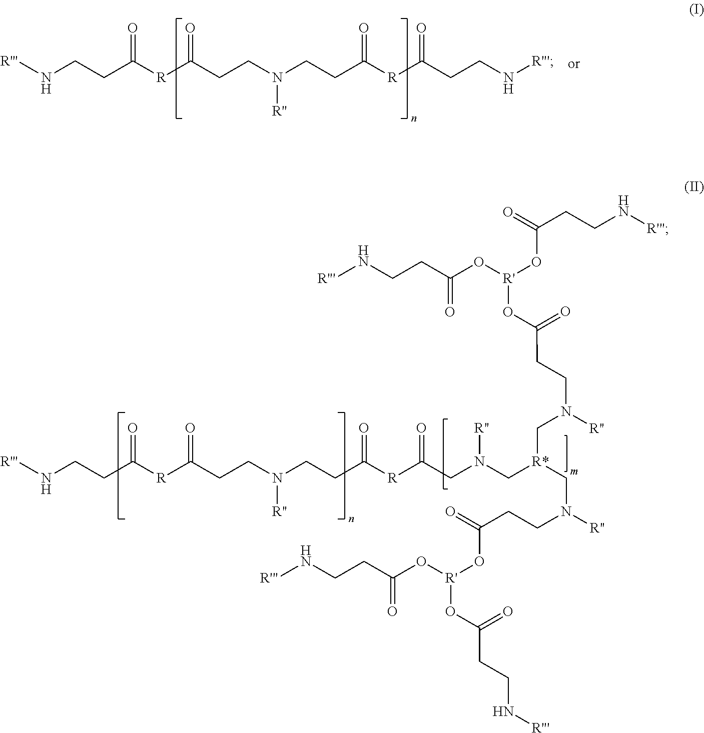

and at least one DNA or RNA molecule comprising a nucleic acid sequence encoding a gene-editing protein or therapeutic protein;

wherein:

n and m are each independently an integer from 1 to 10,000;

each R is independently a diacrylate monomer of the following structure:

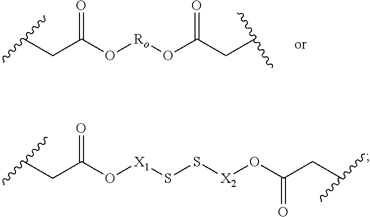

wherein Rₒ comprises a linear or branched C₁-C₃₀ alkylene chain, which may further comprise one or more heteroatoms or one or more carbocyclic, heterocyclic, or aromatic groups and X₁ and X₂ are each independently a linear or branched C₁-C₃₀ alkylene chain;

each R* is a triacrylate, quanternary, or hexafunctional acrylate monomer selected from the group consisting of:

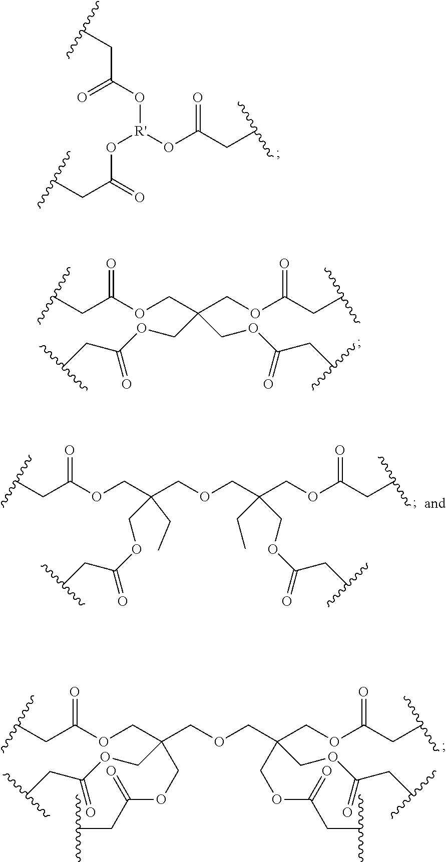

wherein each R′ is independently a trivalent group; each R″ is independently a side chain monomer comprising a primary, secondary, or tertiary amine; and each R″ is independently an end group monomer comprising a primary, secondary, or tertiary amine.

In other aspects, the presently disclosed subject matter provides a pharmaceutical formulation comprising the composition of formula (I) or formula (II) in a pharmaceutically acceptable carrier. In particular aspects, the formulation comprises a nanoparticle or microparticle of the PBAE of formula (I) or formula (II).

In yet other aspects, the presently disclosed subject matter provides a kit comprising the composition of formula (I) or formula (II). In certain aspects, the kit comprises one of more of multiple dosage units of the composition, a pharmaceutically acceptable carrier, a device for administration of the composition, instructions for use, and combinations thereof.

In some aspects, the presently disclosed subject matter provides a method for gene editing, comprising contacting a cell with a composition of formula (I) or formula (II), wherein the composition comprises at least one DNA plasmid comprising a nucleic acid sequence encoding a gene-editing protein.

In other aspects, the presently disclosed subject matter provides a method for treating a retinal eye disease, the method comprising administering to a subject in need of treatment thereof, a composition of formula (I) or formula (II), wherein the composition comprises a therapeutic protein for treating retinal eye disease.

In certain aspects, the retinal eye disease comprises a hereditary retinal eye disease. In particular aspects, the retinal eye disease is selected from the group consisting of age-related macular degeneration (AMD), including wet macular degeneration and dry macular degeneration, Leber's congenital amaurosis (LCA2) type 2, choroideremia, achromatopsia, retinitis pigmentosa (RP), Stargardt disease (STGD), Usher syndrome, juvenile X-linked retinoschisis (XLRS), and diabetic retinopathy.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1A is a schematic showing knockout of the eGFP gene when the gene is contacted with the presently disclosed PBAE nanoparticles (“PBAE NPs”) that carry nucleic acids encoding sgRNA (“sgGFP”) and nucleic acids encoding Cas9;

FIG. 1B is a schematic showing excision by a CRISPR-nanoparticle transfection of a 600 bp STOP cassette. Sg1 indicates the sgRNA-directed cut-sites. Excision of the STOP cassette allows expression of the coding sequence for Red-enhanced NanoLantern (ReNL);

FIG. 2A is a graph showing percentage knockout of the eGFP gene in cells transfected with a presently disclosed PBAE nanoparticle carrying either a nucleic acid encoding Cas9, a nucleic acid encoding sgRNA, or both nucleic acids, i.e., a nucleic acid encoding Cas9 and a nucleic acid encoding sgRNA;

FIG. 2B is a gel image showing Surveyor® mismatch enzyme cuts of PCR amplicons of edited cells providing evidence of CRISPR/Cas9 cutting when Cas9 and sgRNA were present. Bands at 370 bp and 240 bp show evidence of genomic DNA cleavage;

FIG. 2C is a graph comparing decreases in GFP signal in cells treated with an anti-GFP siRNA (squares) and in cells treated with a presently disclosed PBAE nanoparticle comprising CRISPR components (circles);

FIG. 2D provides representative sequences of Sanger sequenced genomic DNA (SEQ ID NOs: 13; 43-45) of cells edited by CRISPR/Cas9 via PBAE nanoparticles where only small indels were observed;

FIG. 2E shows includes flow cytometry histograms of the cells described in FIG. 2C. The left panel shows a minimal shift in the number of fluorescent cells resulting from the siRNA treatment at Day 1, whereas the right panel shows a large shift in the number of fluorescent cells resulting from the CRISPR-nanoparticle transfection at Day 3. The x- and y-axes relate to eGFP fluorescence intensity and the number of cells exhibiting fluorescence intensity, respectively;

FIG. 3A is a schematic showing excision by a CRISPR-nanoparticle transfection of >400 bp STOP cassette. Sg1 indicates the sgRNA-directed cut-sites. Excision of the STOP cassette allows expression of the coding sequence for Red-enhanced NanoLantern (ReNL);

FIG. 3B is a graph comparing the percentage of cells having an excised STOP cassette for untreated (UT) cells and for cells transfected with the PBAE nanoparticles encapsulating various sgRNAs (sg1), (sg2), (sg3), and (sg2+sg3);

FIG. 3C is a gel image showing a truncated ReNL PCR product following excision of the STOP cassette. UT is “Untreated”, and “sg1” is sgRNA-directed editing;

FIG. 3D is a fluorescent micrograph of cells with an sg1-excised STOP cassette; the excision results in a florescent signal via the ReNL protein. Scale bar=200 μm;

FIG. 4 shows a complete ReNL system schematic;

FIG. 5 shows microscopic images of ReNL gene deletion using various sgRNAs;

FIG. 6A shows the structure of a representative linear poly(beta-amino ester) (PBAE) polymer;

FIG. 6B is a schematic representing the preparation of nanoparticles carrying only Cas9 plasmids (identified by “*”);

FIG. 6C is a schematic representing the preparation of nanoparticles carrying only sgRNA plasmids (identified by “†”); and

FIG. 6D shows preparation of nanoparticles carrying Cas9 plasmids and sgRNA plasmids;

FIG. 7A and FIG. 7B illustrate a BGDA-series of hyperbranched PBAEs. Polymers are constructed from diacrylate monomers (BGDA; “*”), triacrylate monomers (TMPTA; “†”), side-chain monomer S4 (“‡”), and end-cap E6 (※) to synthesize a series of poly(β-amino esters) (PBAEs) with increasing triacrylate mole fraction and degree of branching. Linear PBAEs possess two end-cap E6 moieties per molecule, whereas each triacrylate monomer in branched PBAEs results in an additional endcap E6 moiety for every branch point;

FIG. 7C illustrates a one-pot synthesis of acrylate-terminated base polymers. In exemplary embodiments, a diacrylate monomer B7 and triacrylate monomer B8 were mixed with side-chain monomer S4 to synthesize a series of BEAQs with increasing triacrylate mole fraction and degree of branching. Linear polymers possess two end-cap structures per molecule, while each triacrylate monomer in branched polymers results in an additional end-cap moiety for every branch point. One-pot synthesis of acrylate terminated base polymers was performed at 90° C. and 200 mg/mL in DMF for 24 h. Polymers were then end-capped with monomer E6 at room temperature for 1 h to yield the final product;

FIG. 7D illustrates representative transmission electron microscopy (TEM) images of BGDA nanoparticles containing plasmid DNA. Scale bar=10 nm;

FIGS. 8A, 8B, 8C, 8D, 8E, and 8F show representative polymer characteristics. FIG. 8A shows the predicted properties of partition coefficient (log P) and distribution coefficient (log D) for variably branched BGDA PBAEs. FIG. 8B shows competition binding assay of polymer and Yo-Pro-1 iodide at low pH. (n=3 wells, mean±SEM). FIG. 8C shows competition DNA binding assay in isotonic, neutral buffer. (n=3 wells, mean±SEM); FIG. 8D shows the titration of PBAEs. FIG. 8E shows the effective pKa value of maximum buffering point between pH 4.5-8.5 of variably branched PBAEs. FIG. 8F shows the effective solubility of variably branched PBAEs at low pH and in isotonic, neutral buffer. Blending multiple monomers enables fine-tuning of polymer properties mid-way between the states of either monomer. Properties include hydrophobicity (assessed computationally via log P and log D), DNA binding, buffering capacity and effective pKa value;

FIGS. 9A, 9B, and 9C show BGDA nanoparticle properties. FIG. 9A shows the Z-average hydrodynamic diameter measurements in 25 mM NaAc buffer, pH 5.0 and after dilution into 150 mM PBS at a 40 w/w ratio. FIG. 9B shows the Zeta potential measurements assessed in 150 mM PBS, pH 7.4. (n=3 preparations, mean±SEM). FIG. 9C shows TEM images of dried particles. Scale bar 100 nm for all images. Nanoparticles have effectively the same properties for the tested polymer series regardless of degree of branching;

FIG. 10A, FIG. 10B, FIG. 10C, FIG. 10D, FIG. 10E, FIG. 10F, FIG. 10G, and FIG. 10H show the in vitro transfection of HEK239T cells or ARPE-19 cells with BGDA PBAEs in 10% serum media. FIG. 10A shows the transfection efficacy in HEK293T cells. FIG. 10B shows the normalized geometric mean expression. FIG. 10C shows the viability and FIG. 10D shows a fluorescent microscope image. FIG. 10E shows the transfection efficacy in ARPE-19 cells. FIG. 10F shows the normalized geometric mean expression. FIG. 10G shows the viability and FIG. 10H shows a fluorescent microscope image. Scale bars 200 μm. (n=4 wells, mean±SEM); Transfection efficacy of retinal ARPE-19 cells is notably much higher than both commercial transfection reagents Lipofectamine 2000 and jetPrime as well as the previously optimized PBAE 557;

FIG. 11A, FIG. 11B, FIG. 11C, and FIG. 11D demonstrate challenging transfection conditions with BGDA PBAEs. High serum (50%) transfection of HEK293T (FIG. 11A) and ARPE-19 cells (FIG. 11B) with 20 w/w nanoparticles. Low nanoparticle dose transfection with 40 w/w nanoparticles of HEK293T (5 ng) (FIG. 11C) and ARPE-19 (10 ng) (FIG. 11D) doses in 384 well plates. Branching notably improves transfection efficacy in both cell lines in high serum conditions and at low nanoparticle doses.

FIG. 12A, FIG. 12B, FIG. 12C, FIG. 12D, FIG. 12E, FIG. 12F, FIG. 12G, and FIG. 12H shows the correlation between polymer properties and transfection efficacy. (FIG. 12A-D) HEK293T cells and (FIG. 12E-H) ARPE-19 cells;

FIG. 13A and FIG. 13B show the chemical properties of the presently disclosed BGDA polymer series. FIG. 13A shows NMR spectra of the presently disclosed BGDA series of acrylate terminated PBAE polymers ¹H NMR (500 MHz, CDCl₃-ch, 0.05% v/v TMS) spectra. Note that some peaks are from residual solvent for diethyl ether (3.48, 1.2 ppm) and DMSO (2.62 ppm). Relevant peaks for determination of MN and triacrylate mole fraction are as follows. BGDA phenyl (4H each) 6.81 and 7.11 ppm in green; TMPTA methyl (3H) 0.83 ppm in red; S4 (2H/repeat) 2.38 ppm;

FIG. 13B shows gel permeation chromatography refractive index detector traces for the BGDA series of polymers. GPC and analysis in Waters2 software was used to calculate MN, Mw and PDI of each polymer relative to a third order curve fit of eight linear polystyrene standards (R²=0.9987) ranging in molecular weight from 580 Da to 3.15 MDa;

FIG. 14A, FIG. 14B, FIG. 14C, FIG. 14D, and FIG. 14E show the aqueous properties of the presently disclosed BGDA polymer series. FIG. 14A shows Marvin predicted log D values assessing polymer hydrophobicity at different pH values. Computed for 140 mM Cl—, Na/K+ conditions with NMR value MN matched polymer structures; FIG. 14B shows the method for calculation of effective buffering capacity at each pH point (between 4.5-8); FIG. 14C shows calculated normalized buffering capacity from individual polymer titrations enabled effective pKa value of each polymer to be determined; FIG. 14D shows the absorbance spectra of polymer BGDA-20 dissolved into 150 mM PBS, pH 7 at 10 mg/mL to determine 600 nm wavelength to approximate solubility measurements. The solubility of BGDA polymers (FIG. 14E) with absorbance >0.5 at 600 nm defined as insoluble was calculated from dilution series in (FIG. 14F) 150 mM PBS, pH 7.4 and (FIG. 14G) 25 mM NaAc, pH 5.0. Solubility increased as predicted with branching due to the increase in the number of hydrophilic endcap moieties;

FIG. 15A, FIG. 15B, and FIG. 15C show the DNA binding properties of the presently disclosed BGDA polymer series. For both buffer conditions the plots show fluorescence quenching as a function of polymer concentration, quenching normalized to number of secondary amines, normalized to number of tertiary amines and normalized to the total number of amines (FIG. 15A) Under acidic conditions at pH 5.0 and low salt, degree of DNA binding is best proportional to the number of tertiary amines per base pair (bp) of DNA. (FIG. 15B) In contrast, under neutral, isotonic conditions at pH 7.4, the degree of DNA binding is best proportional to the number of secondary amines per bp DNA. (FIG. 15C) The difference in binding between pH 5 to pH 7.4 for the linear (0% triacrylate), moderately branched polymer (40% triacrylate) and highly branched polymer (90% triacrylate) were compared;

FIG. 16A, FIG. 16B, FIG. 16C, FIG. 16D, FIG. 16E, and FIG. 16F show BGDA nanoparticle uptake in HEK293T and ARPE-19 cells. Branching does not strongly improve nanoparticle uptake compared to linear BGDA polymer nanoparticles at the same w/w ratios. HEK293T high dose nanoparticle uptake (600 ng dose, 20% labeled Cy5-DNA) as (FIG. 16A) percent uptake and (FIG. 16B) geometric mean. HEK293T low dose nanoparticle uptake (300 ng, 20% labeled Cy5-DNA) as (FIG. 16C) percent uptake and (FIG. 16D) geometric mean. ARPE-19 low dose nanoparticle uptake (300 ng, 20% labeled Cy5-DNA) as (FIG. 16E) percent uptake and (FIG. 16F) geometric mean;

FIG. 17A, FIG. 17B, and FIG. 17C shows BGDA series nanoparticle transfection in high serum (50%) conditions. HEK293T cells (FIG. 17A) transfection efficacy up to 97% and (FIG. 17B) geometric mean expression. ARPE-19 (FIG. 17C) transfection efficacy up to 67%. Moderately branched BGDA PBAEs outperformed the linear BGDA polymer when level of expression was taken into account; this effect was especially evident at low w/w ratios;

FIG. 18A, FIG. 18B, FIG. 18C, FIG. 18D, and FIG. 18E shows BGDA nanoparticle transfection at low doses in HEK239T cells and ARPE-19 cells. FIG. 18A shows extremely low volume distribution of nanoparticles achieved via Echo 550 acoustic liquid handling with nanoparticle dose titration. FIG. 18B shows transfection efficacy in HEK239T cells and FIG. 18C shows untreated normalized cell counts in HEK239T cells. FIG. 18D shows transfection efficacy in ARPE-19 cells and FIG. 18E shows untreated normalized cell counts in ARPE-19 cells. Branched BGDA polymers with 40-60% triacrylate mole-fraction were statistically more effective than the linear BGDA polymer tested for low dose nanoparticle transfection. No nanoparticle formulations showed high cytotoxicity (>30% reduction in cell count) when cell counts were compared relative to the mean cell count of eight untreated wells. Values show mean±SEM of three wells for each condition. Differences in transfection efficacy between polymers were assessed over all tested conditions by One-way ANOVA with multiple comparisons to the linear BGDA polymer BGDA-0 using matched values for w/w ratio and DNA dose. One-way ANOVA was performed with Geisser-Greenhouse corrections for sphericity and Dunnet corrections for multiple comparisons. P values shown are multiplicity adjusted;

FIG. 19 shows HEK293T transfection correlated with w/w scaled polymer characteristics. The number of secondary amines, tertiary amines, total amines and buffering capacity between pH 5-7.4 were calculated for each polymer at the tested w/w ratios. For viability, linear regression trend lines were calculated to assess if a single curve fit data for all polymers in the series;

FIG. 20 shows ARPE-19 transfection correlated with w/w scaled polymer characteristics. The number of secondary amines, tertiary amines, total amines and buffering capacity between pH 5-7.4 were calculated for each polymer at the tested w/w ratios. For viability, linear regression trend lines were calculated to assess if a single curve fit data for all polymers in the series;

FIG. 21A, FIG. 21B, FIG. 21C, FIG. 21D, FIG. 21E, and FIG. 21F show ARPE-19 transfection with Linear and branched PEI of various molecular weights were tested for optimal w/w ratio in (FIGS. 21A-21C) HEK293T and (FIG. 21D-21F) ARPE-19 cells. Geometric mean expression is shown normalized to untreated control cells (value of 1). Normalized viability is shown as a percentage of untreated control wells. (Error bars show n=4 wells, mean±SEM);

FIG. 22A and FIG. 23B show ARPE-19 transfection with control nanoparticle materials. To fairly identify optimal conditions for in vitro transfection, both a (FIG. 22A) 600 ng dose of DNA with two-hour incubation and a (FIG. 22B) 100 ng dose with 24-hour incubation were tested for control reagents. PBAE 557 was shown previously to be generally effective for transfection of ARPE-19 cells, which we reproduced, showing at most 40% transfection. JetPRIME likewise enabled transfection of up to 40% of cells, while Lipofectamine-2000 gave a transfection efficacy of only 20%;

FIG. 23 shows flow cytometry gating analysis. FlowJo 10 was used for gating cells analyzed from an Accuri C6 flow cytometer. Singlet cell populations were identified and 2D gated for GFP expression or uptake of Cy5 labeled plasmid DNA. For gating, untreated populations were set to be <0.5% false positive;

FIG. 24 show ineffective endcap monomers. Endcap structures shown were tested and confirmed to effectively react with acrylate terminated PBAE polymer 4-4-Ac, but the resulting polymers were wholly ineffective for delivery of plasmid DNA to HEK293T cells.

These E-monomers were excluded from large library endcapping for transfection efficacy studies in harder-to-transfect RPE monolayers;

FIG. 25 shows the characterization of base polymer PBAEs via ¹H NMR (500 Mhz) following 2× diethyl ether precipitated to verify that base polymer structures were acrylate terminated. The ratio of integrated acrylate peak area to s-monomer carbon area was used to determine molecular weight MN of base polymers. Calibration and contamination peaks include CDCl₃ 7.26; DMSO 2.62, diethyl ether 3.48 and 1.2 tetramethyl silane (TMS) 0;

FIG. 26A and FIG. 26B show gel permeation chromatography characterization of the presently disclosed PBAEs. PBAEs were characterized via gel permeation chromatography to assess molecular weight against linear polystyrene standards following synthesis and after dissolved in DMSO and washed with diethyl ether twice. Washing with diethyl ether was shown to remove unreacted monomers units as well as oligomers, (FIG. 26A) increasing polymer number average weight MN and (FIG. 26B) reducing the polydispersity index (PDI);

FIG. 27A and FIG. 27B show the post-mitotic status of differentiated RPE monolayers. Human iPS cells seeded in 384 plates were allowed to differentiate over 25 days in culture in 384 well plates. (FIG. 27A) Cell number per well increases through day 10, at which point cell number peaked and cells began to differentiate. (FIG. 27B) Cells are visibly more densely growing at day 25 post-seeding compared to day 3 post-seeding. RPE monolayer at day 25 additionally possessed textured appearance. Bars show mean±SEM of four wells for each condition. Scale bar 100 μm for 20× images;

FIG. 28A, FIG. 28B, and FIG. 28C show full differentiation from embryonic stem cells changes cell phenotype and optimal PBAE polymer structure. Scale bars are 100 μm. (FIG. 28A) Representative images of D3 RPE cells after plating transfected with 4-4-E2. (FIG. 28B) Heat map of transfection of D3 RPE with full PBAE library; (FIG. 28C) D3 viability heat map with full PBAE library;

FIG. 29A, FIG. 29B, FIG. 29C, FIG. 29D, FIG. 29E, and FIG. 29F show commercial reagent transfection efficacy optimization. Lipofectamine 3000 and DNA-In were tested under 2-hour and 24-hour incubation conditions at varying reagent ratio and DNA doses to identify the optimal condition for each. (FIG. 29A) Lipofectamine 3000 transfected at most 3% of cells and (FIG. 29B) resulted in minimal cytotoxicity compared to untreated cells at a 50 ng, 2× reagent concentration dose with a 24-hour incubation period. (FIG. 29C) Microscope images show constitutive nuclear GFP expression and low number of mCherry expressing transfected cells. (FIG. 29D) DNA-In resulted in at most 12% transfection efficacy with (FIG. 29E) manageable cytotoxicity at a 150-ng dose and 24-hour incubation time. (FIG. 29F) DNA-In visibly transfected a higher fraction of cells, but the majority remain untransfected. Bars show mean±SEM of four wells for each condition. Scale bar 200 μm for 10× images;

FIG. 30 shows the transfection efficacy and the relative cell count to untreated for the GL261 high throughput screening of base polymer endcaps. 20% triacrylate mole fraction BGDA-TMPTA-B4 polymer (7,8-4-Ac). 384 well plates, 75-ng DNA/well with 2-hr incubation. Transfection efficacy was assessed by cellomics;

FIG. 31 shows the transfection efficacy and the relative cell count to untreated for the B16-F10 high throughput screening of base polymer endcaps. 20% triacrylate mole fraction BGDA-TMPTA-B4 polymer (7,8-4-Ac). 384 well plates, 75-ng DNA/well with 2-hr incubation. Transfection efficacy was assessed by cellomics;

FIG. 32 shows the transfection efficacy, normalized geometric mean expression, and relative viability for GL261 mouse glioma cells, where 96-well transfection efficacy was assessed by flow cytometry, with 400 ng/well, and 2-hr incubation. 7,8-4-XX polymers are 20% branching monomer with the new, expanded endcap library. The new polymers yield up to 80% transfection, even at 20 w/w ratio (see 7,8-4-A11 polymer) compared to canonical PBAE 446, which required at least 40 w/w ratio and only gave 55% transfection. Geometric mean expression also increased with new polymers, while viability was maintained;

FIG. 33 shows the transfection efficacy, normalized geometric mean expression, and relative viability for B16-F10 mouse melanoma cells, where 96-well transfection efficacy was assessed by flow cytometry, with 600 ng/well, and 2-hr incubation. 7,8-4-XX polymers are 20% or 40% branching monomer with the new, expanded endcap library. The new polymers yield up to 95% transfection, even at 10 w/w ratio (see 7,8-4-A7 polymer) compared to canonical PBAE 446, which required at least 40 w/w ratio and only gave approximately 55% transfection. Geometric mean expression also increased with new polymers, while viability was maintained;

FIG. 34 shows images of B16-F10 cells transfected in 96-well plate at a 600 ng DNA dose, 2-hr incubation;

FIG. 35 shows images of GL261 cells transfected in 96-well plate at 400 ng DNA does, 2-hr incubation;

FIG. 36 shows normalized DNA binding (see also FIG. 8 for related data);

FIG. 37 shows the optimal w/w ratio relative to triacrylate mole fraction (top) and optimal amine density relative to triacrylate mole fraction (bottom) (see also FIG. 10 for related data);

FIG. 38 shows gene expression and nanoparticle property correlation for ARPE-19 cells;

FIG. 39A and FIG. 39B show combinatorial end-cap monomer library BEAQ synthesis. FIG. 39A shows high-throughput screening. FIG. 39B shows top hit confirmation;

FIG. 40A, FIG. 40B, FIG. 40C, FIG. 40D, and FIG. 40F show a schematic of combinatorial PBAE library construction (FIG. 40A) Linear base polymer PBAEs were synthesized in vials to be acrylate terminated, then characterized via 1H NMR and GPC (FIG. 40B) Synthesized polymers are dispensed into a 384 well round bottom plate using Viaflo 96/384 microplate dispenser and end-capped with each base polymer. A total of 4 different base polymers as shown in different color scheme are end capped per master plate containing 36 end-cap monomers each. (FIG. 40C) Source plates were then replicated from one master plate and stored them at −80° C. for future use. (FIG. 40D) End capped linear polymers (left 12 columns of the plate) were mixed with plasmid DNA (right 12 columns of the plate) to formulate NPs. (FIG. 40E) The RPE monolayers were transfected using automated Viaflo microplate dispenser and incubated for 48 hours with NPs. (FIG. 40F) Images were captured using Cellomics;

FIG. 41A, FIG. 41B, FIG. 41C, FIG. 41D, and FIG. 41E show sequential poly(beta-amino ester)s (PBAEs) library construction and synthesis scheme (FIG. 41A) Synthesis scheme of linear PBAEs from diacrylate and primary amine small monomers to yield acrylate terminated polymers followed by end-capping to yield linear end-capped PBAEs. (FIG. 41B) Example PBAE 5-3-A12 formed from monomers B5, S3 and end-cap A12. (FIG. 41C) Five diacrylate monomers and (FIG. 41D) three side-chain amino alcohols utilized in library synthesis. (FIG. 41E) 36 end-cap monomers identified as effective for transfection;

FIG. 42A and FIG. 42B show in vitro high throughput screening of PBAE nanoparticles in confluent D25 RPE monolayer. (FIG. 42A) Heat maps showing the percentage transfected RPE cells and (FIG. 42B) percentage survival rate following the introduction of a combinations of 140 different nanoparticles to confluent RPE monolayer at day 25 post seeding. The color scale bar refers to the percentage transfection efficiency and percentage survival that was calculated based on the number of mCherry positive cells detected from total number of cell population;

FIG. 43A, FIG. 43B, FIG. 43C, FIG. 43D and FIG. 43E show PBAE 5-3-A12 Characterization PBAE 5-3-A12 characterization. (FIG. 43A) Diameter measurements assessed via DLS z-average and (FIG. 43B) NTA showed that average diameter decreased as polymer: DNA w/w ratio increased. DLS z-average measurements were statistically lower for 90 w/w nanoparticles, compared to 30 w/w nanoparticles (FIG. 43C) Nanoparticle zeta-potential did not statistically differ between the nanoparticles at different w/w ratios. (FIG. 43D) End-capping with monomer A12 improved DNA binding compared to acrylate-terminated polymers. PBAE 5-3-A12 fully retarded DNA at w/w ratios down to 5 w/w, in contrast to the acrylate terminated polymer, which was only effective down to a 10 w/w ratio. (FIG. 43E) TEM showed 5-3-A12 nanoparticles as spherical. Graphs show mean of three independently prepared samples. *p<0.01, **p<0.001, based on one-way ANOVA with Tukey's post hoc test;

FIG. 44A, FIG. 44B, FIG. 44C, and FIG. 44D show in vitro transfection of confluent D25 RPE monolayer with top PBAE nanoparticles hits obtained from the preliminary high throughput screening. (FIG. 44A) Representative Z-stack confocal micrographs showing transfected RPE cells (red) and the epical localization of ZO-1 protein (green) of RPE monolayers transfected with a PBAE nanoparticle (5-3-A12) that yielded highest transfection efficacy in D25 RPE monolayer. The nuclei were counterstained with DAPI (blue). Histogram showing (FIG. 44B) transfection efficacy (FIG. 44C) relative viability and (FIG. 44D) mean fluorescent intensity of top 3 hits obtained from preliminary screen (5-3-A12, 5-3-F3 and 5-3-F4) along with commercial transfection reagents (lipofectamine 3000 and DNA-In). Transfection efficiency shown by percentage of mCherry positive cells, quantified using a specific algorithm designed for transfection assay in a High Content Analysis platform. ****p<0.001, based on student t-test. Confocal micrograph Scale bar: 50 μm;

FIG. 45A, FIG. 45B, FIG. 45C, and FIG. 45D show the transfection efficacy as measured in a co-transfection assay (FIG. 45A) Representative Cellomics images of RPE monolayers co-transfected with both mCherry (red) and GFP (green) constructs. Histogram showing (FIG. 45B) % cells (FIG. 45C) cell body area and (FIG. 45D) cell body size of cells that were introduced with either mCherry or GFP alone or cotransfected with both the construct;

FIG. 46A, FIG. 46B, and FIG. 46C show in vitro high throughput screening of PBAE nanoparticles in subconfluent D3 RPE monolayer. (FIG. 46A) Representative images showing mCherry transfected RPE cells. Heat maps showing the (FIG. 46B) percentage transfected RPE cells and (FIG. 46C) percentage survival rate following the introduction of a combinations of 140 different nanoparticles to confluent RPE monolayer at day 3 post seeding. The color scale bar refers to the percentage transfection efficiency and percentage survival that was calculated based on the number of mCherry positive cells detected from total number of cell population;

FIG. 47A, FIG. 47B, FIG. 47C, FIG. 47D, FIG. 47E, and FIG. 47F show the rBEAQs form nanoparticles with siRNA and enable gene knockdown. FIG. 47A shows knockdown and cell viability of rBEAQ-siRNA nanoparticles on HEK293 Ts. FIG. 47B shows cellular uptake. FIG. 47C shows nanoparticle hydrodynamic diameter as measured by NTA. FIG. 47D shows nanoparticle zeta potential as measured by DLS. FIG. 47E shows that when intracellular glutathione is blocked using the drug BSO, nanoparticle-mediated cytotoxicity increased. FIG. 47F shows TEM images of rBEAQ-siRNA nanoparticles;

FIG. 48A, FIG. 48B, and FIG. 48C show rBEAQ siRNA binding and release kinetics. FIG. 48A shows Yo-Pro-1 siRNA binding assay indicating that polymer branching increased siRNA binding strength. FIG. 48B shows that siRNA knockdown plotted against the EC50 of binding showed a biphasic response. FIG. 48C shows a gel retardation assay of rBEAQ nanoparticles incubated over time in 5 mM glutathione reducing environment.

FIG. 49A, FIG. 49B, FIG. 49C. FIG. 49D, and FIG. 49E show rBEAQs containing monomer B7 enabled efficient co-delivery of DNA and siRNA to HEK293T and Huh7 cells. Hydrophobic R6,7,8-4-6 polymer series enables efficient codelivery of DNA and siRNA. Codelivery efficacies of R6,8-4-6 (0% B7) and R6,7,8-4-6 nanoparticles encapsulating 400 ng of total nucleic acid in 293T (FIG. 49A) and Huh7 (FIG. 49B). N=4. (FIG. 49C) Fluorescence microscopy images of HEK-293T cells treated with R6,7,8_16 nanoparticles codelivering 200 ng of siRNA and 200 ng of DNA (10 w/w formulation). Scale bar 100 μm. (FIG. 49D) R6,7,8_64 completely encapsulated plasmid DNA and siRNA at 10 w/w as seen by a gel retardation assay. (FIG. 49E) Confocal microscopy images of 293T cells treated with R6,7,8_64 nanoparticles codelivering Cy3-siRNA, Cy5-DNA, and unlabeled GFP plasmid DNA (0.5:0.4:0.1 composition by weight) at 3 and 24 h post-uptake. Cy3 and Cy5 signal colocalization could be seen at 3 h post-uptake (white arrows). At 24 h post-uptake, a diffuse Cy3-siRNA signal could be seen in the cytosol (white asterisk), whereas some Cy5-DNA signal was detected in the nucleus (yellow arrows) and some cells were visibly expressing GFP. Scale bar 20 μm;

FIG. 50A. FIG. 50B, and FIG. 50C show codelivery of anti-GFP sgRNA and Cas9 plasmid enables CRISPR-mediated gene knockout. (FIG. 50A) HEK-293T cells were transfected with R6,7,8_64 10 w/w nanoparticles encapsulating Cas9 DNA and sgRNA at the indicated nucleic acid molar ratios. N=4. (FIG. 50B) Flow cytometry histograms of CRISPR- or siRNA-treated cells. CRISPR treatment produced a completely GFP-negative population (null), whereas siRNA treatment mainly resulted in a general population shift to lower GFP fluorescence (low). (FIG. 50C) Gene suppression kinetics of CRISPR and siRNA-treated cells. N=4;

FIG. 51A, FIG. 51B, FIG. 51C, and FIG. 51D show representative monomers for synthesizing polymers to include alkyl or fluorinated side-chain monomers containg primary amines to improve colloidal stability;

FIG. 52A, FIG. 52B, FIG. 52C, and FIG. 52D show lysosome colocalization assessment with confocal microscopy. (FIG. 52A) Cells transfected with B8-0% and B8-50% at low (20 w/w) and high (40 w/w) nanoparticles and assessed by confocal microscopy show statistically significant differences in the degree of lysosome colocalization. Assessed by one-way ANOVA with Dunnett corrected multiple comparisons to the B8-50:40% w/w conditions. (FIG. 52B) Representative 2D scattergrams of HEK293T cells at 24 h post-treatment using 20 w/w nanoparticles. Region 3 represents colocalized pixel intensities. (FIG. 52C) All conditions in both cell lines showed statistically significant (Holm-Sidak corrected multiple t tests) increases in the degree of lysosome colocalization between 4 and 24 h following transfection (bars show mean±SEM of n>100 cells). (FIG. 52D) Representative maximum intensity projection images of cells transfected with 20 w/w nanoparticles 24 h following transfection, showing lysosome colocalization in white;

FIG. 53A, FIG. 53B, and FIG. 53C show nuclear localization of plasmid DNA and expression of eGFP assessed by confocal microscopy. HEK293T cells were transfected 24 h prior with B8-50:20% w/w nanoparticles containing 80% noncoding, Cy5-labeled plasmid DNA and 20% coding eGFPN1 plasmid DNA. (FIG. 53A) Maximum intensity projection demonstrating high level of labeled plasmid DNA remaining in the cells with minimal lysosome colocalization. (FIG. 53B) Strong eGFP expression from the 20% of unlabeled plasmid DNA. (FIG. 53C) A single z-slice shows Cy5-labeled plasmid DNA localized to the nucleus in select cells (white arrows);

FIG. 54A, FIG. 54B, FIG. 54C, FIG. 54D, FIG. 54E, FIG. 54D, FIG. 54E, FIG. 54F, FIG. 54G, FIG. 54H, FIG. 54I, and FIG. 54 J show correlation between BEAQ properties and viability normalized geometric mean expression. Geometric mean expression plots were normalized to the maximum expression for each polymer and scaled by viability at that w/w ratio for (A-E) HEK293T cells and (F-J) ARPE-19 cells. Dashed-gray curves show a single quadratic fit of all data points for that cell line with calculated R2. Plots showing dotted-gray curves in addition to dashed-gray curves were statistically determined to require two fitted quadratic curves to adequately describe the data;

FIG. 55 shows Gel retention assay of DNA binding capacity. Gel retention assays of nanoparticles formed in acidic, low salt NaAc, pH 5 and isotonic, pH 7.4 PBS showed greater binding associated with more highly branched nanoparticles with B8-90% nanoparticles showing the highest degree of binding compared to B8-0%, B8-20% or B8-50%;

FIG. 56A, FIG. 56B, FIG. 56C, and FIG. 56D show representative BEAQ series polymers tested under matched conditions in 10% and 50% serum conditions transfected effectively the same percentages of cells (A,C) in both HEK293T and ARPE-19 cells. B) For level of expression, however, the linear polymer (B8-0%) suffered a 75% reduction of polymer matched max geometric mean expression with increase in serum content, whereas the B8-60% triacrylate mole fraction polymer only suffered a 21% reduction in geometric mean expression in HEK293T cells. D) Similarly, in ARPE-19 cells, the linear polymer (B8-0%) suffered a 68% reduction in geometric mean expression, whereas the B8-20% triacrylate mole fraction branched polymer geometric mean expression was only reduced by 30%. (Error bars show n=4 wells, mean±SEM);

FIG. 57A, FIG. 57B, and FIG. 57C show low dose BEAQ nanoparticle transfection in HEK-293T cells. FIG. 57A) Extremely low volume distribution of nanoparticles achieved via Echo 550 acoustic liquid handling with nanoparticle dose titration. FIG. 57B) Transfection efficacy and FIG. 57C) cell counts normalized to untreated for varied w/w ratio and overall nanoparticle dose (as function of total DNA per well). BEAQs with 40-60% triacrylate mole-fraction were statistically more effective than the linear B8-0% polymer tested for low dose nanoparticle transfection. No nanoparticle formulations showed high cytotoxicity (>30% reduction in normalized cell count). Values show mean±SEM of three wells for each condition. Differences in transfection efficacy between polymers were assessed over all tested conditions by One-way ANOVA with multiple comparisons to B8-O % using matched values for w/w ratio and DNA dose, One-way ANOVA was performed with Geisser-Greenhouse corrections for sphericity and Dunnet corrections for multiple comparisons. P values shown are multiplicity adjusted. (Error bars show n=4 wells, mean±SEM);

FIG. 58A and FIG. 58B show confocal microscopy Z-stack analysis of nanoparticle location and colocalization. We hypothesized that the location of assessing lysosome colocalization within the cell may influence the measured degree of colocalization as endosomes closer to the glass surface may be more mature and lower in pH. For this purpose, Z-stacks were acquired with confocal microscopy and individual contribution of the colocalization coefficient was scaled by the area of Cy5-DNA detectable for that slice at 4 hours) and 24 hours) post-transfection;

FIG. 59 shows confocal microscopy maximum intensity projections of HEK293T cells 4 hours following nanoparticle uptake. Nanoparticles were 80% covalently labeled with Cy5 and 20% coding for eGFP. All conditions showed high internalization of nanoparticles with minimal lysosomal colocalization. Lysosomal indicator pKa 4.6. Scale bar 50 μm;

FIG. 60 shows confocal microscopy maximum intensity projections of ARPE-19 cells 4 hours following nanoparticle uptake. Nanoparticles were 80% covalently labeled with Cy5 and 20% unlabeled coding for eGFP. All conditions showed high internalization of nanoparticles with minimal lysosomal colocalization. Lysosomal indicator pKa 4.6. Scale bar 50 μm;

FIG. 61 shows confocal microscopy of HEK293T 24 h following nanoparticle uptake. Nanoparticles were 80% covalently labeled with Cy5 and 20% unlabeled coding for eGFP still yielded robust expression of eGFP detectable at 24 hours. All conditions showed high internalization of nanoparticles, which much greater lysosomal accumulation for linear polymers than the B8-50%; 40 w/w nanoparticles in particular. Lysosomal indicator pKa 4.6. Scale bar 50 μm;

FIG. 62 shows confocal microscopy maximum intensity projections of ARPE-19 cells 24 hours following nanoparticle uptake. Nanoparticles were 80% covalently labeled with Cy5 and 20% unlabeled coding for eGFP still yielded robust expression of eGFP detectable at 24 hours. All conditions showed high internalization of nanoparticles, which much greater lysosomal accumulation for linear polymers than the B8-50%; 40 w/w nanoparticles in particular. Lysosomal indicator pKa 4.6. Scale bar 50 μm;

FIG. 63A and FIG. 63B show polymer structural information. (FIG. 63A) H1-NMR spectra of acrylate-terminated and end-capped R6,8_20 polymer (CDCl3, 500 MHz). Red box indicates the presence of acrylate peaks, which disappeared after end-capping. (FIG. 63B) Chemical structure of end-capped R6,8_20;

FIG. 64A and FIG. 64B show knockdown (FIG. 64A) and cytotoxicity (FIG. 64B) of R6,8-4-6 nanoparticles at lower w/w formulations. Nanoparticles encapsulated 100 nM siRNA dosage. Knockdown of GFP fluorescence was normalized against cells treated with non-targeting scrambled RNA (scRNA); n=4;

FIG. 65A and FIG. 65B show Yo-Pro binding assay for acrylate-terminated polymers. (FIG. 65A) Increasing polymer branching increased binding affinity for acrylate-terminated polymers. (FIG. 65B) Endcapped polymers (E6) showed higher binding affinity than acrylate-terminated polymers (Ac). N=4;

FIG. 66A, FIG. 66B, FIG. 66C, and FIG. 66D show nanoparticle characterization. Hydrodynamic diameter (FIG. 66A) and zeta potential (FIG. 66B) confirm that B7-containing polymers formed smaller, more positively charged nanoparticles at low w/w formulations. Size and zeta potential measurements done via DLS using nanoparticles diluted in PBS. N=3. (FIG. 66C) TEM image of R6,7,8_64 nanoparticles containing DNA and siRNA. (FIG. 66D) R6,7,8_64 nanoparticles (10 w/w) only moderately aggregated over the time-span of four hours in 10% serum-containing medium. N=2;

FIG. 67 shows confocal microscopy of co-delivered DNA and siRNA. HEK-293T cells were transfected with polymer R6,7,8_64 nanoparticles formed at a 10 w/w ratio between polymer and nucleic acids. Cy3-siRNA, Cy5-DNA and eGFP-DNA were pre-mixed before nanoparticle encapsulation at a mass ratio of 50:40:10. At 3 hours after nanoparticle exposure, many endosomes visibly contain both Cy3 and Cy5 signal for siRNA and DNA respectively. At 24 hours post-treatment, diffuse Cy3-siRNA fluorescence is detectable while Cy5-DNA fluorescence is punctate and GFP is visibly being expressed by some cells. Scale bar 50 μm;

FIG. 68 shows DNA and siRNA co-delivery with leading commercially-available transfection reagents. R6,7,8_64 nanoparticles (10 w/w) and non-viral transfection reagents Lipofectamine 2,000™, Lipofectamine 3,000™, jetPrime®, and 25 kD bPEI (1 w/w) were used to co-deliver DNA and siRNA to HEK-293T and Huh7 cells. R6,7,8_64 nanoparticles generally performed better or as well as leading commercially-available reagents at co-delivery. N=4. Statistical analysis was assessed by one-way ANOVA with Tukey post-hoc tests;

FIG. 69 shows nucleic acid co-encapsulation outperforms DNA and siRNA delivery with their respective previously-optimized nanoparticle formulation. R6,7,8_64 nanoparticles were formulated with 200 ng each of pre-mixed plasmid DNA and siRNA at 10 w/w before nanoparticles were added to cells (Single NP). Polymer 446 (optimal for DNA delivery) and polymer R646 (optimal for siRNA delivery) were formulated separately with their respective cargos and each nanoparticle formulation was added separately to cells, with 200 ng of DNA and siRNA, respectively, delivered (dual NP). The single NP strategy outperformed the dual NP strategy when NPs were formulated at high w/w (60 w/w for 446 and 120 w/w for R646) as well as at low w/w (10 w/w for 446 and R646, respectively). R6,7,8_64 polymers were always used at 10 w/w, demonstrating its higher delivery efficiency. Huh 7 cells were used in this experiment. N=4. Statistical analysis was assessed by one-way ANOVA with Tukey post-hoc tests;

FIG. 70A and FIG. 70B show R6,7,8_64 nanoparticle delivery efficacy in serum-containing medium. R6,7,8_64 nanoparticles (10 w/w) containing (A) siRNA or (B) Cas9 DNA and sgRNA were administered to cells in cell culture medium with or without 10% FBS. The presence of serum significantly decreased transfection in both cases. The addition of NaHCO₃ solution to increase the pH of nanoparticles prior to adding to cells led to recovery in transfection efficacy. N=4 for all experiments. Statistical analysis was assessed by one-way ANOVA with Tukey post-hoc tests;

FIG. 71A, FIG. 71B, FIG. 71C, and FIG. 71D show PBAEs form nanoparticles with plasmid DNA and enable transfection in HEK293T and B16-F10 cells. (FIG. 71A) Polymer structures for 446 and 7,8-4-J11, which were used to transfect HEK-293T and B16-F10 cells, respectively. (FIG. 71B) Nanoparticle hydrodynamic diameter and zeta potentials as measured by dynamic light scattering. 446 nanoparticles were formulated at 60 w/w while 7,8-4-J11 nanoparticles were formulated at 30 w/w. (FIG. 71C) Transfection efficacy as measured by nanoparticles delivering GFP; 600 ng/well dose was used. Bars show mean+SEM; N=4. (FIG. 71D) TEM images of 446 and 7,8-4-J11 nanoparticles;

FIG. 72A, FIG. 72B, FIG. 72C, and FIG. 72D show expression kinetics of CRISPR components after co-delivery of Cas9 and sg1 plasmids. Cas9 mRNA (FIG. 72A, red curve) and protein expression (FIG. 72A, blue curve; FIG. 72B) were measured over time in HEK-293T cells. (FIG. 72C) sgRNA and (FIG. 72D) ReNL mRNA expression kinetics. N=2 (data shown as mean+/−SEM) for qRT-PCR experiments; N=1 for western blots;

FIG. 73A, FIG. 73B, FIG. 73C, and FIG. 73D show DNA dosage titration reveals different threshold expression requirements for 1-cut and 2-cut edits. (FIG. 73A) DNA dosage decrease from 600 ng to 300 ng did not change the overall percentage of GFP-positive cells but significantly decreased the geometric mean of expression. Dosage decrease significantly decreased the efficacy of 2-cut gene deletion edits (FIG. 73B) but not 1-cut iRFP knockout edits (FIG. 73C). Statistical significance determined by Holm-Sidak corrected multiple t tests; **p<0.01, ***p<0.001. Data shown as mean+SEM; N=4. (FIG. 73D) Flow cytometry histograms of cells treated with different DNA doses;

FIG. 74A, FIG. 74B, FIG. 74C, and FIG. 74D show 1-cut and 2-cut edits in easy-to-transfect HEK-293T cells and hard-to-transfect B16-F10 cells. (FIG. 74A) 1-cut edit efficiency correlated logarithmically with level of transfection as indicated by geometric mean fluorescence of a GFP reporter gene while 2-cut edit efficiency correlated linearly in 293T cells. (FIG. 74B) In B16 cells, transient cold shock after transfection significantly increased transfection efficacy as well as 2-cut editing efficiency but no significant change was seen in 1-cut editing efficiency as assessed by Holm-Sidak corrected multiple t tests; **p<0.01, ***p<0.001. (FIG. 74C) B16 cells achieved minimal levels of 2-cut edits; 1-cut edits were lower compared to 293T cells, but the difference is smaller. Data in (FIG. 74B) and (FIG. 74C) shown as mean+SEM; N=4. Differences in editing are observed in flow cytometry histograms (FIG. 74D-FIG. 74E);

FIG. 75A and FIG. 75B show a tRNA-gRNA expression system for multiplex editing.

(FIG. 75A) Schematic of a multiplex sgRNA expression system in which multiple tRNA-gRNA units are arrayed in tandem. The primary RNA transcript is processed by the endogenous tRNA machinery, releasing mature sgRNAs. (FIG. 75B) The tRNA-gRNA plasmid coding for sg2 and sg3 results in similar levels of 2-cut editing compared to a plasmid in which each sgRNA is governed by an individual U6 promoter (sg2+sg3). Statistical analysis was assessed by one-way ANOVA with Tukey post-hoc tests. Data presented as mean+/−SEM; N=4;

FIG. 76A and FIG. 76B show GFP transfection screen results for B16-F10 cells. Fluorescence microscopy images (FIG. 76A) and flow cytometry results (FIG. 76B) show that branched PBAE polymer 7,8-4-J11 (30 w/w) transfects B16 cells more efficiently than canonical linear PBAE polymer 446 (40 w/w). Data presented as mean+/−SEM; N=4;

FIG. 77 shows microscopy images of ReNL gain of expression. 2-cut CRISPR cleavage with sg1 or combination of sg2+sg3 turn on expression of ReNL by removal of two SV40 polyA sequences;

FIG. 78A, FIG. 78B, FIG. 78C, and FIG. 78D show expression kinetics of CRISPR components in B16 cells. mRNA expression levels of sgRNA (FIG. 78A) and ReNL (FIG. 78B). Cas9 mRNA (C, red curve) and protein (FIG. 78C, blue curve; FIG. 78D) expression levels over time. N=2 (data shown as mean+/−SEM) for qRT-PCR experiments; N=1 for western blots;

FIG. 79A and FIG. 79B show a comparison to commercial transfection reagents. Gain of expression from 2-cut edits and viability of cells using commercial reagents and PBAEs in (FIG. 79A) 293T and (FIG. 79B) B16 cells as measured by ReNL luminescence. Data presented as mean+SEM; N=4. Statistical significance assessed by one-way ANOVA with Dunnett post-hoc tests as compared to the PBAE treated group (446 or J11, respectively); *p<0.05, ****p<0.0001;

FIG. 80A, FIG. 80B, FIG. 80C, and FIG. 80D show differential transfection level sensitivity of 1-cut vs. 2-cut edits. Flow cytometry results of eGFP expression (FIG. 80A), ReNL gain-of-function expression (FIG. 80B), and iRFP knockout (FIG. 80C) correlated semi-logarithmically with DNA dosage delivered. mRNA expression of Cas9 and sgRNA, respectively, correlated logarithmically with DNA dose (FIG. 80D). Data shown as mean+/−SEM; N=4; and

FIG. 81A and FIG. 81B show polymer-mediated cytotoxicity for R6,7,8-4-6 nanoparticles co-delivering DNA and siRNA in HEK-293T and Huh7 cells. (FIG. 81A) Cytotoxicity mediated by optimal formulations of R6,7,8-4-6 nanoparticles as well as R6,8-4-6 nanoparticles co-delivering 400 ng total nucleic acid. (FIG. 81B) R6,7,8-4-6 nanoparticles mediated high levels of toxicity at higher w/w formulations. N=4.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Poly(β-Amino Ester) Nanoparticles for the Non-Viral Delivery of Plasmid DNA for Gene Editing and Retinal Gene Therapy

In some embodiments, the presently disclosed subject matter provides biodegradable particles for delivering nucleic acids to cells, including nucleic acids encoding gene-editing factors or a therapeutic protein. In particular embodiments, the particles comprise poly(beta-amino ester) (PBAE) polymers that self-assemble with nucleic acid, including DNA or RNA. The PBAE particles are biodegradable, e.g., they degrade in water or an aqueous solution. In certain embodiments the degradation is pH-dependent.

In some embodiments, the particles comprise linear or branched PBAE polymers having a backbone constructed from diacrylate monomers, and, optionally in combination with triacrylate monomers, to provide polymers with variable branching. The polymers can be prepared by condensing side chain monomers comprising secondary amines or primary amines with acrylate ester monomers, e.g, diacrylate and triacrylate monomers. For example, in some embodiments, the PBAE comprises a backbone of a diacrylate, e.g., bisphenol A glycerolate (1 glycerol/phenol) diacrylate (BGDA), and a triacrylate, e.g., trimethylolpropane triacrylate (TMPTA).

In some embodiments, the polymers comprise tertiary amines in their backbone and/or in some embodiments, the polymers comprise side chains and/or end groups comprising primary, secondary, and/or tertiary amines to complex with a nucleic acid. In some embodiments, the secondary or tertiary amines comprise bivalent amine-containing heterocyclic groups. In some embodiments, the side chain monomers comprise a primary amine, but may also comprise secondary and tertiary amines. In some embodiments, the end group terminates with a primary amine and a hydroxyl, with an internally placed secondary amine.

The particles may be complexed with plasmid DNA encoding the gene-editing endonuclease, and in some embodiments, a gRNA, a plasmid DNA encoding a gRNA, and/or replacement DNA template. In some embodiments, the particles are complexed with a polypeptide (e.g., a gene-editing endonuclease). In such embodiments, the presently disclosed subject matter provides biodegradable nanoparticles to direct efficient site-target disruption, mutation, deletion, or repair of a nucleic acid (e.g., a DNA and/or an RNA). Thus, the presently disclosed subject matter provides an efficient gene therapy platform, involving either ex vivo or in vivo gene and/or transcript editing.

### A. Compositions Comprising Poly(beta-amino esters) (PBAEs) of Formula (I) and Formula (II)

In some embodiments, the presently disclosed subject matter provides compositions, including particles, comprising multicomponent degradable cationic polymers for gene delivery to cells. The presently disclosed polymers have the property of biphasic degradation and modifications to the polymer structure can result in a change in the release of therapeutic agents, e.g., a DNA plasmid. In some embodiments, the presently disclosed polymers include a minority structure, e.g., an endcapping group, which differs from the majority structure comprising most of the polymer backbone. In other embodiments, the bioreducible oligomers form block copolymers with hydrolytically degradable oligomers. In yet other embodiments, the end group/minority structure comprises an amino acid or chain of amino acids, while the backbone degrades hydrolytically and/or is bioreducible.

Small changes in the monomer ratio used during polymerization, in combination with modifications to the chemical structure of the end-capping groups used post-polymerization, can affect the efficacy of delivery of a gene to a cell. Further, changes in the chemical structure of the polymer, either in the backbone of the polymer or end-capping groups, or both, can change the efficacy of gene delivery to a cell. In some embodiments, small changes to the molecular weight of the polymer or changes to the endcapping groups of the polymer, while leaving the main chain, i.e., backbone, of the polymer the same, can enhance or decrease the overall delivery of the gene to a cell. Further, the “R” groups that comprise the backbone or main chain of the polymer can be selected to degrade via different biodegradation mechanisms within the same polymer molecule. Such mechanisms include, but are not limited to, hydrolytic, bioreducible, enzymatic, and/or other modes of degradation.

The properties of the presently disclosed multicomponent degradable cationic polymers can be tuned to impart one or more of the following characteristics to the composition: independent control of cell-specific uptake and/or intracellular delivery of a particle; independent control of endosomal buffering and endosomal escape; independent control of DNA release; triggered release of an active agent; modification of a particle surface charge; increased diffusion through a cytoplasm of a cell; increased active transport through a cytoplasm of a cell; increased nuclear import within a cell; increased transcription of an associated DNA within a cell; increased translation of an associated DNA within a cell; and/or increased persistence of an associated therapeutic agent within a cell.

If a hydrophilic peptide/protein is to be encapsulated, a hydrophilic polymer is chosen as the multicomponent material. If a hydrophobic peptide/protein is to be encapsulated than a hydrophobic polymer is chosen. The polymer backbone, side chain, and/or terminal group can be modified to increase the hydrophobic or hydrophilic character of the polymer. The peptide/protein to be encapsulated can be first dissolved in a suitable solvent, such as DMSO or PBS. Then, it is combined with the polymer in, for example, sodium acetate (NaAc). This solution is then diluted with either sodium acetate, OptiMem, DMEM, PBS, or water depending on the particle size desired. The solution in vortexed to mix and then left to incubate for a period of time for particle assembly to take place. The particles can self-assemble with nucleic acid, including plasmid DNA, to form nanoparticles that can be in the range of 50 nm to 500 nm in size. The particles provide for efficient transfection of cells with plasmid DNA, either in vivo or ex vivo.

Representative multicomponent degradable cationic polymers are disclosed in the following U.S. patents and U.S. patent application publications, each of which is incorporated herein by reference in its entirety: U.S. Patent Application Publication No. 20180177881 for Multicomponent Degradable Cationic Polymers, to Green et al., published Jun. 28, 2018; U.S. Patent Application Publication No. 20150250881 for Multicomponent Degradable Cationic Polymers, to Green et al., published Sep. 10, 2015; U.S. Patent Application Publication No. 20120128782 for Multicomponent Degradable Cationic Polymers, to Green et al., published May 24, 2012; U.S. Patent Application Publication No. 20180112038 for Poly(beta-amino ester)-co-polyethylene glycol (PEG-PBAE-PEG) Polymers for Gene and Drug Delivery, to Green et al., published Apr. 26, 2018; U.S. Patent Application Publication No. 20180028455 for Peptide/Particle Delivery Systems, to Green et al., published Feb. 1, 2018; U.S. Patent Application Publication No. 20160374949 for Peptide/Particle Delivery Systems, to Green et al., published Dec. 29, 2016; U.S. Patent Application Publication No. 20120114759 for Peptide/Particle Delivery Systems, to Green et al., published Dec. 29, 2016; U.S. Patent Application Publication No. 20160122390 for A Biomimetic Peptide and Biodegradable Delivery Platform for the Treatment of Angiogenesis- and Lymphangiogenesis-Dependent Diseases, to Popel, et al, published May 5, 2016; U.S. Patent Application Publication No. 20150273071 for Bioreducible Poly (Beta-Amino Ester)s for siRNA Delivery, to Green et al., published Oct. 1, 2015; U.S. Pat. No. 9,884,118 for Multicomponent Degradable Cationic Polymers, to Green, et al., issued Feb. 6, 2018; U.S. Pat. No. 9,717,694 for Peptide/particle Delivery Systems, Green, et al., issued Aug. 1, 2017; and U.S. Pat. No. 8,992,991 for Multicomponent Degradable Cationic Polymers, to Green, et al., issued Mar. 31, 2015; U.S. Pat. No. 8,287,849 for Biodegradable Poly(beta-amino esters) and Uses Thereof, to Langer, et al., issued Oct. 16, 2012. Other exemplary PBAE polymers are described in WO/2012/0128782, WO/2012/0114759, WO/2014/066811, WO/2014/066898, and US2016/0122390, each of which is incorporated herein by reference in its entirety. In embodiments, a particle comprises a polymer blend of PBAE, e.g., a mixture of PBAE polymers.

The presently disclosed multicomponent degradable cationic polymers can be prepared by the following reaction scheme:

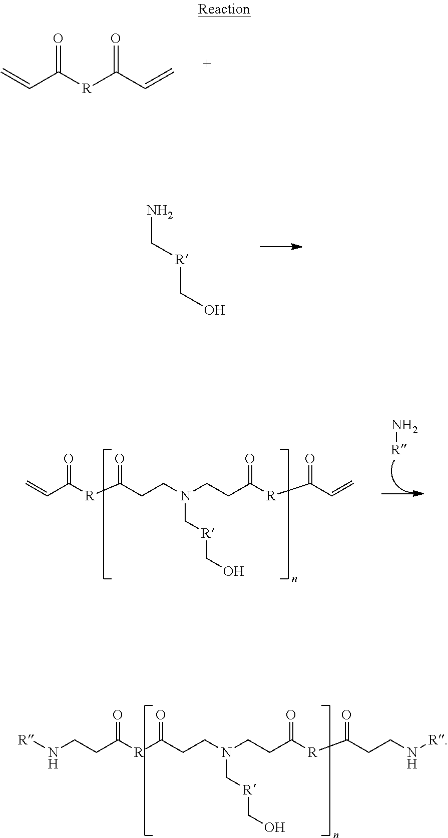

Generally, the presently disclosed multicomponent degradable cationic polymers include a backbone derived from a diacrylate monomer (designated herein below as “B”), an amino-alcohol side chain monomer (designated herein below as “S”), and an amine-containing end-cap monomer (designated herein below as “E”). The end group structures are distinct and separate from the polymer backbone structures and the side chain structures of the intermediate precursor molecule for a given polymeric material. The presently disclosed PBAE compositions can be designated, for example, as B5-S4-E7 or 547, in which R is B5, R″ is S4, and R′″ is E7, and the like, where B is for backbone and S is for the side chain, followed by the number of carbons in their hydrocarbon chain. Endcapping monomers, E, are sequentially numbered according to similarities in their amine structures.

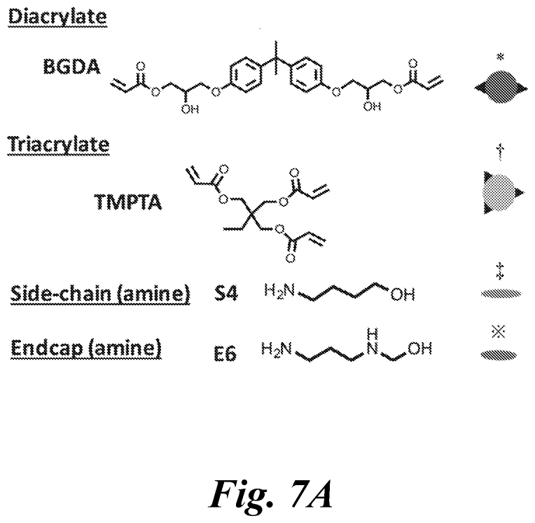

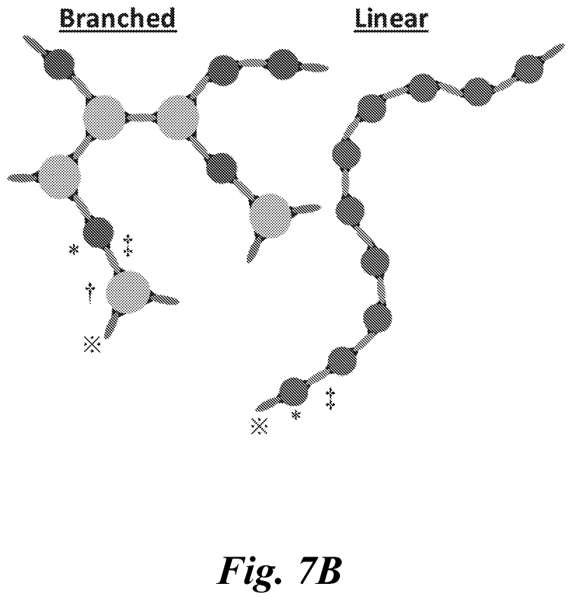

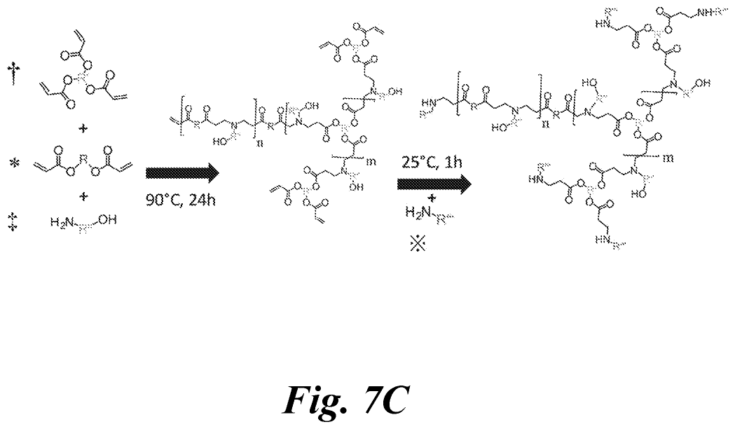

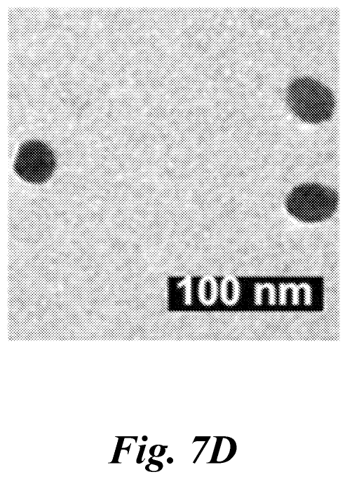

In other embodiments, the presently disclosed polymers have a backbone constructed from a diacrylate, and optionally with a triacrylate monomer, to provide polymers with variable branching. See, for example FIG. 7C.

More particularly, in some embodiments, the presently disclosed subject matter provides a composition comprising a poly(beta-amino ester) (PBAE) of formula (I) or formula (II):

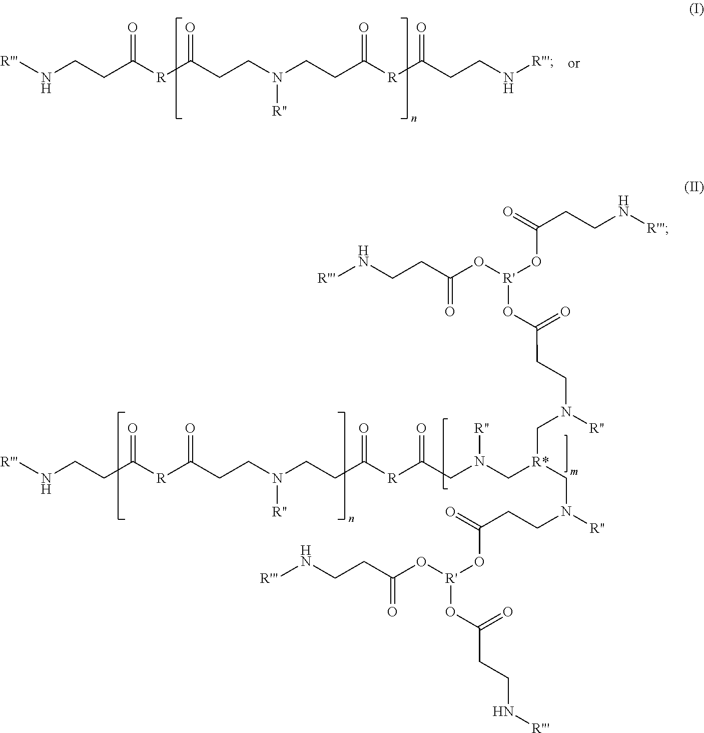

and at least one DNA or RNA molecule comprising a nucleic acid sequence encoding a gene-editing protein or therapeutic protein;

wherein:

n and m are each independently an integer from 1 to 10,000;

each R is independently a diacrylate monomer of the following structure:

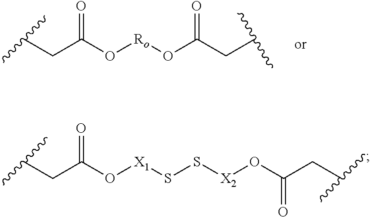

wherein Rₒ comprises a linear or branched C₁-C₃₀ alkylene chain, which may further comprise one or more heteroatoms or one or more carbocyclic, heterocyclic, or aromatic groups and X₁ and X₂ are each independently a linear or branched C₁-C₃₀ alkylene chain;

each R* is a triacrylate, quanternary, or hexafunctional acrylate monomer selected from the group consisting of:

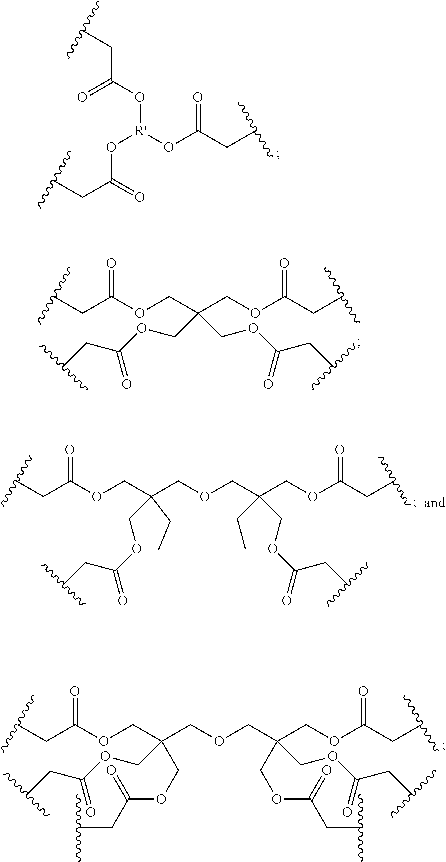

wherein each R′ is independently a trivalent group; each R″ is independently a side chain monomer comprising a primary, secondary, or tertiary amine; and each R′″ is independently an end group monomer comprising a primary, secondary, or tertiary amine.

In some embodiments, the gene-editing protein is selected from the group consisting of CRISPR-associated nuclease, Cre recombinase, Flp recombinase, a meganuclease, a Transcription Activator-Like Effector Nuclease (TALEN), a Zinc-Finger Nuclease (ZFN), or a natural or engineered variant, family-member, orthologue, fragment or fusion construct thereof.

In particular embodiments, the gene-editing protein is a Cas9 endonuclease.

In some embodiments, the composition further comprises a gRNA or DNA encoding a gRNA. In certain embodiments, the Cas9 endonuclease and the gRNA are encoded on the same plasmid. In other embodiments, the Cas9 endonuclease and the gRNA are encoded on different plasmids.

As provided in more detail herein below, in some embodiments, the therapeutic protein is selected from the group consisting of CNGA3, CNGB3, GNAT2, sFLT01, Rab Escort Protein (REP-1), RS-1, RPE65, RPGR, MY07A, MERTK, ATP-binding cassette transporter 4 (ABCA4), and SAR-421869.

In some embodiments, the composition further comprises a promoter. In such embodiments, the nucleic acid is operably linked to a promoter.

In some embodiments, R is selected from the group consisting of:

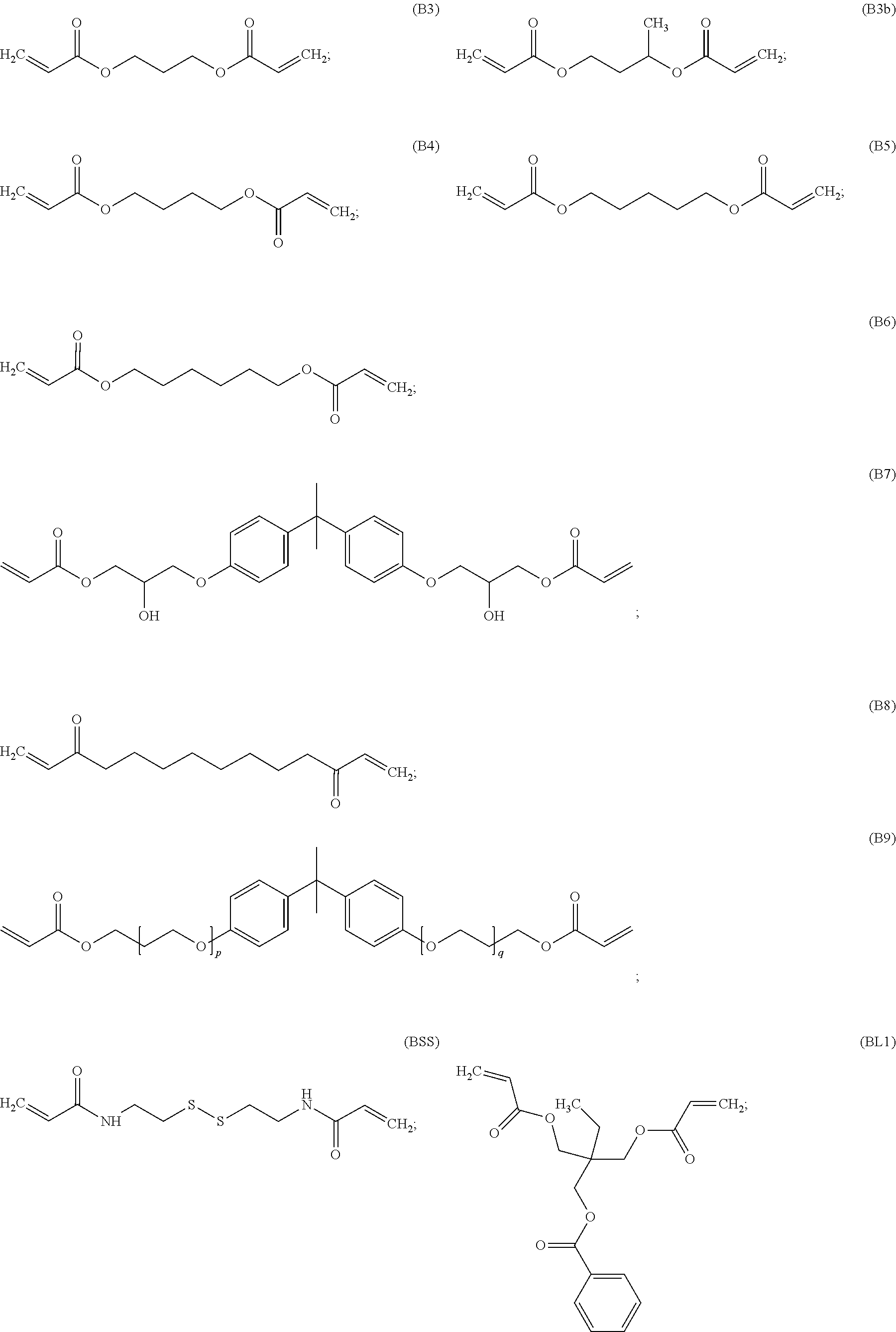

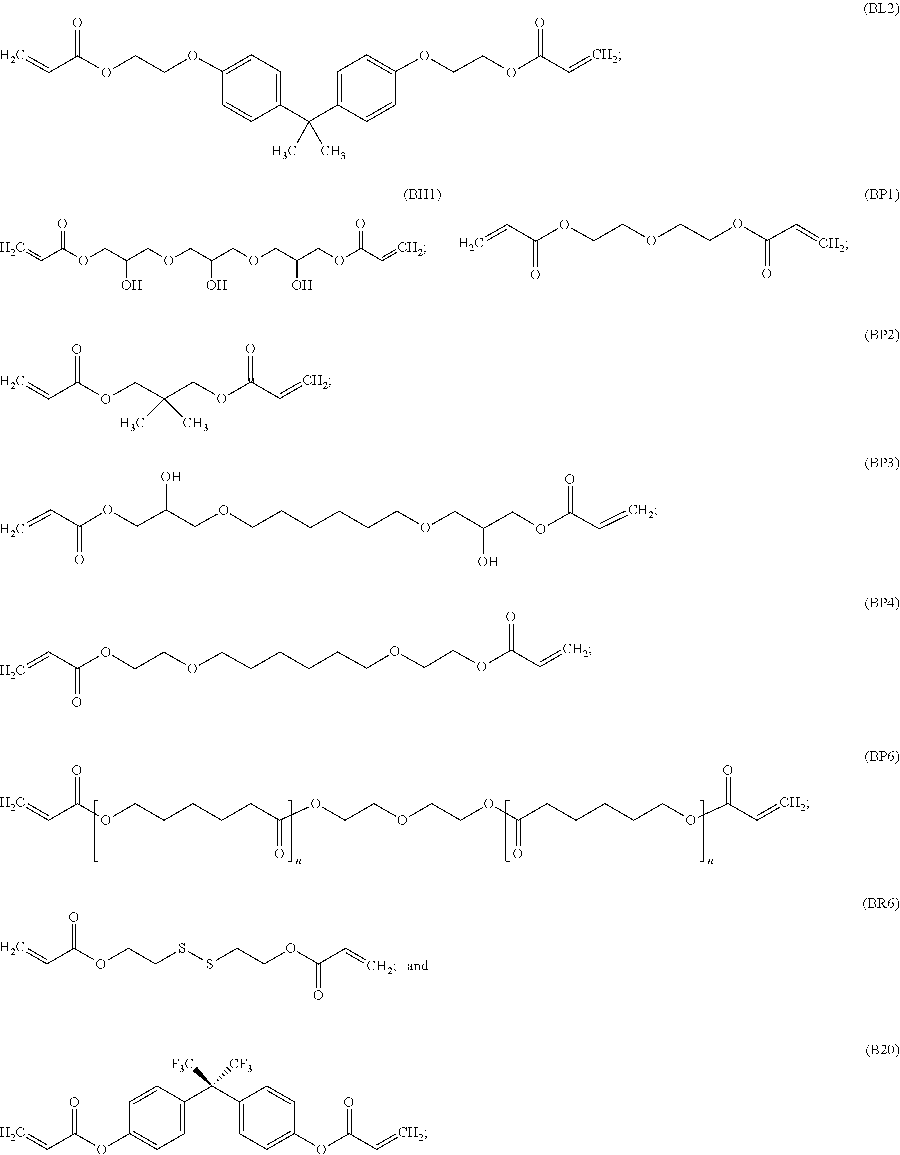

wherein p, q, and u are each independently an integer from 1 to 10,000.

In particular embodiments, R is selected from the group consisting of:

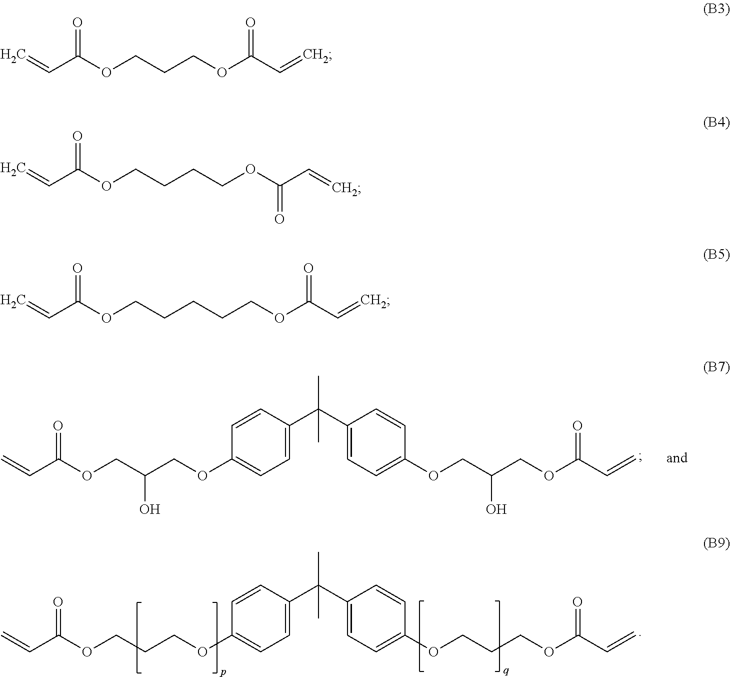

In particular embodiments, the diacrylate is bisphenol A glycerolate diacrylate (BGDA) (B7).

As shown in the reaction scheme provided hereinabove, diacrylate monomers can be condensed with amine-containing side chain monomers. In some embodiments, the side chain monomers comprise a primary amine, but, in other embodiments, comprise secondary and tertiary amines Side chain monomers may further comprise a C₁ to C20 linear or branched alkylene, including C₁-C₂₀ straightchain or branched alkylene, including C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀ alkylene, which is optionally substituted. Illustrative substituents include hydroxyl, alkyl, alkenyl, thiol, amine, carbonyl, halogen, and fluorinated alkylene, including, but not limited to, 2,2,3,3,4,4,5,5,6,6,7,7,8,8,9,9,9-Heptadecafluorononylamine.

In some embodiments, the side chain monomer, R″, is selected from the group consisting of:

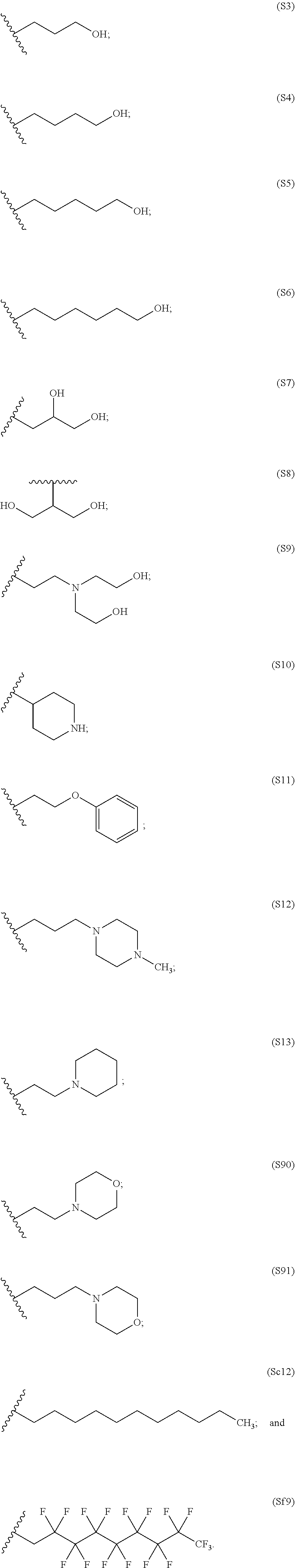

In particular embodiments, the side chain monomer, R″, is selected from the group consisting of:

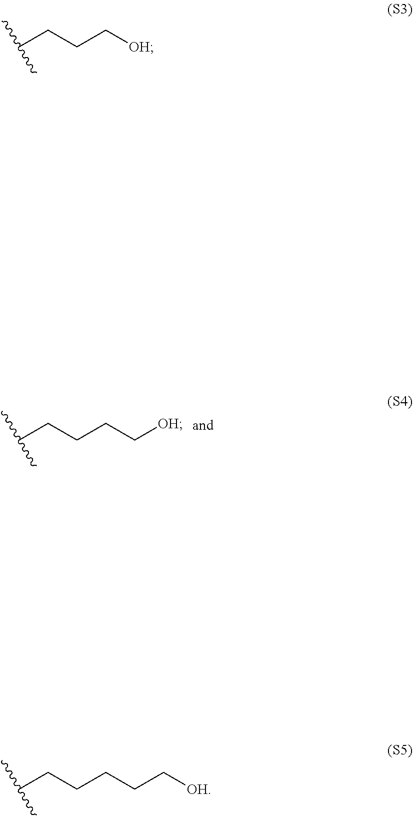 [SMILES: *CCCO ; *CCCCO ; *CCCCCO]

The PBAE polymer further comprises an end group, which may include one or more primary, secondary or tertiary amines, and may include aromatic and non-aromatic carbocyclic and heterocyclic groups, such as carbocyclic and heterocyclic groups of 5 or 6 atoms. The end group in some embodiments may comprise one or more ether, thioether, or disulfide linkages.

Representative end groups include, but are not limited to:

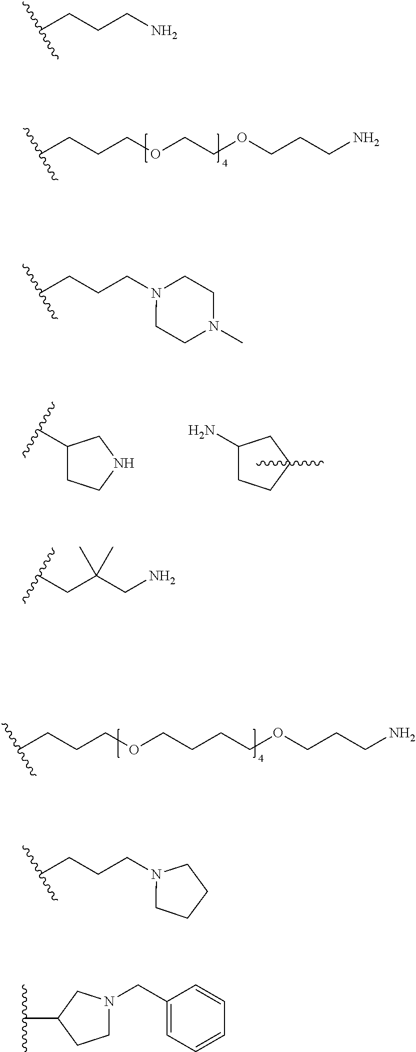

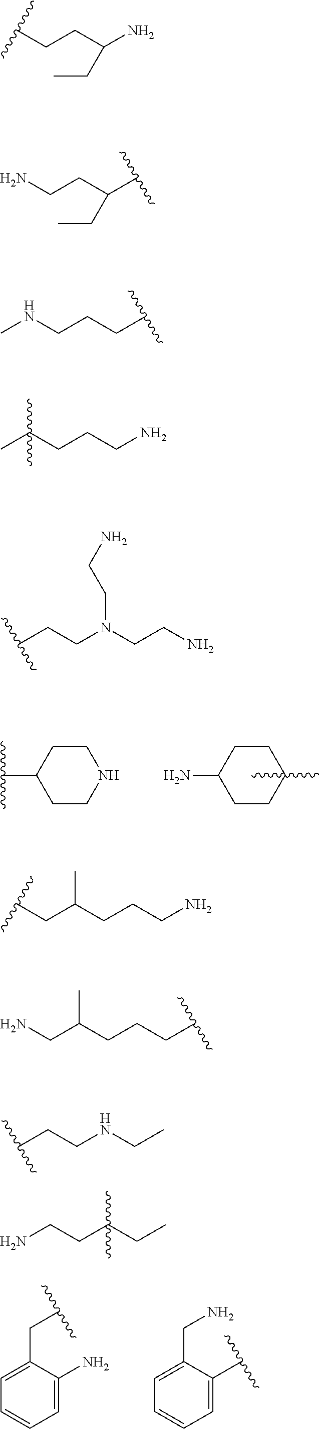

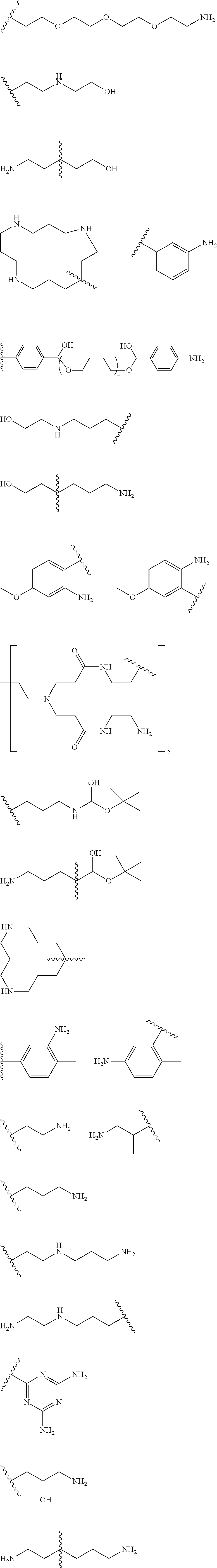

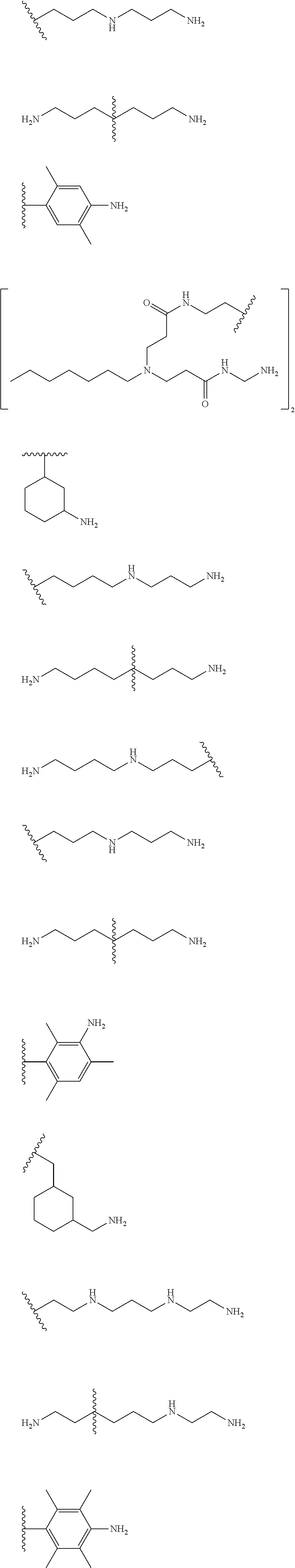

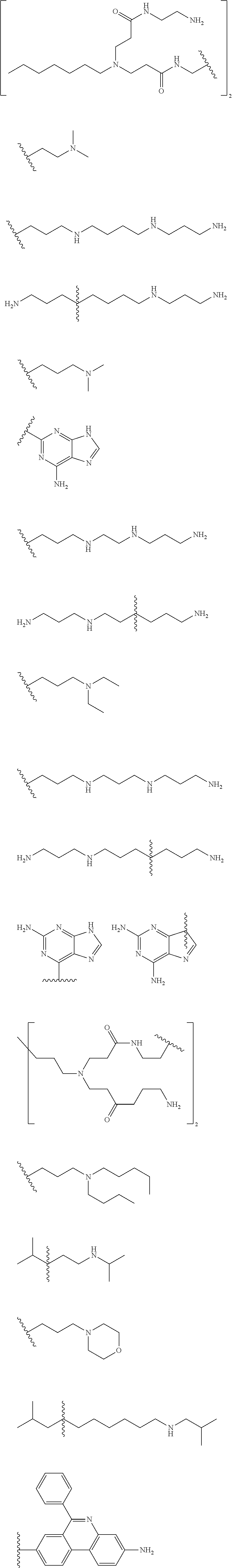

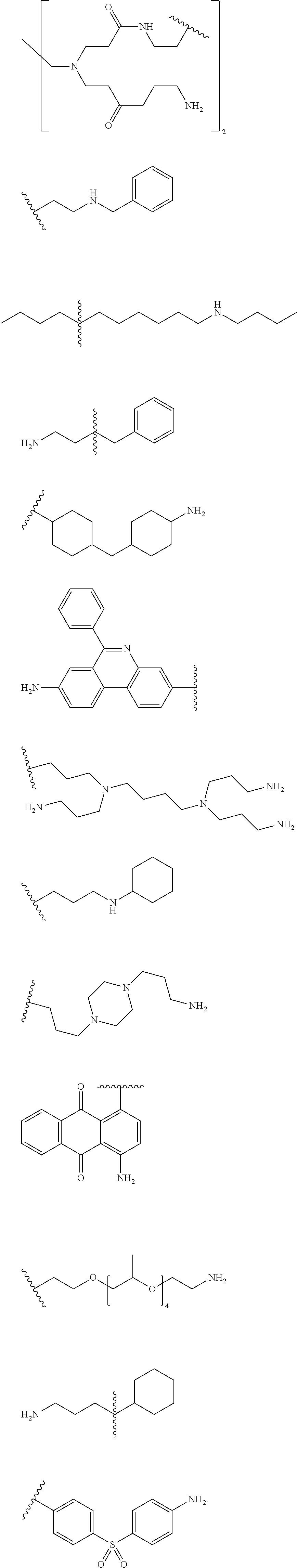

In particular embodiments, the PBAE is constructed with an end group monomer selected from:

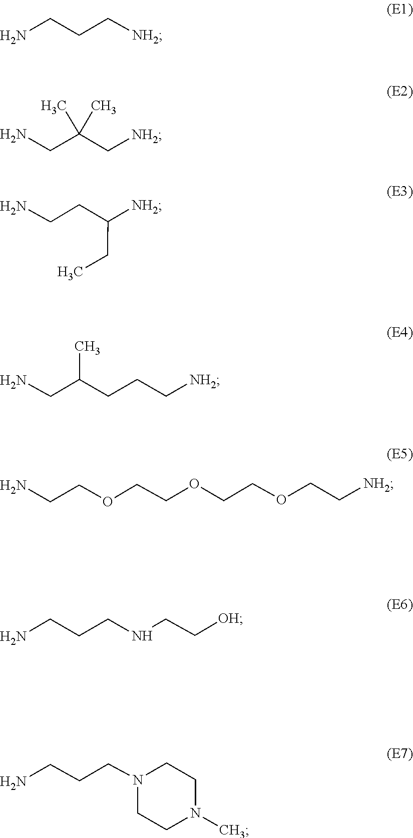

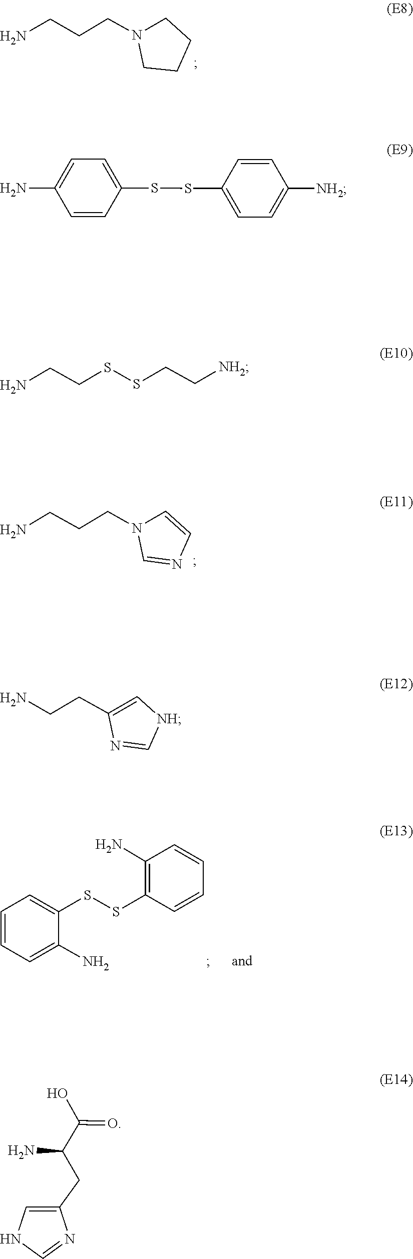

In some embodiments, R′″ is an end group monomer selected from the group consisting of:

Amino Alkanes

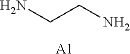

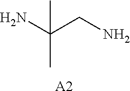

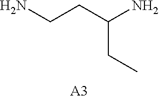

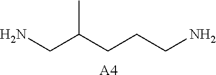

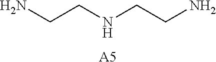

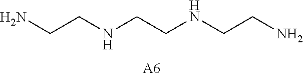

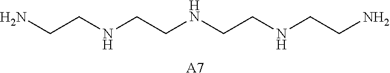

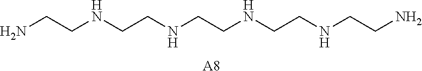

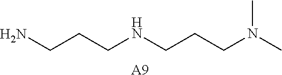

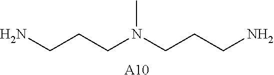

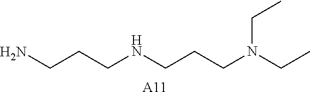

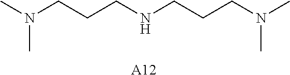

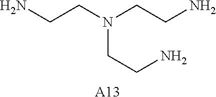

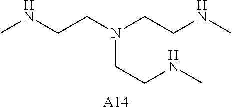

Amino Piperidines

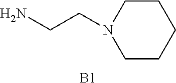

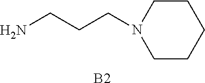

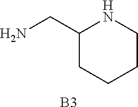

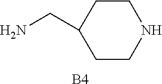

Amino Piperizines

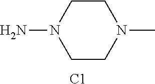

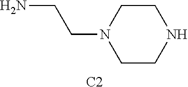

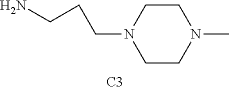

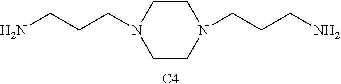

Amino Pyrrolidines

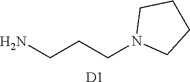

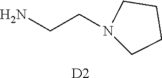

Amino Alcohols

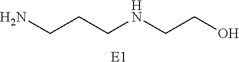

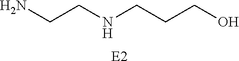

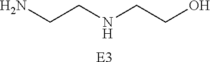

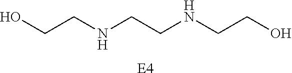

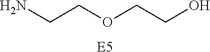

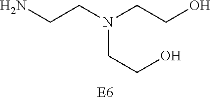

Diamino ethers

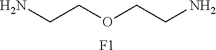

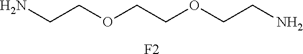

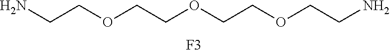

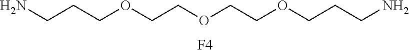

Amino morpholinos

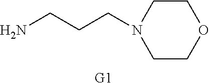

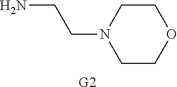

In other embodiments, R′″ is an end group monomer selected from the group consisting of:

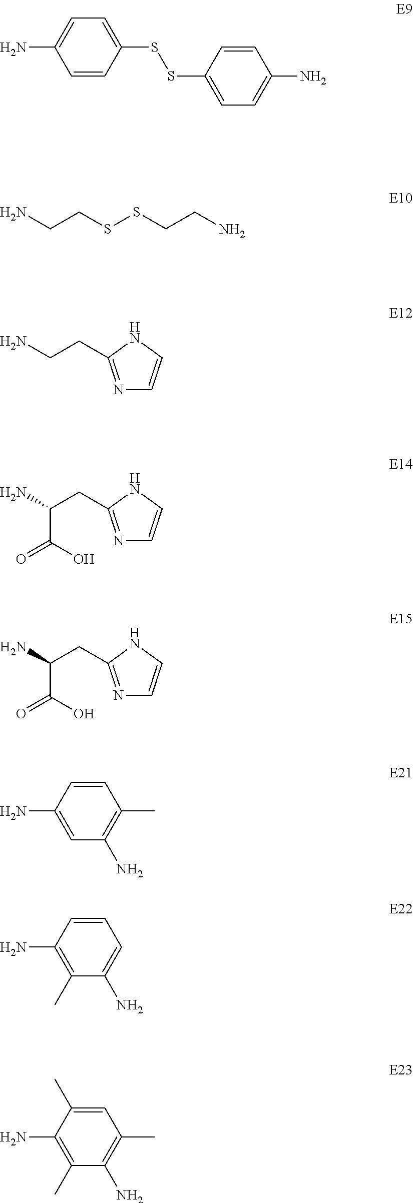

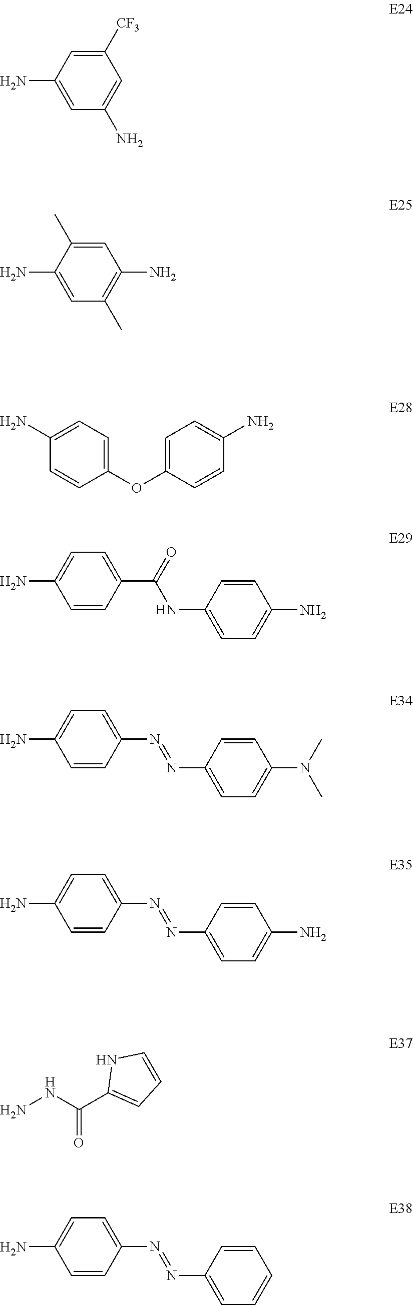

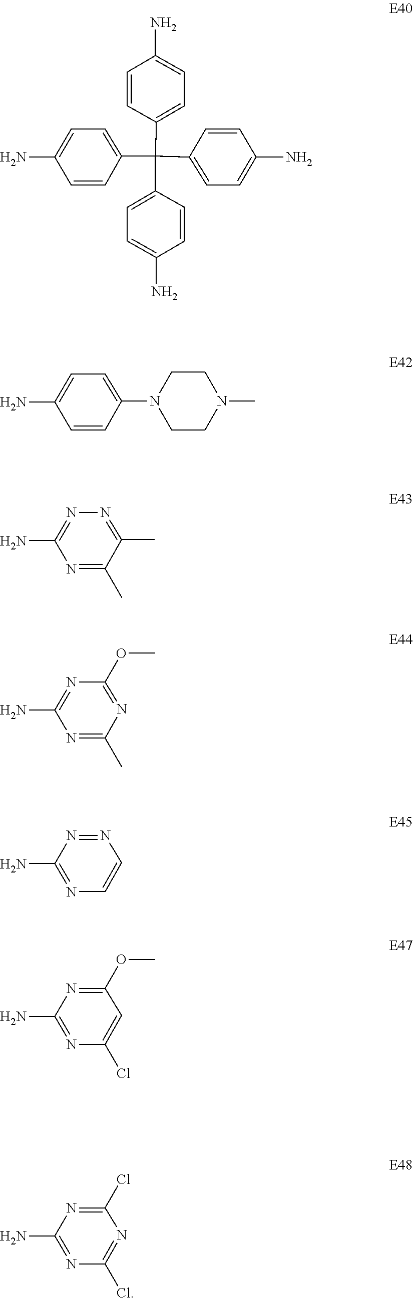

In even more particular embodiments, a combination of R′, and R′″ is selected from the group consisting of:

| Compound | | | |
|---|---|---|---|
| Code | R | R″ | R′′′ |
| 446 | 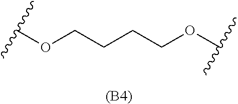 | 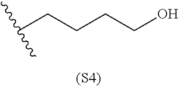 [SMILES: *CCCCO ; "(S4)"] | 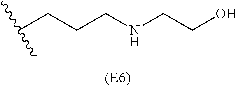 [SMILES: *CCCNCCO ; "(E6)"] |
| 447 | 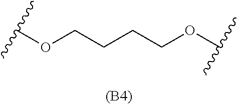 | 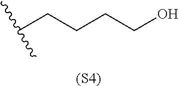 [SMILES: *CCCCO ; "(S4)"] | 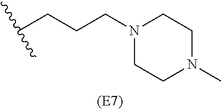 |
| 453 | 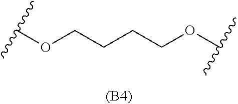 | 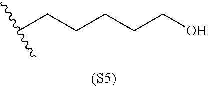 [SMILES: *CCCCCO ; "(S5)"] | 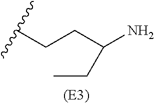 |
| 454 | 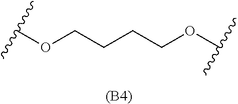 | 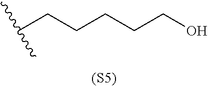 [SMILES: *CCCCCO ; "(S5)"] | 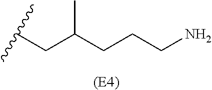 |
| 456 | 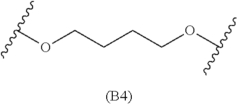 | 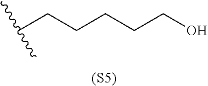 [SMILES: *CCCCCO ; "(S5)"] | 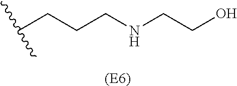 [SMILES: *CCCNCCO ; "(E6)"] |
| 457 | 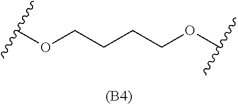 | 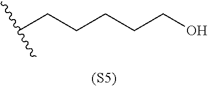 [SMILES: *CCCCCO ; "(S5)"] | 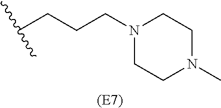 |
| 534 | 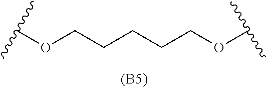 | 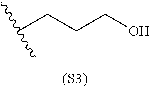 [SMILES: *CCCO ; "(S3)"] | 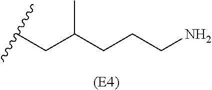 |
| 536 | 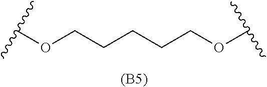 | 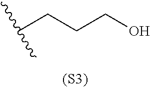 [SMILES: *CCCO ; "(S3)"] | 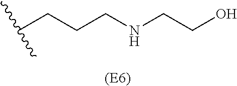 [SMILES: *CCCNCCO ; "(E6)"] |
| 537 | 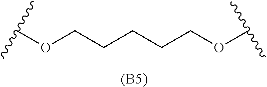 | 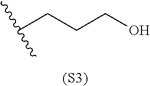 [SMILES: *CCCO ; "(S3)"] | 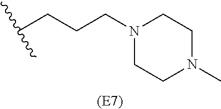 |
| 543 | 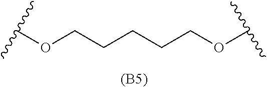 | 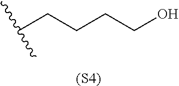 [SMILES: *CCCCO ; "(S4)"] | 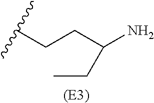 |
| 544 | 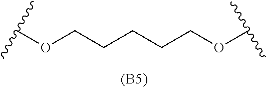 | 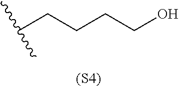 [SMILES: *CCCCO ; "(S4)"] | 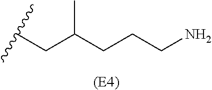 |
| 546 | 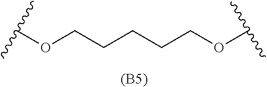 | 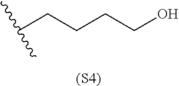 [SMILES: *CCCCO ; "(S4)"] | 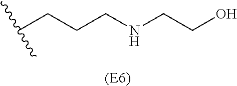 [SMILES: *CCCNCCO ; "(E6)"] |
| 547 | 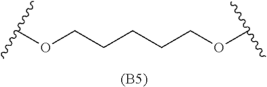 | 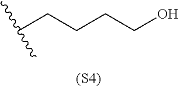 [SMILES: *CCCCO ; "(S4)"] | 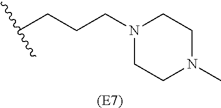 |
| 557 | 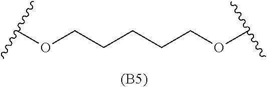 | 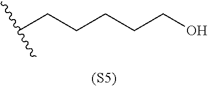 [SMILES: *CCCCCO ; "(S5)"] | 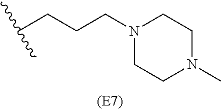 |

In even yet more particular embodiments, the PBAE of formula (I) is selected from the group consisting of:

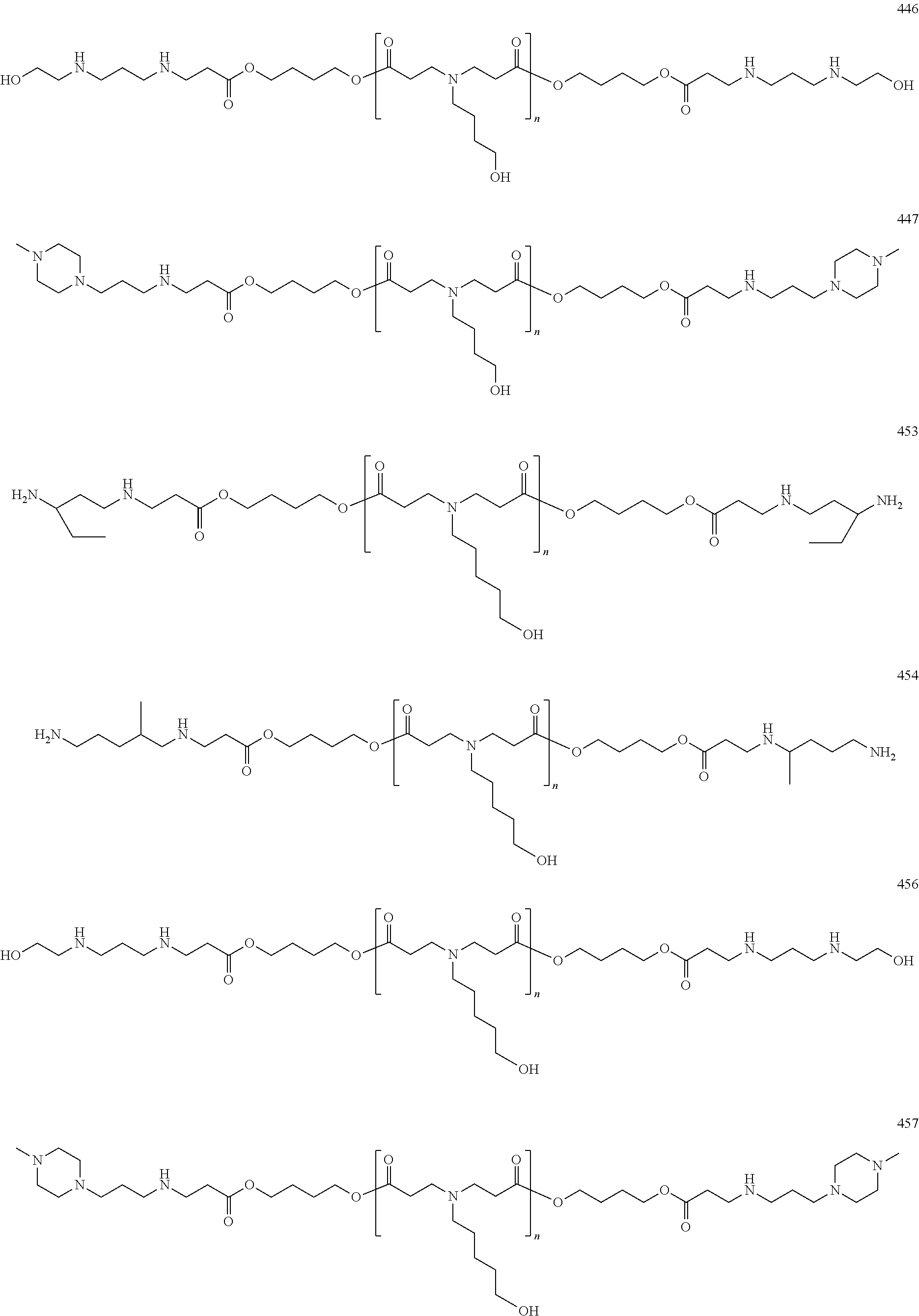

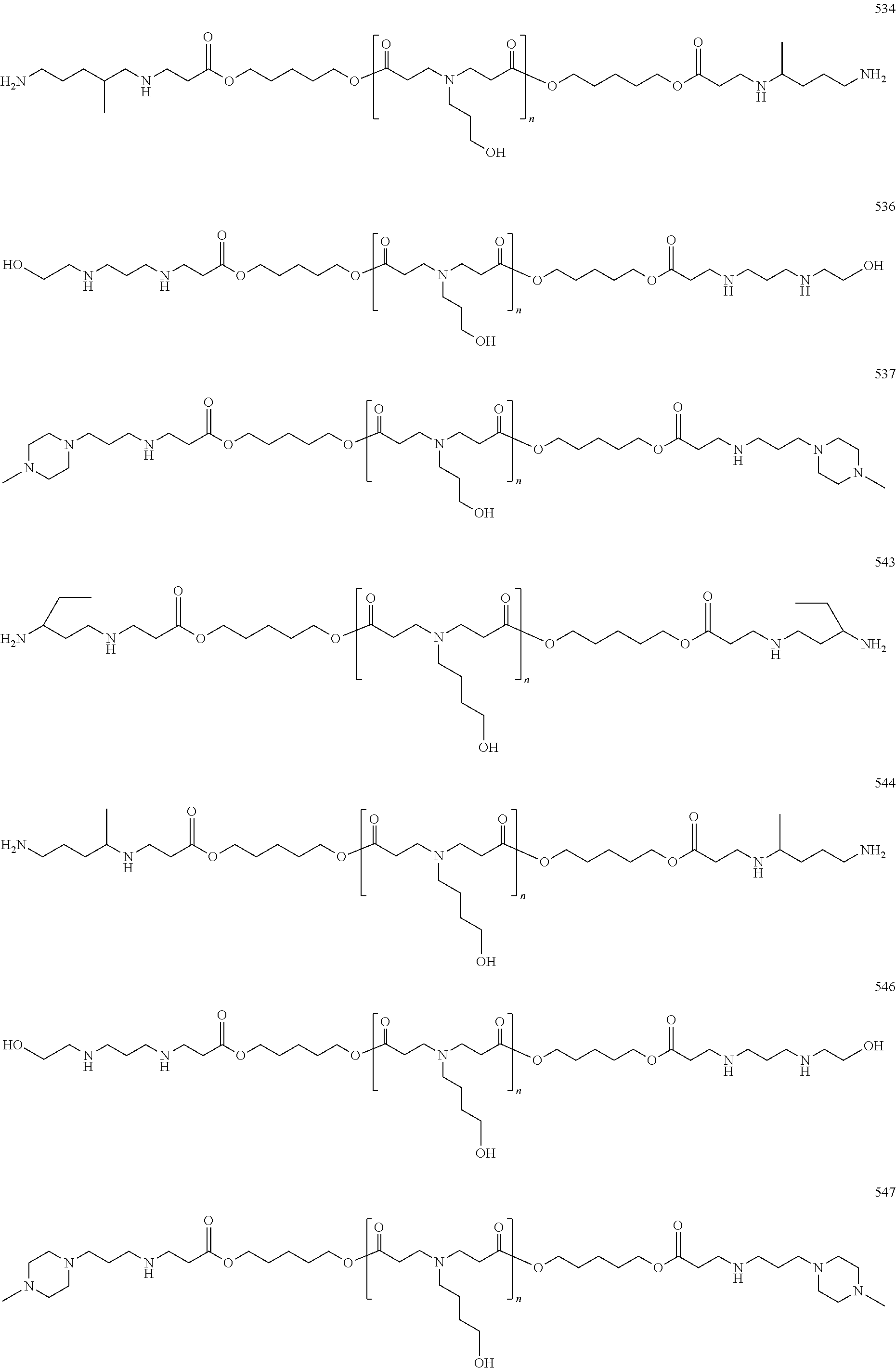

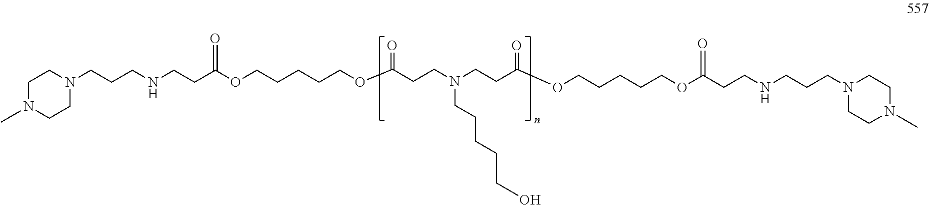

In certain embodiments, the PBAE of formula (I) is:

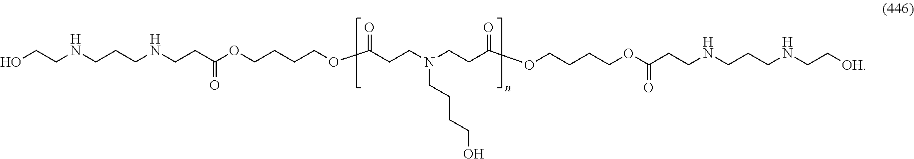

In other embodiments, the PBAE of formula (I) is:

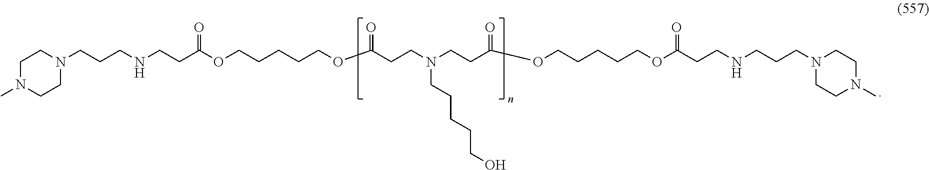

In yet other embodiments, the PBAE of formula (I) is:

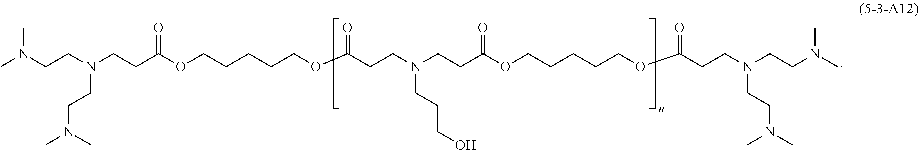

In some embodiments, the PBAE of formula (II) is:

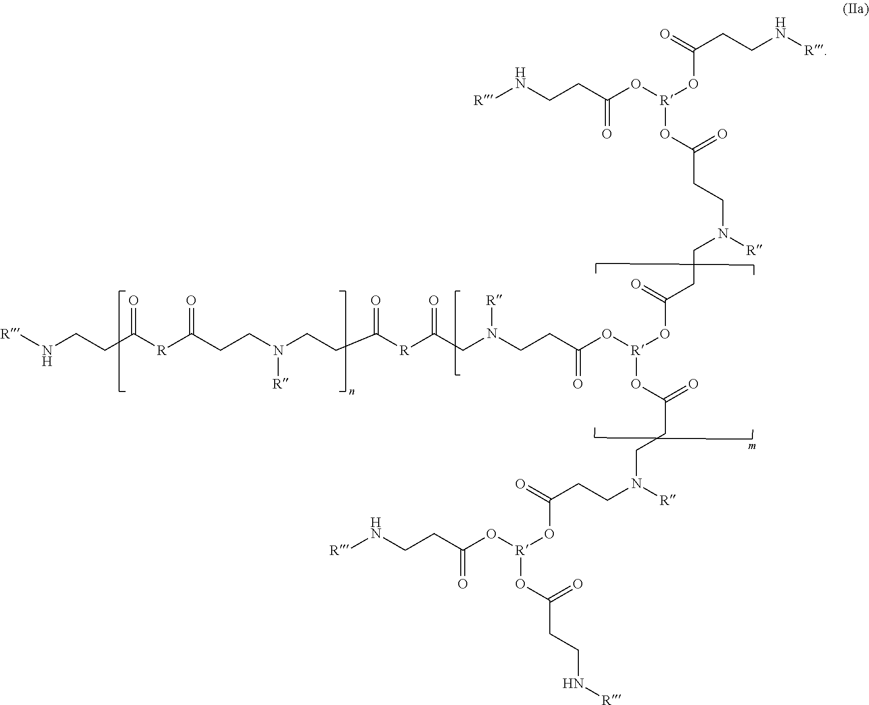

In some embodiments, the tertiary acrylate monomer is trimethylolpropane triacrylate (TMPTA):

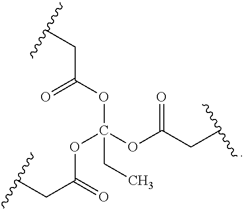 [SMILES: *CC(=O)OC(CC)(OC(=O)C*)OC(=O)C*]

One of ordinary skill in the art would appreciate that other triacrylate structures may be used to provide the requisite polymer branching.

In certain embodiments, the PBAE of formula (I) is 547:

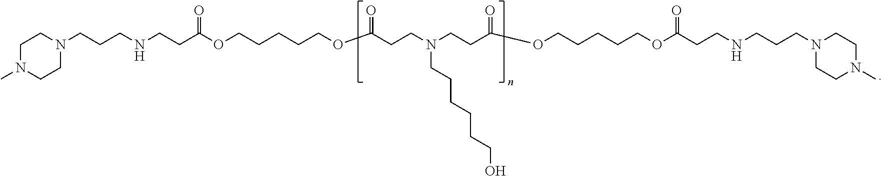

In some embodiments, n is selected from the group consisting of: an integer from 1 to 1,000; an integer from 1 to 100; an integer from 1 to 30; an integer from 5 to 20; an integer from 10 to 15; and an integer from 1 to 10.

In particular embodiments, the composition has a PBAE-to-DNA weight-to-weight ratio (w/w) selected from the group consisting of 75 w/w, 50 w/w, and 25 w/w.

In certain embodiments, the linear and/or branched PBAE polymer has a molecular weight of from 5 to 10 kDa, or a molecular weight of from 10 to 15 kDa, or a molecular weight of from 15 to 25 kDa, or a molecular weight of from 25 to 50 kDa.

In certain embodiments, the presently disclosed subject matter provides a pharmaceutical formulation of comprising the PBAE composition of formula (I) in a pharmaceutically acceptable carrier.

As used herein, “pharmaceutically acceptable carrier” is intended to include, but is not limited to, water, saline, dextrose solutions, human serum albumin, liposomes, hydrogels, microparticles and nanoparticles. The use of such media and agents for pharmaceutically active compositions is well known in the art, and thus further examples and methods of incorporating each into compositions at effective levels need not be discussed here.

In particular embodiments, the pharmaceutical formulation further comprises one or more therapeutic agents.

In yet other embodiments, the pharmaceutical formulation further comprises a nanoparticle or microparticle of the PBAE of formula (I). The PBAE polymers in some embodiments can self-assemble with nucleic acid, including plasmid DNA, to form nanoparticles which may be in the range of 50 to 500 nm in size, e.g., about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 nm in size.

In embodiments, the particle has at least one dimension in the range of about 50 nm to about 500 nm, or from about 50 to about 200 nm. Exemplary particles may have an average size (e.g., average diameter) of about 50, about 75, about 100, about 125, about 150, about 200, about 250, about 300, about 400 or about 500 nm. In some embodiments, the nanoparticle has an average diameter of from about 50 nm to about 500 nm, from about 50 nm to about 300 nm, or from about 50 nm to about 200 nm, or from about 50 nm to about 150 nm, or from about 70 to 100 nm. In embodiments, the nanoparticle has an average diameter of from about 200 nm to about 500 nm. In embodiments, the nanoparticle has at least one dimension, e.g., average diameter, of about 50 to about 100 nm. Nanoparticles are usually desirable for in vivo applications. For example, a nanoparticle of less than about 200 nm will better distribute to target tissues in vivo.

In some embodiments, the presently disclosed particles may comprise other combinations of cationic polymeric blends or block co-polymers. Additional polymers include polycaprolactone (PCL), polyglycolic acid (PGA), polylactic acid (PLA), poly(acrylic acid) (PAA), poly-3-hydroxybutyrate (P3HB), poly(hydroxybutyrate-co-hydroxyvalerate), and polyethylene glycol (PEG). In embodiments, a particle includes blends of other polymer materials to modulate a particle's surface properties. For example, the blend may include non-degradable polymers that are used in the art, such as polystyrene. Thus, in embodiments, a degradable polymer or polymers from above are blended to create a copolymer system. In yet other embodiments, the presently disclosed particle comprises a polymer blend of PBAE, e.g., a mixture of PBAE polymers.

In embodiments, the particles are spherical in shape. In embodiments, the particles have a non-spherical shape. In embodiments, the particles have an ellipsoidal shape with an aspect ratio of the long axis to the short axis between 2 and 10.

In certain embodiments, nanoparticles formed through the presently disclosed procedures that encapsulate active agents, such as DNA plasmid, are themselves encapsulated into a larger nanoparticle, microparticle, or device. In some embodiments, this larger structure is degradable and in other embodiments it is not degradable and instead serves as a reservoir that can be refilled with the nanoparticles. These larger nanoparticles, microparticles, and/or devices can be constructed with any biomaterials and methods that one skilled in the art would be aware. In some embodiments they can be constructed with multi-component degradable cationic polymers as described herein. In other embodiments, they can be constructed with FDA-approved biomaterials, including, but not limited to, poly(lactic-co-glycolic acid) (PLGA). In the case of PLGA and the double emulsion fabrication process as an example, the nanoparticles are part of the aqueous phase in the primary emulsion. In the final PLGA nano- or microparticles, the nanoparticles will remain in the aqueous phase and in the pores/pockets of the PLGA nano- or microparticles. As the microparticles degrade, the nanoparticles will be released, thereby allowing sustained release of the nanoparticles comprising the active agents. In particular embodiments, the nanoparticle or microparticle of the PBAE of formula (I) is encapsulated in a poly(lactic-co-glycolic acid) (PLGA) nanoparticle or microparticle.

In embodiments, a particle of the present technology comprises a ligand on its surface which specifically targets the particle to a cell of interest. Thus, such a particle delivers its cargo, i.e., the nucleic acid encoding a gene-editing protein, primarily to a cell that is need of gene editing.

In embodiments, the ligand is an antibody or fragment or portion thereof. The antibody or fragment or portion thereof having binding specificity for a receptor or other target on the surface of the cell of interest. As used herein, the term “antibody” includes antibodies and antigen-binding portions thereof. In some embodiments, the ligand is an antibody (e.g., a monoclonal or polyclonal antibody) or an antibody mimetic, such as a single-domain antibody, a recombinant heavy-chain-only antibody (VHH), a single-chain antibody (scFv), a shark heavy-chain-only antibody (VNAR), a microprotein (cysteine knot protein, knottin), a DARPin, a Tetranectin, an Affibody; a Transbody, an Anticalin, an AdNectin, an Affilin, a Microbody, a peptide aptamer, a phylomer, a stradobody, a maxibody, an evibody, a fynomer, an armadillo repeat protein, a Kunitz domain, an avimer, an atrimer, a probody, an immunobody, a triomab, a troybody, a pepbody, a vaccibody, a UniBody, a DuoBody, a Fv, a Fab, a Fab′, a F(ab′)2, a peptide mimetic molecule, or a synthetic molecule, or as described in US Patent Nos. or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317.

In embodiments, the ligand specifically binds to a tumor-associated antigen or epitope thereof. Tumor-associated antigens include unique tumor antigens expressed exclusively by the tumor from which they are derived, shared tumor antigens expressed in many tumors but not in normal adult tissues, and tissue-specific antigens expressed also by the normal tissue from which the tumor arose. Tumor-associated antigens can be, for example, embryonic antigens, antigens with abnormal post-translational modifications, differentiation antigens, products of mutated oncogenes or tumor suppressors, fusion proteins, or oncoviral proteins. Tumor-associated antigens also include altered glycolipid and glycoprotein antigens, such as neuraminic acid-containing glycosphingolipids (e.g., GM2 and GD2, expressed in melanomas and some brain tumors); blood group antigens, particularly T and sialylated Tn antigens, which can be aberrantly expressed in carcinomas; and mucins, such as CA-125 and CA-19-9 (expressed on ovarian carcinomas) or the underglycosylated MUC-1 (expressed on breast and pancreatic carcinomas). Use of ligand that binds to a tumor-associated antigen or epitope thereof, allows delivery of a nucleic acid expressing a gene-editing protein to a cancer cell; in the cancer cell, the gene-editing protein may delete or inactivate a gene responsible for the cancer cell's proliferation, for example.

Ligands can be chemically conjugated to a particle using any available process. Functional groups for ligand binding include COOH, NH₂, SH, maleimide, pyridyl disulfide and acrylate. See, e.g., Hermanson, BIOCONJUGATE TECHNIQUES, Academic Press, New York, 1996. Activating functional groups include alkyl and acyl halides, amines, sulfhydryls, aldehydes, unsaturated bonds, hydrazides, isocyanates, isothiocyanates, ketones, azide, alkyne-derivatives, anhydrides, epoxides, carbonates, aminoxy, furan-derivatives and other groups known to activate for chemical bonding. In some embodiments, a ligand can be bound to the particle through the use of a small molecule-coupling reagent. Non-limiting examples of coupling reagents include carbodiimides, maleimides, N-hydroxysuccinimide esters, bischloroethylamines, and functional aldehydes such as glutaraldehyde, anhydrides and the like. In other embodiments, a ligand is coupled to a particle through affinity binding such as a biotin-streptavidin linkage or coupling. For example, streptavidin can be bound to a particle by covalent or non-covalent attachment, and a biotinylated ligand can be synthesized using methods that are well known in the art.

In embodiments, ligands are conjugated to a particle through use of cross-linkers containing n-hydro-succinimido (NHS) esters which react with amines on proteins. Alternatively, the cross-linkers are employed that contain active halogens that react with amine-, sulfhydryl-, or histidine-containing proteins, or cross-linkers containing epoxides that react with amines or sulfhydryl groups, or between maleimide groups and sulfhydryl groups. In embodiments, ligands and protein complexes are conjugated, e.g., functionalized, to the particles using EDC/NHS (1-ethyl-3-(3-dimethylaminopropyl)carbodiimide hydrochloride/N-hydroxysuccinimide) chemistry, which conjugates carboxyl groups of protein ligands to PLGA. In some embodiments, ligands can be engineered with site-specific functional groups (example, such as a free cysteine), to provide consistent, site-directed, attachment to particles. Site directed attachment can be to functional groups of the selected polymers, including amines. In these embodiments, functional domains of ligands can be directed toward the environment and away from the particle surface. These embodiments further provide a controlled orientation more suitable for off-the-shelf pharmaceutical products.

The resulting nanoparticles are non-cytotoxic and are biodegradable with a half-life between 1 and 7 h in aqueous conditions. Moreover, freeze-dried nanoparticles are stable for up to two years when stored at room temperature, 4° C., or −20° C.

In some embodiments, the presently disclosed subject matter also includes a method of using and storing the polymers and particles described herein whereby a cryoprotectant (including, but not limited to, a sugar) is added to the polymer and/or particle solution and it is lyophilized and stored as a powder. Such a powder is designed to remain stable and be reconstituted easily with aqueous buffer as one skilled in the art could utilize.

### B. Pharmaceutical Formulations

In some embodiments, the presently disclosed subject matter provides a pharmaceutical formulation comprising the composition comprising a poly(beta-amino ester) (PBAE) of formula (I) or formula (II) and at least one DNA or RNA molecule comprising a nucleic acid sequence encoding a gene-editing protein or therapeutic protein in a pharmaceutically acceptable carrier.

In some embodiments, the pharmaceutical formulation further comprises a nanoparticle or microparticle of the PBAE of formula (I) or formula (II). In certain embodiments, the nanoparticle or microparticle of the PBAE of formula (I) or formula (II) is encapsulated in a poly(lactic-co-glycolic acid) (PLGA) nanoparticle or microparticle.

### C. Kits

In some embodiments, the presently disclosed subject matter provides a kit comprising the composition comprising a poly(beta-amino ester) (PBAE) of formula (I) or formula (II) and at least one DNA or RNA molecule comprising a nucleic acid sequence encoding a gene-editing protein or therapeutic protein in a pharmaceutically acceptable carrier. In certain embodiments, the kit further comprises one of more of multiple dosage units of the composition, a pharmaceutically acceptable carrier, a device for administration of the composition, instructions for use, and combinations thereof.

### D. Methods for Gene Editing

In some embodiments, the presently disclosed subject matter provides a method for gene editing comprising contacting a cell with the composition comprising a poly(beta-amino ester) (PBAE) of formula (I) or formula (II), and at least one DNA or RNA molecule comprising a nucleic acid sequence encoding a gene-editing protein or therapeutic protein. In particular embodiments, the gene-editing endonuclease directs site-specific target DNA disruption, mutation, deletion, or repair. In some embodiments, the composition and cell are contacted in vivo. In other embodiments, the composition and cell are contacted ex vivo.

Accordingly, the presently disclosed particles provide for efficient transfection of cells with nucleic acid, for example, nucleic acid encoding gene editing factors to provide effective gene editing either in vivo or ex vivo. Accordingly, in some embodiments, the particles carry DNA or mRNA encoding a gene editing protein (a gene-editing endonuclease). For example, the particles may carry plasmid DNA encoding a gene editing protein, and where necessary a guide RNA. Guide RNA (e.g., gRNA) may be encoded on a plasmid or provided in RNA form. In some embodiments, the nanoparticles further provide a template nucleic acid for recombination or insertion into a genome (e.g., for a knock-in).

The target DNA may be the cause of a disease or disorder, e.g., due to a genetic mutation (including but not limited to a single nucleotide polymorphism or SNP). In some embodiments, the particle delivers gene editing factors to direct production of one or more substituted (e.g., mutated), corrected, truncated, loss-of-function, gain-of function, and/or frameshifted proteins. In some embodiments, the nanoparticles carry DNA encoding gene editing factors that direct deletion of a gene segment.

The call can be a eukaryotic cell, such as an animal cell or plant cell, including a mammalian cell, such as a human cell. In some embodiments, including for ex vivo nucleic acid delivery, the cell is a stem cell or progenitor cell. The cell may be multipotent or pluripotent. In some embodiments, the cell is a stem cell, such as an embryonic stem cell or adult stem cell. In some embodiments, the cell is a hematopoietic stem cell. In some embodiments, including for in vivo nucleic acid delivery, the cell (e.g., target cell) is a cancer cell, malignant cell, or diseases cell.

In some embodiments, the particles are delivered directly to an organism, such as mammalian subject, to thereby direct gene editing in vivo. For gene editing in vivo, particles can be formulated for a variety of modes of administration, including systemic and topical or localized administration.

Thus, the pharmaceutical compositions can be formulated for administered to patients by any appropriate routes, including intravenous administration, intra-arterial administration, subcutaneous administration, intradermal administration, intralymphatic administration, and intra-tumoral administration.

In some embodiments, the composition is lyophilized, and reconstituted prior to administration.

In various embodiments, the nanoparticles carry a nucleic acid (e.g., DNA or RNA (e.g., mRNA)) encoding a gene editing protein (a gene-editing endonuclease). For example, in some embodiments, the nanoparticles carry plasmid DNA encoding a gene editing protein and, in some embodiments, a guide RNA (e.g., gRNA). Guide RNA may be encoded on a plasmid or provided in RNA form. In some embodiments, the nanoparticles further provide a nucleic acid (e.g., a template) that is a functional gene or portion thereof for recombination or insertion into a genome (e.g., to provide a “knock-in”). Factors for gene editing can be provided on a single plasmid, or in some embodiments, are encoded on distinct plasmids.

In some embodiments, the nanoparticles comprise a ribonucleoprotein. That is, in some embodiments, nanoparticles comprise a polypeptide (e.g., a gene-editing endonuclease (e.g., a CRISPR protein (e.g., a Cas9 or Cas9-like protein))) and a gRNA.

A gene-editing endonuclease creates a nick or a double-strand break in a target DNA molecule, which inactivates a gene or results in expression (from the gene) of an inactive, reduced-activity, or dominant-negative form of a protein. In some embodiments, the gene-editing protein repairs one or more mutations in a gene or deletes a gene segment, which can be guided by a gRNA with the CRISPR/Cas9 system.

The present technology provides particles comprising nucleic acids that encode gene-editing proteins. The gene-editing protein may be one or more of a Clustered Regularly Interspaced Short Palindromic Repeats (CRISPR)-associated nuclease, Cre recombinase, Flp recombinase, a meganuclease, a Transcription Activator-Like Effector Nuclease (TALEN), a Zinc-Finger Nuclease (ZFN), or a natural or engineered variant, family-member, orthologue, fragment or fusion construct thereof.

In embodiments, the gene-editing protein relates to CRISPR. CRISPR is described, at least in U.S. Pat. Nos. 8,697,359 and 9,637,739, each of which is hereby incorporated by reference in its entirety. In embodiments of the present technology, a particle as provided herein comprises a nucleic acid encoding a CRISPR-associated nuclease, e.g., the Cas9 endonuclease. While various CRISPR/Cas systems have been used extensively for genome editing in cells of various types and species, recombinant and engineered nucleic acid-binding proteins, such as Cas9 and Cas9-like proteins, find use (e.g., in vitro) in the present technology. The Cas9 protein was discovered as a component of the bacterial adaptive immune system (see, e.g., Barrangou et al. (2007) “CRISPR provides acquired resistance against viruses in prokaryotes” Science 315: 1709-1712, incorporated herein by reference). Cas9 is an RNA-guided endonuclease that targets and digests foreign DNA in bacteria using RNA:DNA base-pairing between a guide RNA (gRNA) and foreign DNA to provide sequence specificity. Recently, Cas9/gRNA complexes (e.g., a Cas9/gRNA RNP) have found use in genome editing (see, e.g., Doudna et al. (2014) “The new frontier of genome engineering with CRISPR-Cas9” Science 346: 6213, incorporated herein by reference).

In some embodiments, different CRISPR proteins (e.g., Cas9 proteins (e.g., Cas9 proteins from various species and modified versions thereof)) may be advantageous to use in the various provided methods in order to capitalize on various characteristics of the different CRISPR proteins (e.g., for different PAM sequence preferences; for no PAM sequence requirement; for increased or decreased binding activity; for an increased or decreased level of cellular toxicity; for increase or decrease efficiency of in vitro RNP formation; for increase or decrease ability for introduction into cells (e.g., living cells, e.g., living primary cells), etc.). CRISPR proteins from various species may require different PAM sequences in the target DNA. Thus, for a particular CRISPR protein of choice, the PAM sequence requirement may be different than the 5′-XGG-3′ sequence described above. In some embodiments, the protein is an xCas protein having an expanded PAM compatibility (e.g., a Cas9 variant that recognizes a broad range of PAM sequences including NG, GAA and GAT), e.g., as described in Hu et al. (2018) “Evolved Cas9 variants with broad PAM compatibility and high DNA specificity” Nature 556: 57-63, incorporated herein by reference in its entirety.

In some embodiments, the technology comprises use of other RNA-guided gene-editing nucleases (e.g., Cpf1 and modified versions thereof, Cas13 and modified versions thereof). For example, in some embodiments, use of other RNA-guided nucleases (e.g., Cpf1 and modified versions thereof) provides advantages—e.g., in some embodiments, the characteristics of the different nucleases are appropriate for methods as described herein (e.g., other RNA-guided nucleases have preferences for different PAM sequence preferences; other RNA-guided nucleases operate using single crRNAs other than cr/tracrRNA complexes; other RNA-guided nucleases operate with shorter guide RNAs, etc.) In some embodiments, the technology comprises use of a Cpf1 protein, e.g., as described in U.S. Pat. No. 9,790,490, which is incorporated herein by reference in its entirety.

Many Cas9 orthologs from a wide variety of species have been identified and the proteins share only a few identical amino acids. All identified Cas9 orthologs have the same or similar domain architecture comprising a central HNH endonuclease domain and a split RuvC/RNaseH domain. Cas9 proteins share 4 motifs with a conserved architecture. Motifs 1, 2, and 4 are RuvC like motifs and motif 3 is an HNH motif. In some embodiments, a suitable polypeptide (e.g., a Cas9) comprises an amino acid sequence having 4 motifs, each of motifs 1-4 having at least about 75%, at least about 80%, at least about 85%, at least about 90%, at least about 95%, at least about 99% or 100% amino acid sequence identity to the motifs 1˜4 of a known Cas9 and/or Csn1 amino acid sequence.

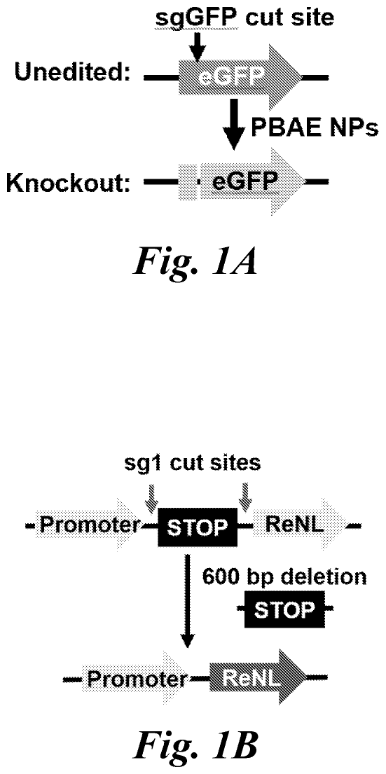

A number of bacteria express Cas9 protein variants. The Cas9 from Streptococcus pyogenes is presently the most commonly used; some of the other Cas9 proteins have high levels of sequence identity with the S. pyogenes Cas9 and use the same guide RNAs. Others are more diverse, use different gRNAs, and recognize different PAM sequences as well (the 2-5 nucleotide sequence specified by the protein which is adjacent to the sequence specified by the RNA). Chylinski et al. classified Cas9 proteins from a large group of bacteria (RNA Biology 10:5, 1-12; 2013, incorporated herein by reference), and a large number of Cas9 proteins are listed in supplementary FIG. 1 and supplementary table 1 thereof, which are incorporated by reference herein. Additional Cas9 proteins are described in Esvelt et al., Nat Methods. 2013 November; 10(11):1116-21 and Fonfara et al., “Phylogeny of Cas9 determines functional exchangeability of dual-RNA and Cas9 among orthologous type II CRISPR-Cas systems.” Nucleic Acids Res. 42: 2577-90 (2014), each of which is incorporated herein by reference.

Cas9 proteins, and thus modified Cas9 proteins, from a variety of species find use in the technology described herein. While the S. pyogenes and S. thermophilus Cas9 molecules are widely used, Cas9 molecules of, derived from, or based on the Cas9 proteins of other species listed herein find use in embodiments of the technology. Accordingly, the technology provides for the replacement of S. pyogenes and S. thermophilus Cas9 and modified CRISPR (e.g., Cas9) protein molecules with Cas9 and modified CRISPR protein molecules from the other species, e.g.:

| GenBank Acc No. | Bacterium |
|---|---|
| 303229466 | Veillonella atypica ACS-134-V-Col7a |
| 34762592 | Fusobacterium nucleatum subsp. vincentii |
| 374307738 | Filifactor alocis ATCC 35896 |
| 320528778 | Solobacterium moorei F0204 |
| 291520705 | Coprococcus catus GD-7 |
| 42525843 | Treponema denticola ATCC 35405 |
| 304438954 | Peptoniphilus duerdenii ATCC BAA-1640 |
| 224543312 | Catenibacterium mitsuokai DSM 15897 |
| 24379809 | Streptococcus mutans UA159 |
| 15675041 | Streptococcus pyogenes SF370 |
| 16801805 | Listeria innocua Clip 11262 |
| 116628213 | Streptococcus thermophilus LMD-9 |
| 323463801 | Staphylococcus pseudintermedius ED99 |
| 352684361 | Acidaminococcus intestini RyC-MR95 |
| 302336020 | Olsenella uli DSM 7084 |
| 366983953 | Oenococcus kitaharae DSM 17330 |
| 310286728 | Bifidobacterium bifidum S17 |
| 258509199 | Lactobacillus rhamnosus GG |
| 300361537 | Lactobacillus gasseri JV-V03 |
| 169823755 | Finegoldia magna ATCC 29328 |
| 47458868 | Mycoplasma mobile 163K |
| 284931710 | Mycoplasma gallisepticum str. F |
| 363542550 | Mycoplasma ovipneumoniae SC01 |
| 384393286 | Mycoplasma canis PG 14 |
| 71894592 | Mycoplasma synoviae 53 |
| 238924075 | Eubacterium rectale ATCC 33656 |
| 116627542 | Streptococcus thermophilus LMD-9 |
| 315149830 | Enterococcus faecalis TX0012 |
| 315659848 | Staphylococcus lugdunensis M23590 |
| 160915782 | Eubacterium dolichum DSM 3991 |
| 336393381 | Lactobacillus coryniformis subsp. torquens |
| 310780384 | Ilyobacter polytropus DSM 2926 |
| 325677756 | Ruminococcus albus 8 |
| 187736489 | Akkermansia muciniphila ATCC BAA-835 |
| 117929158 | Acidothermus cellulolyticus 11B |
| 189440764 | Bifidobacterium longum DJO10A |
| 283456135 | Bifidobacterium dentium Bd1 |
| 38232678 | Corynebacterium diphtheriae NCTC 13129 |
| 187250660 | Elusimicrobium minutum Pei 191 |
| 319957206 | Nitratifractor salsuginis DSM 16511 |
| 325972003 | Sphaerochaeta globus str. Buddy |
| 261414553 | Fibrobacter succinogenes subsp. succinogenes |
| 60683389 | Bacteroides fragilis NCTC 9343 |
| 256819408 | Capnocytophaga ochracea DSM 7271 |
| 90425961 | Rhodopseudomonas palustris BisB18 |
| 373501184 | Prevotella micans F0438 |
| 294674019 | Prevotella ruminicola 23 |
| 365959402 | Flavobacterium columnare ATCC 49512 |
| 312879015 | Aminomonas paucivorans DSM 12260 |
| 83591793 | Rhodospirillum rubrum ATCC 11170 |
| 294086111 | Candidatus Puniceispirillum marinum IMCC1322 |
| 121608211 | Verminephrobacter eiseniae EF01-2 |
| 344171927 | Ralstonia syzygii R24 |
| 159042956 | Dinoroseobacter shibae DFL 12 |
| 288957741 | Azospirillum sp- B510 |
| 92109262 | Nitrobacter hamburgensis X14 |
| 148255343 | Bradyrhizobium sp- BTAi1 |
| 34557790 | Wolinella succinogenes DSM 1740 |
| 218563121 | Campylobacter jejuni subsp. jejuni |
| 291276265 | Helicobacter mustelae 12198 |
| 229113166 | Bacillus cereus Rock1-15 |
| 222109285 | Acidovorax ebreus TPSY |
| 189485225 | uncultured Termite group 1 |
| 182624245 | Clostridium perfringens D str. |
| 220930482 | Clostridium cellulolyticum H10 |
| 154250555 | Parvibaculum lavamentivorans DS-1 |
| 257413184 | Roseburia intestinalis L1-82 |
| 218767588 | Neisseria meningitidis Z2491 |
| 15602992 | Pasteurella multocida subsp. multocida |
| 319941583 | Sutterella wadsworthensis 3 1 |
| 254447899 | gamma proteobacterium HTCC5015 |
| 54296138 | Legionella pneumophila str. Paris |
| 331001027 | Parasutterella excrementihominis YIT 11859 |
| 34557932 | Wolinella succinogenes DSM 1740 |
| 118497352 | Francisella novicida U112 |

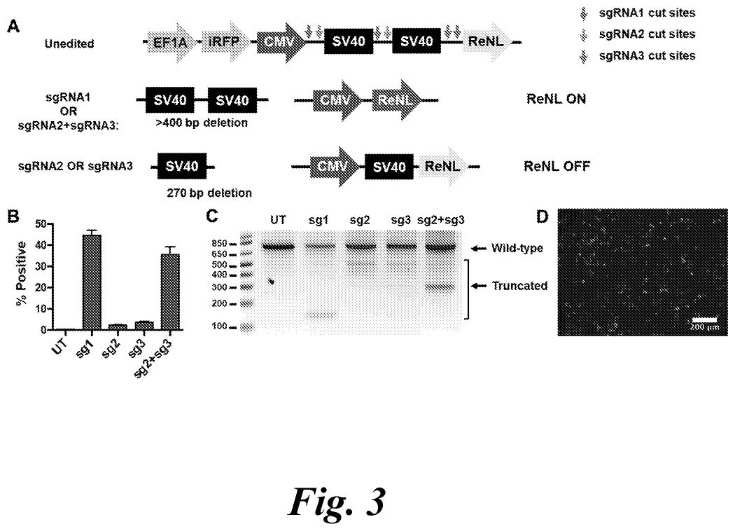

See also U.S. Pat. App. Pub. No. 20170051312 at FIGS. 3, 4, 5, incorporated herein by reference.

In some embodiments, the technology described herein encompasses the use of a CRISPR protein and/or a CRISPR protein derived from any Cas9 protein (e.g., as listed above) and their corresponding guide RNAs or other guide RNAs that are compatible. The Cas9 from the Streptococcus thermophilus LMD-9 CRISPR1 system has been shown to function in human cells (see, e.g., Cong et al. (2013) Science 339: 819, incorporated herein by reference). Additionally, Jinek showed in vitro that Cas9 orthologs from S. thermophilus and L. innocua, can be guided by a dual S. pyogenes gRNA to cleave target plasmid DNA.

In some embodiments, the present technology comprises the Cas9 protein from S. pyogenes, e.g., as encoded in a bacterium or codon-optimized for expression in microbial or mammalian cells. For example, in some embodiments, the Cas9 used herein is at least approximately 50% identical to the sequence of S. pyogenes Cas9, e.g., at least 50% identical to the following sequence provided by GenBank Accession Number WP_010922251, incorporated herein by reference (SEQ ID NO: 2):

>Type II CRISPR RNA-guided endonuclease Cas9
[Streptococcus pyogenes]
MDKKYSIGLDIGTNSVGWAVITDEYKVPSKKFKVL
GNTDRHSIKKNLIGALLFDSGETAEATRLKRTARR
RYTRRKNRICYLQEIFSNEMAKVDDSFFHRLEESF
LVEEDKKHERHPIFGNIVDEVAYHEKYPTIYHLRK
KLVDSTDKADLRLIYLALAHMIKFRGHFLIEGDLN
PDNSDVDKLFIQLVQTYNQLFEENPINASGVDAKA
ILSARLSKSRRLENLIAQLPGEKKNGLFGNLIALS
LGLTPNFKSNFDLAEDAKLQLSKDTYDDDLDNLLA
QIGDQYADLFLAAKNLSDAILLSDILRVNTEITKA
PLSASMIKRYDEHHQDLTLLKALVRQQLPEKYKEI
FFDQSKNGYAGYIDGGASQEEFYKFIKPILEKMDG
TEELLVKLNREDLLRKQRTFDNGSIPHQIHLGELH
AILRRQEDFYPFLKDNREKIEKILTFRIPYYVGPL
ARGNSRFAWMTRKSEETITPWNFEEVVDKGASAQS
FIERMTNFDKNLPNEKVLPKHSLLYEYFTVYNELT
KVKYVTEGMRKPAFLSGEQKKAIVDLLFKTNRKVT
VKQLKEDYFKKIECFDSVEISGVEDRFNASLGTYH
DLLKIIKDKDFLDNEENEDILEDIVLTLTLFEDRE
MIEERLKTYAHLFDDKVMKQLKRRRYTGWGRLSRK
LINGIRDKQSGKTILDFLKSDGFANRNFMQLIHDD
SLTFKEDIQKAQVSGQGDSLHEHIANLAGSPAIKK
GILQTVKVVDELVKVMGRHKPENIVIEMARENQTT
QKGQKNSRERMKRIEEGIKELGSQILKEHPVENTQ
LQNEKLYLYYLQNGRDMYVDQELDINRLSDYDVDH
IVPQSFLKDDSIDNKVLTRSDKNRGKSDNVPSEEW
KKMKNYWRQLLNAKLITQRKFDNLTKAERGGLSEL
DKAGFIKRQLVETRQITKHVAQILDSRMNTKYDEN
DKLIREVKVITLKSKLVSDFRKDFQFYKVREINNY
HHAHDAYLNAVVGTALIKKYPKLESEFVYGDYKVY
DVRKMIAKSEQEIGKATAKYFFYSNIMNFFKTEIT
LANGEIRKRPLIETNGETGEIVWDKGRDFATVRKV
LSMPQVNIVKKTEVQTGGFSKESILPKRNSDKLIA
RKKDWDPKKYGGFDSPTVAYSVLVVAKVEKGKSKK
LKSVKELLGITIMERSSFEKNPIDFLEAKGYKEVK
KDLIIKLPKYSLFELENGRKRMLASAGELQKGNEL
ALPSKYVNFLYLASHYEKLKGSPEDNEQKQLFVEQ
HKHYLDEIIEQISEFSKRVILADANLDKVLSAYNK
HRDKPIREQAENIIHLFTLTNLGAPAAFKYFDTTI
DRKRYTSTKEVLDATLIHQSITGLYETRIDLSQLG
GD

In some embodiments, the technology comprises use of a nucleotide sequence that is approximately 50%, 55%, 60%, 65%, 70%, 75%, 80%, 85%, 90%, 95%, 99% or 100% identical to a nucleotide sequence that encodes a protein described by SEQ ID NO: 2.

In some embodiments, the Cas9 portion of the CRISPR protein used herein is at least about 50% identical to the sequence of the S. pyogenes Cas9, e.g., at least 50%, 55%, 60%, 65%, 70%, 75%, 80%, 85%, 90%, 95%, 99%, or 100% identical to SEQ ID NO: 2.

In some embodiments, the polypeptide (e.g., the gene-editing nuclease) is a Cas protein, CRISPR protein, or Cas-like protein. “Cas protein” and “CRISPR protein” and “Cas-like protein”, as used herein, includes polypeptides, enzymatic activities, and polypeptides having activities similar to proteins known in the art as, or encoded by genes known in the art as, e.g., Cas1, Cas1B, Cas2, Cas3, Cas4, Cas5, Cas6, Cas7, Cas8, Cas9 (also known as Csn1 and Csx12), Cas10, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c2, homologs thereof, or modified versions thereof, e.g., including any of these Cas proteins, CRISPR proteins, and/or Cas-like proteins known in the art.

In embodiments, the technology comprises use of a polypeptide (e.g., a Type V/Type VI protein) such as Cpf1 or C2c1 or C2c2 and homologs and orthologs of a Type V/Type VI protein such as Cpf1 or C2c1 or C2c2 to provide a CRISPR protein. Embodiments encompass Cpf1, modified Cpf1 (e.g., a modified Cpf1), and CRISPR systems related to Cpf1, modified Cpf1, and chimeric Cpf1. In some embodiments, the polypeptide (e.g., a Type V/Type VI protein) such as Cpf1 or C2c1 or C2c2 is from a genus that is, e.g., Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter; Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium, or Acidaminococcus. In some embodiments, the polypeptide (e.g., a Type V/Type VI protein) such as Cpf1 or C2c1 or C2c2 is from an organism that is, e.g., S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, or C. sordellii. See, e.g., U.S. Pat. No. 9,790,490, incorporated herein by reference in its entirety. In some embodiments, a Cpf1 protein finds use as described in U.S. Pat. App. Pub. No. 20180155716, which is incorporated herein by reference.

In some embodiments, differences from SEQ ID NO: 2 are in non-conserved regions, as identified by sequence alignment of sequences set forth in Chylinski et al., RNA Biology 10:5, 1-12; 2013 (e.g., in supplementary FIG. 1 and supplementary table 1 thereof); Esvelt et al., Nat Methods. 2013 November; 10(11):1116-21 and Fonfara et al., Nucl. Acids Res. (2014) 42 (4): 2577-2590, each of which is incorporated herein by reference.

Thus, in some embodiments, the Cas9 polypeptide is a naturally-occurring polypeptide. In some embodiments, the Cas9 polypeptide is not a naturally-occurring polypeptide (e.g., a chimeric polypeptide, a naturally-occurring polypeptide that is modified, e.g., by one or more amino acid substitutions produced by an engineered nucleic acid comprising one or more nucleotide substitutions, deletions, insertions).

In some embodiments, the technology relates to a protein that is a CRISPR protein derivative. In some embodiments, the protein is a Type II Cas9 protein. In some embodiments, the Cas9 has been engineered to partially remove the nuclease domain (e.g., a “dead Cas9” or a “Cas9 nickase”; see, e.g., Nature Methods 11: 399-402 (2014), incorporated herein by reference). In some embodiments, the RNP protein is a protein from a CRISPR system other than the S. pyogenes system, e.g., a Type V Cpf1, C2c1, C2c2, C2c3 protein and derivatives thereof.

In some embodiments, the polypeptide is a chimeric or fusion polypeptide, e.g., a polypeptide that comprises two or more functional domains. For example, in some embodiments, a chimeric polypeptide interacts with (e.g., binds to) an RNA to form an RNP (described above). The RNA guides the polypeptide to a target sequence within target nucleic acid. Thus, in some embodiments a chimeric polypeptide binds target nucleic acid.

In some embodiments, the technology comprises use of an RNA-targeting protein (e.g., Cas13 and/or a modified Cas13), which works according to a similar mechanism as Cas9. In addition to targeting genomic DNA, Cas9 and other CRISPR related proteins (e.g., Cas13) also target RNAs directed by gRNAs (see, e.g., Abudayyeh et al. (2017) “RNA targeting with CRISPR-Cas13” Nature 550: 280, incorporated herein by reference). Thus, in some embodiments, gRNAs complex with Cas9 or other RNA-guided nucleases (e.g., a class 2 type VI RNA-guided RNA-targeting CRISPR-Cas effector (e.g., Cas13), a Cpf1, etc.) to modify (e.g., edit) an RNA (e.g., RNA transcripts and non-coding RNAs). Accordingly, in some embodiments, the technology relates to modifying (e.g., editing) a target RNA using guide RNAs in complex with a CRISPR protein (e.g., an RNA-targeting affinity-tagged Cas13).

In embodiments, the particle further comprises nucleic acid(s) encoding or comprising one or both of crRNA and/or tracrRNA. crRNA contains the guide RNA that locates a specific region of a target DNA along with a region that binds to tracrRNA; together these form an active complex. In embodiments, the crRNA and tracrRNA are combined into a single-guide RNA (sgRNA).

Accordingly, in some embodiments, the technology relates to CRISPR protein/RNA RNP complexes comprising two RNA molecules: (1) a CRISPR RNA (crRNA), possessing a nucleotide sequence complementary to a target nucleotide sequence; and (2) a trans-activating crRNA (tracrRNA). In this mode, the CRISPR protein (e.g., Cas9) functions as an RNA-guided nuclease that uses both the crRNA and tracrRNA to recognize and cleave a target sequence. Recently, a single chimeric guide RNA (sgRNA) mimicking the structure of the annealed crRNA/tracrRNA has become more widely used than crRNA/tracrRNA because the gRNA approach provides a simplified system with only two components (e.g., the CRISPR protein and the sgRNA). Thus, sequence-specific binding of the RNP to a nucleic acid can be guided by a dual-RNA complex (e.g., a “dgRNA”), e.g., comprising a crRNA and a tracrRNA in two separate RNAs or by a chimeric single-guide RNA (e.g., a “sgRNA”) comprising a crRNA and a tracrRNA in a single RNA. (see, e.g., Jinek et al. (2012) “A Programmable Dual-RNA-Guided DNA Endonuclease in Adaptive Bacterial Immunity” Science 337:816-821, incorporated herein by reference).

As used herein, the targeting region of a crRNA (2-RNA dgRNA system) or a sgRNA (single guide system) is referred to as the “guide RNA” (gRNA). In some embodiments, the gRNA comprises, consists of, or essentially consists of 10 to 50 bases, e.g., 15 to 40 bases, e.g., 15 to 30 bases, e.g., 15 to 25 bases (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases). Methods are known in the art for determining the length of the gRNA that provides the most efficient target recognition for a CRISPR protein. See, e.g., Lee et al. (2016) “The Neisseria meningitidis CRISPR-Cas9 System Enables Specific Genome Editing in Mammalian Cells” Molecular Therapy 24: 645 (2016), incorporated herein by reference.

Accordingly, in some embodiments, the gRNA is a short synthetic RNA comprising a “scaffold sequence” (protein-binding segment) for binding to a CRISPR protein (e.g., a modified CRISPR protein) and a user-defined “DNA-targeting sequence” (nucleic acid-targeting segment) that is approximately 20-nucleotides long and is complementary to the target site of the target nucleic acid.

In some embodiments, nucleic acid targeting specificity is determined by two factors: 1) a nucleic acid (e.g., DNA) sequence matching the gRNA targeting sequence and a protospacer adjacent motif (PAM) directly downstream of the target sequence. Some RNP complexes (e.g., CRISPR protein/gRNA (e.g., Cas9/gRNA or modified Cas9/gRNA)) recognize a DNA sequence comprising a protospacer adjacent motif (PAM) sequence and an adjacent sequence comprising approximately 20 bases complementary to the gRNA. Canonical PAM sequences are NGG or NAG for Cas9 from Streptococcus pyogenes and NNNNGATT for the Cas9 from Neisseria meningitidis. In some embodiments, the technology comprises use of a Cas9 having an expanded PAM recognition (e.g., an xCas9 protein; see, e.g., Hu et al. (2018) “Evolved Cas9 variants with broad PAM compatibility and high DNA specificity” Nature 556: 57, incorporated herein by reference). Following recognition of a target site in a target nucleic acid by hybridization of the gRNA to the target sequence, the CRISPR protein (e.g., Cas9) cleaves the nucleic acid sequence via an intrinsic nuclease activity. For genome editing and other purposes, the CRISPR/Cas system from S. pyogenes has been used most often. Using this system, one can target a given target nucleic acid (e.g., for editing or other manipulation) by designing a gRNA comprising a nucleotide sequence complementary to a DNA sequence (e.g., a DNA sequence comprising approximately 20 nucleotides) that is 5′-adjacent to the PAM. Methods are known in the art for determining a PAM sequence that provides efficient target recognition for a Cas9 (and thus for a modified Cas9). See, e.g., Zhang et al. (2013) “Processing-independent CRISPR RNAs limit natural transformation in Neisseria meningitidis” Molecular Cell 50: 488-503, incorporated herein by reference; Lee et al., supra, incorporated herein by reference.

In some exemplary embodiments, the crRNA comprises a sequence according to SEQ ID NO: 1

NNNNNNNNNNNNrGrUrUrUrArArGrArGr
CrUrArUrGrCrUrGrUrUrUrUrG

where the “NNNNNNNNNNNN” represents the nucleic acid-targeting sequence that is complementary to the target sequence.

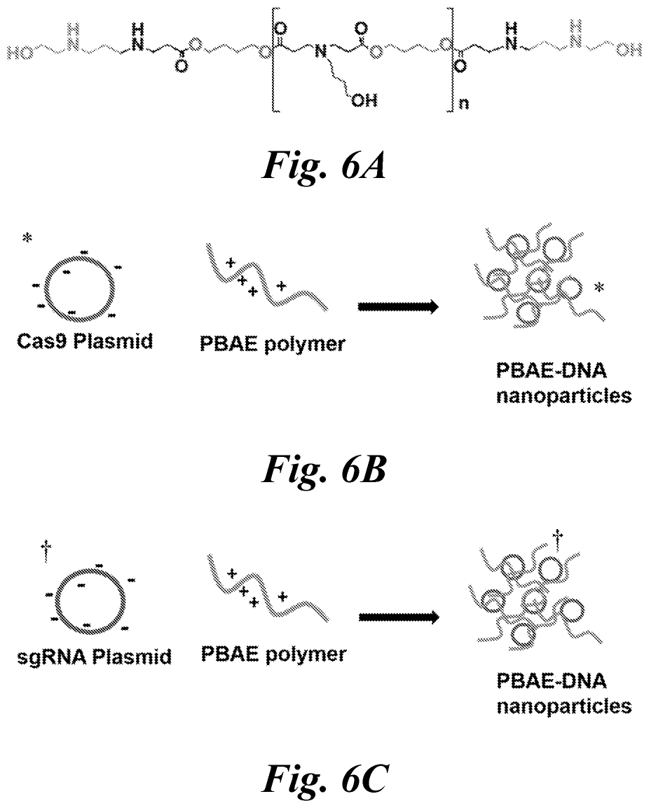

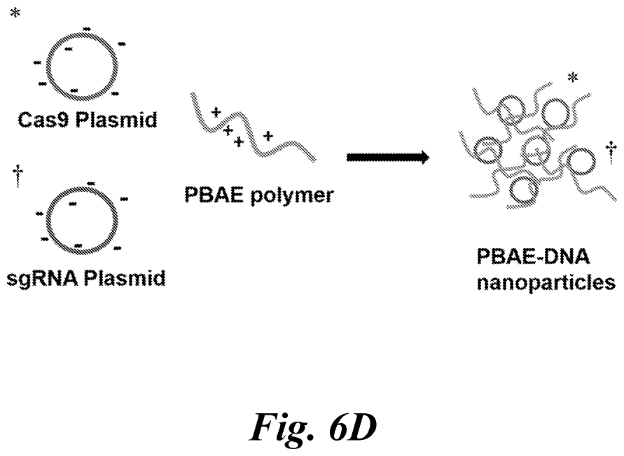

In some embodiments, the tracrRNA comprises a sequence of a naturally occurring tracrRNA, e.g., a provided by FIGS. 6, 35, and 37, and by SEQ ID NOs: 267-272 and 431-562 of U.S. Pat. App. Pub. No. 20170051312, incorporated herein by reference.

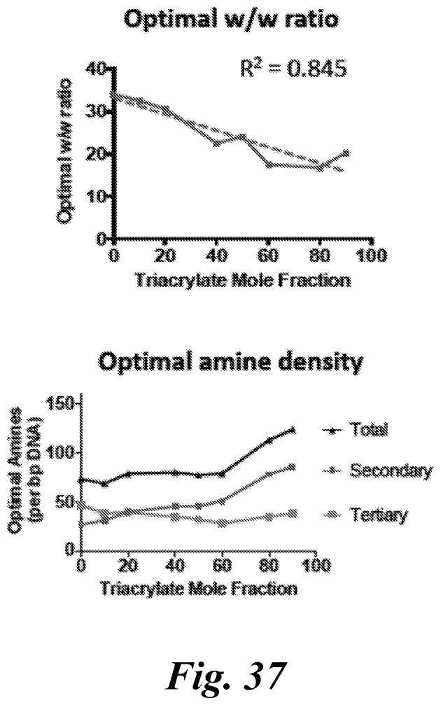

In some embodiments, the crRNA comprises a sequence that hybridizes to a tracrRNA to form a duplex structure, e.g., a sequence provided by FIG. 7 and SEQ ID NOs: 563-679 of U.S. Pat. App. Pub. No. 20170051312, incorporated herein by reference. In some embodiments, a crRNA comprises a sequence provided by FIG. 37 of U.S. Pat. App. Pub. No. 20170051312, incorporated herein by reference. In some embodiments, the duplex-forming segment of the crRNA is at least about 60% identical to one of the tracrRNA molecules set forth in SEQ ID NOs: 431-679 of U.S. Pat. App. Pub. No. 20170051312, incorporated herein by reference, or a complement thereof.

Thus, in some embodiments, exemplary (but not limiting) nucleotide sequences that are included in a dgRNA system include either of the sequences set forth in U.S. Pat. App. Pub. No. 20170051312, incorporated herein by reference, as SEQ ID NOs: 431-562, or complements thereof pairing with any sequences set forth in U.S. Pat. App. Pub. No. 20170051312, incorporated herein by reference, SEQ ID NOs: 563-679, or complements thereof that can hybridize to form a protein binding segment.

In some embodiments, a single-molecule gRNA (e.g., a sgRNA) comprises two complementary stretches of nucleotides that hybridize to form a dsRNA duplex. In some embodiments, the sgRNA (or a DNA encoding the sgRNA) is at least about 60% identical to one of the tracrRNA molecules set forth in U.S. Pat. App. Pub. No. 20170051312, incorporated herein by reference, as SEQ ID NOs: 431-562, or a complement thereof, over at least 8 contiguous nucleotides. In some embodiments, the sgRNA (or a DNA encoding the sgRNA) is at least about 60% identical to one of the tracrRNA molecules set forth in U.S. Pat. App. Pub. No. 20170051312, incorporated herein by reference, as SEQ ID NOs: 563-679, or a complement thereof, over at least 8 contiguous nucleotides. Appropriate naturally occurring pairs of crRNAs and tracrRNAs can be routinely determined by taking into account the species name and base-pairing (for the dsRNA duplex of the protein-binding domain) when determining appropriate cognate pairs.

In some embodiments, a gRNA comprises a first segment (also referred to herein as a “nucleic acid-targeting segment” or a “nucleic acid-targeting sequence”) and a second segment (also referred to herein as a “protein-binding segment” or a “protein-binding sequence”). In some embodiments, the nucleic acid-targeting segment is and/or comprises a DNA-targeting segment. In some embodiments, the nucleic acid-targeting sequence is and/or comprises a DNA-targeting sequence. In some embodiments, the nucleic acid-targeting segment is and/or comprises an RNA-targeting segment. In some embodiments, the nucleic acid-targeting sequence is and/or comprises an RNA-targeting sequence.

The nucleic acid-targeting segment (e.g., DNA-targeting segment or RNA-targeting segment) of a gRNA comprises a nucleotide sequence that is complementary to a sequence in a target nucleic acid (e.g., at the target site in a DNA or RNA). In other words, the nucleic acid-targeting segment of a gRNA interacts with a target nucleic acid (e.g., DNA or RNA) in a sequence-specific manner via hybridization (e.g., complementary base pairing). As such, the nucleotide sequence of the nucleic acid-targeting segment may vary and determines the location within the target nucleic acid (e.g., DNA or RNA) that the nucleic acid-targeting RNA and the target nucleic acid (e.g., DNA or RNA) will interact. The nucleic acid-targeting segment of a gRNA can be modified (e.g., by genetic engineering) to hybridize to any desired sequence within a target nucleic acid (e.g., DNA or RNA).

In some embodiments, the nucleic acid-targeting segment (e.g., DNA-targeting segment or RNA-targeting segment) has a length of from about 8 nucleotides to about 100 nucleotides. In some embodiments, the nucleic acid-targeting segment (e.g., DNA-targeting segment or RNA-targeting segment) comprises the nucleic acid-targeting sequence (e.g., DNA-targeting sequence or RNA-targeting sequence) and, in some embodiments, additional nucleic acid. For example, the nucleic acid-targeting segment can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the nucleic acid-targeting segment can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt.

In some embodiments, the nucleotide sequence (the nucleic acid-targeting sequence) of the nucleic acid-targeting segment (e.g., DNA-targeting segment or RNA-targeting segment) that is complementary to a nucleotide sequence (target sequence) of the target nucleic acid can have a length at least about 12 nt. For example, the nucleic acid-targeting sequence of the nucleic acid-targeting segment that is complementary to a target sequence of the target nucleic acid can have a length at least about 12 nt, at least about 15 nt, at least about 18 nt, at least about 19 nt, at least about 20 nt, at least about 25 nt, at least about 30 nt, at least about 35 nt or at least about 40 nt. For example, the nucleic acid-targeting sequence of the nucleic acid-targeting segment that is complementary to a target sequence of the target nucleic acid can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. The nucleotide sequence (the nucleic acid-targeting sequence) of the nucleic acid-targeting segment that is complementary to a nucleotide sequence (target sequence) of the target nucleic acid can have a length at least about 12 nt.

In additional embodiments, the nucleotide sequence (the nucleic acid-targeting sequence) of the nucleic acid-targeting segment (e.g., DNA-targeting segment or RNA-targeting segment) that is complementary to a nucleotide sequence (target sequence) of the target nucleic acid can have a length of from about 8 nucleotides to about 30 nucleotides. For example, the nucleic acid-targeting segment can have a length of from about 8 nucleotides (nt) to about 30 nt, from about 8 nt to about 30 nt, from about 8 nt to about 25 nt, from about 8 nt to about 20 nt, from about 8 nt to about 18 nt, from about 8 nt to about 15 nt, or from about 8 nt to about 12 nt, e.g., 8 nt, 9 nt, 10 nt, 11 nt, or 12 nt.

In some embodiments, the nucleic acid-targeting sequence of the nucleic acid-targeting segment (e.g., DNA-targeting segment or RNA-targeting segment) that is complementary to a target sequence of the target nucleic acid is 8-20 nucleotides in length. In some embodiments, the nucleic acid-targeting sequence of the nucleic acid-targeting segment that is complementary to a target sequence of the target nucleic acid is 9-12 nucleotides in length.

The percent complementarity between the nucleic acid-targeting sequence of the nucleic acid-targeting segment (e.g., DNA-targeting segment or RNA-targeting segment) and the target sequence of the target nucleic acid (e.g., DNA or RNA) can be at least 60% (e.g., at least 65%, at least 70%, at least 75%, at least 80%, at least 85%, at least 90%, at least 95%, at least 97%, at least 98%, at least 99%, or 100%). In some embodiments, the percent complementarity between the nucleic acid-targeting sequence of the nucleic acid-targeting segment and the target sequence of the target nucleic acid is 100% over the seven contiguous 5′-most nucleotides of the target sequence of the complementary strand of the target nucleic acid. In some embodiments, the percent complementarity between the nucleic acid-targeting sequence of the nucleic acid-targeting segment and the target sequence of the target nucleic acid is at least 60% over about 20 contiguous nucleotides. In some embodiments, the percent complementarity between the nucleic acid-targeting sequence of the nucleic acid-targeting segment and the target sequence of the target nucleic acid is 100% over the fourteen contiguous 5′-most nucleotides of the target sequence of the complementary strand of the target DNA and as low as 0% over the remainder. In such a case, the nucleic acid-targeting sequence can be considered to be 14 nucleotides in length. In some embodiments, the percent complementarity between the nucleic acid-targeting sequence of the nucleic acid-targeting segment and the target sequence of the target nucleic acid is 100% over the seven contiguous 5′-most nucleotides of the target sequence of the complementary strand of the target nucleic acid and as low as 0% over the remainder. In such a case, the nucleic acid-targeting sequence can be considered to be 7 nucleotides in length.

The protein-binding segment of a gRNA interacts with a polypeptide, e.g., a CRISPR protein or an modified CRISPR protein (e.g., a Cas9 or Cas9-like polypeptide and/or modified versions thereof). The gRNA guides the bound polypeptide to a specific nucleotide sequence within target nucleic acid (e.g., target DNA or target RNA) via the above mentioned nucleic acid-targeting segment. The protein-binding segment of a gRNA comprises two segments comprising nucleotide sequences that are complementary to one another. The complementary nucleotides of the protein-binding segment hybridize to form a double stranded RNA duplex.

A dgRNA comprises two separate RNA molecules. Each of the two RNA molecules of a dgRNA comprises a segment is complementary to one another such that the complementary nucleotides of the two RNA molecules hybridize to form the double stranded RNA duplex of the protein-binding segment.

In some embodiments, the duplex-forming segment of the activator-RNA is at least about 60% identical to one of the activator-RNA (tracrRNA) molecules set forth in U.S. Pat. App. Pub. No. 20170051312, incorporated herein by reference, as SEQ ID NOs: 431-562, or a complement thereof, over a segment of at least 8 contiguous nucleotides. For example, the duplex-forming segment of the activator-RNA (or the DNA encoding the duplex-forming segment of the activator-RNA) is at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical, to one of the tracrRNA sequences set forth in U.S. Pat. App. Pub. No. 20170051312, incorporated herein by reference, as SEQ ID NOs: 431-562, or a complement thereof, over a segment of at least 8 contiguous nucleotides.

In some embodiments, the duplex-forming segment of the targeter-RNA is at least about 60% identical to one of the targeter-RNA (crRNA) sequences set forth in U.S. Pat. App. Pub. No. 20170051312, incorporated herein by reference, as SEQ ID NOs: 563-679, or a complement thereof, over a segment of at least 8 contiguous nucleotides. For example, the duplex-forming segment of the targeter-RNA (or the DNA encoding the duplex-forming segment of the targeter-RNA) is at least about 65% identical, at least about 70% identical, at least about 75% identical, at least about 80% identical, at least about 85% identical, at least about 90% identical, at least about 95% identical, at least about 98% identical, at least about 99% identical or 100% identical to one of the crRNA sequences set forth in U.S. Pat. App. Pub. No. 20170051312, incorporated herein by reference, as SEQ ID NOs: 563-679, or a complement thereof, over a segment of at least 8 contiguous nucleotides.

Non-limiting examples of nucleotide sequences that can be included in a two-molecule nucleic acid-targeting RNA (dgRNA) include any of the sequences set forth in U.S. Pat. App. Pub. No. 20170051312, incorporated herein by reference, as SEQ ID NOs: 431-562, or complements thereof pairing with any sequences set forth in U.S. Pat. App. Pub. No. 20170051312, incorporated herein by reference, as SEQ ID NOs: 563-679, or complements thereof that can hybridize to form a protein binding segment.

A single-molecule nucleic acid-targeting RNA (sgRNA) comprises two segments of nucleotides (a targeter-RNA and an activator-RNA) that are complementary to one another, are covalently linked by intervening nucleotides (“linkers” or “linker nucleotides”), and hybridize to form the double stranded RNA duplex (dsRNA duplex) of the protein-binding segment, thus resulting in a stem-loop structure. The targeter-RNA and the activator-RNA can be covalently linked via the 3′ end of the targeter-RNA and the 5′ end of the activator-RNA. Alternatively, targeter-RNA and the activator-RNA can be covalently linked via the 5′ end of the targeter-RNA and the 3′ end of the activator-RNA.

The linker of a single-molecule nucleic acid-targeting RNA can have a length of from about 3 nucleotides to about 100 nucleotides. For example, the linker can have a length of from about 3 nucleotides (nt) to about 90 nt, from about 3 nucleotides (nt) to about 80 nt, from about 3 nucleotides (nt) to about 70 nt, from about 3 nucleotides (nt) to about 60 nt, from about 3 nucleotides (nt) to about 50 nt, from about 3 nucleotides (nt) to about 40 nt, from about 3 nucleotides (nt) to about 30 nt, from about 3 nucleotides (nt) to about 20 nt or from about 3 nucleotides (nt) to about 10 nt. For example, the linker can have a length of from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. In some embodiments, the linker of a single molecule nucleic acid-targeting RNA is 4 nt.

An exemplary single-molecule nucleic acid-targeting RNA comprises two complementary segments of nucleotides that hybridize to form a dsRNA duplex. In some embodiments, one of the two complementary segments of nucleotides of the single-molecule nucleic acid-targeting RNA (or the DNA encoding the segment) is at least about 60% identical to one of the activator-RNA (tracrRNA) molecules set forth in U.S. Pat. App. Pub. No. 20170051312, incorporated herein by reference, as SEQ ID NOs: 431-562, or a complement thereof, over a segment of at least 8 contiguous nucleotides. For example, one of the two complementary segments of nucleotides of the single-molecule nucleic acid-targeting RNA (or the nucleic acid (e.g., DNA) encoding the segment) is at least about 65% identical, at least about 70% identical, at least about 75% identical, at least about 80% identical, at least about 85% identical, at least about 90% identical, at least about 95% identical, at least about 98% identical, at least about 99% identical or 100% identical to one of the tracrRNA sequences set forth in U.S. Pat. App. Pub. No. 20170051312, incorporated herein by reference, as SEQ ID NOs: 431-562, or a complement thereof, over a segment of at least 8 contiguous nucleotides.

In some embodiments, one of the two complementary segments of nucleotides of the single molecule nucleic acid-targeting RNA (or the nucleic acid encoding the segment) is at least about 60% identical to one of the targeter-RNA (crRNA) sequences set forth in U.S. Pat. App. Pub. No. 20170051312, incorporated herein by reference, as SEQ ID NOs: 563-679, or a complement thereof, over a segment of at least 8 contiguous nucleotides. For example, one of the two complementary segments of nucleotides of the single-molecule DNA-targeting RNA (or the DNA encoding the segment) is at least about 65% identical, at least about 70% identical, at least about 75% identical, at least about 80% identical, at least about 85% identical, at least about 90% identical, at least about 95% identical, at least about 98% identical, at least about 99% identical or 100% identical to one of the crRNA sequences set forth in U.S. Pat. App. Pub. No. 20170051312, incorporated herein by reference, as SEQ ID NOs: 563-679, or a complement thereof, over a stretch of at least 8 contiguous nucleotides.

With regard to both a sgRNA and a dgRNA, artificial sequences that share a wide range of identity (approximately at least 50% identity) with naturally occurring tracrRNAs and crRNAs function with CRISPR proteins and modified CRISPR proteins (e.g., Cas9, Cas9-like proteins, modified Cas9, and modfied Cas9-like proteins) to deliver RNP to target nucleic acids with sequence specificity, particularly provided that the structure of the protein-binding domain of the nucleic acid-targeting RNA is conserved. Thus, information and modeling relating to RNA folding and RNA secondary structure of a naturally occurring protein-binding domain of a nucleic acid-targeting RNA provides guidance to design artificial protein-binding domains (either in dgRNA or sgRNA). As a non-limiting example, a functional artificial nucleic acid-targeting RNA may be designed based on the structure of the protein-binding segment of a naturally occurring nucleic acid-targeting segment of an RNA (e.g., including the same or similar number of base pairs along the RNA duplex and including the same or similar “bulge” region as present in the naturally occurring RNA). Structures can readily be produced by one of ordinary skill in the art for any naturally occurring crRNA:tracrRNA pair from any species; thus, in some embodiments an artificial nucleic acid-targeting-RNA is designed to mimic the natural structure for a given species when using the Cas9 (or a related Cas9) from that species. Thus, in some embodiments, a suitable nucleic acid-targeting RNA is an artificially designed RNA (non-naturally occurring) comprising a protein-binding domain that was designed to mimic the structure of a protein-binding domain of a naturally occurring nucleic acid-targeting RNA. In exemplary embodiments, the protein-binding segment has a length of from about 10 nucleotides to about 100 nucleotides; e.g., the protein-binding segment has a length of from about 15 nucleotides (nt) to about 80 nt, from about 15 nt to about 50 nt, from about 15 nt to about 40 nt, from about 15 nt to about 30 nt or from about 15 nt to about 25 nt.

Nucleic acids can be analyzed and designed using a variety of computer tools, e.g., Vector NTI (Invitrogen) for nucleic acids and AlignX for comparative sequence analysis of proteins. Further, in silico modeling of RNA structure and folding can be performed using the Vienna RNA package algorithms and RNA secondary structures and folding models can be predicted with RNAfold and RNAcofold, respectively, and visualized with VARNA. See, e.g., Denman (1993), Biotechniques 15, 1090; Hofacker and Stadler (2006), Bioinformatics 22, 1172; and Darty and Ponty (2009), Bioinformatics 25, 1974, each of which is incorporated herein by reference.

Thus, as described herein, in some embodiments, the technology provides methods, systems, kits, compositions, uses, etc. comprising and/or comprising use of a RNP comprising a polypeptide and one or more RNAs. In some embodiments, the RNA comprises a segment (e.g., comprising 6-10 nucleotides, e.g., comprising 6, 7, 8, 9, or 10 nucleotides) that is complementary (e.g., at least 80, 81, 82, 83, 84, 85, 86, 87, 88, 89, 90, 91, 92, 93, 94, 95, 96, 97, 98, 98.5, 99, 99.1, 99.2, 99.3, 99.4, 99.5, 99.6, 99.7, 99.8, 99.9, or 100% complementary) to a nucleotide sequence in the target nucleic acid.

In some embodiments, the RNA comprises a segment comprising a nucleotide sequence (e.g., a scaffold sequence, e.g., a sequence that interacts with (e.g., binds to) the polypeptide) that is at least 60% identical over at least 8 contiguous nucleotides to any one of the nucleotide sequences set forth in SEQ ID NOs: 431-682 (e.g., SEQ ID NOs: 431-562) of U.S. Pat. App. Pub. No. 20170051312, incorporated herein by reference. In some embodiments, the RNA comprises a nucleotide sequence (e.g., a scaffold sequence, e.g., a sequence that interacts with (e.g., binds to) the polypeptide) that is at least 60% identical over at least 8 contiguous nucleotides to any one of the nucleotide sequences set forth in SEQ ID NOs: 563-682 of U.S. Pat. App. Pub. No. 20170051312, incorporated herein by reference.

In some embodiments, the polypeptide comprises a segment comprising an amino acid sequence that is at least approximately 75% amino acid identical to amino acids 7-166 or 731-1003 of any of the amino acid sequences set forth as SEQ ID NOs: 1-256 and 795-1346 of U.S. Pat. App. Pub. No. 20170051312, incorporated herein by reference.

When Cas9 is associated with its gRNA (or components thereof), e.g., to form a ribonucleoprotein (RNP), it is able to modify a specific region of a nucleic acid (e.g., a DNA and/or an RNA) by single-strand nicking, double-strand break, and/or DNA binding.

Accordingly, in some embodiments, the technology comprises use of a ribonucleoprotein (RNP) comprising a CRISPR protein. In some embodiments, the technology comprises use of a RNP complex comprising a Cas9 or Cas9-like protein and one or more RNA molecules (e.g., a gRNA (e.g., a nucleic acid-targeting RNA, an activator-RNA and a targeter-RNA, a crRNA and a tracrRNA; a dgRNA; a sgRNA)). In some embodiments, the technology comprises use of a ribonucleoprotein (RNP) complex comprising a Cas9 or Cas9-like protein as described herein and one or more RNA molecules (e.g., a gRNA (e.g., a nucleic acid-targeting RNA, an activator-RNA and a targeter-RNA, a crRNA and a tracrRNA; a dgRNA; a sgRNA)).

In some embodiments, the technology comprises use of a plurality of RNPs, e.g., to produce multiple double-stranded breaks in a nucleic acid. For instance, in some embodiments the technology comprises use of a first RNP comprising a CRISPR protein (e.g., Cas9 or Cas9-like protein) and a first RNA molecule or first set of RNA molecules (e.g., a gRNA (e.g., a nucleic acid-targeting RNA, an activator-RNA and a targeter-RNA, a crRNA and a tracrRNA; a dgRNA; a sgRNA)) and a second RNP comprising a CRISPR protein (e.g., a Cas9 or Cas9-like protein) and a second RNA molecule or second set of RNA molecules (e.g., a gRNA (e.g., a nucleic acid-targeting RNA, an activator-RNA and a targeter-RNA, a crRNA and a tracrRNA; a dgRNA; a sgRNA)).

The RNA provides target specificity to the RNP complex by comprising a nucleotide sequence that is complementary to a target sequence of a target nucleic acid. The polypeptide of the complex (e.g., a CRISPR protein) provides binding and nuclease activity. In other words, the polypeptide is guided to a nucleic acid sequence (e.g., a DNA sequence (e.g., a chromosomal sequence, an extrachromosomal sequence (e.g., an episomal sequence, a minicircle sequence, a mitochondrial sequence, a chloroplast sequence, etc.), a cDNA sequence) or an RNA sequence (e.g., a transcript sequence, a functional RNA sequence)) by virtue of its association with at least the protein-binding segment of the nucleic acid-targeting RNA.

In embodiments, a particle of the present technology further comprises a nucleic acid comprising a homologous template (e.g., a “donor nucleic acid”). The homologous template may be a repair template which comprises a wild-type version of a target DNA or it may comprise a mutated version of the target DNA. For example, a homologous template may comprise a polynucleotide that is at least about 70% homologous with a sequence that is within 10 kb of a target site of a gene-editing endonuclease. When a homologous template is used, CRISPR allows insertion of the homologous sequence into a specific target DNA location, thereby repairing a mutated gene and/or otherwise modifying a genomic sequence.

In some embodiments, the technology comprises use of a donor nucleic acid, e.g., a DNA molecule. In some embodiments, the donor molecule participates in the homology directed repair (HDR) pathway to “repair” a double-stranded break with a sequence from the donor. In this way, CRISPR finds use to make targeted insertions of a particular nucleic acid sequence at a target site, e.g., to produce a “knock-in”.

In some embodiments, the donor nucleic acid is double stranded. In some embodiments, the donor nucleic acid is single stranded. In some embodiments, a donor DNA molecule is a linear molecule (e.g., not a circular molecule such as a plasmid DNA).

A donor DNA molecule can have any desired sequence. In some embodiments, the donor nucleic acid comprises a portion comprising a nucleic acid to be knocked-in at a target locus (e.g., in some embodiments, the donor nucleic acid comprises a portion comprising an insertion sequence). In some embodiments, the 3′ most nucleotide on at least one end of the donor DNA molecule is a C. In some embodiments, the 3′ most nucleotide on one and only one end of the donor DNA molecule is a C. In some embodiments, the 3′ most nucleotide on at least one end of the donor DNA molecule is a G. In some embodiments, the 3′ most nucleotide on one and only one end of the donor DNA molecule is a G. In some embodiments, the 3′ most nucleotide on at least one end of the donor DNA molecule is an A. In some embodiments, the 3′ most nucleotide on one and only one end of the donor DNA molecule is an A. In some embodiments, the 3′ most nucleotide on at least one end of the donor DNA molecule is a T. In some embodiments, the 3′ most nucleotide on one and only one end of the donor DNA molecule is a T.

In some embodiments, the linear donor (e.g., DNA) molecule has a length in a range of from 10 to 1000 nucleotides (nt) (e.g., 15 to 500, 20 to 500, 30 to 500, 33 to 500, 35 to 500, 40 to 500, 45 to 500, 50 to 500, 15 to 250, 20 to 250, 30 to 250, 33 to 250, 35 to 250, 40 to 250, 45 to 250, 50 to 250, 15 to 150, 20 to 150, 30 to 150, 33 to 150, 35 to 150, 40 to 150, 45 to 150, 50 to 150, 15 to 100, 20 to 100, 30 to 100, 33 to 100, 35 to 100, 40 to 100, 45 to 100, 50 to 100, 15 to 50, 20 to 50, 30 to 50, 33 to 50, 35 to 50, 40 to 50, or 45 to 50 nt). In some embodiments, the linear donor nucleic acid has a length of 1 Kbp or more (e.g., 1 to 10 Kbp (e.g., 1, 1.5, 2, 2.5, 3, 3.5, 4, 4.5, 5, 5.5, 6, 6.5, 7, 7.5, 8, 8.5, 9, 9.5, or 10 Kbp).

In some embodiments, a method includes introducing into a cell (e.g., according to the nanoparticle technology provide herein) a subject linear donor DNA molecule.

In some embodiments, the linear donor DNA molecule includes a 3′-overhang. For example, In some embodiments, the linear donor DNA molecule includes a 3′-overhang having a length in a range of from 1 to 6 nucleotides (nt) (e.g., 1 to 5 nt, 1 to 4 nt, 1 to 3 nt, 1 to 2 nt, 2 to 6 nt, 2 to 5 nt, 2 to 4 nt, 2 to 3 nt, 3 to 6 nt, 3 to 5 nt, 3 to 4 nt, 4 to 6 nt, 4 to 5 nt, 5 to 6 nt, 1 nt, 2 nt, 3 nt, 4 nt, 5 nt, or 6 nt). In some embodiments, the linear donor DNA molecule does not have a 3′-overhang. Thus, In some embodiments, the linear donor DNA molecule includes a 3′-overhang having a length in a range of from 0 to 6 nucleotides (nt) (e.g., 0 to 5 nt, 0 to 4 nt, 0 to 3 nt, 0 to 2 nt, 0 to 1 nt, 1 to 6 nt, 1 to 5 nt, 1 to 4 nt, 1 to 3 nt, 1 to 2 nt, 2 to 6 nt, 2 to 5 nt, 2 to 4 nt, 2 to 3 nt, 3 to 6 nt, 3 to 5 nt, 3 to 4 nt, 4 to 6 nt, 4 to 5 nt, 5 to 6 nt, 1 nt, 2 nt, 3 nt, 4 nt, 5 nt, or 6 nt).

In embodiments, the nucleic acid encodes Cre-Recombinase or FLP-Recombinase. These two enzymes target specific recognition sequences (LoxP sites for Cre and FRT sites for FLP) and delete/excise the DNA located between recognition sequences. Cre and FLP are useful for knocking out gene activity in model organisms, which have been previously been engineered to contain LoxP or FRT sites in their genome. Accordingly, the present technology is useful for creating knockout animals.

In embodiments, the nucleic acid encodes a meganuclease. Meganucleases are described, at least, in U.S. Pat. No. 7,842,489, which is hereby incorporated by reference. Meganucleases are endodeoxyribonucleases characterized by a large recognition site of 12 to 40 base pairs, which statistically, should occur only once in a given genome. By customizing its target recognition domain through protein engineering, a meganuclease can replace, eliminate, or modify sequences in a highly specific way.

In some embodiments, the nucleic acid encodes a TALEN. TALENs are described, at least, in US 2011/0145940 and U.S. Pat. No. 9,393,257, which are hereby incorporated by reference. TALENs are fusion proteins comprising a transcription activator-like effectors (TALE) DNA-binding domain and a DNA nuclease domain (which cuts DNA strands). TALEs can be engineered to bind any desired DNA sequence. Thus, the TALEN, via its DNA nuclease domain, cuts DNA at specific locations.

In embodiments, the nucleic acid encodes a ZFN. ZFNs are described, at least, in US 2005/0208489, which is hereby incorporated by reference. ZFNs are artificial restriction enzymes generated by fusing a zinc finger DNA-binding domain to a DNA-cleavage domain. The zinc finger domain is designed to target a specific DNA sequence. Thus, the ZFN, via its DNA-cleavage domain, is able to precisely modify genes and/or genomic sequences. In embodiments, a particle of the present technology further comprises a nucleic acid comprising a homologous template, which may be a repair template that comprises a wild-type version of a target DNA or may comprise a mutated version of the target DNA. Thus, in embodiments, the ZFN allows insertion of the homologous sequence into a specific target DNA location, thereby repairing a mutated gene and/or otherwise modifying a genomic sequence.

In embodiments a gene-editing protein comprises a nuclear-localization sequence or a mitochondrial-localization sequence.

### E. Gene Therapy Methods for Treating a Retinal Eye Disease

Generally, gene therapy involves the therapeutic delivery of a gene or gene-modifying technology to a cell to treat an underlying disease or condition. Such technology includes replacing a mutated gene that cause the disease or condition with a healthy copy of it, inactivating, or “knocking out” a mutated gene, or introducing a new gene that acts against the disease or mediates the condition. In some embodiments, the presently disclosed subject matter provides a method for treating a retinal eye disease, including a hereditary retinal eye disease.

In certain embodiments, the nanoparticle targeting (through biomaterial selection, nanoparticle biophysical properties, and/or a targeting ligand) is combined with transcriptional targeting of a therapeutic gene to a particular cell type (e.g., cancer cells). Transcriptional targeting includes designing nucleic acid cargo which comprises a promoter that is active in cells or tissue types of interest so that the delivered nanoparticles express the nucleic acid cargo in a tissue-specific manner. To this end, in some embodiments, the nucleic acid is operably linked to a constitutive promoter or a cancer-specific promoter.

A “promoter” is a DNA sequence that directs the binding of RNA polymerase and thereby promotes RNA synthesis. A nucleic acid sequence is “operably linked” to a promoter when the promoter is capable of directing transcription of that nucleic acid sequence. A promoter can be native or non-native to the nucleic acid sequence to which it is operably linked. Techniques for operably linking sequences together are well known in the art. The term “constitutive promoter,” as used herein, refers to an unregulated promoter that allows for continual transcription of its associated gene in a variety of cell types. Suitable constitutive promoters are known in the art and can be used in connection with the present disclosure.

In some embodiments, the cell is transfected with the particles for ex vivo gene therapy. In some embodiments, the particles are delivered directly to an organism, such as mammalian subject, to thereby direct gene therapy in vivo.

In particular embodiments, the presently disclosed particles carry plasmid DNA comprising a nucleic acid sequence encoding a SR39 thymidine kinase to a cancer cell. The cell may be a eukaryotic cell, such as an animal cell or plant cell. In further embodiments, the animal cell is a mammalian cell (e.g., a human cell).

In some embodiments, including for delivery of nucleic acids to cells ex vivo, the cell is a stem cell or progenitor cell. The cell may be multipotent or pluripotent. In some embodiments, the cell is a stem cell, such as an embryonic stem cell or adult stem cell. In some embodiments, the cell is a hematopoietic stem cell.

For in vivo gene therapy, particles can be formulated for a variety of modes of administration, including systemic and topical or localized administration. Thus, the pharmaceutical compositions can be formulated for administration to patients by any appropriate route, including intravenous administration, intra-arterial administration, subcutaneous administration, intradermal administration, intralymphatic administration, and intra-tumoral administration. In some embodiments, the composition is lyophilized and reconstituted prior to administration.

In particular embodiments, the retinal eye disease is selected from age-related macular degeneration (AMD), including wet macular degeneration and dry macular degeneration, Leber's congenital amaurosis (LCA2) type 2, choroideremia, achromatopsia, retinitis pigmentosa (RP), Stargardt disease (STGD), Usher syndrome, juvenile X-linked retinoschisis (XLRS), and diabetic retinopathy.

The presently disclosed composition can be administered via direct injection into the anterior chamber (intra-cameral injection), sub-conjunctival injection, intravitreal injection, and subretinal injection. In particular, the composition is delivered to one or more cells of the retinal pigmented epithelium (RPE).

Representative approaches to gene therapy for retinal eye diseases are summarized in Table 1. See Samiy, Gene Therapy for Retinal Diseases, J. Ophthalmic Vis Res. 2014 October-December; 9(4):506-509; Campa et al., The Role of Gene Therapy in the Treatment of Retinal Diseases: A Review, Current Gene Therapy, 2017, 17, 194-213.

TABLE 1
Approaches to Gene Therapy for Retinal Eye Disease

| Disease | Agent |
|---|---|
| Achromatopsia (ACHM) | CNGA3 |
| | CNGB3 |
| | GNAT2 |
| Age-related macular degeneration (AMD) | sFLT01 |
| Choroideremia (CHM) | Rab Escort Protein (REP-1) |
| Juvenile X-linked retinoschisis (XLRS) | RS-1 |
| Leber's congenital amaurosis (LCA) | hRPE65 v2 cDNA |
| Retinitis pigmentosa (RP) | RPGR |
| | MY07A |
| | MERTK |
| Stargardt Disease (STGD) | ATP-binding cassette |
| | transporter 4 (ABCA4) |
| Usher's syndrome (USH) | SAR-421869 (UshStat ® |
| | MY07A |

More particularly, in some embodiments, the presently disclosed subject matter provides a method for treating a retinal eye disease, the method comprising administering to a subject in need of treatment thereof, a composition of Formula (I) or Formula (II), wherein the composition comprises a therapeutic protein for treating retinal eye disease.

In some embodiments, the retinal eye disease comprises a hereditary retinal eye disease. In particular embodiments, the retinal eye disease is selected from the group consisting of age-related macular degeneration (AMD), including wet macular degeneration and dry macular degeneration, Leber's congenital amaurosis (LCA2) type 2, choroideremia, achromatopsia, retinitis pigmentosa (RP), Stargardt disease (STGD), Usher syndrome, juvenile X-linked retinoschisis (XLRS), and diabetic retinopathy.

In certain embodiments, the therapeutic protein is selected from the group consisting of CNGA3, CNGB3, GNAT2, sFLT01, Rab Escort Protein (REP-1), RS-1, RPE65, RPGR, MY07A, MERTK, ATP-binding cassette transporter 4 (ABCA4), and SAR-421869.

In yet more certain embodiments, the nucleic acid associated with retinal eye disease is administered via an injection technique selected from the group consisting of intra-cameral injection, sub-conjunctival injection, intravitreal injection, and subretinal injection. In particular embodiments, the composition is delivered to one or more cells of a retinal pigmented epithelium (RPE) of the subject.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

As used herein, the term “inhibit,” and grammatical derivations thereof, refers to the ability of a presently disclosed compound, e.g., a presently disclosed compound of formula (I), to block, partially block, interfere, decrease, or reduce the growth and/or metastasis of a cancer cell. Thus, one of ordinary skill in the art would appreciate that the term “inhibit” encompasses a complete and/or partial decrease in the growth and/or metastasis of a cancer cell, e.g., a decrease by at least 10%, in some embodiments, a decrease by at least 20%, 30%, 50%, 75%, 95%, 98%, and up to and including 100%.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly a compound of formula (I) and at least one therapeutic agent and/or imaging agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compositions of formula (I) or formula (II) described herein can be administered alone or in combination with adjuvants that enhance stability of the compositions of formula (I) or formula (II), alone or in combination with one or more therapeutic agents and/or imaging agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a composition of formula (I) or formula (II) and at least one additional therapeutic agent can be varied so long as the beneficial effects of the combination of these agents are achieved. Accordingly, the phrase “in combination with” refers to the administration of a composition of formula (I) or formula (II) and at least one additional therapeutic agent either simultaneously, sequentially, or a combination thereof. Therefore, a subject administered a combination of a composition of formula (I) a or formula (II) nd at least one additional therapeutic agent can receive composition of formula (I) or formula (II) and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the composition of formula (I) and at least one additional therapeutic agent are administered simultaneously, they can be administered to the subject as separate pharmaceutical compositions, each comprising either a composition of formula (I) or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a composition of formula (I) and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

Qₐ/Q_{A}+Q_{b}/Q_{B}=Synergy Index (SI)

wherein:

Q_{A} is the concentration of a component A, acting alone, which produced an end point in relation to component A;

Qₐ is the concentration of component A, in a mixture, which produced an end point;

Q_{B} is the concentration of a component B, acting alone, which produced an end point in relation to component B; and

Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

### F. Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

As used herein, the term “CRISPR activity” refers to an activity associated with a CRISPR system. Examples of such activities are sequence-specific binding, double-stranded nuclease activity, nickase activity, transcriptional activation, transcriptional repression, nucleic acid methylation, nucleic acid demethylation, and recombinase.

Furthermore, as used herein, the term “CRISPR system” refers to a collection of CRISPR proteins and nucleic acid that, when combined (e.g., to form a RNP (e.g., a CRISPR complex)), result in at least CRISPR-associated activity (e.g., the target locus specific, double-stranded cleavage of double-stranded DNA). For instance, in some embodiments, “CRISPR system” refers collectively to transcripts and other elements involved in the expression of and/or directing the activity of CRISPR-associated (“Cas”) genes, including sequences encoding a Cas gene, dCas gene, Cas nickase, Cas homolog, Cpf1 gene, Cas13, and/or modified versions of any of the foregoing; a tracr (trans-activating CRISPR) sequence (e.g., tracrRNA or an active partial tracrRNA); a cr (CRISPR) sequence (e.g., crRNA or an active partial crRNA); and/or other sequences and transcripts from a CRISPR locus. In some embodiments of the technology, the terms “guide sequence” and “guide RNA” (gRNA) are used interchangeably. In some embodiments, one or more elements of a CRISPR system is/are derived from a type I, type II, or type III CRISPR system. In some embodiments, one or more elements of a CRISPR system is/are derived from a particular organism comprising an endogenous CRISPR system, such as Streptococcus pyogenes. In general, a CRISPR system is characterized by elements that promote the formation of a CRISPR RNP complex (e.g., in vitro or in vivo) and direct it to the site of a target sequence (e.g., in a cell (e.g., after introduction of the RNP) and/or in vitro).

As used herein, the term “CRISPR complex” refers to the CRISPR proteins and nucleic acid (e.g., RNA) that associate with each other to form an aggregate (e.g., an RNP) that has functional activity. An example of a CRISPR complex is a wild-type Cas9 (sometimes referred to as Csn1) protein or Cas9-like protein that is bound to a guide RNA specific for a target locus.

As used herein, the term “CRISPR protein” refers to a protein comprising a nucleic acid (e.g., RNA (e.g., gRNA)) binding domain and an effector (e.g., nuclease) domain (e.g., Cas9 (e.g., Streptococcus pyogenes Cas9) and modified versions thereof). The nucleic acid binding domains interact with a first nucleic acid molecule either having a region capable of hybridizing to a desired target nucleic acid (e.g., a guide RNA) or that associates with a second nucleic acid having a region capable of hybridizing to the desired target nucleic acid (e.g., a crRNA). In some embodiments, a CRISPR protein comprises a nuclease domain (e.g., DNase or RNase domain), one or more additional DNA binding domains, a helicase domain, a protein-protein interaction domain, a dimerization domain, an affinity tag, as well as one or more other domains. In some embodiments, “CRISPR protein” refers to a plurality of proteins that form a complex that binds the first nucleic acid molecule referred to above. Thus, one CRISPR protein may bind to, for example, a guide RNA and another protein may have endonuclease activity. These are all considered to be CRISPR proteins because they function as part of a complex that performs the same functions as a single protein such as Cas9. In some embodiments, CRISPR proteins comprise nuclear localization signals (NLS) that allow them to be transported to the nucleus.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982), incorporated herein by reference). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 2002, 41(14), 4503-4510, incorporated herein by reference) and U.S. Pat. No. 5,034,506, incorporated herein by reference), locked nucleic acid (LNA; see Wahlestedt et al., Proc. Natl. Acad. Sci. U.S.A., 2000, 97, 5633-5638, incorporated herein by reference), cyclohexenyl nucleic acids (see Wang, J. Am. Chem. Soc., 2000, 122, 8595-8602, incorporated herein by reference), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand.

Furthermore, the terms “nucleic acid”, “polynucleotide”, “nucleotide sequence”, and “oligonucleotide” are used interchangeably. They refer to a polymeric form of nucleotides of any length, either deoxyribonucleotides or ribonucleotides, or analogs thereof. Polynucleotides may have any three dimensional structure and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996, each of which is incorporated herein by reference. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “nucleotide analog” as used herein refers to modified or non-naturally occurring nucleotides including but not limited to analogs that have altered stacking interactions such as 7-deaza purines (e.g., 7-deaza-dATP and 7-deaza-dGTP); base analogs with alternative hydrogen bonding configurations (e.g., such as Iso-C and Iso-G and other non-standard base pairs described in U.S. Pat. No. 6,001,983, herein incorporated by reference); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242, B. A. Schweitzer and E. T. Kool, J. Am. Chem. Soc., 1995, 117, 1863-1872; each of which is herein incorporated by reference); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152, each of which is incorporated herein by reference).

Nucleotide analogs include nucleotides having modification on the sugar moiety, such as dideoxy nucleotides and 2′-O-methyl nucleotides. Nucleotide analogs include modified forms of deoxyribonucleotides as well as ribonucleotides.

As used herein, the term “peptide nucleic acid” means a DNA mimic that incorporates a peptide-like polyamide backbone.

As used herein, the term “% sequence identity” refers to the percentage of nucleotides or nucleotide analogs in a nucleic acid sequence that is identical with the corresponding nucleotides in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. Methods and computer programs for alignment are well known in the art, including BLAST, Align 2, and FASTA.

The term “homology” and “homologous” refers to a degree of identity. There may be partial homology or complete homology. A partially homologous sequence is one that is less than 100% identical to another sequence.

The term “sequence variation” as used herein refers to a difference or multiple differences in nucleic acid sequence between two nucleic acids. For example, a wild-type structural gene and a mutant form of this wild-type structural gene may vary in sequence by the presence of one or more single base substitutions or by deletions and/or insertions of one or more nucleotides. These two forms of the structural gene are said to vary in sequence from one another. A second mutant form of the structural gene may exist. This second mutant form is said to vary in sequence from both the wild-type gene and the first mutant form of the gene.

As used herein, the terms “complementary” or “complementarity” are used in reference to polynucleotides (e.g., a sequence of nucleotides such as an oligonucleotide or a target nucleic acid) related by the base-pairing rules. For example, for the sequence “5′-A-G-T-3′” is complementary to the sequence “3′-T-C-A-5′.” Complementarity may be “partial,” in which only some of the nucleic acid bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

In some contexts, the term “complementarity” and related terms (e.g., “complementary”, “complement”) refers to the nucleotides of a nucleic acid sequence that can bind to another nucleic acid sequence through hydrogen bonds, e.g., nucleotides that are capable of base pairing, e.g., by Watson-Crick base pairing or other base pairing. Nucleotides that can form base pairs, e.g., nucleotides that are complementary to one another, are the pairs: cytosine and guanine, thymine and adenine, adenine and uracil, and guanine and uracil. The percentage complementarity need not be calculated over the entire length of a nucleic acid sequence. The percentage of complementarity may be limited to a specific region of which the nucleic acid sequences that are base-paired, e.g., starting from a first base-paired nucleotide and ending at a last base-paired nucleotide. The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

It is understood in the art that the sequence of a polynucleotide need not be 100% complementary to that of its target nucleic acid to be “hybridizable” or “specifically hybridizable” to the target nucleic acid. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted. For example, a nucleic acid in which 18 of 20 nucleotides of the nucleic acid are complementary to a target region, and would therefore specifically hybridize, would represent 90 percent complementarity. In this example, the remaining non-complementary nucleotides may be clustered or interspersed with complementary nucleotides and need not be contiguous to each other or to complementary nucleotides. Percent complementarity between particular segments of nucleic acid sequences within nucleic acids can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656, each of which is incorporated herein by reference) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489, incorporated herein by reference).

Thus, in some embodiments, “complementary” refers to a first nucleobase sequence that is at least 60%, 65%, 70%, 75%, 80%, 85%, 90%, 95%, 97%, 98%, or 99% identical to the complement of a second nucleobase sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases, or that the two sequences hybridize under stringent hybridization conditions. “Fully complementary” means each nucleobase of a first nucleic acid is capable of pairing with each nucleobase at a corresponding position in a second nucleic acid. For example, in certain embodiments, an oligonucleotide wherein each nucleobase has complementarity to a nucleic acid has a nucleobase sequence that is identical to the complement of the nucleic acid over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases.

As used herein, the term “mismatch” means a nucleobase of a first nucleic acid that is not capable of pairing with a nucleobase at a corresponding position of a second nucleic acid.

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tm of the formed hybrid. “Hybridization” methods involve the annealing of one nucleic acid to another, complementary nucleic acid, e.g., a nucleic acid having a complementary nucleotide sequence. The ability of two polymers of nucleic acid containing complementary sequences to find each other and “anneal” or “hybridize” through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960), each of which is incorporated herein by reference, have been followed by the refinement of this process into an essential tool of modern biology. For example, hybridization and washing conditions are now well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001), each of which is incorporated herein by reference. The conditions of temperature and ionic strength determine the “stringency” of the hybridization.

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A single-stranded nucleic acid having secondary structure (e.g., base-paired secondary structure) and/or higher order structure (e.g., a stem-loop structure) comprises a “double-stranded nucleic acid”. For example, triplex structures are considered to be “double-stranded”. In some embodiments, any base-paired nucleic acid is a “double-stranded nucleic acid”.

As used herein, the term “genomic locus” or “locus” (plural “loci”) is the specific location of a gene or nucleic acid (e.g., DNA or RNA) sequence on a chromosome.

The term “gene” refers to a DNA sequence that comprises control and coding sequences necessary for the production of an RNA having a non-coding function (e.g., a ribosomal or transfer RNA), a polypeptide, or a precursor. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this technology it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the term “knockout” is a genetic modification resulting from the disruption of the genetic information encoded in a chromosomal locus.

As used herein, the term “knockin” is a genetic modification resulting from the replacement of the genetic information encoded in a chromosomal locus with a different nucleic acid sequence.

As used herein, the term “knockout organism” is an organism in which a significant proportion of the organism's cells harbor a knockout.

As used herein, the term “knockin organism” is an organism in which a significant proportion of the organism's cells harbor a knockin.

As used herein, the term “functional derivative” of a polypeptide is a compound having a qualitative biological property in common with said polypeptide. “Functional derivatives” include, but are not limited to, fragments of polypeptide and derivatives of a polypeptide and its fragments, provided that they have a biological activity in common with a corresponding polypeptide. The term “derivative” encompasses both amino acid sequence variants of polypeptide, covalent modifications, and fusions thereof. A “fusion” polypeptide is a polypeptide comprising a polypeptide or portion (e.g., one or more domains) thereof fused or bonded to another heterologous polypeptide.

As used herein the term “variant” should be taken to mean the exhibition of qualities that have a pattern that deviates from what occurs in nature.

The terms “non-naturally occurring” or “engineered” are used interchangeably and indicate the involvement of the hand of man. The terms, when referring to nucleic acid molecules or polypeptides, mean that the nucleic acid molecule or the polypeptide is at least substantially free from at least one other component with which they are naturally associated in nature and as found in nature.

As used herein, the term “nuclease-deficient” refers to a protein comprising reduced nuclease activity, minimized and/or eliminated nuclease activity, altered nuclease activity (e.g., a nickase), undetectable nuclease activity, and/or having no nuclease activity, e.g., as a result of amino acid substitutions that reduce, minimize, alter, and/or eliminate the nuclease activity of a protein. In some embodiments, a nuclease-deficient protein is described as a “dead” protein and may be designated a “d” appended to the protein name (e.g., a dCas9).

The term “oligonucleotide” as used herein is defined as a molecule comprising two or more deoxyribonucleotides or ribonucleotides, preferably at least 5 nucleotides, more preferably at least about 10 to 15 nucleotides and more preferably at least about 15 to 50 nucleotides (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more nucleotides). The exact size will depend on many factors, which in turn depend on the ultimate function or use of the oligonucleotide. The oligonucleotide may be generated in any manner, including chemical synthesis, DNA replication, reverse transcription, PCR, or a combination thereof.

Because mononucleotides are reacted to make oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. A first region along a nucleic acid strand is said to be upstream of another region if the 3′ end of the first region is before the 5′ end of the second region when moving along a strand of nucleic acid in a 5′ to 3′ direction.

When two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide. Similarly, when two overlapping oligonucleotides are hybridized to the same linear complementary nucleic acid sequence, with the first oligonucleotide positioned such that its 5′ end is upstream of the 5′ end of the second oligonucleotide, and the 3′ end of the first oligonucleotide is upstream of the 3′ end of the second oligonucleotide, the first oligonucleotide may be called the “upstream” oligonucleotide and the second oligonucleotide may be called the “downstream” oligonucleotide.

The terms “peptide” and “polypeptide” and “protein” are used interchangeably herein, and refer to a polymeric form of amino acids of any length, which can include coded and non-coded amino acids, chemically or biochemically modified or derivatized amino acids, and polypeptides having modified peptide backbones.

As used herein, the term “ribonucleoprotein”, abbreviated “RNP” refers to a multimolecular complex comprising a polypeptide (e.g., a CRISPR protein or a protein having CRISPR activity or an activity similar to a CRISPR protein (e.g., a Cas9, Cpf1, or other Cas9-like protein, a Cas9 homolog, Cas13, and/or any modified version of any of the foregoing)) and a ribonucleic acid (e.g., a gRNA (e.g., sgRNA, a dgRNA)). In some embodiments, the polypeptide and ribonucleic acid are bound by a non-covalent interaction.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine/isoleucine, phenylalanine-tyrosine, lysine-arginine, alanine-valine, and asparagine-glutamine.

As used herein, the term “recombinant” means that a particular nucleic acid (DNA or RNA) is the product of various combinations of cloning, restriction, polymerase chain reaction (PCR), and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms). Alternatively, DNA sequences encoding RNA (e.g., DNA-targeting RNA) that is not translated may also be considered recombinant Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence.

A “vector” or “expression vector” is a replicon, such as plasmid, phage, virus, bacterial artificial chromosome (BAC), or cosmid, to which another DNA segment, e.g., an “insert”, may be attached so as to bring about the replication of the attached segment in a cell.

A cell has been “genetically modified” or “transformed” or “transfected” by exogenous DNA, e.g. a recombinant expression vector, when such DNA has been introduced inside the cell (e.g., according to the technology provided herein). In some embodiments, the presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, and/or nanoparticle-mediated nucleic acid delivery (e.g., according to the biodegradable polymer nanoparticle technology described herein; see also, e.g., Panyam and Labhasetwar (2012), Advanced Drug Delivery Reviews, 64 (supplement): 61-71, incorporated herein by reference). The choice of method of genetic modification is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995, incorporated herein by reference.

A “target nucleic acid” (e.g., a “target DNA” or a “target RNA”) as used herein is a polynucleotide (nucleic acid (e.g., DNA or RNA), gene, chromosome, genome, etc.) that comprises a “target site” or “target locus”, a “target sequence”, and/or a “target fragment”. The terms “target site”, “target sequence”, and “target locus” are used interchangeably herein to refer to a nucleic acid sequence present in a target DNA or target RNA to which a nucleic acid-targeting segment of a nucleic acid-targeting RNA will bind, provided sufficient conditions for binding exist. Suitable DNA/RNA or RNA/RNA binding conditions include physiological conditions normally present in a cell. Other suitable DNA/RNA or RNA/RNA binding conditions (e.g., conditions in a cell-free system) are known in the art; see, e.g., Sambrook, referenced herein and incorporated by reference. The strand of the target DNA or RNA that is complementary to and hybridizes with the nucleic acid-targeting RNA is referred to as the “complementary strand” and the strand of the target nucleic acid that is complementary to the “complementary strand” (and is therefore not complementary to the nucleic acid-targeting RNA) is referred to as the “noncomplementary strand” or “non-complementary strand”.

As used herein the term “target site”, “target sequence”, and “target locus” refer to a site within a nucleic acid molecule that is recognized (e.g., complementary to the gRNA) and cleaved by a nucleic acid cutting entity (e.g., an RNP (e.g., a CRISPR complex or CRISPR system comprising a CRISPR protein (e.g., a Cas9 or modified Cas9 or other Cas9-like CRISPR protein and/or modified versions thereof))).

As used herein, the term “target fragment” or “target nucleic acid fragment” is a nucleic acid flanked by two “target sites” or “target loci” or “target sequences” in a target nucleic acid. In some embodiments, the target fragment is produced by making double-stranded breaks in a target nucleic acid at two target sites, thus excising and liberating the target fragment from the target nucleic acid.

The RNA molecule that binds to the polypeptide in the RNP and targets the polypeptide to a specific location within the target nucleic acid is referred to herein as the “nucleic acid-targeting RNA” or “nucleic acid-targeting RNA polynucleotide” (also referred to herein as a “guide RNA” or “gRNA”). A nucleic acid-targeting RNA comprises two segments, a “nucleic acid-targeting segment” and a “protein-binding segment.” In some embodiments, the gRNA comprises two RNAs (e.g., a dgRNA, e.g., a crRNA and a tracrRNA) and in some embodiments the gRNA comprises one RNA (e.g., a sgRNA).

By “segment” it is meant a segment or section or portion or region of a molecule, e.g., a contiguous segment of nucleotides in an RNA, DNA, or protein. A segment can also mean a segment or section or portion or region of a complex such that a segment may comprise regions of more than one molecule. For example, in some embodiments the protein-binding segment (described below) of a nucleic acid-targeting RNA is one RNA molecule and the protein-binding segment therefore comprises a region of that RNA molecule. In other cases, the protein-binding segment (described below) of a nucleic acid-targeting RNA comprises two separate molecules that are hybridized along a region of complementarity. As an illustrative, non-limiting example, a protein-binding segment of a nucleic acid-targeting RNA that comprises two separate molecules can comprise (i) base pairs 40-75 of a first RNA molecule that is 100 base pairs in length; and (ii) base pairs 10-25 of a second RNA molecule that is 50 base pairs in length. The definition of “segment,” unless otherwise specifically defined in a particular context, is not limited to a specific number of total base pairs, is not limited to any particular number of base pairs from a given RNA molecule, is not limited to a particular number of separate molecules within a complex, and may include regions of RNA molecules that are of any total length and may or may not include regions with complementarity to other molecules.

The nucleic acid-targeting segment (or “nucleic acid-targeting sequence”) comprises a nucleotide sequence that is complementary to a specific sequence within a target nucleic acid (the complementary strand of the target nucleic acid). In some embodiments, the nucleic acid-targeting segment is a DNA-targeting segment that comprises a nucleotide sequence that is complementary to a specific sequence within a target DNA (the complementary strand of the target DNA). In some embodiments, the nucleic acid-targeting segment is an RNA-targeting segment that comprises a nucleotide sequence that is complementary to a specific sequence within a target RNA (the complementary strand of the target RNA). The protein-binding segment (or “protein-binding sequence”) interacts with a polypeptide of the RNP. The protein-binding segment of a nucleic acid-targeting RNA comprises two complementary segments of nucleotides that hybridize to one another to form a double stranded RNA duplex (dsRNA duplex).

A nucleic acid-targeting RNA and a polypeptide form a RNP complex (e.g., bind via non-covalent interactions). The nucleic acid-targeting RNA provides target specificity to the RNP complex by comprising a nucleotide sequence that is complementary to a sequence of a target nucleic acid. The polypeptide of the RNP complex provides site-specific binding and, in some embodiments, a nuclease activity (e.g., for producing double-stranded breaks in the target nucleic acid and/or for producing single-stranded breaks (“nicks”) in the target nucleic acid). In other words, the polypeptide of the RNP is guided to a target nucleotide sequence in the target nucleic acid (e.g., a target sequence in a chromosomal nucleic acid; a target sequence in an extrachromosomal nucleic acid (e.g., an episomal nucleic acid, a minicircle, etc.); a target sequence in a mitochondrial nucleic acid; a target sequence in a chloroplast nucleic acid; a target sequence in a plasmid; a target sequence in a transcript; a target sequence in a function RNA; a target sequence in an RNA genome; etc.) by virtue of its association with the protein-binding segment of the nucleic acid-targeting RNA.

In some embodiments, a nucleic acid-targeting RNA comprises two separate RNA molecules (e.g., two RNA polynucleotides, e.g., an “activator-RNA” and a “targeter-RNA”) and is referred to herein as a “double-molecule nucleic acid-targeting RNA” or a “two-molecule nucleic acid-targeting RNA” or a “double guide RNA” or a “dgRNA”. In other embodiments, the nucleic acid-targeting RNA is a single RNA molecule (e.g., a single RNA polynucleotide) and is referred to herein as a “single-molecule nucleic acid-targeting RNA,” a “single guide RNA,” or an “sgRNA.” The term “nucleic acid-targeting RNA” or “guide RNA” or “gRNA” is inclusive, referring both to double-molecule nucleic acid-targeting RNAs (dgRNAs) and to single-molecule nucleic acid-targeting RNAs (sgRNAs).

An exemplary two-molecule nucleic acid-targeting RNA comprises a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA”) molecule. A crRNA-like molecule (targeter-RNA) comprises both the nucleic acid-targeting segment (single stranded) of the nucleic acid-targeting RNA and a region (“duplex-forming segment”) that forms one half of the dsRNA duplex of the protein-binding segment of the nucleic acid-targeting RNA. A corresponding tracrRNA-like molecule (activator-RNA) comprises a region (duplex-forming segment) that forms the other half of the dsRNA duplex of the protein-binding segment of the nucleic acid-targeting RNA. In other words, a portion of the crRNA-like molecule is complementary to and hybridizes with a portion of a tracrRNA-like molecule to form the dsRNA duplex of the protein-binding domain of the nucleic acid-targeting RNA. As such, each crRNA-like molecule can be said to have a corresponding tracrRNA-like molecule. The crRNA-like molecule additionally provides the single stranded DNA-targeting segment.

Thus, a crRNA-like molecule (e.g., a crRNA) and a tracrRNA-like molecule (e.g., a tracrRNA) hybridize (as a corresponding pair) to form a nucleic acid-targeting RNA. The exact sequence of a given crRNA or tracrRNA molecule is characteristic of the species in which the RNA molecules are found. Various crRNAs and tracrRNAs are known in the art. A double molecule nucleic acid-targeting RNA (dgRNA) can comprise any corresponding crRNA and tracrRNA pair. A single molecule nucleic acid-targeting RNA (sgRNA) can comprise any corresponding crRNA and tracrRNA pair.

The term “activator-RNA” is used herein to mean a tracrRNA-like molecule of a double molecule nucleic acid-targeting RNA (e.g., a tracrRNA). The term “targeter-RNA” is used herein to mean a crRNA-like molecule of a double-molecule nucleic acid-targeting RNA (e.g., a crRNA). The term “duplex-forming segment” is used herein to mean the segment of an activator-RNA or a targeter-RNA that contributes to the formation of the dsRNA duplex by hybridizing to a segment of a corresponding activator-RNA or targeter-RNA molecule. In other words, an activator-RNA comprises a duplex-forming segment that is complementary to the duplex-forming segment of the corresponding targeter-RNA. As such, an activator-RNA comprises a duplex-forming segment while a targeter-RNA comprises both a duplex-forming segment and the nucleic acid-targeting segment of the DNA-targeting RNA. Therefore, a double-molecule nucleic acid-targeting RNA can be comprised of any corresponding activator-RNA and targeter-RNA pair.

The term “sample” in the present specification and claims is used in its broadest sense. On the one hand it is meant to include a specimen or culture (e.g., microbiological cultures). On the other hand, it is meant to include both biological and environmental samples. A sample may include a specimen of synthetic origin. In some embodiments, a sample comprises a nucleic acid (e.g., a DNA and/or an RNA) and, optionally, buffer, salts, preservatives, stabilizers, dyes, etc.

As used herein, a “biological sample” refers to a sample of biological tissue or fluid or fraction or component thereof (e.g., a molecule (e.g., a protein, amino acid, nucleic acid, nucleotide, lipid, metabolite, sugar, cofactor, etc.), organelle, membrane, etc.). For instance, a biological sample may be a sample obtained from an animal (including a human); a fluid, solid, or tissue sample; as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagomorphs, rodents, etc. Examples of biological samples include sections of tissues, blood, blood fractions, plasma, serum, urine, or samples from other peripheral sources or cell cultures, cell colonies, single cells, or a collection of single cells. Furthermore, a biological sample includes pools or mixtures of the above mentioned samples. A biological sample may be provided by removing a sample of cells from a subject, but can also be provided by using a previously isolated sample. For example, a tissue sample can be removed from a subject suspected of having a disease by conventional biopsy techniques. In some embodiments, a blood sample is taken from a subject. A biological sample from a patient means a sample from a subject suspected to be affected by a disease.

Environmental samples include environmental material such as surface matter, soil, water, and industrial samples, as well as samples obtained from food and dairy processing instruments, apparatus, equipment, utensils, disposable and non-disposable items. These examples are not to be construed as limiting the sample types applicable to the present invention.

As used herein, “moiety” refers to one of two or more parts into which something may be divided, such as, for example, the various parts of an oligonucleotide, a molecule, a chemical group, a domain, a probe, etc.

As used herein, the word “presence” or “absence” (or, alternatively, “present or “absent”) is used in a relative sense to describe the amount or level of a particular entity (e.g., a nucleic acid). For example, when a nucleic acid is said to be “present” in a test sample, it means the level or amount of this nucleic acid is above a pre-determined threshold; conversely, when a nucleic acid is said to be “absent” in a test sample, it means the level or amount of this nucleic acid is below a pre-determined threshold. The pre-determined threshold may be the threshold for detectability associated with the particular test used to detect the nucleic acid or any other threshold. When a nucleic acid is “detected” in a sample it is “present” in the sample; when a nucleic acid is “not detected” it is “absent” from the sample. Further, a sample in which a nucleic acid is “detected” or in which the nucleic acid is “present” is a sample that is “positive” for the nucleic acid. A sample in which a nucleic acid is “not detected” or in which the nucleic acid is “absent” is a sample that is “negative” for the nucleic acid.

As used herein, an “increase” or a “decrease” refers to a detectable (e.g., measured) positive or negative change in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least 90% of the previously measured value of the variable, the pre-established value, and/or the value of a standard control. Other terms indicating quantitative changes or differences, such as “more” or “less,” are used herein in the same fashion as described above.

A “system” denotes a set of components, real or abstract, comprising a whole where each component interacts with or is related to at least one other component within the whole.

As used herein the term “monomer” refers to a molecule that can undergo polymerization, thereby contributing constitutional units to the essential structure of a macromolecule or polymer.

A “polymer” is a molecule of high relative molecule mass, the structure of which essentially comprises the multiple repetition of unit derived from molecules of low relative molecular mass, i.e., a monomer.

As used herein, an “oligomer” includes a few monomer units, for example, in contrast to a polymer that potentially can comprise an unlimited number of monomers. Dimers, trimers, and tetramers are non-limiting examples of oligomers.

Further, as used herein, the term “nanoparticle,” refers to a particle having at least one dimension in the range of about 1 nm to about 1000 nm, including any integer value between 1 nm and 1000 nm (including about 1, 2, 5, 10, 20, 50, 60, 70, 80, 90, 100, 200, 500, and 1000 nm and all integers and fractional integers in between). In some embodiments, the nanoparticle has at least one dimension, e.g., a diameter, of about 100 nm. In some embodiments, the nanoparticle has a diameter of about 200 nm. In other embodiments, the nanoparticle has a diameter of about 500 nm. In yet other embodiments, the nanoparticle has a diameter of about 1000 nm (1 μm). In such embodiments, the particle also can be referred to as a “microparticle. Thus, the term “microparticle” includes particles having at least one dimension in the range of about one micrometer (μm), i.e., 1×10⁻⁶ meters, to about 1000 μm. The term “particle” as used herein is meant to include nanoparticles and microparticles.

It will be appreciated by one of ordinary skill in the art that nanoparticles suitable for use with the presently disclosed methods can exist in a variety of shapes, including, but not limited to, spheroids, rods, disks, pyramids, cubes, cylinders, nanohelixes, nanosprings, nanorings, rod-shaped nanoparticles, arrow-shaped nanoparticles, teardrop-shaped nanoparticles, tetrapod-shaped nanoparticles, prism-shaped nanoparticles, and a plurality of other geometric and non-geometric shapes. In particular embodiments, the presently disclosed nanoparticles have a spherical shape.

“Associated with”: When two entities are “associated with” one another as described herein, they are linked by a direct or indirect covalent or non-covalent interaction. Preferably, the association is covalent. Desirable non-covalent interactions include hydrogen bonding, van der Waals interactions, hydrophobic interactions, magnetic interactions, electrostatic interactions, etc.

“Biocompatible”: The term “biocompatible”, as used herein is intended to describe compounds that are not toxic to cells. Compounds are “biocompatible” if their addition to cells in vitro results in less than or equal to 20% cell death, and their administration in vivo does not induce inflammation or other such adverse effects.

“Biodegradable”: As used herein, “biodegradable” compounds are those that, when introduced into cells, are broken down by the cellular machinery or by hydrolysis into components that the cells can either reuse or dispose of without significant toxic effect on the cells (i.e., fewer than about 20% of the cells are killed when the components are added to cells in vitro). The components preferably do not induce inflammation or other adverse effects in vivo. In certain preferred embodiments, the chemical reactions relied upon to break down the biodegradable compounds are uncatalyzed.

“Peptide” or “protein”: A “peptide” or “protein” comprises a string of at least three amino acids linked together by peptide bonds. The terms “protein” and “peptide” may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Polynucleotide” or “oligonucleotide”: Polynucleotide or oligonucleotide refers to a polymer of nucleotides. Typically, a polynucleotide comprises at least three nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C₅-propynylcytidine, C₅-propynyluridine, C₅-bromouridine, C₅-fluorouridine, C₅-iodouridine, C₅-methylcytidine, 7-deazaadenosine, 7-deazaguanosine, 8-oxoadenosine, 8-oxoguanosine, 0(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

“Small molecule”: As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds. Known naturally-occurring small molecules include, but are not limited to, penicillin, erythromycin, taxol, cyclosporin, and rapamycin. Known synthetic small molecules include, but are not limited to, ampicillin, methicillin, sulfamethoxazole, and sulfonamides.

While the following terms in relation to compounds of formula (I) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R2, and the like, or variables, such as “m” and “n”), can be identical or different. For example, both R₁ and R2 can be substituted alkyls, or R₁ can be hydrogen and R2 can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁₋₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, cyano, and mercapto.

The term “heteroalkyl,” by itself or in combination with another term, means, unless otherwise stated, a stable straight or branched chain having from 1 to 20 carbon atoms or heteroatoms or a cyclic hydrocarbon group having from 3 to 10 carbon atoms or heteroatoms, or combinations thereof, consisting of at least one carbon atom and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)— CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or—S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NRR″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkylene moiety, also as defined above, e.g., a C₁₋₂₀ alkylene moiety. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated hydrocarbon has one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₂₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₂₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

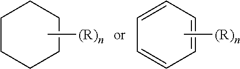

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

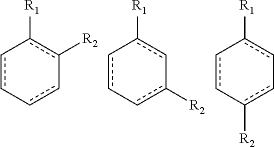

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN, CF₃, fluorinated C₁₋₄ alkyl, and —NO₂ in a number ranging from zero to (2m′+1), where m′ is the total number of carbon atoms in such groups. R′, R″, R″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″—S(O)R′, —S(O)₂R′, —S(O)₂NR′R″, —NRSO₂R′, —CN and —NO₂, —R′, —N₃, —CH(Ph)₂, fluoro(C₁₋₄alkoxo, and fluoro(C₁₋₄)alkyl, in a number ranging from zero to the total number of open valences on aromatic ring system; and where R′, R″, R″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′— (C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂. “Alkylcarbamoyl” refers to a R′RN—C(═O)— group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′ R″, wherein R′ and R″ are each independently selected from the group consisting of alkyl groups. The term trialkylamino refers to a group having the structure —NR′R″R′″, wherein R′, R″, and R″ are each independently selected from the group consisting of alkyl groups. Additionally, R′, R″, and/or R″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The term “cyano” refers to the group.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁₋₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R

The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)-or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

The term “protecting group” refers to chemical moieties that block some or all reactive moieties of a compound and prevent such moieties from participating in chemical reactions until the protective group is removed, for example, those moieties listed and described in T. W. Greene, P.G.M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(O)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typical blocking/protecting groups include, but are not limited to the following moieties:

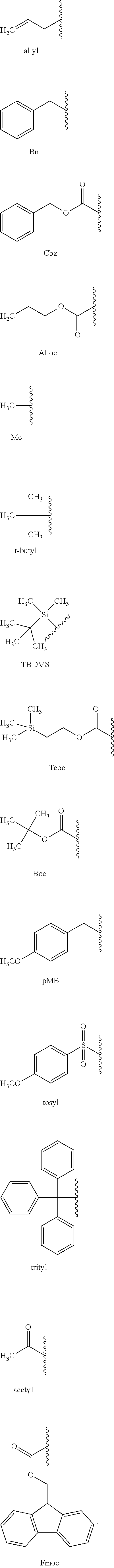

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration and are not to be construed as limiting in any manner to make compounds of the disclosure by other methods.

### Example 1

### Poly(β-Amino Ester) Nanoparticles for the Non-Viral Delivery of CRISPR/Cas9 For Efficient Gene Editing in vitro and in vivo

1.1 Overview. The CRISPR/Cas9 system is a powerful genome editing tool that can direct site-specific gene disruption. The Cas9 endonuclease introduces double stranded breaks at sites specified by a single guide RNA (sgRNA), and gene disruption occurs by the introduction of indels that cause frame-shift mutations (gene knockout) or by the removal of large segments of the gene (gene deletion). The Cas9-sgRNA complex recognizes a target site in genomic DNA, then Cas9 cuts genomic DNA. The CRISPR/Cas9 system holds great potential as a gene therapy platform. Safe and effective delivery, however, remains a challenge.

Poly(β-amino ester)s (PBAEs) are a class of biodegradable, cationic polymers that self-assemble into nanoparticles upon complexation with nucleic acids. Accordingly, in some embodiments, the presently disclosed matter provides PBAE nanoparticles for co-delivering plasmid DNA encoding Cas9 and sgRNA to a cell to mediate gene knockout and deletion. FIG. 6 depicts the formation of PBAE-DNA nanoparticles. Also shown in FIG. 6 is a representative PBAE polymer, designated as 446.

1.2 Methods. To assess gene knockout efficiency, PBAE nanoparticles were used to deliver two plasmids encoding the Cas9 protein and an anti-eGFP sgRNA, respectively, to HEK-293T cells constitutively expressing a destabilized form of eGFP. Knockout of eGFP was assessed by flow cytometry and confirmed by Surveyor® nuclease assay and Sanger sequencing. To assess gene deletion efficacy, a HEK-293T cell line constitutively expressing a red-enhanced nanolantern (ReNL) reporter gene downstream of a transcription stop cassette consisting of two SV40 terminator sequences was generated. PBAE nanoparticles were used to deliver plasmids encoding Cas9 and sgRNAs targeting the stop cassette. Gene deletion was assessed by the quantification of ReNL expression, which occurred after successful deletion of the stop cassette.

1.3 Results. The presently disclosed nanoparticle system achieved a high level of eGFP knockout (>70% as assessed by the geometric mean of fluorescence) three days post-transfection and sustained this level of gene silencing for the entirety of the experiment (over 3 weeks post-transfection). Interestingly, CRISPR-mediated knockout resulted in a population of cells that were completely eGFP-negative. This binary turning-off-of gene expression is in stark contrast to the downregulation of gene expression achieved through the delivery of short-interfering RNA (siRNA), which resulted in lowered gene expression on a population basis and only had a temporary effect.

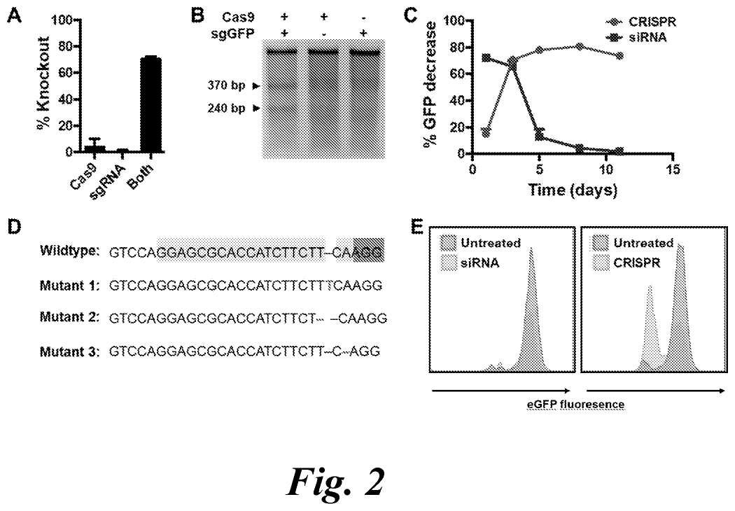

Referring now to FIG. 2A-E, the presently disclosed subject matter, in some embodiments, demonstrates that PBAE nanoparticles enable gene knockout through small indels after NHEJ and produce a sustained, binary effect compared to siRNA-mediated gene silencing. Importantly, PBAE nanoparticle-mediated gene knockout results in permanent and binary gene silencing.

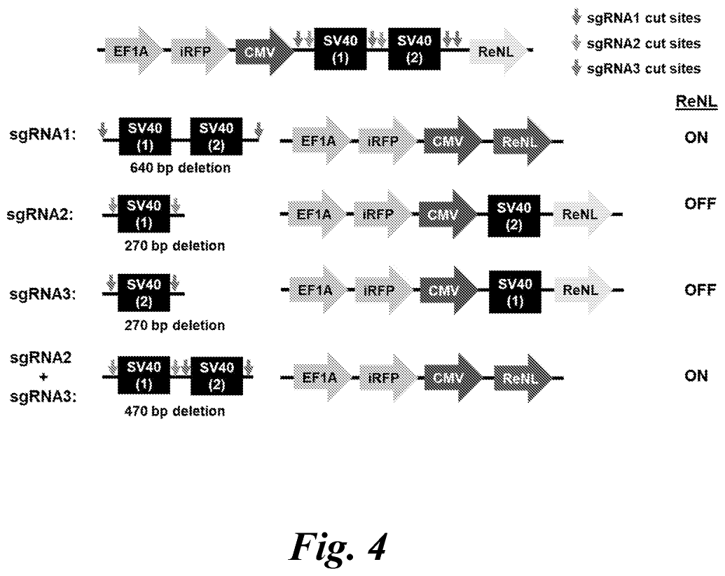

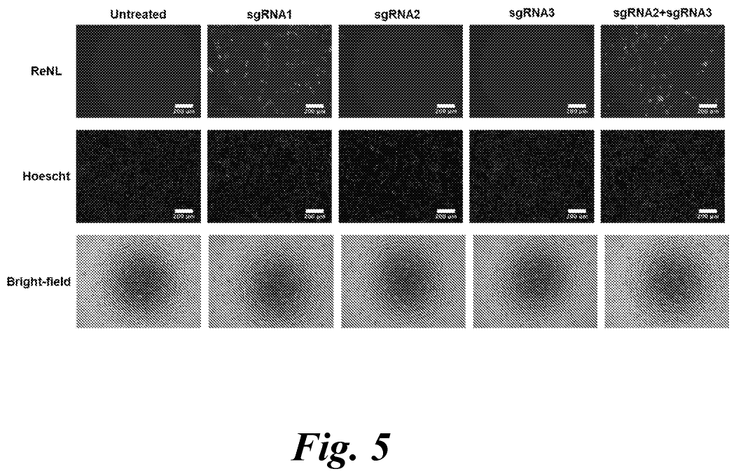

The presently disclosed PBAE nanoparticles also achieved successful gene deletion. The top sgRNA sequence resulted in the deletion of a 600 bp DNA segment, which turned on detectable ReNL expression in 45% of treated cells. PCR amplicons of the edited region confirmed that ReNL expression required the deletion of the entire stop cassette. See FIG. 3A-D. Further, as shown in FIG. 4 and FIG. 5, co-delivery of two sgRNAs flanking a gene segment enables gene deletion and gain-of-function ReNL expression in a novel reporter system in vitro. This system allows for identification of effective CRISPR editing both in vitro and in vivo with bioluminescence imaging of ReNL.

Gene-knockout efficiency was assessed using HEK-293T cells which constitutively express a destabilized form of eGFP (See, “Unedited” in FIG. 1A). Cells were transfected with poly(beta-amino ester) (PBAE) nanoparticles carrying two plasmids encoding the Cas9 protein and an anti-eGFP gRNA, respectively (FIG. 2E). Knockout of eGFP (“Knockout” in FIG. 1A) was assessed by flow cytometry and confirmed by Surveyor® nuclease assay and Sanger sequencing.

Gene-deletion efficacy was assessed using a HEK-293T cell line which includes a Red-enhanced NanoLantern (ReNL) reporter gene downstream of a transcription STOP cassette consisting of two SV40 terminator sequences (see, top construct in FIG. 1B). Cells were transfected with PBAE nanoparticles carrying plasmids encoding the Cas9 protein and an anti-STOP cassette gRNA. Gene deletion was assessed by ReNL reporter gene activity, which occurred after successful deletion of the STOP cassette (see, bottom construct in FIG. 1B).

In a gene-knockout experiment (schematized in FIG. 1A), the nanoparticle system achieved a high level of eGFP knockout (>70% as assessed by the geometric mean of fluorescence, FIG. 2A), three days after transfection. This level of gene silencing was sustained for the entirety of the experiment: three weeks following transfection. Interestingly, CRISPR-mediated knockout resulted in a population of cells that were completely eGFP-negative. This binary silencing of gene expression is in stark contrast to the down-regulation of gene expression achieved through the delivery of short-interfering RNA (siRNA), which resulted in lowered gene expression on a population basis and only provided a temporary effect. (FIG. 3C and FIG. 2E).

The PBAE nanoparticle system was also effective in gene-deletion studies (schematized in FIG. 1B). An anti-STOP cassette gRNA efficiently deleted the entire 600 bp STOP cassette, which was confirmed by PCR amplification (FIG. 2B). Deletion of the entire STOP cassette turned on detectable ReNL expression in 45% of treated cells (FIG. 3D and FIG. 3B). This system identifies effective CRISPR-editing both in vitro and in vivo using bioluminescence imaging of ReNL.

1.4 Summary. The presently disclosed subject matter demonstrates that PBAE nanoparticles co-delivering plasmids encoding Cas9 and sgRNA, respectively, can achieve a high degree of gene knockout and deletion. The system is versatile, as sgRNAs targeting any gene (or another genomic sequence) can be designed and incorporated into nanoparticles for gene knockout. Further, the presently disclosed subject matter showed that the PBAE nanoparticles can achieve the more challenging genome editing procedure of gene deletion, which is important in inducing a loss of function in non-coding genes. The presently disclosed PBAE nanoparticles represent a promising tool for gene therapy applications and useful approach as a reporter system for CRISPR editing in vitro and in vivo.

Additionally, in some embodiments, the presently disclosed subject matter demonstrates CRISPR editing in vivo. For example, murine melanoma (B16-F10) and glioblastoma (GL261) cells were induced to express an iRFP-STOP-ReNL reporter system. Successful editing of these cells in vivo could be visualized using ReNL bioluminescence. Dual delivery of separate Cas9 and sgRNA plasmids to B16-F10 and GL261 cells yielded low gene deletion (<5% ReNL fluorescence by flow cytometry). In yet other embodiments, cloning Cas9 and sgRNA into single vector can boost efficiency. As provided herein below, a large combinatorial library of novel hyper-branched PBAE nanoparticle formulations also have been screened and can exhibit higher transfection efficacy compared to canonical PBAEs.

### Example 2

### Hyperbranched Polyesters with Amphiphilic and pH Sensitive Properties for Effective Nucleic Acid Delivery

FIG. 7A to FIG. 7C illustrate synthesis of a BGDA-series of hyperbranched PBAE polymers for nanoparticle assembly. A diacrylate monomer (bisphenol A glycerolate diacrylate, BGDA; “*”) and triacrylate monomer (trimethylolpropane triacrylate, TMPTA; “†”) are mixed with side-chain monomer S4 (“‡”) to synthesize a series of Poly(β-amino ester) (PBAE) with increasing triacrylate mole fraction and degree of branching. See FIG. 7A. Linear PBAEs possess two end-cap E6 moieties (※) per molecule (FIG. 7B, “Linear”), whereas each triacrylate monomer in branched PBAEs results in an additional endcap E6 moiety (※) for every branch point (FIG. 7B, “Branched”).

FIG. 7C illustrates one-pot synthesis of acrylate terminated base polymers, performed at 90° C. and 200 mg/mL in DMSO for 24 hours. Polymers are then end-capped with the endcap E6 (※) at room temperature for one hour to yield end-capped, hyperbranched PBAEs.

More particularly, the synthesis of the BGDA series of hyperbranched PBAEs is provided in FIGS. 7A-C. As shown in FIG. 7A, the diacrylate monomer BGDA and triacrylate monomer TMPTA were mixed with side-chain monomer S4 to synthesize a series of PBAEs with increasing triacrylate mole fraction and degree of branching. As shown in FIG. 7B, linear PBAEs possess two end-cap structures per molecule (red), while each triacrylate monomer in branched PBAEs results in an additional endcap moiety for every branch point. FIG. 7C shows the one-pot synthesis of acrylate terminated base polymers, which is performed at 90° C. and 200 mg/mL in DMSO for 24 hours. Polymers were then endcapped with monomer E6 at room temperature for one hour to yield end-capped, hyperbranched PBAEs.

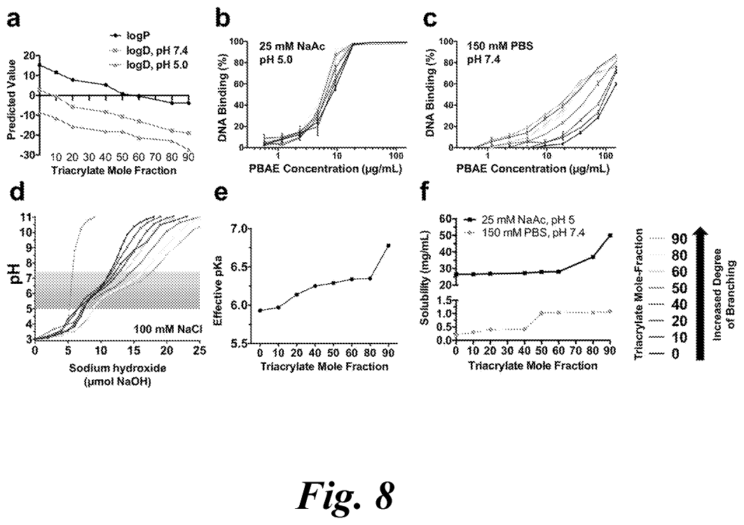

Representative polymer characteristics are illustrated in FIGS. 8A-F. FIG. 8A shows the predicted properties of partition coefficient (log P) and distribution coefficient (log D) for variably branched BGDA PBAEs. FIG. 8B shows competition binding assay of polymer and Yo-Pro-1 iodide at low pH. (n=3 wells, mean±SEM). FIG. 8C shows competition DNA binding assay in isotonic, neutral buffer. (n=3 wells, mean±SEM); FIG. 8D shows the titration of PBAEs. FIG. 8E shows the effective pKa value of maximum buffering point between pH 4.5-8.5 of variably branched PBAEs. FIG. 8F shows the effective solubility of variably branched PBAEs at low pH and in isotonic, neutral buffer. Blending multiple monomers enables fine-tuning of polymer properties mid-way between the states of either monomer. Properties include hydrophobicity (assessed computationally via log P and log D), DNA binding, buffering capacity and effective pKa value.

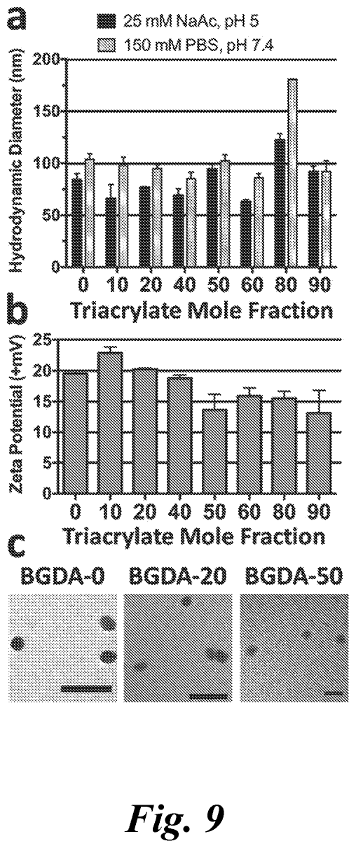

Additional BGDA nanoparticle properties are shown in FIGS. 9A-C. FIG. 9A shows the Z-average hydrodynamic diameter measurements in 25 mM NaAc buffer, pH 5.0 and after dilution into 150 mM PBS at a 40 w/w ratio. FIG. 9B shows the Zeta potential measurements assessed in 150 mM PBS, pH 7.4. (n=3 preparations, mean±SEM). FIG. 9C shows TEM images of dried particles. Scale bar 100 nm for all images. Nanoparticles have effectively the same properties for the tested polymer series regardless of degree of branching.

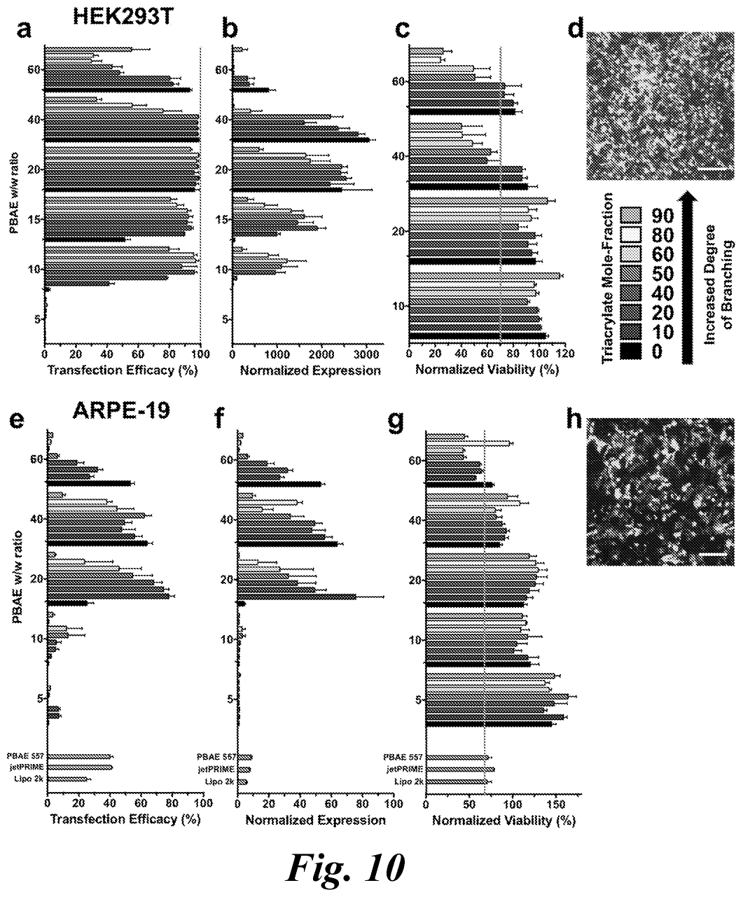

In vitro transfection of HEK239T cells or ARPE-19 cells with BGDA PBAEs in 10% serum media is shown in FIGS. 10A-H. FIG. 10A shows the transfection efficacy. FIG. 10B shows the normalized geometric mean expression. FIG. 10C shows the viability and FIG. 10D shows a fluorescent microscope image. FIG. 10E shows the transfection efficacy in ARPE-19 cells. FIG. 10F shows the normalized geometric mean expression. FIG. 10G shows the viability and FIG. 10H shows a fluorescent microscope image. Scale bars 200 μm. (n=4 wells, mean±SEM). Transfection efficacy of retinal ARPE-19 cells is notably much higher than both commercial transfection reagents Lipofectamine 2000 and jetPrime as well as the previously optimized PBAE 557.

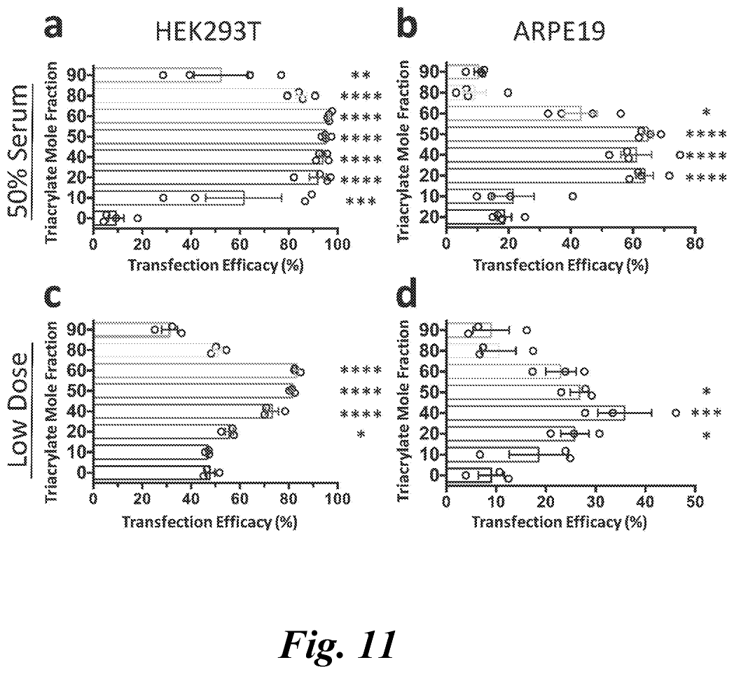

FIGS. 11A-D demonstrates challenging transfection conditions with BGDA PBAEs. High serum (50%) transfection of HEK293T (FIG. 11A) and ARPE-19 cells (FIG. 11B) with 20 w/w nanoparticles. Low nanoparticle dose transfection with 40 w/w nanoparticles of HEK293T (5 ng) (FIG. 11C) and ARPE-19 (10 ng) (FIG. 11D) doses in 384 well plates. Branching notably improves transfection efficacy in both cell lines in high serum conditions and at low nanoparticle doses.

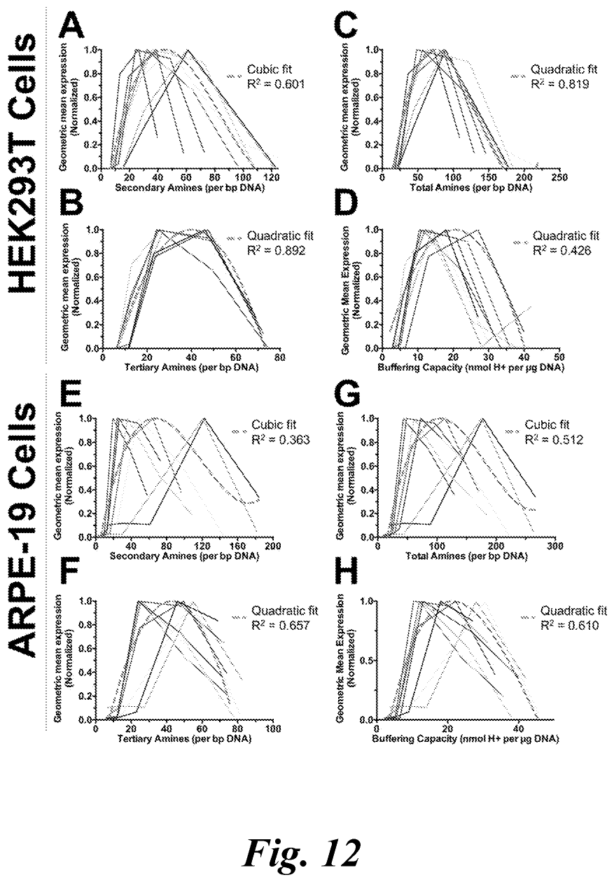

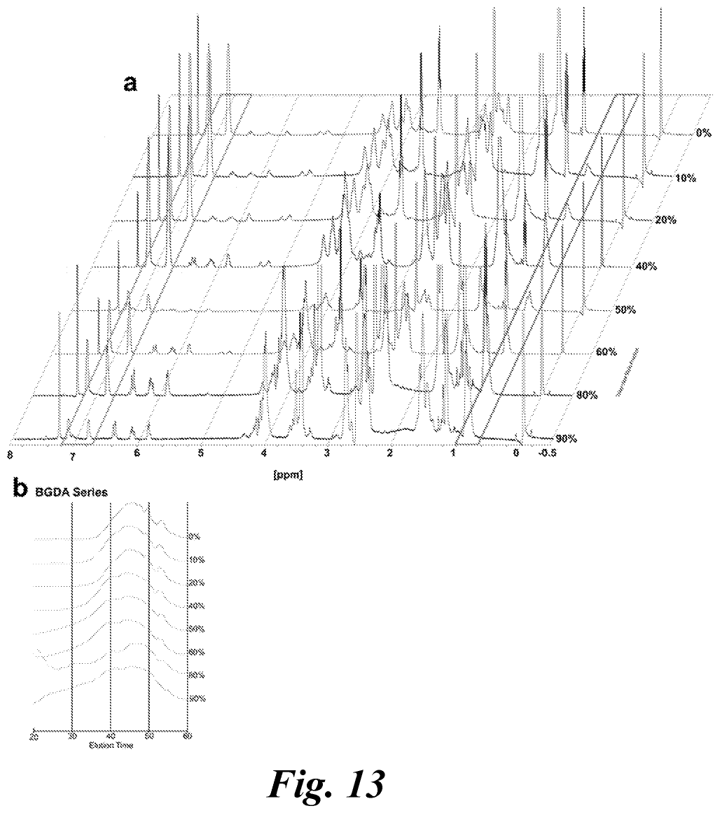

FIG. 12A-H shows the correlation between polymer properties and transfection efficacy. (FIG. 12A-D) HEK293T cells and (FIG. 12E-H) ARPE-19 cells.

Additional structural properties of the BGDA series of polymers are provided in Table 2.

TABLE 2
BGDA series polymer structural properties.

| Triacrylate Mol % | | Number Mean # | Endcap Molecule | NMR M_{N} | GPC M_{N} | GPC M_{W} | |
|---|---|---|---|---|---|---|---|
| Planned | Actual | Endcaps/molecule | Mass fraction (%) | (Da) | (Da) | (Da) | GPC PDI |
| 0 | 0.0 | 2.0 | 5.9 | 4000 | 3200 | 7200 | 2.3 |
| 10 | 15.1 | 3.0 | 8.8 | 4100 | 3200 | 10000 | 3.1 |
| 20 | 22.8 | 3.4 | 10.9 | 3700 | 3600 | 12400 | 3.5 |
| 40 | 34.8 | 3.9 | 14.2 | 3200 | 4000 | 30200 | 7.6 |
| 50 | 47.1 | 4.9 | 16.2 | 3600 | 4400 | 62800 | 14.5 |
| 60 | 58.5 | 4.5 | 21.2 | 2500 | 4400 | 59800 | 13.8 |
| 80 | 83.3 | 5.5 | 27.0 | 2400 | 3800 | 107000 | 27.8 |
| 90 | 91.7 | 6.5 | 28.0 | 2800 | 4800 | 113400 | 23.7 |

Additional characteristics of the presently disclosed polymer series are illustrated in FIGS. 13 through 24. FIGS. 13A-B shows the chemical properties of the presently disclosed BGDA polymer series. FIG. 13A shows NMR spectra of the presently disclosed BGDA series of acrylate terminated PBAE polymers ¹H NMR (500 MHz, CDCl₃-ch, 0.05% v/v TMS) spectra. Note that some peaks are from residual solvent for diethyl ether (3.48, 1.2 ppm) and DMSO (2.62 ppm). Relevant peaks for determination of MN and triacrylate mole fraction are as follows. BGDA phenyl (4H each) 6.81 and 7.11 ppm in green; TMPTA methyl (3H) 0.83 ppm in red; S4 (2H/repeat) 2.38 ppm.

FIG. 13B shows gel permeation chromatography refractive index detector traces for the BGDA series of polymers. GPC and analysis in Waters2 software was used to calculate MN, Mw and PDI of each polymer relative to a third order curve fit of eight linear polystyrene standards (R²=0.9987) ranging in molecular weight from 580 Da to 3.15 MDa.

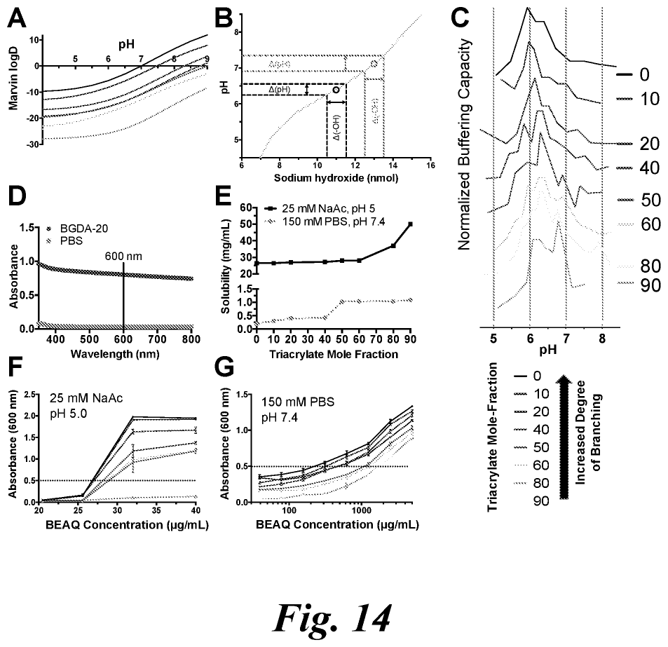

FIG. 14A, FIG. 14B, FIG. 14C, FIG. 14D, and FIG. 14E show the aqueous properties of the presently disclosed BGDA polymer series. FIG. 14A shows Marvin predicted log D values assessing polymer hydrophobicity at different pH values. Computed for 140 mM Cl—, Na/K+ conditions with NMR value MN matched polymer structures; FIG. 14B shows the method for calculation of effective buffering capacity at each pH point (between 4.5-8); FIG. 14C shows calculated normalized buffering capacity from individual polymer titrations enabled effective pKa value of each polymer to be determined; FIG. 14D shows the absorbance spectra of polymer BGDA-20 dissolved into 150 mM PBS, pH 7 at 10 mg/mL to determine 600 nm wavelength to approximate solubility measurements. The solubility of BGDA polymers (FIG. 14E) with absorbance >0.5 at 600 nm defined as insoluble was calculated from dilution series in (FIG. 14F) 150 mM PBS, pH 7.4 and (FIG. 14G) 25 mM NaAc, pH 5.0. Solubility increased as predicted with branching due to the increase in the number of hydrophilic endcap moieties.

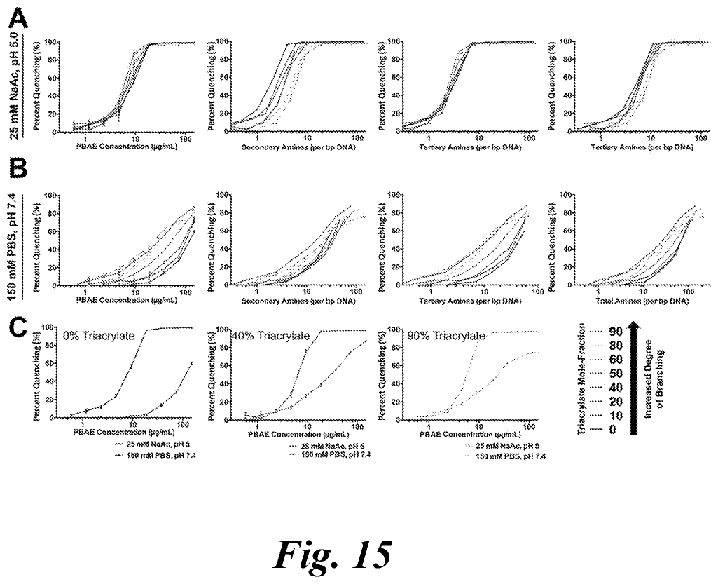

FIGS. 15A-C show the DNA binding properties of the presently disclosed BGDA polymer series. For both buffer conditions the plots show fluorescence quenching as a function of polymer concentration, quenching normalized to number of secondary amines, normalized to number of tertiary amines and normalized to the total number of amines (FIG. 15A) Under acidic conditions at pH 5.0 and low salt, degree of DNA binding is best proportional to the number of tertiary amines per base pair (bp) of DNA. (FIG. 15B) In contrast, under neutral, isotonic conditions at pH 7.4, the degree of DNA binding is best proportional to the number of secondary amines per bp DNA. (FIG. 15C) The difference in binding between pH 5 to pH 7.4 for the linear (0% triacrylate), moderately branched polymer (40% triacrylate) and highly branched polymer (90% triacrylate) were compared.

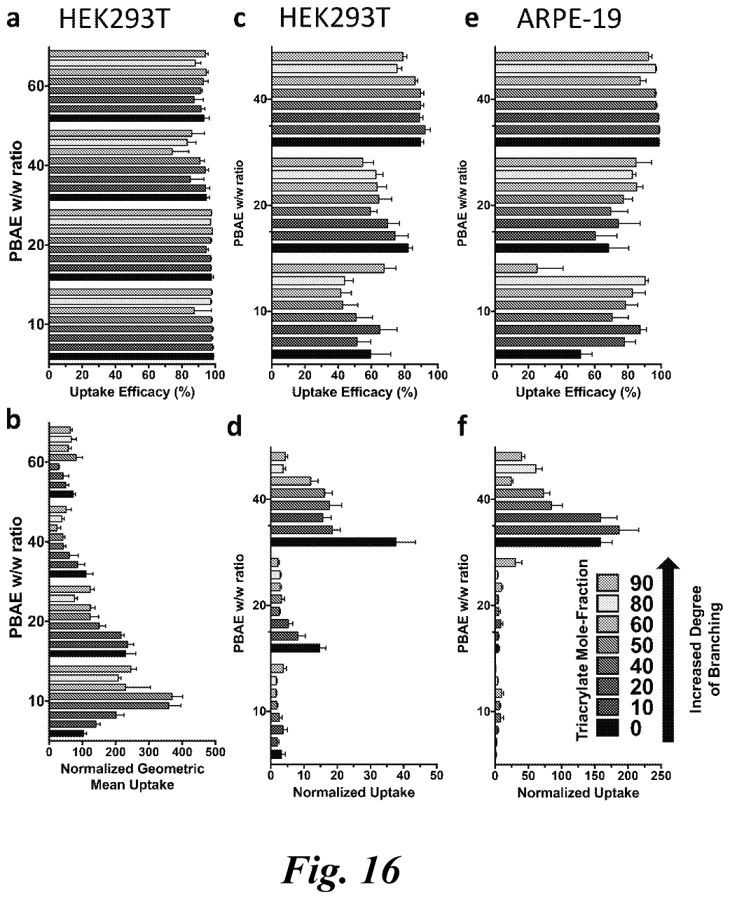

FIGS. 16A-F show BGDA nanoparticle uptake in HEK293T and ARPE-19 cells. Branching does not strongly improve nanoparticle uptake compared to linear BGDA polymer nanoparticles at the same w/w ratios. HEK293T high dose nanoparticle uptake (600 ng dose, 20% labeled Cy5-DNA) as (FIG. 16A) percent uptake and (FIG. 16B) geometric mean. HEK293T low dose nanoparticle uptake (300 ng, 20% labeled Cy5-DNA) as (FIG. 16C) percent uptake and (FIG. 16D) geometric mean. ARPE-19 low dose nanoparticle uptake (300 ng, 20% labeled Cy5-DNA) as (FIG. 16E) percent uptake and (FIG. 16F) geometric mean.

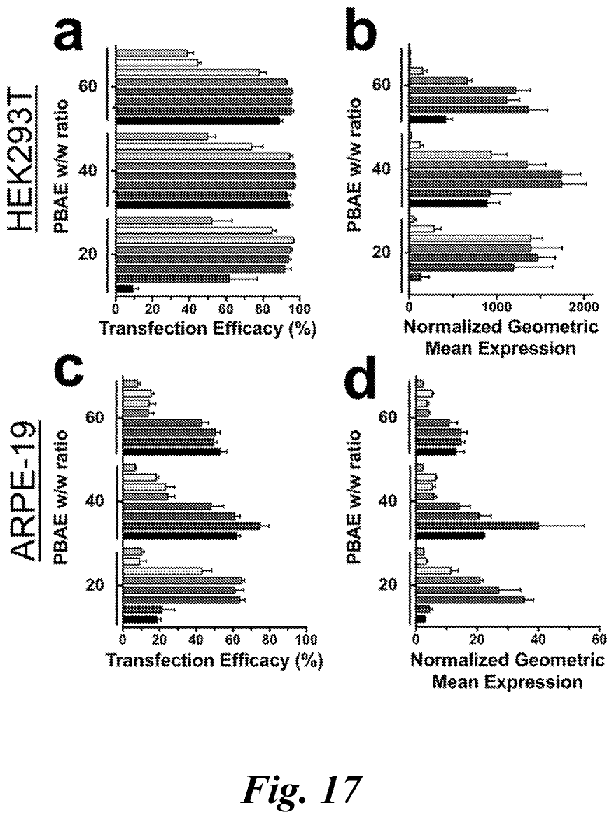

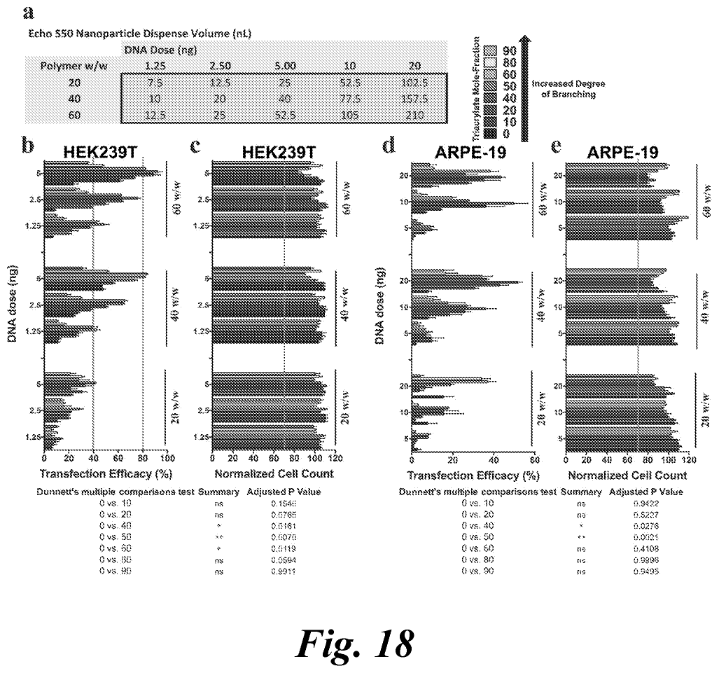

FIG. 17A-C shows BGDA series nanoparticle transfection in high serum (50%) conditions. HEK293T cells (17A) transfection efficacy up to 97% and (17B) geometric mean expression. ARPE-19 (17C) transfection efficacy up to 67%. Moderately branched BGDA PBAEs outperformed the linear BGDA polymer when level of expression was taken into account; this effect was especially evident at low w/w ratios.

FIGS. 18A-E shows BGDA nanoparticle transfection at low doses in HEK239T cells and ARPE-19 cells. FIG. 18A shows extremely low volume distribution of nanoparticles achieved via Echo 550 acoustic liquid handling with nanoparticle dose titration. FIG. 18B shows transfection efficacy in HEK239T cells and FIG. 18C shows untreated normalized cell counts in HEK239T cells. FIG. 18D shows transfection efficacy in ARPE-19 cells and FIG. 18E shows untreated normalized cell counts in ARPE-19 cells. Branched BGDA polymers with 40-60% triacrylate mole-fraction were statistically more effective than the linear BGDA polymer tested for low dose nanoparticle transfection. No nanoparticle formulations showed high cytotoxicity (>30% reduction in cell count) when cell counts were compared relative to the mean cell count of eight untreated wells. Values show mean±SEM of three wells for each condition. Differences in transfection efficacy between polymers were assessed over all tested conditions by One-way ANOVA with multiple comparisons to the linear BGDA polymer BGDA-0 using matched values for w/w ratio and DNA dose. One-way ANOVA was performed with Geisser-Greenhouse corrections for sphericity and Dunnet corrections for multiple comparisons. P values shown are multiplicity adjusted.

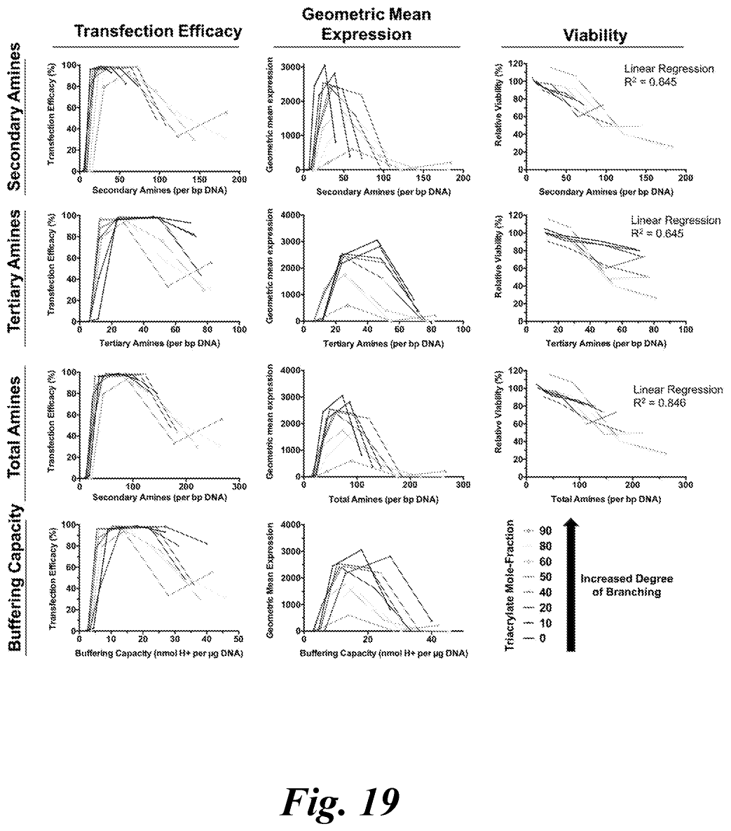

FIG. 19 shows HEK293T transfection correlated with w/w scaled polymer characteristics. The number of secondary amines, tertiary amines, total amines and buffering capacity between pH 5-7.4 were calculated for each polymer at the tested w/w ratios. For viability, linear regression trend lines were calculated to assess if a single curve fit data for all polymers in the series.

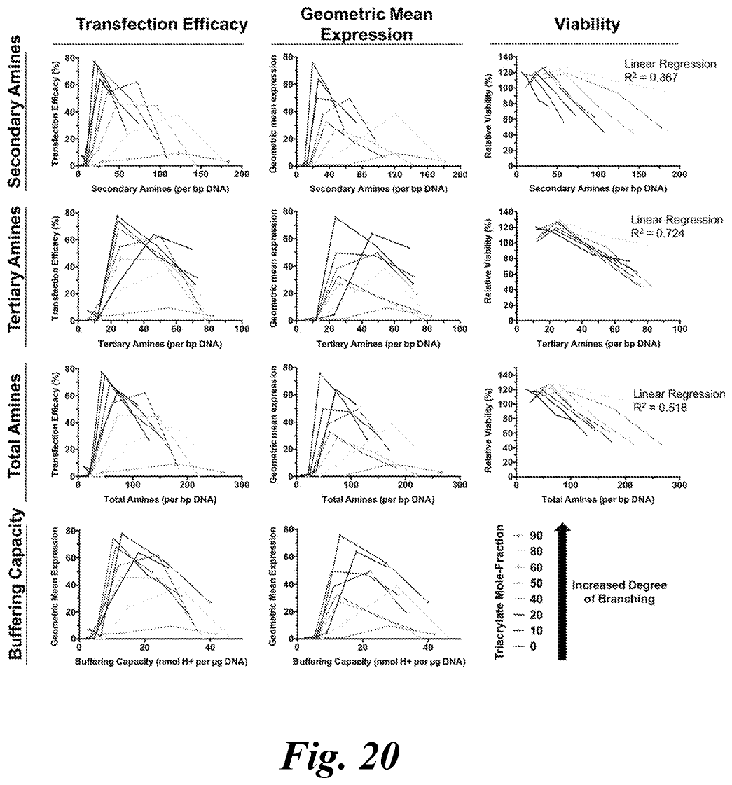

FIG. 20 shows ARPE-19 transfection correlated with w/w scaled polymer characteristics. The number of secondary amines, tertiary amines, total amines and buffering capacity between pH 5-7.4 were calculated for each polymer at the tested w/w ratios. For viability, linear regression trend lines were calculated to assess if a single curve fit data for all polymers in the series.

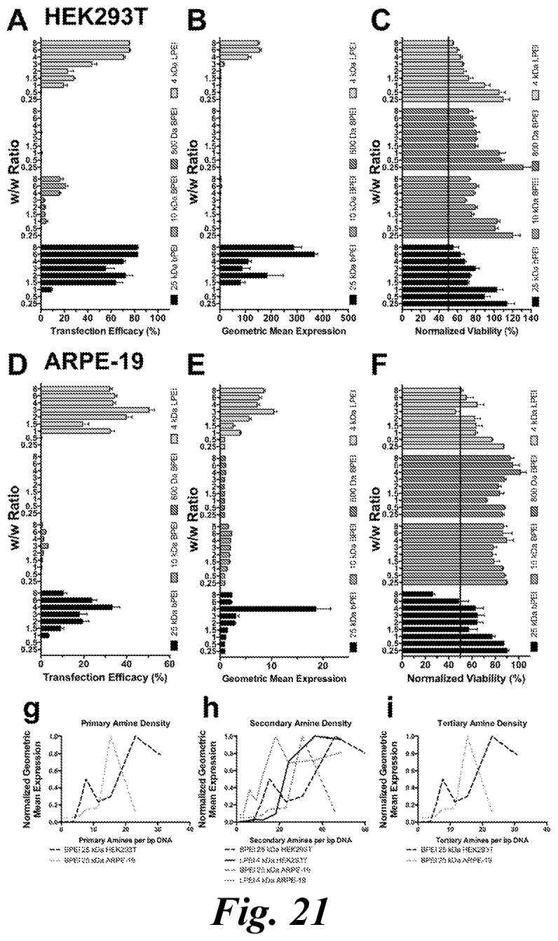

FIG. 21 shows ARPE-19 transfection with control nanoparticle materials.

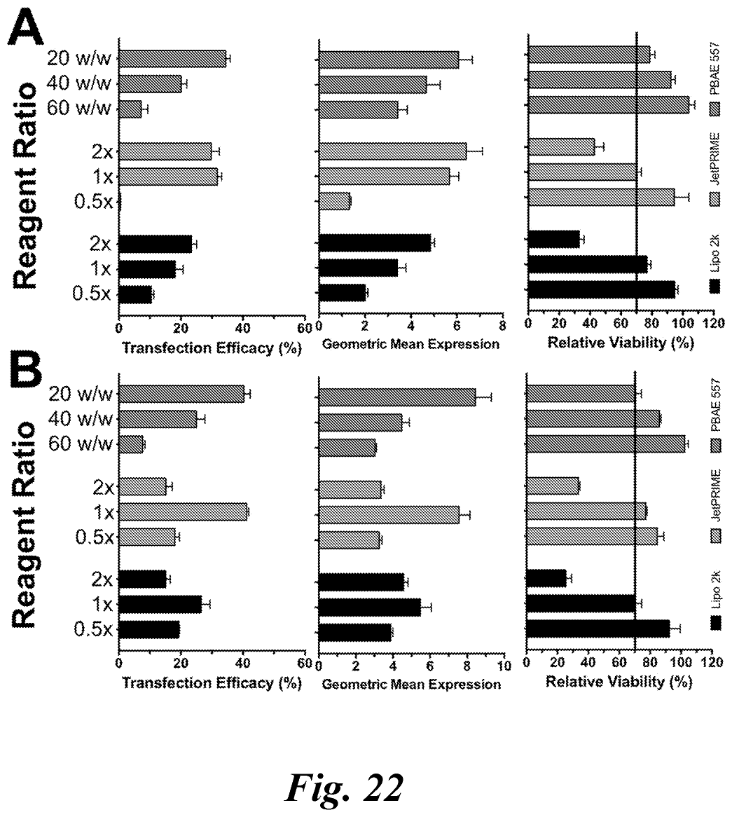

FIG. 22A and FIG. 22B show ARPE-19 transfection with control nanoparticle materials. To fairly identify optimal conditions for in vitro transfection, both a (FIG. 22A) 600 ng dose of DNA with two-hour incubation and a (FIG. 22B) 100 ng dose with 24-hour incubation were tested for control reagents. PBAE 557 was shown previously to be generally effective for transfection of ARPE-19 cells, which we reproduced, showing at most 40% transfection. JetPRIME likewise enabled transfection of up to 40% of cells, while Lipofectamine-2000 gave a transfection efficacy of only 20%.

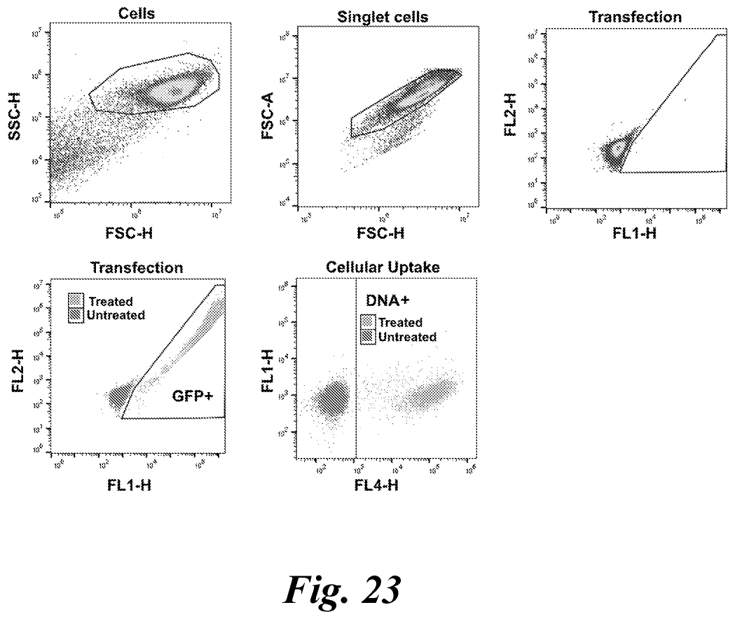

FIG. 23 shows flow cytometry gating analysis. FlowJo 10 was used for gating cells analyzed from an Accuri C₆ flow cytometer. Singlet cell populations were identified and 2D gated for GFP expression or uptake of Cy5 labeled plasmid DNA. For gating, untreated populations were set to be <0.5% false positive.

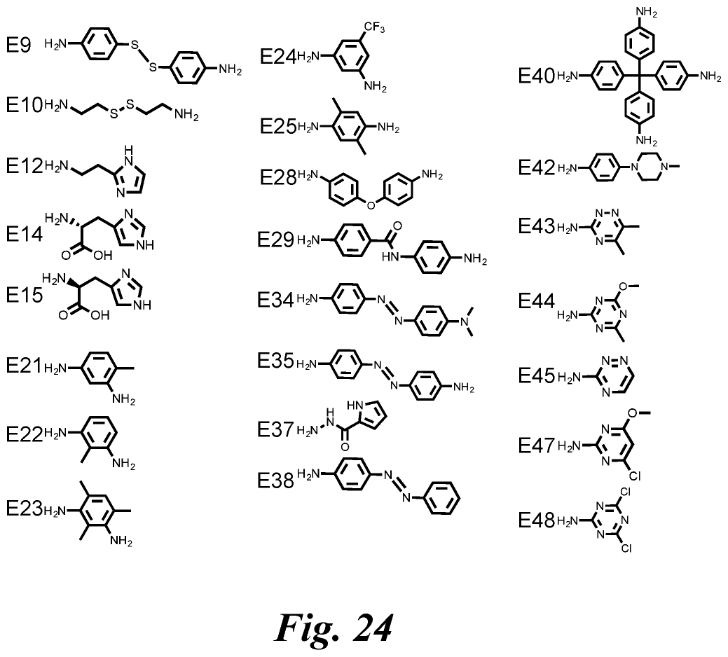

Ineffective endcap monomers are shown in FIG. 24. Endcap structures shown were tested and confirmed to effectively react with acrylate terminated PBAE polymer 4-4-Ac, but the resulting polymers were wholly ineffective for delivery of plasmid DNA to HEK293T cells. These E-monomers were excluded from large library endcapping for transfection efficacy studies in harder-to-transfect RPE monolayers.

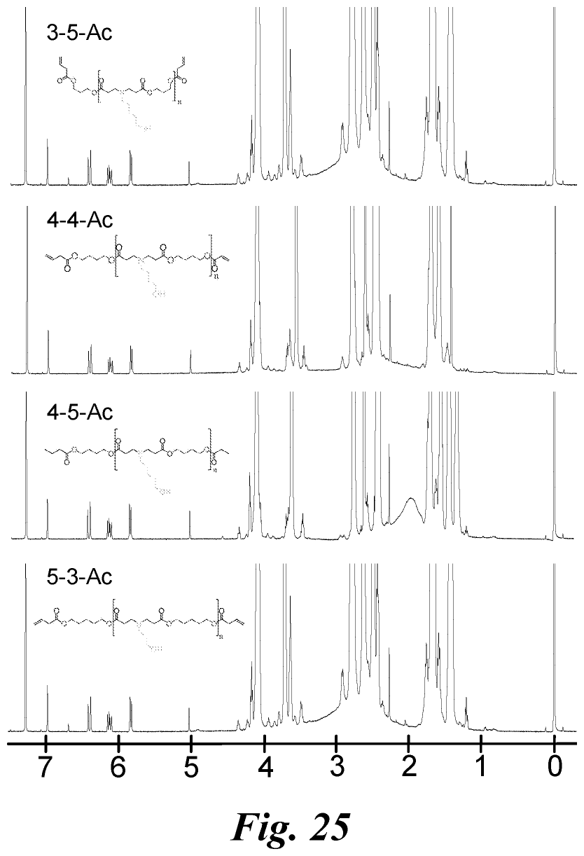

FIG. 25 shows the characterization of base polymer PBAEs via ¹H NMR (500 Mhz) following 2× diethyl ether precipitated to verify that base polymer structures were acrylate terminated. The ratio of integrated acrylate peak area to s-monomer carbon area was used to determine molecular weight MN of base polymers. Calibration and contamination peaks include CDCl₃ 7.26; DMSO 2.62; diethyl ether 1.2 & 3.48; tetramethyl silane (TMS) 0.

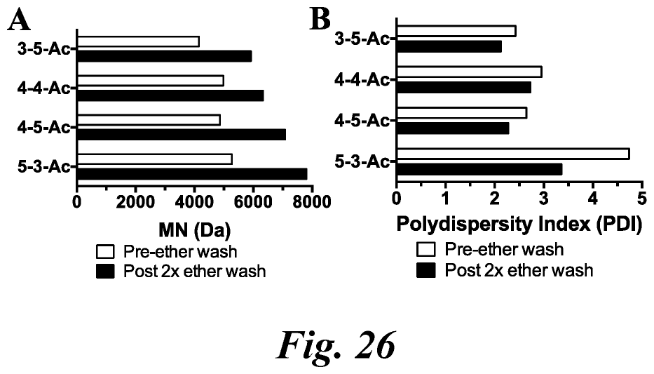

FIGS. 26A-B show gel permeation chromatography characterization of the presently disclosed PBAEs. PBAEs were characterized via gel permeation chromatography to assess molecular weight against linear polystyrene standards following synthesis and after dissolved in DMSO and washed with diethyl ether twice. Washing with diethyl ether was shown to remove unreacted monomers units as well as oligomers, (FIG. 26A) increasing polymer number average weight MN and (FIG. 26B) reducing the polydispersity index (PDI).

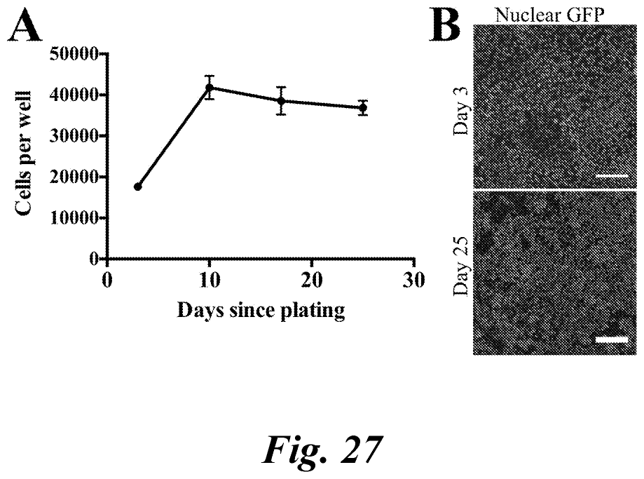

FIGS. 27A-B show the post-mitotic status of differentiated RPE monolayers. Human iPS cells seeded in 384 plates were allowed to differentiate over 25 days in culture in 384 well plates. (FIG. 27A) Cell number per well increases through day 10, at which point cell number peaked and cells began to differentiate. (FIG. 27B) Cells are visibly more densely growing at day 25 post-seeding compared to day 3 post-seeding. RPE monolayer at day 25 additionally possessed textured appearance. Bars show mean±SEM of four wells for each condition. Scale bar 100 μm for 20× images.

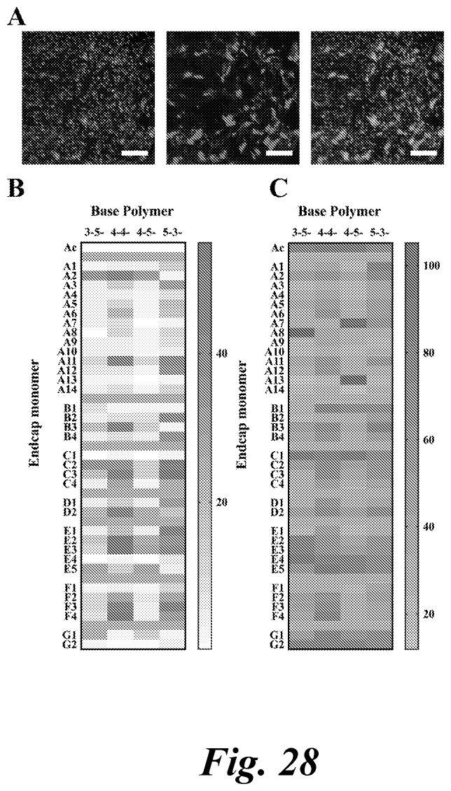

FIG. 28A, FIG. 28B, and FIG. 28C show full differentiation from embryonic stem cells changes cell phenotype and optimal PBAE polymer structure. Scale bars are 100 μm. (FIG. 28A) Representative images of D3 RPE cells after plating transfected with 4-4-E2. (FIG. 28B) Heat map of transfection of D3 RPE with full PBAE library; (FIG. 28C) D3 viability heat map with full PBAE library;

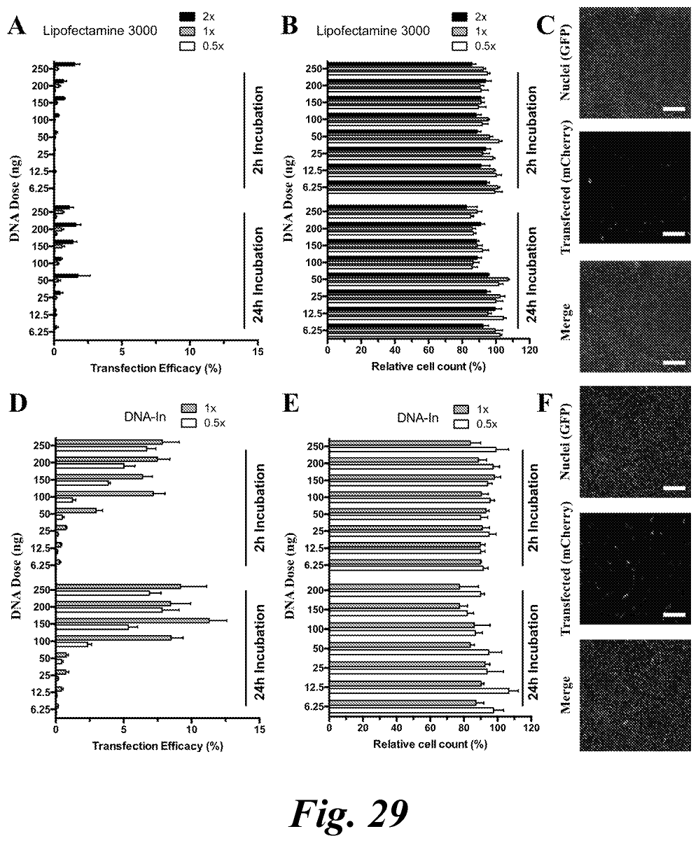

FIGS. 29A-F show commercial reagent transfection efficacy optimization. Lipofectamine 3000 and DNA-In were tested under 2-hour and 24-hour incubation conditions at varying reagent ratio and DNA doses to identify the optimal condition for each. (FIG. 29A) Lipofectamine 3000 transfected at most 3% of cells and (FIG. 29B) resulted in minimal cytotoxicity compared to untreated cells at a 50 ng, 2× reagent concentration dose with a 24-hour incubation period. (FIG. 29C) Microscope images show constitutive nuclear GFP expression and low number of mCherry expressing transfected cells. (FIG. 29D) DNA-In resulted in at most 12% transfection efficacy with (FIG. 29E) manageable cytotoxicity at a 150-ng dose and 24-hour incubation time. (FIG. 29F) DNA-In visibly transfected a higher fraction of cells, but the majority remain untransfected. Bars show mean±SEM of four wells for each condition. Scale bar 200 μm for 10× images.

Representative base monomers used to prepare the presently disclosed branched polymers are shown in FIG. 7A. The polymers can be designated, for example, as 7,8-4 acrylate for monomers BGDA, TMPTA-S4-acrylate as the base polymer.

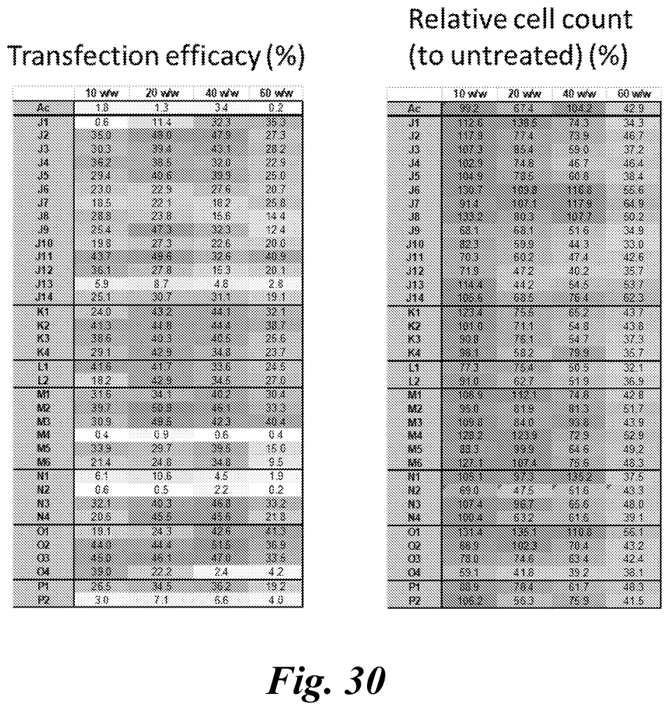

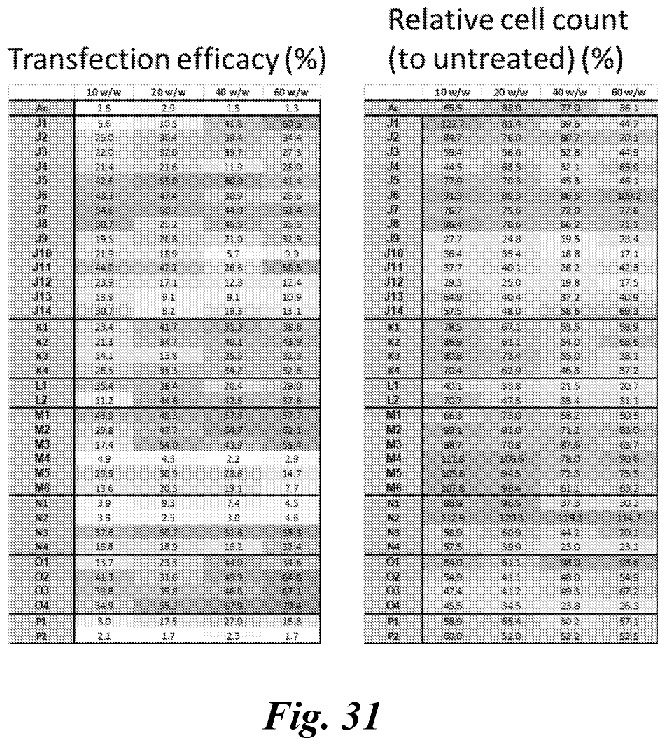

FIG. 30 shows the transfection efficacy and the relative cell count to untreated for the GL261 high throughput screening of base polymer endcaps. 20% triacrylate mole fraction BGDA-TMPTA-B4 polymer (7,8-4-Ac). 384 well plates, 75-ng DNA/well with 2-hr incubation. Transfection efficacy was assessed by cellomics.

FIG. 31 shows the transfection efficacy and the relative cell count to untreated for the B16-F10 high throughput screening of base polymer endcaps. 20% triacrylate mole fraction BGDA-TMPTA-B4 polymer (7,8-4-Ac). 384 well plates, 75-ng DNA/well with 2-hr incubation. Transfection efficacy was assessed by cellomics.

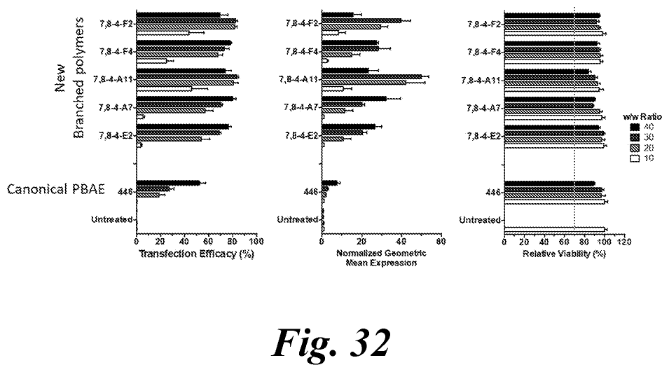

FIG. 32 shows the transfection efficacy, normalized geometric mean expression, and relative viability for GL261 mouse glioma cells, where 96-well transfection efficacy was assessed by flow cytometry, with 400 ng/well, and 2-hr incubation. 7,8-4-XX polymers are 20% branching monomer with the new, expanded endcap library. The new polymers yield up to 80% transfection, even at 20 w/w ratio (see 7,8-4-A11 polymer) compared to canonical PBAE 446, which required at least 40 w/w ratio and only gave 55% transfection. Geometric mean expression also increased with new polymers, while viability was maintained.

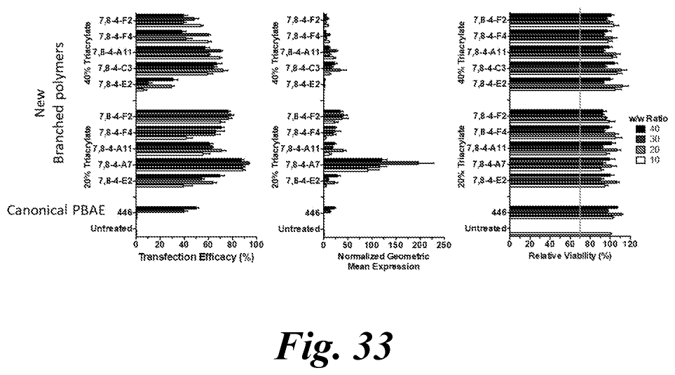

FIG. 33 shows the transfection efficacy, normalized geometric mean expression, and relative viability for B16-F10 mouse melanoma cells, where 96-well transfection efficacy was assessed by flow cytometry, with 600 ng/well, and 2-hr incubation. 7,8-4-XX polymers are 20% or 40% branching monomer with the new, expanded endcap library. The new polymers yield up to 95% transfection, even at 10 w/w ratio (see 7,8-4-A7 polymer) compared to canonical PBAE 446, which required at least 40 w/w ratio and only gave approximately 55% transfection. Geometric mean expression also increased with new polymers, while viability was maintained.

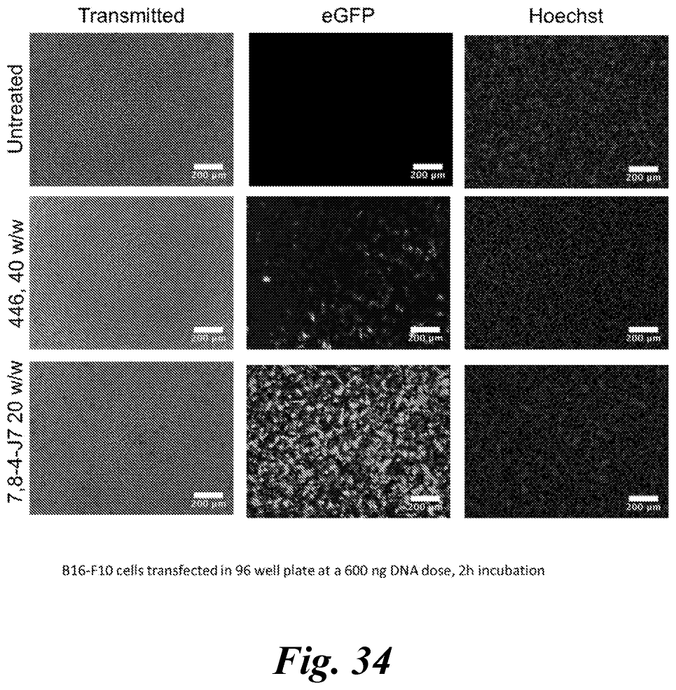

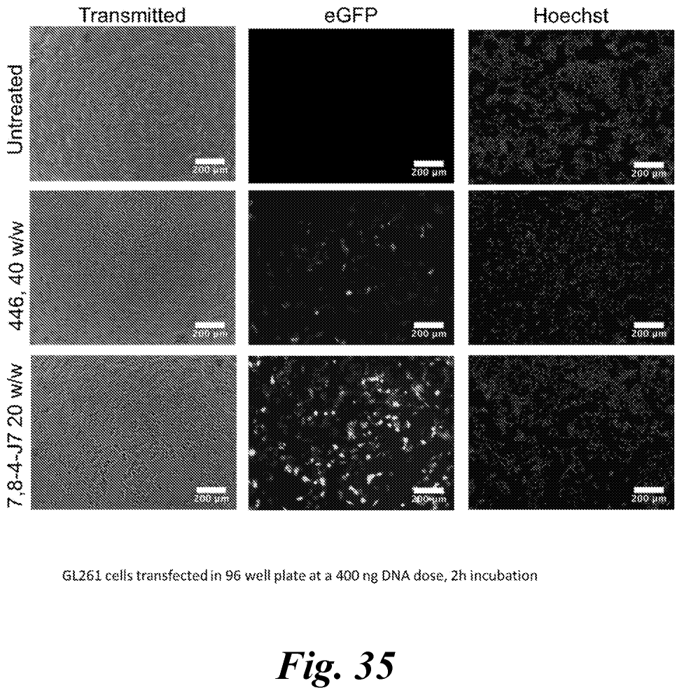

FIG. 34 shows images of B16-F10 cells transfected in 96-well plate at a 600 ng DNA dose, 2-hr incubation.

FIG. 35 shows images of GL261 cells transfected in 96-well plate at 400 ng DNA does, 2-hr incubation.

Monomer ratios for polymer synthesis are provided in Table 3. ¹H NMR integration values for the presently disclosed BGDA series of polymers are provided in Table 4. Tertiary amine density calculations are provided in Table 5. For the data presented in Table 5, the molecular weight for polymer repeat units consisting of monomers BGDA+S4, TMPTA+2*S4 and ethylenimine were calculated Amine density was then determined as the number of amines per polymer backbone molecular weight in Da. The branching monomer TMPTA gives rise to polymers with the highest tertiary amine density while BGDA monomers give rise to polymers with a lower tertiary amine density.

TABLE 3
Monomer mole ratios for synthesis of BGDA PBAE series.

| Planned Triacrylate | Diacrylate | Triacrylate | Amine |
|---|---|---|---|
| Mole Fraction (%) | Ratio | Ratio | Ratio |
| 0 | 1.1 | 0.00 | 1 |
| 10 | 0.99 | 0.07 | 1 |
| 20 | 0.88 | 0.15 | 1 |
| 40 | 0.66 | 0.29 | 1 |
| 50 | 0.55 | 0.37 | 1 |
| 60 | 0.44 | 0.44 | 1 |
| 80 | 0.22 | 0.59 | 1 |
| 90 | 0.11 | 0.66 | 1 |
| 100 | 0 | 0.73 | 1 |

TABLE 4
¹H NMR integrations for BGDA series
normalized to acrylate peaks (3H)

| Planned | BGDA Phenyl | B8 methyl | S4 |
|---|---|---|---|
| Triacrylate | 7.11 & 6.8 ppm | 0.83 ppm | 2.38 ppm |
| Mole % | (4H each) | (3H) | (2H) |
| 0 | 9.42 | 0 | 4.62 |
| 10 | 7.16 | .91 | 5.05 |
| 20 | 6.41 | 1.25 | 4.62 |
| 40 | 6.27 | 1.87 | 3.70 |
| 50 | 4.08 | 2.58 | 4.63 |
| 60 | 2.19 | 2.02 | 3.40 |
| 80 | .951 | 3.10 | 3.19 |
| 90 | .611 | 4.16 | 3.57 |

TABLE 5
Backbone polymer amine density calculations.

| | Molecular Weight | Amine density |
|---|---|---|
| Repeat Unit | (Da) | (Amines/Da) |
| BGDA + S4 | 573 | 0.00175 |
| TMPTA + 2*S4 | 474 | 0.00422 |
| Ethylenimine | 43 | 0.02326 |

Table 6 presents monomers used for PBAE library synthesis for screening RPE cells. Acrylate terminated polymers were synthesized from small molecule diacrylate and primary amine monomers followed by high-throughput endcapping with 37 monomers organized into different structural categories.

TABLE 6
Selected Monomers used for Polymer Synthesis

| | Monomer | Internal | MW | CAS | |
|---|---|---|---|---|---|
| Chemical Name | Name | Name | (Da) | number | Supplier |
| Base-Monomers (B) | | | | | |
| 1,3-propanediol diacrylate | B3 | B3 | 184.19 | 24493-53-6 | Monomer- |
| | | | | | Polymer and |
| | | | | | Dajac Labs |
| 1,4-Butanediol diacrylate | B4 | B4 | 198.22 | 1070-70-8 | Alfa Aesar |
| 1,5-Pentanediol diacrylate | B5 | B5 | 212.24 | 36840-85-4 | Monomer- |
| | | | | | Polymer and |
| | | | | | Dajac Labs |
| Side-chain-Monomers (S) | | | | | |
| 3-amino-1-propanol | S3 | S3 | 75.11 | 156-87-6 | Alfa Aesar |
| 4-amino-1-butanol | S4 | S4 | 89.14 | 13325-10-05 | Fisher |
| | | | | | Scientific |
| 5-amino-1-pentanol | S5 | S5 | 103.16 | 2508-29-4 | Alfa Aesar |
| Endcap Monomers | | | | | |
| 1,3-diaminopropane | A1 | E1 | 74.12 | 109-76-2 | Sigma |
| | | | | | Aldrich |
| 2,2-dimethyl-1,3- | A2 | E2 | 102.18 | 7328-91-8 | Sigma |
| propanediamine | | | | | Aldrich |
| 1,3-diaminopentane | A3 | E3 | 102.18 | 589-37-7 | TCI America |
| 2-methyl-1,5-diaminopentane | A4 | E4 | 116.2 | 15520-10-02 | TCI America |
| Diethylentriamine | A5 | E63 | 103.17 | 111-40-0 | EMD |
| | | | | | Millipore |
| Triethylenetetramine | A6 | E30 | 146.23 | 112-24-3 | Sigma |
| | | | | | Aldrich |
| Tetraethylenepentamine | A7 | E31 | 189.3 | 1112-57-2 | Sigma |
| | | | | | Aldrich |
| Pentaethylenehexamine | A8 | E60 | 232.44 | 4067-16-7 | Santa Cruz |
| N,N- | A9 | E49 | 159.27 | 10563298 | Sigma |
| Dimethyldipropylenetriamine | | | | | Aldrich |
| 3,3′-Diamino-N- | A10 | E52 | 145.25 | 105-83-9 | MP |
| methyldipropylamine | | | | | Biomedicals |
| N,N- | A11 | E58 | 159.27 | 24426-16-2 | Sigma |
| Diethyldiethylenetriamine | | | | | Aldrich |
| 3,3′-Iminobis(N,N- | A12 | E56 | 187.33 | 6711484 | Santa cruz |
| dimethylpropylamine) | | | | | |
| Tris(2-aminoethyl)amine | A13 | E32 | 146.23 | 4097-89-6 | Sigma |
| | | | | | Aldrich |
| Tris[2- | A14 | E54 | 188.31 | 65604-89-9 | Sigma |
| (methylamino)ethyl]amine | | | | | Aldrich |
| 1-(2-Aminoethyl)piperidine | B1 | E53 | 128.22 | 27578-60-5 | Alfa Aesar |
| N-(3-Aminopropyl)piperidine | B2 | E61 | 128.22 | 3529-08-6 | Sigma |
| | | | | | Aldrich |
| 2-(Aminomethyl)piperidine | B3 | E50 | 114.19 | 22990-77-8 | Sigma |
| | | | | | Aldrich |
| 4-(Aminomethyl)piperidine | B4 | E64 | 114.19 | 7144-05-0 | Fisher |
| | | | | | Scientific |
| 1-Amino-4-methylpiperazine | C1 | E40 | 115.18 | 6928-85-4 | Sigma |
| | | | | | Aldrich |
| 1-(2-Aminoethyl)piperazine | C2 | E39 | 129.2 | 140-31-8 | Sigma |
| | | | | | Aldrich |
| 1-(3-Aminopropyl)-4- | C3 | E7 | 157.26 | 4572-031 | Alfa Aesar |
| methylpiperazine | | | | | |
| 1,4-Bis(3- | C4 | E65 | 200.33 | 7209-38-3 | MP |
| aminopropyl)piperazine | | | | | Biomedicals |
| 1-(3-Aminopropyl)pyrrolidine | D1 | E8 | 128.22 | 23159-07-01 | TCI America |
| 1-(2-Aminoethyl)pyrrolidine | D2 | E59 | 114.19 | 7154-73-6 | Santa Cruz |
| 2-(3- | E1 | E6 | 118.18 | 4461-39-6 | Sigma |
| Aminopropylamino)ethanol | | | | | Aldrich |
| N-(3- | E2 | E51 | 118.18 | 56344-32-2 | TCI America |
| Hydroxypropyl)ethylenediamine | | | | | |
| N-(2- | E3 | E62 | 104.15 | 111-41-1 | EMD |
| Hydroxyethyl)ethylenediamine | | | | | Millipore |
| N,N′-Bis(2- | E4 | E16 | 148.2 | 4439-20-7 | TCI America |
| hydroxyethyl)ethylenediamine | | | | | |
| 2-(2-Aminoethoxy)ethanol | E5 | E55 | 105.14 | 929066 | Alfa Aesar |
| N,N-Bis(2- | E6 | E18 | 148.2 | 3197-06-6 | Alfa Aesar |
| hydroxyethyl)ethylenediamine | | | | | |
| 2,2′-Oxybis(ethylamine) | F1 | E57 | 104.15 | 2752-17-2 | Acros |
| | | | | | Organics |
| 2,2′- | F2 | E33 | 148.2 | 929-59-9 | Sigma |
| (Ethylenedioxy)bis(ethylamine) | | | | | Aldrich |
| 1,11-diamino-3,6,9- | F3 | E5 | 192.26 | 929-75-9 | TCI America |
| trioxaundecane | | | | | |
| 4,7,10-Trioxa-1,13- | F4 | E27 | 220.31 | 4246-51-9 | Sigma |
| tridecanediamine | | | | | Aldrich |
| 3-Morpholinopropylamine | G1 | E89 | 144.21 | 123-00-2 | Sigma |
| | | | | | Aldrich |
| 4-(2-Aminoethyl)morpholine | G2 | E90 | 130.19 | 2038-031 | Sigma |
| | | | | | Aldrich |

Table 7 presents minimally effective endcap monomers. Base polymer 4-4-Ac was pre-screened in HEK293T cells following endcapping with monomers in Table 7.

TABLE 7
Minimally Effective Endcap Monomers

| | Monomer | | CAS | |
|---|---|---|---|---|
| Chemical Name | Name | MW | number | Supplier |
| Minimally effective endcap monomers | | | | |
| 4-Aminophenyl disulfide | E9 | 248.37 | 722-27-0 | Sigma |
| | | | | Aldrich |
| Cystamine dihydrochloride | E10 | 250.2 | 56-17-7 | Alfa |
| | | | | Aesar |
| Histamine | E12 | 111.15 | 51-45-6 | Sigma |
| | | | | Aldrich |
| D-Histidine | E14 | 155.15 | 351-50-8 | Sigma |
| | | | | Aldrich |
| L-Histidine | E15 | 155.15 | 71-00-1 | Sigma |
| | | | | Aldrich |
| 2,4-Diaminotoluene | E21 | 122.17 | 95-80-7 | Sigma |
| | | | | Aldrich |
| 2,6-Diaminotoluene | E22 | 122.17 | 823-40-5 | Sigma |
| | | | | Aldrich |
| 2,4,6-Trimethyl-phenylenediamine | E23 | 150.22 | 3102-70-3 | Sigma |
| | | | | Aldrich |
| 5-(Trifluoromethyl)-1,3- | E24 | 176.14 | 368-53-6 | Sigma |
| phenylenediamine | | | | Aldrich |
| p-Phenylenediamine | E25 | 108.14 | 106-50-3 | Sigma |
| | | | | Aldrich |
| 2,5-Dimethyl-1,4-phenylenediamine | E26 | 136.19 | 6393017 | Sigma |
| | | | | Aldrich |
| 4,4′-Oxydianiline | E28 | 200.24 | 101-80-4 | Sigma |
| | | | | Aldrich |
| 4-Diaminobenzanilide | E29 | 227.26 | 785-30-8 | Sigma |
| | | | | Aldrich |
| N,N-Dimethyl-4,4′-azodianiline | E34 | 240.3 | 539-17-3 | Sigma |
| | | | | Aldrich |
| 4-[(E)-(4- | E35 | 212.256 | PH010934 | Sigma |
| aminophenyl)diazenyl]phenylamine | | | | Aldrich |
| 1H-pyrrole-2-carbohydrazide | E37 | 125.13 | 50269-95-9 | Sigma |
| | | | | Aldrich |
| 4-Aminoazobenzene | E38 | 197.24 | 60-09-3 | Sigma |
| | | | | Aldrich |
| Tetrakis(4-aminophenyl)methane | E40 | 380.48 | 60532-63-0 | Sigma |
| | | | | Aldrich |
| 1-(4-Aminophenyl)piperazine | E42 | 177.25 | 67455-41-8 | Sigma |
| | | | | Aldrich |
| 3-Amino-5,6-dimethyl-l,2,4-triazine | E43 | 124.14 | 17584-12-2 | VWR |
| 2-Amino-4-methoxy-6-methyl-1,3,5- | E44 | 140.14 | 1668-54-8 | Sigma |
| triazine | | | | Aldrich |
| 3-Amino-1,2,4-triazine | E45 | 96.09 | 1120-99-6 | Acros |
| | | | | Organics |
| 2-Amino-4-chloro-6-methoxypyrimidine | E47 | 130.19 | 5734-64-5 | Sigma |
| | | | | Aldrich |
| 2-Amino-4,6-dichloro-1,3,5-triazine | E48 | 164.98 | 933-20-0 | Sigma |
| | | | | Aldrich |

In some embodiments, variably hyperbranched PBAEs with expanded endcap molecules were screened. Accordingly, a combination of hyperbranching in PBAEs with more effective endcap molecules were identified through high-throughput screening. More particularly, in some embodiments, a BGDA-40% branched polymer was tested in B16-F10 melanoma cells at low nanoparticle doses to identify optimal endcap structures in hyperbranched polymers that are much more effective at lower w/w ratios. The table below shows transfection efficacy as a percent of all cells in each well expressing CAG-mCherry reporter plasmid DNA two days after transfection with nanoparticles. The heat-map shows the average of two replicate wells per cell analyzed by Cellomics Arrayscan image-based quantification of transfection. B16-F10 melanoma cells were plated in 384 well plates and transfected with the nanoparticle prepared at specified w/w ratios to identify branched polymer structures, end-capped with the expanded end-cap library that yielded transfection at low w/w ratios (particulary 20 w/w or lower).

| Base | 10 w/w | 20 w/w | 40 w/w | 60 w/w |
|---|---|---|---|---|
| Polymer | Mean | Mean | Mean | Mean |
| Ac | 0.1 | 0.1 | 0.1 | 0.2 |
| A1 | 0.0 | 6.6 | 30.4 | 30.8 |
| A2 | 15.3 | 23.9 | 27.6 | 30.9 |
| A3 | 10.4 | 24.9 | 31.1 | 33.7 |
| A4 | 12.7 | 9.9 | 6.4 | 14.2 |
| A5 | 16.9 | 23.1 | 21.6 | 34.5 |
| A6 | 27.4 | 20.3 | 20.2 | 27.9 |
| A7 | 35.5 | 28.0 | 28.7 | 21.2 |
| A8 | 26.9 | 19.8 | 18.6 | 19.8 |
| A9 | 16.6 | 13.8 | 10.4 | 17.6 |
| A10 | 8.9 | 11.6 | 8.9 | 8.8 |
| A11 | 29.8 | 27.2 | 30.0 | 33.3 |
| A12 | 11.4 | 19.9 | 19.7 | 17.0 |
| A13 | 1.3 | 1.3 | 1.6 | 0.2 |
| A14 | 13.1 | 3.5 | 5.1 | 1.5 |
| B1 | 11.8 | 25.2 | 32.8 | 34.9 |
| B2 | 14.3 | 35.8 | 37.2 | 38.4 |
| B3 | 15.9 | 17.1 | 24.6 | 28.2 |
| B4 | 17.5 | 21.7 | 22.4 | 20.6 |
| C1 | 1.1 | 5.5 | 14.5 | 3.0 |
| C2 | 0.0 | 0.0 | 0.1 | 0.0 |
| C3 | 28.5 | 33.6 | 37.7 | 35.9 |
| C4 | 9.1 | 13.0 | 14.2 | 22.1 |
| D1 | 22.8 | 33.0 | 24.4 | 12.5 |
| D2 | 13.4 | 23.5 | 23.5 | 16.3 |
| E1 | 21.9 | 34.8 | 40.1 | 44.0 |
| E2 | 12.4 | 24.9 | 31.4 | 40.8 |
| E3 | 13.4 | 24.6 | 22.9 | 39.1 |
| E4 | 0.1 | 0.1 | 0.2 | 0.1 |
| E5 | 11.1 | 11.9 | 17.6 | 17.4 |
| E6 | 8.7 | 12.9 | 15.0 | 22.3 |
| F1 | 9.4 | 11.7 | 17.2 | 12.7 |
| F2 | 18.5 | 19.8 | 27.0 | 31.2 |
| F3 | 19.6 | 20.7 | 24.3 | 31.2 |
| F4 | 10.0 | 20.7 | 23.4 | 36.0 |
| G1 | 23.3 | 26.6 | 29.5 | 26.1 |
| G2 | 5.0 | 4.8 | 5.7 | 3.7 |

### Example 3

### Branched Ester-Amine Quadpolymers (BEAQs) Enhance Efficiency of Intracellular Nucleic Acid Delivery

3.1 Introduction. Highly branched polymers are challenging to synthesize in a reproducible manner, but show great potential for demonstrating improved gene transfection efficiency compared to more commonly used linear polyester amines Zhao T et al. (2014). The molecular flexibility of branched polycations allows for stronger interactions with nucleic acids, which can improve nanoparticle formation. Cutlar L, Zhou D, et al. (2015).

The presently disclosed subject matter provides, in part, the synthesis of a library of highly branched poly(beta-amino ester)s) (PBAEs) that can self-assemble with plasmid DNA to form polyplex nanoparticles capable of high transfection efficacy with significant improvements over linear polyester amines known in the art.

3.2 Methods

BEAQs were synthesized in DMSO with an overall 2.2:1 overall vinyl:amine monomer ratio using step-growth Michael addition reactions followed by end-capping and ether purification. The synthesized BEAQs were characterized by ¹H-NMR spectroscopy with a Bruker 500 MHz NMR spectrometer in CDCl₃. Gel permeation chromatography (GPC) was conducted with MW, MN and PDI relative to linear polystyrene standards. The DNA competition binding assay included Yo-Pro-1 iodide and plasmid DNA at 1 μM. For polyplex formation, DNA and PBAE polymer were diluted in 25 mM NaAc, pH 5.0, then mixed in a 1:1 volumetric ratio to allow for nanoparticle self-assembly.

HEK293T, ARPE-19, B16-F10, GL261 cells were tested for transfection. Cell uptake and transfection was assessed using flow cytometry with Cy5 labeled plasmid DNA or reporter gene constructs.

3.3. Results. BEAQs more effectively bind nucleic acids as a function of endcap moiety density and branching structure. BEAQs demonstrate vastly greater transfection efficacy compared to equivalent linear and lowly branched polymers and greater than two times transfection efficacy in RPE cells compared to commercial reagents and previous generation PBAE nanoparticles. BEAQs exhibit a consistent optimal tertiary amine density necessary for transfection, while optimal secondary amine density varied with polymer structure. The expanded library of BEAQs enables high transfection in variety of other cell types, including B16-F10, GL261, A549 greater efficacy at low w/w ratios.

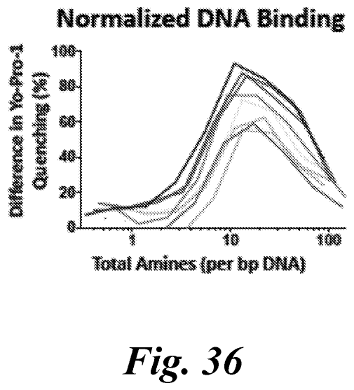

FIG. 36 shows normalized DNA binding (see also FIG. 8 for related data).

FIG. 37 (top) shows the optimal w/w ratio relative to triacrylate mole fraction.

FIG. 37 (bottom) shows the optimal amine density relative to triacrylate mole fraction (see also FIG. 10 for related data).

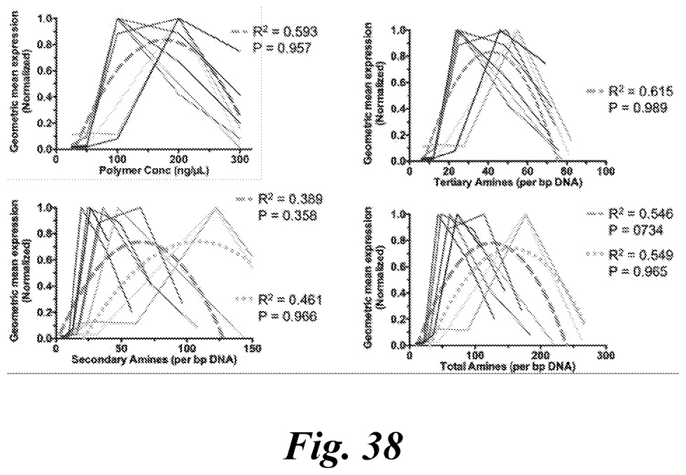

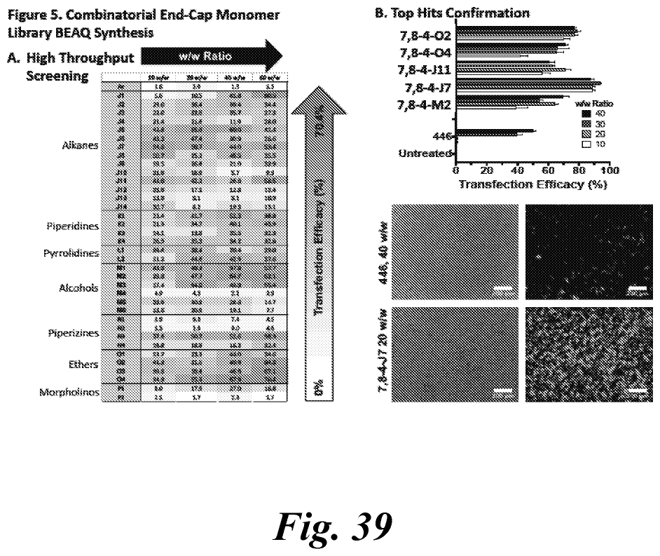

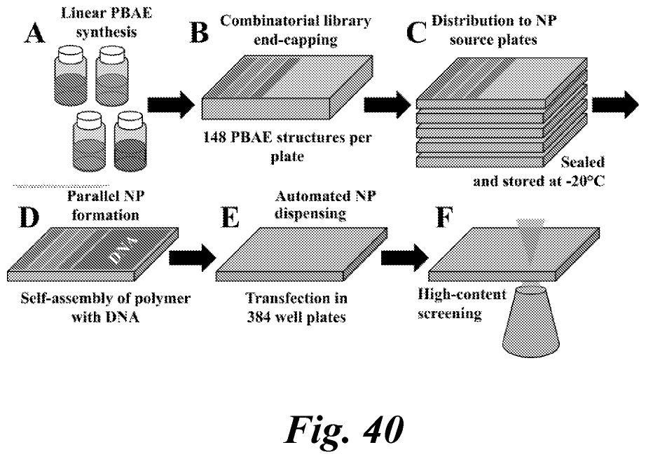

FIG. 38 shows gene expression and nanoparticle property correlation for ARPE-19 cells.

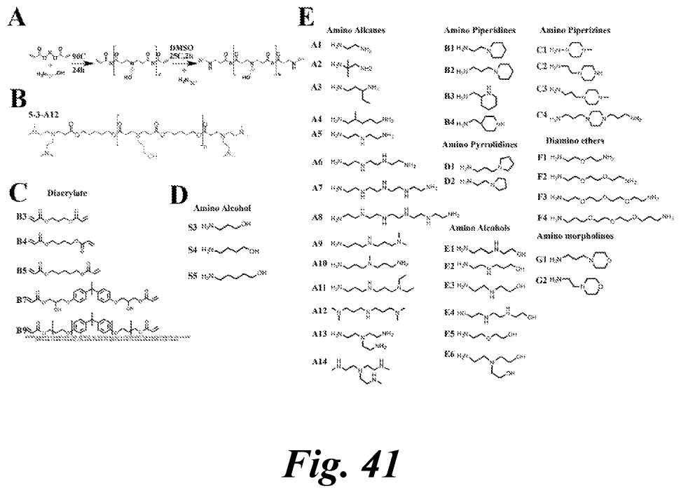

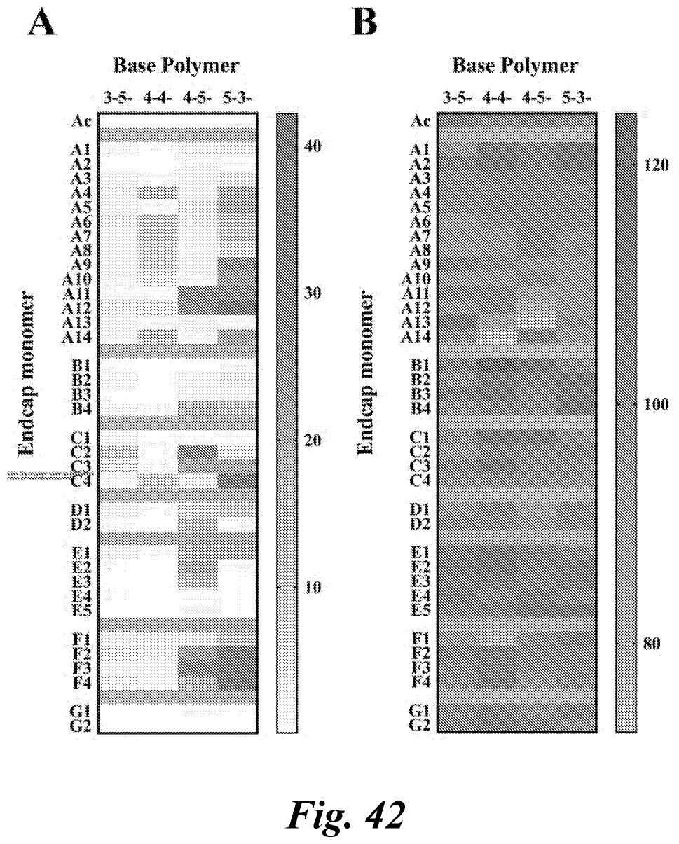

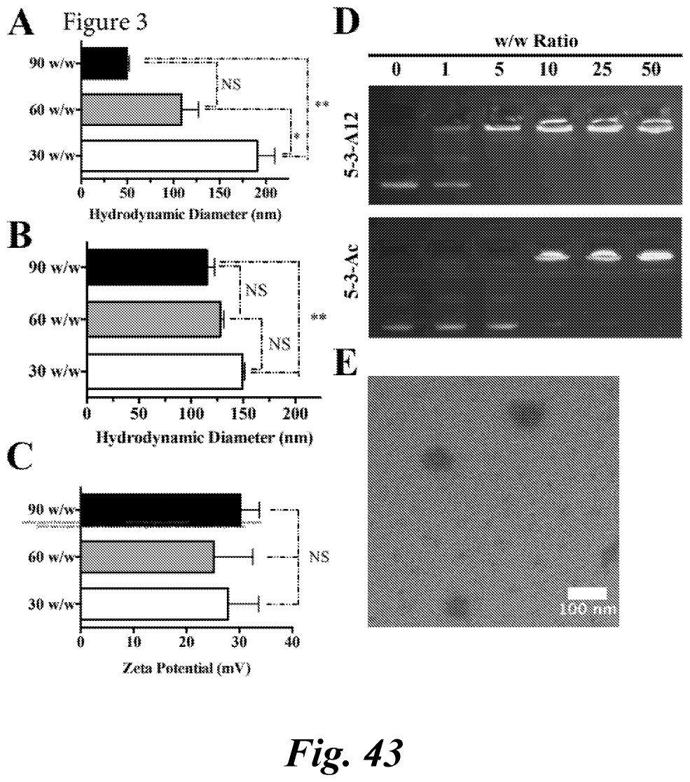

FIG. 41A and FIG. 41B show combinatorial end-cap monomer library BEAQ synthesis. FIG. 41A shows high-throughput screening. FIG. 41B shows top hit confirmation.

### Example 4

### Reducible Branched Poly(Ester Amine) Quadpolymers (rBEAQs) Co-Delivering Plasmid DNA and RNA oligonucleotides Enable CRISPR/Cas9 Genome Editing

4.1 Introduction. Functional co-delivery of plasmid DNA and RNA oligonucleotides in the same nanoparticle system is challenging due to differences in cargo size, stiffness, and intracellular sites of function. Co-delivery of plasmid DNA upregulating gene expression with short RNAs such as short interfering RNA (siRNA) to knockdown gene expression or short guide RNA (sgRNA) to enable CRISPR/Cas9 gene editing may be useful for novel combinatorial gene therapies.

The presently disclosed subject matter provides the synthesis of a library of bio-reducible branched poly(beta-amino ester)s) (PBAEs) that can self-assemble with plasmid DNA and short RNAs such as siRNA or sgRNA to form polyplex nanoparticles capable of high transfection efficacy with significant improvements over linear polyester amines known in the art.

4.2 Methods.

rBEAQs were synthesized in DMSO with an overall 2.2:1 overally vinyl amine monomer ration using step-growth Michael addition reactions followed by end-capping and ether purification. The synthesized rBEAQs were characterized by ¹H-NMR spectroscopy for polymer structure, gel permeation chromatography (GPC) for molecular weight characterization, Yo-Pro-1 iodide competition binding assay for nucleic acid binding strength, and gel retardation assay for nucleic acid release kinetics. For polyplex formation, DNA/RNA oligos and PBAE polymer were diluted in 25 mM NaAc, pH 5.0, then mixed in a 1:1 volumetric ratio to allow for nanoparticle self-assembly.

HEK293T and Huh7 cells constitutively expressing destabilized eGFP were tested for transfection and siRNA knockdown. Cell uptake and transfection were assessed using flow cytometry with Cy5-labeled siRNA or reporter gene constructs.

4.3 Results. rBEAQs exhibited a bi-phasic response in siRNA-induced gene knockdown, cell viability, and cellular uptake; linear and highly branched polymers performed poorly while moderately branched polymers exhibited the optimal performance in all three categories. Addition of BGDA monomer (denoted here as B7) increased co-delivery in both cell lines tested at low w/w ratios, and optimal formulations performed as well or better than commercial reagents. Co-delivery of Cas9 DNA and sgRNA resulted in CRISPR gene knock-out in HEK293T cells.

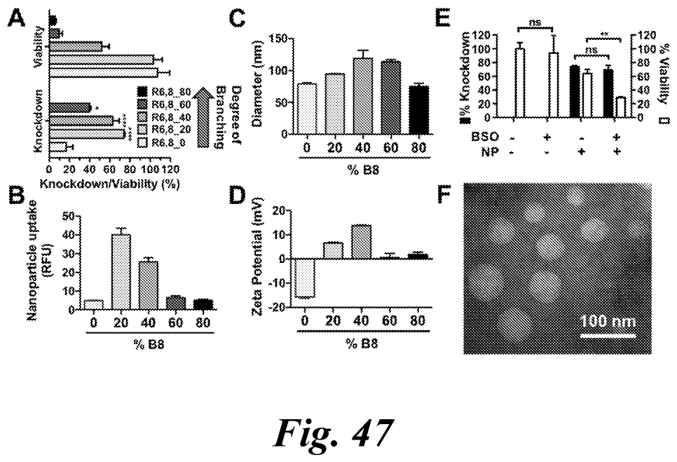

FIGS. 47A, 47B, 47C, 47D, 47E, and 47F show the rBEAQs form nanoparticles with siRNA and enable gene knockdown. FIG. 47A shows knockdown and cell viability of rBEAQ-siRNA nanoparticles on HEK293 Ts. FIG. 47B shows cellular uptake. FIG. 47C shows nanoparticle hydrodynamic diameter as measured by NTA. FIG. 47D shows nanoparticle zeta potential as measured by DLS. FIG. 47E shows that when intracellular glutathione is blocked using the drug BSO, nanoparticle-mediated cytotoxicity increased. FIG. 47F shows TEM images of rBEAQ-siRNA nanoparticles.

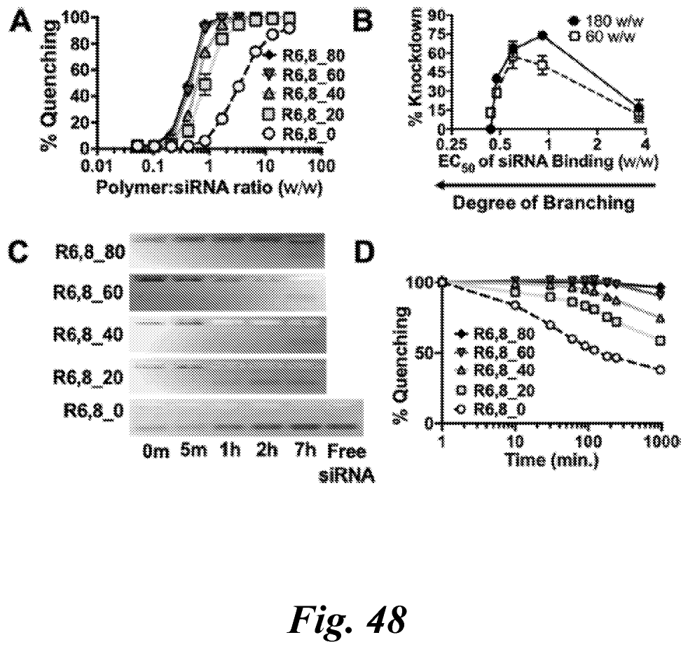

FIGS. 48A, 48B, and 48C show rBEAQ siRNA binding and release kinetics. FIG. 48A shows Yo-Pro-1 siRNA binding assay indicating that polymer branching increased siRNA binding strength. FIG. 48B shows that siRNA knockdown plotted against the EC50 of binding showed a biphasic response. FIG. 48C shows a gel retardation assay of rBEAQ nanoparticles incubated over time in 5 mM glutathione reducing environment.

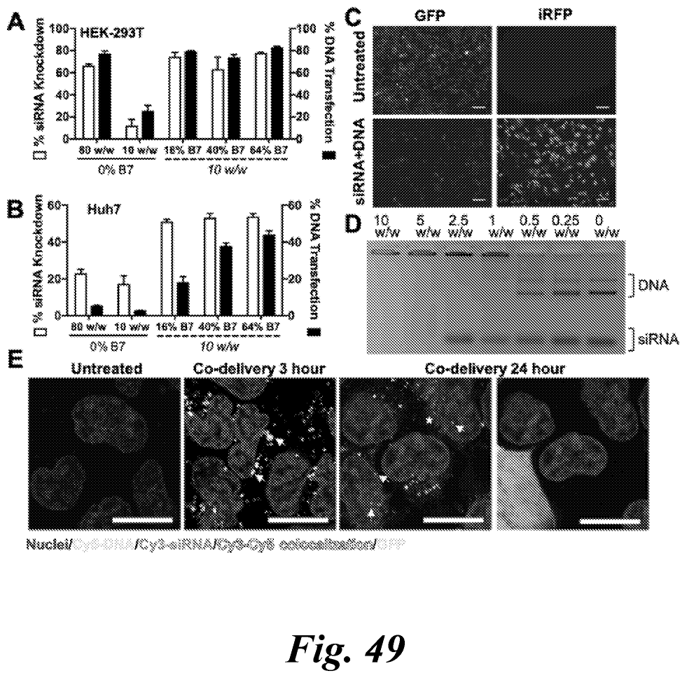

FIGS. 49A, 49B, and 49C show rBEAQs containing monomer B7 enabled efficient co-delivery of DNA and siRNA to HEK293T and Huh7 cells. FIG. 49A shows co-delivery efficacy to HEK293 Ts. FIG. 49B shows co-delivery efficacy to Huh7 cells. FIG. 49C shows fluorescent micrographs of co-delivery to HEK293T cells. Scale bar=100 μm.

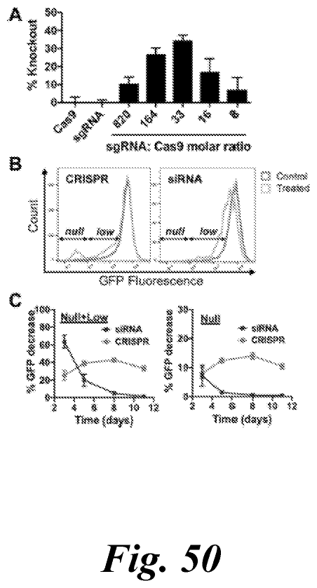

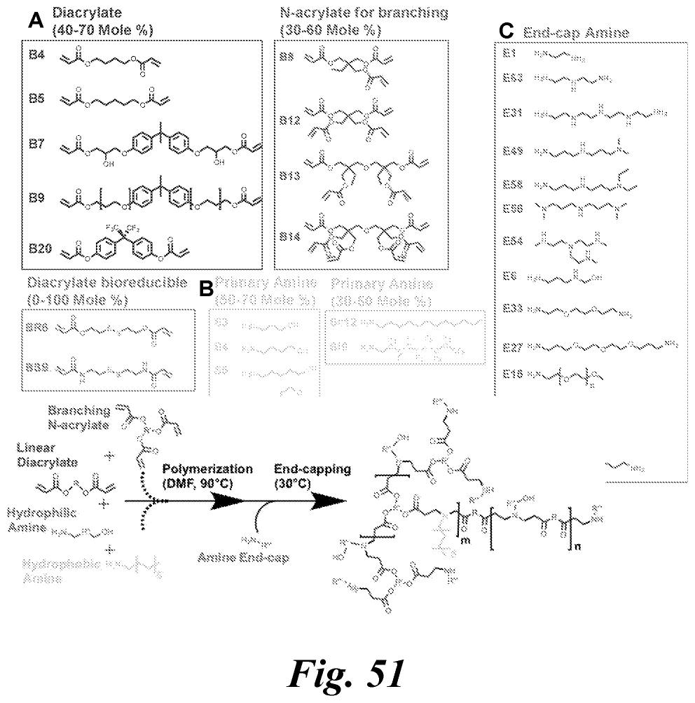

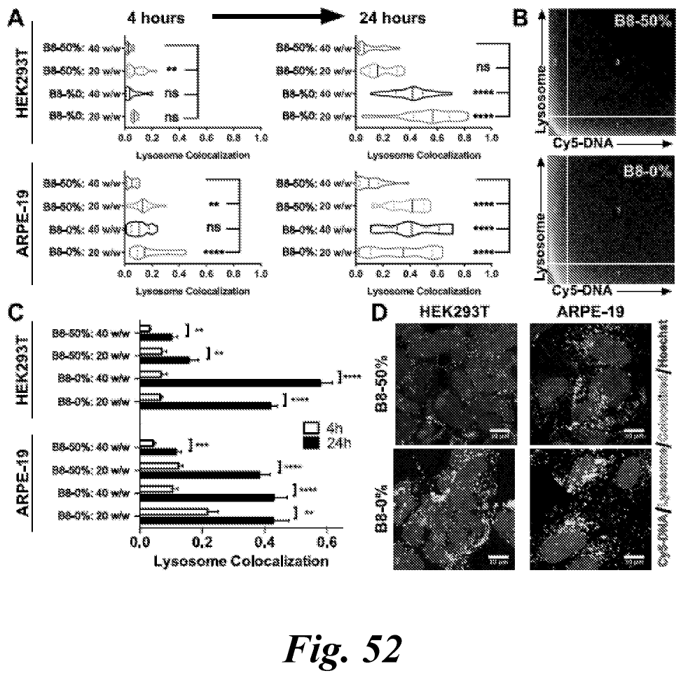

FIG. 50A shows CRISPR gene editing enabled by rBEAQ nanoparticles co-delivering sgRNA and Cas9 plasmid.

### Example 5

### Combinatorial Library of Biodegradable Polyesters Enables Delivery of Plasmid DNA to Polarized Human RPE Monolayers for Retinal Gene Therapy

5.1 Overview. Efficient gene delivery into hard-to-transfect cells is still a challenge despite significant progress in the development of various gene delivery tools. Non-viral and synthetic polymeric nanoparticles offer an array of advantages for gene delivery over the viral vectors and high in demand as they are safe to use, easy to synthesize and highly cell type specific. The presently disclosed subject matter demonstrates the use of a high-throughput screening (HTS) platform to screen for biodegradable polymeric nanoparticles (NPs) that can transfect human retinal pigment epithelial (RPE) cells with high efficiency and low toxicity. The presently disclosed NPs can deliver plasmid DNA (pDNA) to RPE monolayers more efficiently compared to the commercially available transfection reagents without interfering the global gene expression profile of RPE cells. The presently disclosed subject matter establishes an HTS platform and identifies synthetic polymers that can be used for high efficacy non-viral gene delivery to human RPE monolayers, enabling gene loss- and gain-of-function studies of cell signaling and developmental pathways. This platform can be used to identify the optimum polymer, weight-to-weight ratio of polymer to DNA, and the dose of NP for various retinal cell types.

5.2 Introduction. Gene therapy holds potential promise for treating both acquired and inherited blinding disorders as most of the identified disease to date is associated with RPE. See Bainbridge et al., 2006. Modulating specific gene targets simply by turning off or turning on its function has become a standard tool to enhance stem cell differentiation or to reprogram induced pluripotent stem cells (iPSCs) from somatic cells. See Jia et al., 2010; Nauta et al., 2013. Routinely approached gene therapy utilizes viral vectors to deliver pDNA considering their potential for high-efficiency gene delivery. However, to its flip side, this approach is limited by several different factors such as (a) potential for insertional mutagenesis, see Baum et al., 2006, (b) prone to degradation in the cytosol by nucleases, see Sasaki and Kinjo, 2010, or can accommodate only a specific size of pDNA to deliver. See Bitner et al., 2011; den Hollander et al., 2008; and Liu et al., 2007.

To overcome these challenges and to follow an alternative safer approach, significant attempts have been made to formulate and develop biodegradable non-viral vehicles agents to facilitate delivery of the gene of interest to the target sites. As the charge distribution on both the plasmid DNA and the cellular membrane is profoundly negative, cationic polymers often demonstrate efficient intracellular delivery by merely condensing the cargo (pDNA) by strong electrostatic interaction and form NPs. See Mastrobattista and Hennink, 2011. Since this strategy is episomal, it is usually considered as the safest way of delivering genes into the subcellular targets. See Lundstrom, 2003.

To this end, a range of different cationic polymers have been formulated and studied over the years for efficient non-viral gene delivery strategies. See Boylan et al., 2012; Cheng et al., 2013; de la Fuente M et al., 2010; Kim et al., 2004; Read et al., 2005; Wang et al., 2011; Yu et al., 2009. Regardless of the advantages that cationic polymers demonstrate over the viral mode of gene delivery, it's application is limited by a significant factor, i.e., poor transfection efficacy. See Pack et al., 2005. Poly(b-amino ester)s (PBAEs), a class of synthetic, cationic polymers, recently found to be useful as non-viral gene delivery agents. PBAEs are preferred polymers as they are easy to synthesize and demonstrate an efficient binding with its DNA counterpart. PBAEs are also hydrolytically degradable under physiological conditions and hence exhibit minimal cytotoxicity upon cellular administration. PBAEs have been shown to be successful in transfecting human adult and embryonic stem cells, see Yang et al., 2009, and mouse RPE cells in vitro and in vivo. See Sunshine et al., 2012. Besides, previous work also has suggested PBAEs to have cell-type specificity based on their chemical structures. See Shmueli et al., 2012; Sunshine et al., 2009. Hence PBAEs makes an ideal carrier to undertake this study given their structural tenability and simple synthesis scheme. The RPE cells are composed of a monolayer of pigmented and bipolar epithelial cells at the back side of the retina. Any compromise in the cellular environment of RPE cells leads to many hereditary and acquired diseases, including age-related macular degeneration (AMD). See Strauss, 2005. As RPE also dispensable for photoreceptor turnover and maintenance and as both PR and RPE dominate the retinal cell population, RPE cells could be the targets of therapy in many ocular diseases. Moreover, as in many ocular diseases lead to overall genetic imbalance, see Kawa et al., 2014; Wang et al., 2012, gene therapy is vital in restoring the gene expression in the compromised retina. Attempt to deliver a gene either into primary RPE cells or RPE cell lines is not new in the field. However, despite adopting several different non-viral strategies to deliver DNA either by polymeric or by liposomal vectors, the success rate is very low. See Abul-Hassan et al., 2000; Bejjani et al., 2005; Chaum et al., 1999; Jayaraman et al., 2012; Liu et al., 2011; Mannermaa et al., 2005; Mannisto et al., 2005; Mannisto et al., 2002; Peeters et al., 2007; Peng et al., 2011.

The presently disclosed subject matter provides a high throughput screening platform to screen for potential PBAE nanoparticles to access its transfection efficacy in iPS derived human RPE cells in vitro. Without wishing to be bound to any one particular theory, it is thought that cationic PBAE-pDNA NP complex can be delivered to the RPE monolayer efficiency by tuning the hydrophilicity and end group chemistry. Accordingly, a library of four PBAE base polymers with different backbone and end-group chemistry was synthesized. The ability of PBAE to bind to its DNA counterpart was examined by electrophoresis assay. To explore the effects of different PBAE chemical structures on delivery efficiency, 25-day old RPE monolayers were transfected with 140 different combinations of PBAEs using a pDNA encoding mCherry reporter gene under the CAG promoter. The outcomes were evaluated in a High Content Analysis platform where the images were acquired, and the data analysis was performed using specific algorithms

5.3 Results

5.3.1 Polymer Synthesis

Initially, a group of stable nanoparticles were formulated. NP formulation with the different combination of end-capped polymer and pDNA occurs via strong electrostatic interaction. Two different plasmids, expressing either the mCherry reporter or the nuc-GFP reporter driven by the same CMV early enhancer/chicken β actin (CAG) heterologous promoter as described in material and method section for the HTS, were used. Once the linear PBAE synthesis was completed, all the subsequent steps including end-capping reaction, preparation of source plate with end-capped polymers, stable NP formation with the desired pDNA, automated dispensing for transfection, and the HCA image capturing processes were carried out in a 384 well format for all the combination of NPs (FIG. 41). A range of different base polymers end-capped with a variety of different amino-terminal structures was combined to prepare a combinatorial library of 144 different PBAE NP formulations. The polymer nomenclature “N1-N2-XN” in the whole library, denotes base polymer number (N)-side chain number (N)-end cap amino terminal type (X) and number (N) respectively (FIG. 42).

5.3.2 High Throughput Automated NP Transfection to RPE Monolayer

To access the transfection efficacy of the PBAE/pCAGG-mCherry nanoparticles in matured RPE monolayers (Day 25 post seeding), a high throughput screening assay was conducted with all 144 different combinations of nanoparticles as explained in FIG. 41. This allowed a direct visualization of transfection efficacy (FIG. 43A) and viability rate (FIG. 43B) on a HCA platform where images were collected and data analyzed using a specific algorithm suitable to measure either transfection efficacy or viability rate. Cells transfected with the polymer without any end-capping reaction were included as controls. Heat map suggests that transfection efficacy differs significantly depending upon the side-chain end-capping chemistry of the PBAE (FIG. 43A). A few leading PBAE structures 5-3-A12, 5-3-F3 and 5-3-F4 resulted in 42%, 37% and, 34% positively transfected cells respectively. Interestingly, these specific polymers also demonstrated a significantly higher cell survival rate (90%, 97% and, 98% respectively; FIG. 43B). However, cell survival property of these top polymers was not directly proportional to their ability to transfect RPE monolayers, as some other PBAEs demonstrated extremely low transfection efficacy irrespective of their high cell survival property. Different PBAEs pairs with the same end-capping molecules show substantially different transfection efficiency, suggesting that the transfection efficiency also is dependent upon additional parameters, such as the degree of hydrophilicity and overall NP stability (e.g., the transfection efficacy of 3-5-A12 is 3.8% while it is 42% for 5-3-A12). In addition, while the PBAE 5-3-A12 demonstrated highest transfection efficiency (42%) and higher survival rate (90%) to the monolayered RPE cells at day 25, the same polymer yielded a lower transfection rate (33%) and lower survival rate (30%) in differentiating RPE cells at the early phase of differentiation on day 3 (FIG. 28). This result suggests that the overall transfection efficacy and impact on cell survival rate of a particular formulation varies significantly between different phases of a “differentiating” RPE cell.

5.3.3 Biophysical Characterization of 5-3-A12 Nanoparticle

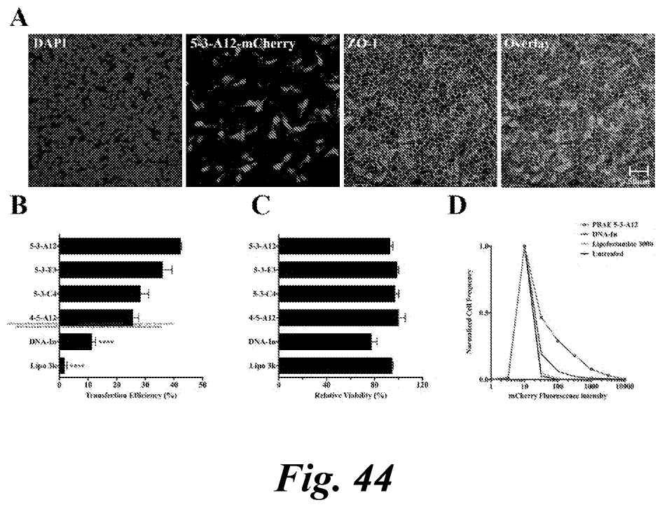

To further investigate the biophysical properties of PBAE nanoparticles that demonstrated high efficiency of pCAGG-mCherry delivery, the particle size of the 5-3-A12 nanoparticle was measured by both dynamic light scattering (DLS) and nanoparticle tracking analysis (NTA) methods. Zeta potential also was measured. All parameters were measured at different weight-to-weight (w/w) ratio. The particle size demonstrated a fairly broad distribution ranging from 49 nm to 191 nm by DLS method, and from 115 nm to 149 nm by NTA method (FIG. 44A, 44B). In corroboration with a previous report, the presently disclosed subject matter suggests that nanoparticles with a smaller size result in increased transfection rate, see Gan et al., 2005, as during the transfection optimization process, higher transfection rates were observed at a lower w/w ratio compared to a higher w/w ratio. Regardless, the transfection efficiency was always higher with the 5-3-A12 nanoparticle at any w/w ratio compared to other lead nanoparticles. Given the comparable transfection efficiency of the 5-3-A12 nanoparticle with different particle size, our results suggest that transfection efficiency of the 5-3-A12 nanoparticle is not solely dominated by particle size. Different than the particle size, 5-3-A12 nanoparticle demonstrated fairly similar surface charge distribution at any given size, which ranges from +25 mV to +30 mV as measured by zeta potential (FIG. 44C). Gel electrophoresis study demonstrated a complete PBAE/pCAGG-mCherry nanoparticle complex formation (FIG. 44D). To further consolidate the biophysical results observed, a transmission electron microscope (TEM) analysis was conducted. TEM imaging confirmed stable PBAE/pCAGGmCherry nanoparticle formation through the self-assembly process, with nanoparticle size consistent with the DLS and NTA results (FIG. 44E).

5.3.4 Validation of 5-3-A12 Nanoparticle Transfection Efficacy

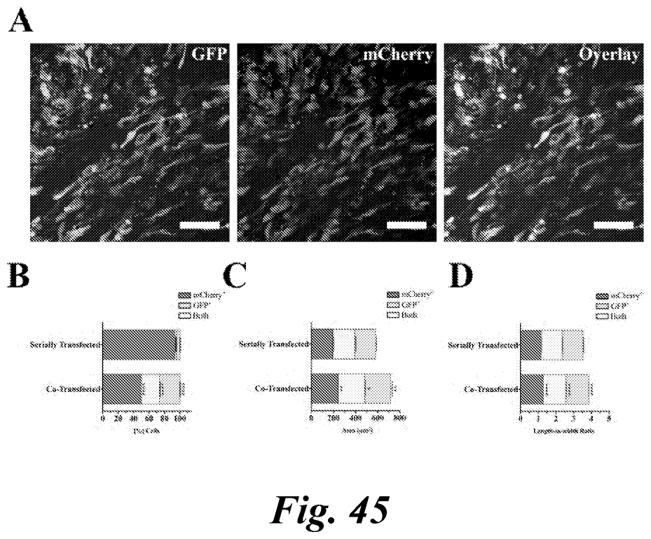

To further examine and validate the ability of the lead PBAE/pCAGG-mCherry nanoparticles to transfect RPE monolayers, 25-day old RPE monolayers were transfected separately in an 8-well chambered cover glass using already optimized transfection condition. RPE monolayers were also transfected with Lipofectamine 3000 and DNA-In for to test (and compare) the transfection efficacy as a control. Transfection efficiency up to 42% was observed with the lead nanoparticle (5-3-A12), which were 26% higher than that of DNA-In and 41% higher than Lipofectamine-3000, as well as comparable transfection efficiency of all the other polymers (FIG. 45A-B). Interestingly, although 5-3-A12 achieved the highest transfection in human RPE monolayers, it was the most inefficient polymer for mouse photoreceptor and human Retinal Ganglion Cells (data not shown) suggesting for its cell type specificity and suitable only for human RPE monolayers. To ensure that the differences in transfection efficiency were not caused by potential toxicity from polymeric nanoparticles, the effects of PBAE/pCAGG-mCherry nanoparticles on cell viability under the optimized transfection doses also were examined. The results indicated that most PBAE/pCAGG-mCherry nanoparticles formulations did not negatively affect cell viability compared to untreated cells alone (FIG. 45C). The only exception was one of the commercial reagents DNA-In, which showed slightly lower cell viability (approximately 80%). Previous work reported a higher dose of PBAE required for the plasmid DNA delivery with weight ratios up to 50:1 to reach optimal transfection efficiency in cancer cells, which may also cause increased cell death. See Sunshine et al., 2009. However, in this study, substantially less PBAE was required (3:1) to form stable nanoparticles with top PBAEs due to the smaller size. Also, because of the cell type specificity, we observed minimal cell death with RPE monolayers. This offers an additional advantage of using PBAE for pDNA delivery given the minimal toxicity effects with different cells. The mean fluorescent intensity also was quantified, which is a measure of total protein production. In this regard, RPE monolayers transfected in any form (via PBAEs or via commercial reagents) demonstrated a substantially similar level of mCherry intensity, despite their comparable level of percentage of cells being transfected (FIG. 45D). Transfection efficiency describes the percentage of cells that have been transfected, regardless of the difference in the level of protein production among individual cells. In contrast, mean fluorescence intensity takes into account of the difference in protein production by individual cells, and normalize that by the total number of cells. Therefore, mean fluorescence intensity is a better prediction of the level of protein production post-transfection. Also, the total number of cells also were counted over time during the differentiation process and the relative cell count and transfection efficacy of lipofectamine and DNA-In on RPE monolayers at different DNA doses were measured. Even though the cell number over time and the overall post-transfection viability rate was acceptable, the transfection efficacy was weak compared to 5-3-A12 PBAE at any given DNA dose.

5.3.5 Multiple Gene Delivery with 5-3-A12 Nanoparticle into RPE Monolayers

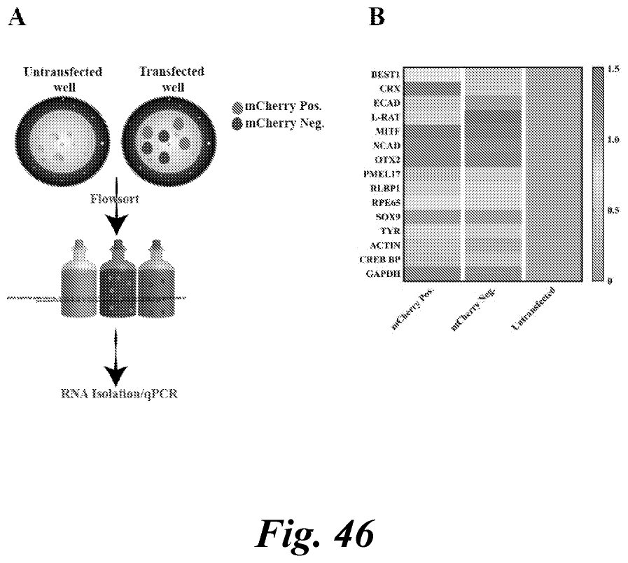

Since multiple gene deliveries using nanoparticle is quite challenging and none of the previous studies report using PBAE nanoparticle for multiple gene deliveries, the transfection efficacy of 5-3-A12 polymers for the delivery of more than one gene into RPE monolayers was evaluated. To this end, two separate pDNA constructs encoding two different reporter genes (mCherry and nuclear GFP) under same promoter (CAGG) were used and a co-transfection assay was optimized. Comparative data for cells that received either one or both of the reporter genes in a co-transfected cell population were generated 48 hours posttransfection (FIG. 46A). Two different strategies for transfection were adopted; either both the constructs were transfected at the same time (Co-transfected) or at the different times (serially transfected). Post-transfection data was analyzed for transfection efficacy (FIG. 46B), cell body area (FIG. 46C), and cell body shape (FIG. 46D) for each condition. The data suggest that under co-transfection condition, about 50% of the cell population received NP containing mCherry pDNA and about 25% of the cell population received NP containing nuc-GFP pDNA and remaining 25% cells obtained both the plasmids. In contrary, serially transfected condition favored more to NP containing mCherry pDNA, where more than 97% cell population received NP containing mCherry pDNA. While the preference of receiving one plasmid over another was significantly different in both the transfection conditions, as expected, no apparent change either in cell body shape or cell body size was observed in either circumstance. These results suggest that 5-3-A12 polymers do not interfere with intrinsic cellular pathways that trigger cellular/nuclear morphology and encourages for non-viral gene delivery applications.

5.4. Discussion

The most successful in vitro plasmid DNA gene delivery studies were established on RPE-derived cell lines, which are easier to transfect than primary RPE monolayers. Vercauteren et al., 2011. In this study, hiPSc-derived RPE cells were used, as they are considered to be much more similar to primary RPE than RPE cell lines, Klimanskaya et al., 2004, to investigate the utility of using a biodegradable and non-viral gene delivery approach for transient protein expression in primary RPE monolayers. To this end, a high-throughput platform was established to screen NPs created from a wide variety of polymers for their ability to deliver a gene into the human stem cell-derived RPE monolayers. Using this system, synthetic polymers were identified that can be used for high efficacy non-viral gene delivery to human RPE monolayers, enabling gene loss- and gain-of-function studies of cell signaling and developmental pathways. Since the self-assembly process of polymers high very complex, Molla and Levkin, 2016, it is very important to combine appropriate physical, chemical and biological properties to produce efficient polymers for gene delivery. Thus, high-throughput parallel generation and screening of large libraries of such nano-carriers is a very efficient and powerful way to identify efficacious and non-toxic gene delivery vectors. Despite the great interest in hiPSC RPE cells as sources for cell therapy and in vitro disease modeling, no studies of gene delivery of these cells using PBAE nanoparticles have been reported. The presently disclosed subject matter demonstrates that, as is the case for RPE cells, hiPSC-RPE cells are very difficult to transfect with plasmid DNA complexed with any commercial transfection reagent (lipofectamine or DNA-In). The highest efficiency of transfection with plasmid DNA using DNA-In was achieved on RPE monolayers with an efficiency of about 10%, and this was even lower (less than 5%) with lipofectamine 3000.

In addition, in the presently disclosed high-throughput screening assay, while most of the PBAE demonstrated a decent range of transfection (˜10% to ˜50%) on the sub-confluent RPE population (day 3 after seeding), most of them fail to transfect confluent RPE monolayer population (day 25 after seeding) when the cells reached a polygonal morphology. In contrast, top hits from the screening (5-3-A12, 5-3-F3 and 5-3-F4) could able to deliver the pDNA efficiently into both sub-confluent, and post-confluent monolayer (polygonal) RPE cells. While the reason for this discrepancy is not clear, it is thought that it is a phase dependent cell-type-specific event where the preferences of the interaction of the cationic polymer changes with the cell membrane structural change over time. Further optimization studies with different transfection agents, ratios of modified PBAEs, and pDNA/PBAE ratios are needed to understand this. Regardless, our results suggest that 5-3-A12 PBAE nanoparticle met all the criteria of a successful non-viral gene therapy agent being readily internalized into the cell, escaped endocytic degradation and successfully delivered the pDNA into the nucleus to be expressed. Even though specific characterization of the uptake of PBAE NPs in vitro was not undertaken; however confocal imaging data with ZO-1 labeling indicated that exclusively RPE monolayers with polygonal shape take up the particles. In the co-transfection assay, while the preference for getting transfected either with pmCherry or pNucGFP was subtle, the results from serially transfected RPE monolayer suggest that already transfected cells are less receptive or more rigid for re-uptake of new nanoparticles. This conclusion is based on the fact that in serially transfected cells the mCherry transfected cell population outnumbered dramatically to that of GFP transfected cell population when the cells were transfected with mCherry construct first. However, regardless of the type of transfection, PBAE nanoparticle has no impact on either cell body shape or size as evident from our co-transfection assay. This observation also suggests that, although PBAE nanoparticles can deliver multiple genes into RPE monolayers, they are often hampered by poor reproducibility and low co-transfection efficiency especially when cells are transfected serially. The results also suggest that the PBAE nanoparticle 5-3-A12 can preferentially deliver pDNA into human RPE monolayers with relatively low cytotoxicity. Even though the mechanism-of-action (MoA) is not known at this time, results from the current work provides important insights and holds promises for translational application of the biodegradable PBAE nanoparticles especially for RPE dysfunction. Since the overall surface charge distribution is an important deciding factor on cellular cytotoxicity, see Frohlich, 2012; Tomita et al., 2011, two different theories that results in low cytotoxicity effect of 5-3-A12 nanoparticle are possible. (1) its overall charge distribution on the surface (ranges from +25 mV to +30 mV) at any given w/w/ratio, which helps in interacting with the negatively charged components at the cell surface and destabilizes the cell membrane more efficiently than any other polymers used during primary screening; and (2) the electrostatic interaction between 5-3-A12 and pDNA introduces sufficient number of available amine group in 5-3-A12 that could results in increased zeta potential value.

The presently disclosed subject matter validates the expression pattern of known RPE markers from both mCherry+ and mCherry− cell population by low throughput (96-well) format using a qRT PCR assay. This purpose was to evaluate the possible PBAE interference with any known intrinsic RPE gene pathway. No change in the gene expression pattern was expected after transfection as the pDNA used expresses exogenous reporter genes without any known function on RPE markers. However, a differential gene expression pattern from the sample collected from transfected wells (regardless of their transfection status) was observed compared to sample collected from the un-transfected wells.

5.5. Materials and Methods:

5.5.1 Polymer Synthesis and Characterization

Monomers were purchased from vendors listed in Table 4. Acrylate monomers were stored with desiccant at 4° C., while amine monomers were stored with desiccant at room temperature. PBAE polymers were synthesized neat at 1.1:1 B:S monomer ratios for polymers 3-5-Ac, 4-4-Ac and 4-5-Ac and 1:1.05 monomer ratio for 5-3-Ac for 24 hours at 90° C. Following synthesis, neat polymers were dissolved at 200 mg/mL in anhydrous DMSO then precipitated in diethyl ether twice at a solvent ratio of 1:10 by vortexing the solvents and centrifuging at 3000 rcf. Polymers were allowed to dry under vacuum for 24 hours, at which point they were massed and dissolved at 200 mg/mL in anhydrous DMSO and allowed to remain under vacuum to remove additional diethyl ether for another 24 hours. Finally, acrylate terminated polymers were aliquoted and stored at −20° C. until use in end capping reactions.

For polymer characterization, samples of the initial neat polymer and neat polymer following diethyl ether removal were set aside for characterization via 1H NMR and gel permeation chromatography (GPC). GPC was performed on polymer samples both before and after double precipitation in diethyl ether using a Waters system with auto sampler, styragel column and refractive index detector to determine MN, MW and PDI relative to linear polystyrene standards. GPC measurements were performed as previously described with minor changes of a flow rate (0.5 mL/min) and increase in sample run time to 75 minutes per sample. See Bishop et al., 2013. Analysis of polymers via 1H NMR (Bruker 500 MHz) following diethyl ether precipitation and drying was performed to confirm the presence of acrylate peaks. For NMR, neat polymer was dissolved in CDCl₃ containing 0.05% v/v tetramethylsilane (TMS) as an internal standard.

5.5.2 Polymer Library Preparation

PBAE polymers were prepared for transfection screening experiments by high throughput, semi-automated synthesis techniques using ViaFlo 384 (Schematic 1B). For end capping reactions, 25 μL of endcap molecules in anhydrous DMSO at a concentration of 0.2 M were distributed to source wells of a deep-well 384 well plate, then distributed to corresponding replicate wells in groups shown in multiple colors of the end capping reaction 384-well deep plate (240 μL volume). Acrylate terminated base polymers at 200 mg/mL in anhydrous DMSO were thawed and distributed to wells containing 36 different endcap molecules and a single well containing DMSO only for the acrylate terminated polymer control. End capping reactions were allowed to proceed for two hours at room temperature on a gentle shaker, after which endcapped PBAE polymers were diluted to 50 mg/mL in anhydrous DMSO and aliquoted to 5 μL per well on the left side of 384-well nanoparticle source plates. Nanoparticle source plates were sealed and stored at −20° C. with desiccant until needed for transfection. Following largescale screening of the PBAE library in 384 well plates, larger batches of top PBAE structures were synthesized from frozen base polymer using the same protocol described above. Endcapped polymers were then aliquoted to individual tubes and stored at −20° C. with desiccant.

For end capping, reaction volumes of 50 μL at 100 mg/mL polymer concentration and 0.1 M were selected as sufficient to enable effective reactivity over a two-hour time period. For initial studies, endcap molecule E1 was titrated between 0.2 and 0.0625 M in reactions with base polymer PBAE 4-5-Ac at 100 mg/mL over two hours. Reacted polymers were then precipitated twice in diethyl ether to remove excess endcap monomer, dried and assessed using 1H NMR to determine efficacy of the end capping reaction by the disappearance of acrylate moiety peaks between 5.5-6.5 ppm. These results demonstrated effective end capping down to a concentration of 0.05 M for endcap molecule E1. To allow for varying levels of reactivity between endcap molecules, an endcap molecule concentration of 0.1 M was used for parallel large-scale end capping reactions.

5.5.3 Nanoparticle Characterization

The hydrodynamic diameter of top PBAE structure 5-3-A12 was characterized at three different w/w ratios to assess the influence of w/w ratio on nanoparticle characteristics. For dynamic light scatter (DLS) measurements, nanoparticles were initially formed in 25 mM NaAc, pH 5.0 then diluted 1:6 into 10% FBS in PBS dynamics and analyzed in disposable micro-cuvettes using a Malvern Zetasizer NanoZS (Malvern Instruments, Marlvern, UK) with a detection angle of 173°. For zeta potential, nanoparticles were prepared and diluted as for DLS, but were analyzed by electrophoretic light scattering was in disposable zeta cuvettes at 25° C. using the same Malvern Zetasizer NanoZS. For nanoparticle tracking analysis, nanoparticles were formed in 25 mM NaAc, pH 5, then diluted 1:500 in 150 mM PBS as previously described using a Nanosight NS300. A gel retention assay to assess PBAE: DNA binding strength was performed as previously described, see Tzeng et al., 2016, using a 1% agarose gel. Acrylate terminated PBAE 5-3-Ac was compared against top PBAE structure 5-3-A12 at w/w ratios from 0 to 50 to demonstrate improved binding of endcapped PBAE structures.

Transmission electron microscopy (TEM) images were acquired using a Philips CM120 (Philips Research, Briarcliffs Manor, New York) on 400 square mesh carbon coated TEM grids. Samples were prepared at a DNA concentration of 0.045 μg/μL and polymer 90 w/w ratio in 25 mM NaAc, pH 5.0 after which 30 μL were allowed to coat TEM grids for 20 minutes. Grids were then dipped briefly in ultrapure water, wicked dry and allowed to fully dry before imaging.

5.5.4 pDNA Design

For the in vitro transfection, a plasmid coding for the mCherry open reading frame was created by PCR amplification of the mCherry-N1 plasmid (Catalog no. 632523; Clontech). Since this plasmid has no start site an initiator, an ATG was added to the forward primer. After PCR amplification, mCherry was inserted into the directional pENTR-D-TOPO gateway entry vector (catalog no. K240020; Invitrogen). Positive colonies were selected by PCR and confirmed by sequencing. 100 ng of purified entry plasmid was mixed with pCAGG-DV destination vector, created by incorporating a gateway cassette containing attR recombination sites flanking a ccdB gene into the pCAGEN vector (Addgene #11160), in the presence of LR clonase II (catalog no. 11791019). After recombination clones were selected and sequenced.

5.5.5 Differentiation and Culture of RPE from hPSCs

RPE monolayers were differentiated as described previously by our laboratory, Maruotti et al., 2013; Maruotti et al., 2015, from the EP1-GFP human iPS cell line that constitutively expresses H2B-nuclear-GFP. In brief, iPS cells to be differentiated were then plated at 60,000 cells per cm² on Matrigelcoated 384 well plates and allowed to grow for 25 days in RPE medium consisting of 70% DMEM (catalog no. 11965092; ThermoFisher Scientific), 30% Ham's F-12 Nutrient Mix (catalog no. 11765-054; Invitrogen), see Gamm et al., 2008, serum free B27 supplement (catalog no. 17504044; ThermoFisher Scientific), and antibiotic-antimycotic (catalog no. 15240062; ThermoFisher Scientific). Coating of plate with Matrigel (25 μL per well), seeding of cells (50 μL per well), and media change every other day (replaced with fresh 25 μL per well) were accomplished using a high throughput Viaflo microplate dispenser (catalog no. 6031; Intergra). Cells were confirmed to possess an RPE monolayer phenotype at day 25 following plating.

5.5.6 In Vitro Nanoparticle Mediated Gene Delivery

On the day of transfection, the old media was discarded and replaced with 25 μL of fresh RPE media. To form PBAE/DNA nanoparticles, pDNA was diluted in 25 mM sodium acetate buffer (NaAc, pH 5) and aliquoted to individual wells on the right half of the 384-nanoparticle-source plate. End capped PBAEs from the left half of the 384 well round bottom source well place (schematic FIG. 1D) were then resuspended in parallel in 25 mM NaAc using a Viaflo microplate dispenser. After a brief centrifugation (1000 rcf for 1 minute) the solutions of unique PBAE structures were then transferred to the right half of the 384 well round bottom source well place containing pDNA (schematic FIG. 1D) in a 3:1 (vol/vol) ratio, resulting in a defined weight-weight (w/w) ratio between 20-100 of PBAE:DNA. The nanoparticle source plate containing the PBAE/DNA mixtures was then briefly centrifuged (1000 rcf for 1 minute). To dispense nanoparticles to cells, 5 μL volumes of the NPs in each well were then added to the RPE monolayer (schematic FIG. 1E) and incubated with cells for 2 hours inside the 37° C. incubator; all nanoparticles and media were then replaced with 50 μL of fresh RPE media. After 48 hours to allow for reporter gene expression, nuclei were stained with Hoechst and images acquired using an automated fluorescence-based imaging system (HCA Cellomics VTI; Thermofisher scientific). Transfected cells were identified as those expressing both the endogenous nuclear GFP and mCherry and the percent of transfected cells, as well as cell viability, was determined for each NP and condition. Commercial transfection reagents Lipofectamine 3000® (catalog no. L3000001; ThermoFisher Scientific) and DNA-In Stem (catalog no. GST-2130; MTI-Globalstem) were prepared according to manufacturer recommendations with pCAGG-mCherry. After particle formation, particles were added to day 25 differentiated RPE monolayer cells in 384 well plates at the specified DNA doses. Both reagents were optimized at multiple reagent:DNA ratios and for incubation times with cells of two hours and 24 hours to identify the optimal condition. After either two or 24 hours, media was entirely replaced with fresh medium and cells were cultured for two additional days, at which point transfection efficacy was assessed by image analysis with Cellomics.

5.5.7 Immunostaining

iPS cells to be differentiated were plated at 2.3 million cells per cm² on Matrigel-coated borosilicate sterile 8-well chambered cover glass (catalog no. 155409; Lab-Tek II;) and allowed to grow for 25 days in RPE medium. On the day of transfection, the old media was discarded and replaced with 300 μL of fresh RPE media. The PBAE 5-3-A12 were then mixed with CAGG mCherry in a 3:1 (vol/vol) ratio, resulting in a defined weight/weight (w/w) ratio of 80:1 of PBAE:DNA. The nanoparticle containing the 5-3-A12/CAGG mCherry mixtures was then briefly centrifuged (1000 rcf for 1 minute). To dispense nanoparticles to cells, 50 μL volumes of the NPs containing 1500 ng DNA were then added to the RPE monolayer and incubated with cells for 2 hours inside the 37° C. incubator; all nanoparticles and media were then replaced with 300 μL of fresh RPE media. After 48 hours to allow for reporter gene expression the cells were fixed with 4% paraformaldehyde in PBS, cells were blocked and permeabilized for 30 min in 5% goat serum, 0.25% Triton X-100 in PBS, and then incubated for 1 h at room temperature with polyclonal mouse anti-ZO-1 (1/500; catalog no. 40-2200; Invitrogen) monoclonal rat anti-mCherry (1/1000; catalog no. M-11217; Molecular Probes). Cells were then incubated for 1 h at room temperature with the corresponding secondary antibody conjugated to Alexa 488 or Alexa 568 (Invitrogen), and counterstained with Hoechst 33342 (Invitrogen). Images were captured with a confocal microscope (Zeiss LSM 710).

5.5.8 Co-expression Assay

To assess the ability of top PBAE nanoparticles to co-deliver two plasmids, EP1 cells that lacked nuclear GFP expression were plated as described above in 384 well plates and differentiated for 25 days to RPE monolayers. Plasmids CAGG-mCherry and CAGGnucGFP were diluted in 25 mM NaAc as described above and used to form PBAE 5-3-A12 nanoparticles at an 80 w/w ratio and DNA dose of 200 ng/well in 384 well plates. For the co-delivered condition, plasmids in 25 mM NaAc were pre-mixed prior to complexation with PBAE and added to RPE monolayers together in the same nanoparticles. For the serial transfection experiment, nanoparticles formed with plasmid CAG-mCherry only were added to cells at a dose of 100 ng/well on day 25 following plating and nanoparticles containing plasmid CAG-GFP only were added to cells on day 26. Media changes were performed as described above. Transfection efficacy for GFP and mCherry was assessed on day 28 following staining of cell nuclei with Hoechst 33342.

5.5.10 Imaging and Analysis Using HCS Studio 2.0 Software

Images were acquired on an ArrayScan VTi HCA Reader (ThermoFisher Scientific) using 10× or 20× magnification. For analysis, the ThermoScientific™ TargetValidationV4.1 application was used. Readout measurements included % transfected cell number, fluorescence intensity, nuclear size, and nuclear shape.

5.5.11 Statistical Analysis

Mean as well as standard deviation (in triplicate) was used for data analysis. One way ANOVA test was used for comparison of the results. For finding the differences between groups, data was analyzed by post-Hoc, Dunnett's multiple comparisons test. The P values of ****p<0.0001; ***p<0.001; **p<0.01; *p<0.05 were considered as statistically significant. Graph pad prism software (v.7.0) was used for data analysis.

5.5.12 Summary

In summary, a high-throughput screening and development of PBAE-based, biodegradable nanoparticles as efficient vehicles for delivering pDNA to human iPSc-RPE monolayers using a combinatorial chemistry approach is disclosed. By screening a total of 140 synthesized PBAEs with varying chemical structures, lead PBAE structures were identified that resulted in markedly increased pDNA delivery efficiency both in vitro. The presently disclosed results suggest that PBAE can effectively complex pDNA into nanoparticles, and protect the pDNA from being degraded by environmental nucleases and eventually deliver effectively to RPE monolayers. Without wishing to be bound to any one particular theory, the presently disclosed results support a hypothesis that PBAE mediated pDNA delivery efficiency can be modulated by tuning PBAE end group chemistry. Using human iPSc-RPE monolayers as model cell types, a few PBAE polymers were identified that allow efficient pDNA delivery at levels that are comparable or even surpassing commercial reagents like Lipofectamine 3000 and DNA-In. Unlike lipofectamine 3000 and DNA-In, which are non-degradable, the biodegradable nature of PBAE-based nanoparticles facilitates in vitro applications and clinical translation. Together, the presently disclosed results highlight the promise of PBAE-based nanoparticles as novel nonviral gene carriers for pDNA delivery into hard-to-transfect cell RPE monolayers.

### Example 6

### Differentially Branched Ester Amine Quadpolymers with Amphiphilic and pH-Sensitive Properties for Efficient Plasmid DNA Delivery

6.1. Overview. Development of highly effective nonviral gene delivery vectors for transfection of diverse cell populations remains a challenge despite utilization of both rational and combinatorial driven approaches to nanoparticle engineering. In this work, multifunctional polyesters are synthesized with well-defined branching structures via A2+B2/B3+C1 Michael addition reactions from small molecule acrylate and amine monomers and then end-capped with amine-containing small molecules to assess the influence of polymer branching structure on transfection. These Branched poly(Ester Amine) Quadpolymers (BEAQs) are highly effective for delivery of plasmid DNA to retinal pigment epithelial cells and demonstrate multiple improvements over previously reported leading linear poly(beta-amino ester)s, particularly for volume-limited applications where improved efficiency is required. BEAQs with moderate degrees of branching are demonstrated to be optimal for delivery under high serum conditions and low nanoparticle doses further relevant for therapeutic gene delivery applications. Defined structural properties of each polymer in the series, including tertiary amine content, correlated with cellular transfection efficacy and viability. Trends that can be applied to the rational design of future generations of biodegradable polymers are elucidated.
6.2. Background. Safe and effective gene delivery to specific cell populations has the potential to revolutionize medicine by enabling gene expression to be turned on or off precisely with the delivery of DNA or RNA. While viral vectors, particularly adenoassociated virus (AAV), have shown gains in the therapeutic delivery of DNA in some diseases, clinical level production of AAV remains an enormous challenge, 1,2 nucleic acid carrying capacity is limited, and patient pre-existing immunity can limit eligible patient populations.3,4 In contrast, nonviral nanoparticle based gene delivery methods have the potential to be both less expensive to produce, less immunogenic, and enable greater nucleic acid cargo capacity than AAV. However, nonviral gene delivery systems have suffered from low delivery efficacy to many cell types due to both systemic and intracellular delivery inefficiencies, which prevent translation to the clinic.5 While nonviral vectors have been demonstrated capable for effective delivery in vivo, there remains a need to develop enhanced nanoparticles that are more efficient, particularly for applications in which the administration route limits the dose.

Polyesters are a class of polymers that have been utilized for nonviral gene delivery with high efficacy both in vitro and in vivo to a variety of cell types.6-9 Synthesis of poly(beta-amino ester)s (PBAEs) in particular via Michael addition reactions is relatively easy to achieve, and vast libraries of linear polymers have been synthesized to explore the solution space of possible polymer structures for purposes of gene delivery. 10-12 Until recently, however, only linear PBAEs have been explored for their ability to deliver nucleic acids to mammalian cells, despite the demonstration that branching polymers are often more effective than their linear counterparts for delivery of plasmid DNA in a variety of polymer systems such as polyethylenimine (PEI)13 and poly(2-dimethylaminoethyl methacrylate) (PDMAEMA).14,15 Recent advances in the use of triacrylate monomers to synthesize branched polymers by Michael addition reaction have yielded polymers highly effective for delivery of nucleic acids to a variety of cell types, including cancer cells,14,15 skin cells, 16 neural cells, and mesenchymal stem cells. 17 Much of this prior work in the synthesis of branched PBAEs has either failed to assess the efficacy of branched polymers against linear polymers across the entire range of possible w/w ratios or has only utilized linear polymer structures of insufficiently high molecular weight and cationicity to achieve effective gene delivery. 16, 19

Polyesters with beta-amino groups are rapidly biodegradable and finely tunable for properties such as hydrophobicity, molecular weight, and cationic charge by selection of constituent monomers. These features enable certain structures to be highly effective for gene delivery but often require large empirical screens to identify effective structures. The biodegradability of PBAEs in aqueous solution is uncharacteristically short for polyesters with typical bond half-lives of 4-6 h for the backbone ester bonds, 18 enabling the polymers to degrade to nontoxic, hydrophilic oligomers within 24 h. Hydrophobicity can be modulated for transfection of different cell types, 19 and molecular weight can be modulated by tuning the overall vinyl to amine ratio. 11,20 Linear acrylate-terminated PBAE polymers can also be end-capped with a variety of small molecule primary amines that increase the cationic charge of the polymer by adding secondary as well as primary amines to the polymer.21

Whereas with polyethylenimine (PEI) branching structure changes the cationic character of the polymer (linear polymers contain mostly secondary amines, while branched polymers contain a tertiary amine at each branch point and a primary amine at each new terminal group), branching in a PBAE synthesis scheme does not dramatically change tertiary amines present in polymer structures of the same molecular weight. However, for PBAEs, branching structure can increase the density of end-capping functional groups, and these molecules have been shown previously to greatly enhance the transfection efficacy of linear polymers. 18,21 Branching in other polymeric systems has been further hypothesized to enhance the “needle effect” of endosomal escape mediated by polymer swelling, which could help explain this increase in efficacy.22-24

Here, we present the synthesis and characterization of a new polymer series, Branched poly(Ester Amine) Quadpolymers (BEAQs). They are composed of four constituent monomers in ratios that influence the cationic character and hydrophobicity of the polymer species in a predictable manner. This work builds on the successes of poly(ester amine) materials such as linear PBAEs,12 poly(amine-co-ester) (PACE) terpolymers,25 and poly(alkylene maleate mercaptamines) (PAMA)s26 that have demonstrated the utility of amines to bind nucleic acids, ester linkages to facilitate nucleic acid release and reduce toxicity as well as the ability to modulate cation density and hydrophobicity. We utilized A2+B2/B3 Michael addition reactions to synthesize primarily acrylate terminated polymers with well-defined degrees of branching that were then end-capped with a C monomer to explore the influence of branching structure on transfection efficacy and nanoparticle properties. This further enabled us to incorporate fine control of small amine-containing molecule end-groups for engineering of polymer and nanoparticle surface properties and hypothesized cell-specific delivery. 18, 27-2.9 Thus, the four components of the quadpolymers control degradability, hydrophobicity, branching, and cationicity, which have large effects on delivery efficacy and cytotoxicity.30 We assessed each polymer quantitatively for plasmid DNA binding under various conditions to demonstrate that increased DNA binding is attributable to increased cationicity resulting from multiple end-caps as well as branching structure. Branching was further shown to improve DNA binding and transfection efficacy under conditions that normally destabilize polyplex nanoparticles.

### 6.3. Experimental Section

6.3.1. Materials. Trimethylolpropane triacrylate (TMPTA/B8, CAS 15625895), bisphenol A glycerolate (1 glycerol/phenol) diacrylate (BGDA/B7, CAS 4687-94-9), and 2-(3-Aminopropylamino)ethanol (E6, CAS 4461-39-6) were purchased from Sigma-Aldrich and used without further purification. 4-Amino-1-butanol (S4, CAS 13325-10-05) was purchased from Alfa Aesar. Acrylate monomers were stored with desiccant at 4° C., while amine monomers were stored with desiccant at room temperature. Plasmid peGFP-N1 (Addgene 2491) was used for transfection efficacy screens. Cy5-amine (23000) was purchased from Lumiprobe (Hallandale Beach, Fla.), dissolved in DMSO at a concentration of 10 μg/μL, and stored at −20° C. in small aliquots. Plasmid DNA (eGFP-N1) was labeled as previously described using NHSPsoralen with the fluorophore Cy5-amine at a density of approximately 1 fluorophore/50 base pairs DNA.31
6.3.2. Polymer Synthesis. BEAQs were synthesized according to the ratios in Table 6-S1 at an overall vinyl/amine ratio of 2.2:1 and monomer concentration of 200 mg/mL in anhydrous DMF. The diacrylate monomer (B7) was first weighed out to a 20 mL scintillation vial, after which triacrylate monomer (B8) was added. Anhydrous DMF was added to the vial and monomers were fully vortexed into solution and heated to 90° C. before adding primary amine monomer S4. Monomer purity was accounted for in synthesis calculations based on the vendor characterization of each lot. Monomer B7 was assumed to be 90% pure in the absence of any reported purity information. Monomer solutions were then stirred at 90° C. for 24 h, after which polymers were removed from the oven and mixed with a solution of monomer E6 (2-β-aminopropylamino)ethanol) in anhydrous DMF (final concentration 0.2 M) in the dark at room temperature for 1 h. End-capped polymer solutions were then precipitated twice in diethyl ether (10× volume followed by 5× volume) and dried under vacuum for 3 days. Polymers were finally redissolved in anhydrous DMSO at 100 mg/mL and stored at −20° C. in small volume aliquots. Polymers were named according to the triacrylate mole fraction; thus B8-50% corresponds to the 50% triacrylate mole fraction polymer formed between the diacrylate (B7), triacrylate (B8), amino (S4), and diamino (E6) monomers with the triacrylate (B8) monomer accounting for 50% of the vinyl moieties in the initial monomer mixture.
6.3.3 Polymer Characterization. Acrylate terminated polymers were sampled from reaction vials prior to endcapping reactions and precipitated twice in 10× volumes of diethyl ether to recover neat polymer. Acrylate terminated polymers were then dried under vacuum for 2 h and analyzed via 1H NMR in CDCl₃ (Bruker 500 MHz) to confirm the presence of acrylate peaks and quantify degree of branching. End-capped polymer likewise was characterized via 1H NMR in CDCl₃ to confirm complete reaction of end-cap monomer with acrylate terminated polymers. End-capped polymer was also characterized via gel permeation chromatography (GPC) using a Waters system with autosampler, styragel column, and refractive index detector to determine MN, MW, and standards. GPC measurements were performed as previously described with minor changes of flow rate (0.5 mL/min) and increase in sample run time to 75 min per sample. 32
6.3.4 Polymer Buffering Capacity. End-capped polymer buffering capacity as a function of polymer structure was assessed by titrating 10 mg (100 μL at 100 mg/mL) of polymer dissolved in 10 mL of acidified, 100 mM NaCl from pH 3.0 to pH 11. For titrations, pH was determined using a SevenEasy pH Meter (Mettler Toledo) with pH assessed after stepwise addition of 100 mM sodium hydroxide.

We have demonstrated previously that 25 kDa branched polyethylenimine possesses a buffering capacity of 6.2 mmol H+/g of polymer in the pH range of 7.4 to 5. See J. C. Sunshine, D. Y. Peng, J. J. Green, Uptake and transfection with polymeric nanoparticles are dependent on polymer end-group structure, but largely independent of nanoparticle physical and chemical properties, Mol. Pharm. 9(11) (2012) 3375-83. This is the equivalent of 6.2 nmol H+ per mg of polyethylenimine, meaning that polyethylenimine would have 6.2 nmol H+ buffering capacity per μg of DNA at a 1 w/w ratio. At the higher than usual optimal w/w ratios of 3 and 4 w/w for HEK293T and ARPE-19 (FIG. 16), PEI would have an optimal buffering capacity of either 24.8 or 37.2 nmol H+/μg DNA depending on cell type. 6.3.5. Polymer Solubility Limit. Polymers were dissolved in pH 7.4, 150 mM PBS or pH 5.0, 25 mM NaAc at the specified maximum concentration and aliquoted (50 μL) to a roundbottom 96-well plate (n=3 wells). Polymers were then diluted stepwise in their respective buffers, and absorbance measurements were acquired with a plate reader (Biotek Synergy 2) at 600 nm (for opacity indicative of solubility limit). Absorbance measurements of 0.5 were defined as the maximum solubility point for purposes of plotting polymer solubility (FIG. 14).

6.3.6. DNA Binding Assays. Yo-Pro-1 iodide binding assays were run similarly to previously published results,33 where DNA and Yo-Pro-1 iodide (Thermo Fisher) were both diluted to a concentration of 1 μM (3.1 μg/mL plasmid) in either 25 mM NaAc, pH 5.0 or 150 mM PBS, pH 7.4 then mixed with polymer to give a 100 μL well volume in opaque black well plates. Green channel fluorescence was then measured using a plate reader after 30 min of incubation (Biotek Synergy 2). Gel electrophoresis binding experiments were run as previously described9 with nanoparticles prepared in either 25 mM NaAc buffer, pH 5.0 or 150 mM PBS, pH 7.4, diluted with 30% glycerol for loading into a 1% agarose gel. 6.3.7 Nanoparticle Characterization. Three samples were independently prepared for each nanoparticle formulation at the same concentrations as outlined in the transfection methods section. Nanoparticle hydrodynamic diameters in 25 mM NaAc, pH 5.0 were then determined by dynamic light scattering (DLS) in disposable microcuvettes using a Malvern Zetasizer NanoZS (Malvern Instruments, Marlvern, UK) with a detection angle of 173°. Samples were then diluted in 150 mM PBS at a dilution factor of 6 and measured again to determine nanoparticle hydrodynamic diameter in neutral, isotonic buffer followed by determination of zeta potential by electrophoretic light scattering in disposable zeta cuvettes at 25° C. using the same Malvern Zetasizer NanoZS. Transmission electron microscopy (TEM) images were acquired using a Philips CM120 (Philips Research, Briarcliffs-Manor, New York) on 400 square mesh carbon coated TEM grids. Samples were prepared at a DNA concentration of 0.045 μg/μL and polymer 40 w/w ratio in 25 mM NaAc, pH 5.0 after which 30 μL were allowed to coat TEM grids for 20 min. Grids were then dipped briefly in ultrapure water to remove excess dried salt, wicked dry, and allowed to fully dry under vacuum before imaging.
6.3.8. Cell Culture. HEK293T and ARPE-19 cells were purchased from ATCC (Manassas, Va.) and cultured in high glucose DMEM or DMEM/F12, respectively, supplemented with 10% heat inactivated fetal bovine serum and 1% penicillin/streptomycin. For noted 96-well plate transfection efficacy experiments, cells were plated in CytoOne 96-well tissue culture plates (USA Scientific, Ocala, Fla.) 24 h prior to transfection with 12,000 cells/well in 100 μL complete media. For noted 384-well plate transfection experiments, cells were plated at 2,500 cells/well in 25 μL of complete media in 384-well tissue culture plates (Santa Cruz, sc-206081) 24 h prior to transfection. Cells were confirmed periodically to be mycoplasma negative via MycoAlert test (Lonza).
6.3.9. Transfection and Cell Uptake. For 96-well plate transfections, nanoparticles were formed by dissolving synthesized polymers and eGFP-N1 plasmid DNA in 25 mM sodium acetate (NaAc) pH 5.0 then mixing in a 1:1 volume ratio. Nanoparticles were incubated at room temperature for 5 min, then 20 μL of the nanoparticle solution were added to each well of cells containing 100 μL of complete media and allowed to incubate for 2 h, at which point the media was replaced. Transfection efficacy was assessed for percent-transfected cells and geometric mean expression approximately 48 h following transfection using flow cytometry with a BD Accuri C6 flow cytometer with HyperCyt autosampler and gated in 2D against untreated cells in FlowJo (FIG. 23). Cell viability was assessed using MTS Celltiter 96 Aqueous One (Promega, Madison, Wis.) cell proliferation assay approximately 24 h following transfection. For 384-well plate transfection of low doses of nanoparticles, synthesized polymers in DMSO were dissolved in 25 mM NaAc buffer to a concentration of 7.5 μg/μL then mixed with DNA dissolved in 25 mM NaAc buffer in a 384 polypropylene nanoparticle source plate. Nanoparticles were then dispensed to plates of cells at low volumes using an Echo 550 liquid handler. After 2 days to allow for reporter expression, plates were scanned and analyzed using Cellomics Arrayscan VTI with live cell imaging module following staining with Hoechst 33342. Flow cytometry based cell uptake studies were performed in 96-well plates using 20% Cy5 labeled DNA as previously described.32 To remove associated nanoparticles that were extracellular membrane associated but had not undergone endocytosis, cells were washed once with 50 μg/mL heparin sulfate in 150 mM PBS following trypsinization and transfer to round-bottom 96-well plates.32.
6.3.10. Confocal Microscopy. Cells were plated on Nunc Lab-Tek 8 chambered borosilicate coverglass well plates (155411; Thermo Fisher) at 50,000 cells/well (ARPE-19) or 25,000 cells/well (HEK293T) 2 days prior to transfection in 250 μL of phenol red free DMEM supplemented with 10% FBS and 1% penicillin/streptomycin. Nanoparticles were prepared as described above 20 or 40 w/w ratios using Cy5 labeled plasmid DNA and eGFP-N1 plasmid DNA at an 0.8/0.2 mass ratio, then added to cells at a total dose of 1500 ng DNA/well and incubated for 2 h. For imaging, cells were stained for 30 mM with Hoechst 33342 at a 1:5,000 dilution (H3570; Thermo Fisher) for nuclei visualization and with Cell Navigator Lysosome Staining dye with pKa 4.6 at a 1:2,500 dilution (AAT Bioquest, 22658) in phenol red free DMEM. Cells were then washed twice with phenol red free DMEM and imaged at 37° C. in a 5% CO2 atmosphere. Images were acquired using a Zeiss LSM 780 microscope with Zen Blue software and 63× oil immersion lens. Specific laser channels used were 405 nm diode, 488 nm argon, 561 nm solid-state, and 639 nm diode lasers. Laser intensity and detector gain settings were maintained across all image acquisitions. All Zstacks were acquired for entire cell volume over scan area of 140 μm at Nyquist limit resolution.
6.3.11. Data Analysis and Figures. FlowJo was used for flow cytometry analysis, and Cellomics HCS Studio (Thermo Fisher) was used for image acquisition based transfection analysis. Polymer structures were characterized in ChemDraw (PerkinElmer, Boston, Mass.) and Marvin (ChemAxon, Cambridge, Mass.) to determine log P and log D values. Calculation of normalized 50% serum transfection efficacy was performed by dividing the percent transfection or geometric mean transfection efficacy achieved in 50% serum media by the same nanoparticle (B8% and w/w ratio) formulation percent transfection or geometric mean transfection efficacy achieved in 10% serum. Confocal microscopy colocalization of plasmid DNA with lysosomes was assessed as intensity weighted colocalization in Zen Blue, then normalized by individual image area of plasmid DNA per image for statistical quantification.
6.3.12. Statistics. Prism 8 (Graphpad, La Jolla, Calif.) was used for all statistical analyses and curve plotting. Unless otherwise specified, statistical tests were performed with a global alpha value of 0.05. Unless otherwise stated, absence of statistical significance markings where a test was stated to have been performed signified no statistical significance. Statistical significance was denoted as follows: *p<0.05; **p<0.01; ***p<0.001; ****p<0.0001.

### 6.4 Results

6.4.1. Branched Poly(ester amine) Quadpolymer Synthesis and Characterization.
6.4.1.1. Synthesis of Acrylate Terminated Polymers. A series of Branched poly(Ester Amine) Quadpolymers (BEAQ) with differential degrees of branching was synthesized via step-growth A2+B2/B3 Michael addition reactions from small molecule diacrylate (BGDA/B7), triacrylate (TMPTA/B8), and amino-alcohol (S4) monomers (FIG. 7) and Table 6-S1). In the synthesis scheme of A2+B2/B3+C, A2 corresponds to the primary amine monomer (S4) that can react twice, B2 corresponds to the diacrylate monomer (termed B7) that can react twice, B3 corresponds to the triacrylate monomer (termed B8) that can react three times, and C refers to the end-cap monomer, which reacts once due to its presence in excess. We confirmed that each polymer was primarily acrylate terminated after 24 h of synthesis via 1H NMR (FIG. 13) by the presence of acrylate peaks between 5.5 and 6.5 ppm. Analysis of the acrylate terminated polymer structures with 1H NMR also enabled determination of polymer properties including the actual triacrylate mole-fraction of each polymer as well as number of end-cap moieties per polymer molecule (Table 6-1).

TABLE 6-1
Structural Properties of Synthesized Polymers
Triacrylate mole fraction (%)

| | | | End-cap | | | |
|---|---|---|---|---|---|---|
| | | Theoretical | polymer | | | |
| | | end-caps | mass | GPC | GPC | |
| | | per | fraction | MN | MW | GPC |
| Theoretical | Actual | molecule | (%) | (Da) | (Da) | PDI |
| 0 | 0.0 | 2.0 | 5.7 | 4700 | 5700 | 1.203 |
| 10 | 15.1 | 3.0 | 9.1 | 4700 | 5700 | 1.216 |
| 20 | 22.8 | 3.4 | 11.5 | 4200 | 5200 | 1.258 |
| 40 | 34.8 | 3.9 | 15.0 | 4200 | 5100 | 1.223 |
| 50 | 47.1 | 4.9 | 17.1 | 4200 | 5800 | 1.369 |
| 60 | 58.5 | 4.5 | 22.0 | 4200 | 5900 | 1.411 |
| 80 | 83.3 | 5.5 | 27.5 | 4800 | 18300 | 3.849 |
| 90 | 91.7 | 6.5 | 28.2 | 3100 | 21600 | 6.952 |

By precisely varying the triacrylate monomer mole fraction, while maintaining the same 2.2:1 vinyl to amine mole ratio, the degree of branching was able to be carefully modulated in the resulting polymers as assessed by 1H NMR. Further, by synthesizing the polymers in each series at the same purity accounted overall vinyl to amine ratio, the number-average (MN) molecular weights within each series of polymers were all very close to 4 kDa as shown by gel permeation chromatography (GPC) (Table 6-1).

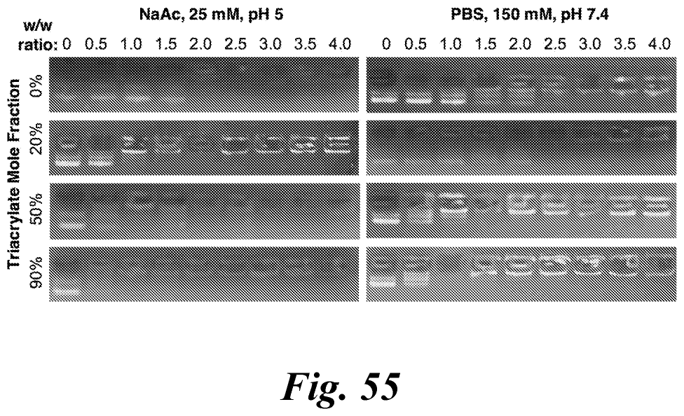

6.4.1.2. End-cap Modification of Polymers. PBAEs have been “end-capped” with small molecule monomers possessing secondary and tertiary amines that increase the overall polymer amine density, resulting in linear polymers with tertiary amines along the polymer backbone and greater amine density at just the two ends of the linear polymers.12,21,34,35 Most of the small molecule end-caps shown previously to increase transfection efficacy with linear PBAE structures21 increase the cationicity of the polymer at both pH 5 and 7 due to the fact that endcapping with primary amine monomers adds at minimum of two secondary amines to linear PBAEs. Here, we utilized monomer 2-β-aminopropylamino)ethanol (termed E6) for end-capping purposes, as it has been shown to be effective as an end-capping group with linear polymers and noncytotoxic to multiple cell lines.33,35 In contrast to previously reported branched polymer schemes, including branched PBAE schemes, this end-capping molecule exclusively increases the secondary amine content of the polymer. All BEAQs were confirmed to be completely end-capped by 1H NMR and the number-average of end-cap moieties per polymer molecule as estimated from NMR spectra ranged from two for the linear polymer to seven for the 90% triacrylate mole fraction polymer (Table 6-1). Notably, end-cap molecular mass fraction contribution in these polymers reaches near 30% for the high triacrylate mole fraction polymers, whereas linear PBAEs have an end-cap monomer mass fraction of approximately 5%, which reduces further for higher molecular weight linear polymers (Table 6-1). Polydispersity in moderately branched BEAQs was minimized by synthesizing at a dilute concentration, while high polydispersity of hyperbranched BEAQs with triacrylate mole fraction >60% is consistent with other hyperbranched polymer synthesis schemes. 36
6.4.1.3. Polymer Series Hydrophobicity. The chemical properties of each polymer in the series with known Mn and monomer composition were predicted in silico to assess the influence of branching with TMPTA on polymer hydrophobicity. Hydrophobicity was assessed as predicted partition coefficient (log P) and ionization influenced distribution coefficient (log D) at neutral and acidic pH values (FIG. 8A and FIG. 14), demonstrating that branching increases BEAQ hydrophilicity for the monomers utilized here and that pH sensitive ionization plays an important role in polymer solubility. Branching was hypothesized to reduce both polymer log P and log D values as a greater number of E6 monomer endcap moieties in branched structures increased the prevalence of hydrophilic hydroxyl groups and charged secondary amines; polymers with a high degree of branching were further subject to reduction in hydrophobicity due to the fact that the mass fraction of the diacrylate monomer B7, which contains a bisphenol group, was likewise reduced. We confirmed this predicted reduction in hydrophobicity experimentally via an absorbance based assay, to show that BEAQs with at least 40% triacrylate mole fraction were over twice as soluble as the linear B8-0% polymer under both low pH and physiological pH conditions (FIG. 14).
6.4.1.4. Polymer Series Buffering Capacity. Titration of the polymers demonstrated buffering capacity in the physiological pH range for hypothesized endosomal escape properties (5 to 7.4), as BEAQs with greater triacrylate mole fraction possessed a larger buffering capacity in this range (FIG. 8B). Effective pKa in the pH range from 5 to 8 was calculated as the pH at the maximum normalized buffering capacity of the derivative of the titration curves defined as Δ(—OH)/Δ(pH) (FIG. 14B). Effective pKa was demonstrated to increase moderately with increased branching from approximately 6.0 to 6.75 (FIG. 14C). These results are due to the combined effects of additional tertiary amine density in the polymer backbone and the presence of additional secondary amines in end-groups as the branching increases.18 Tertiary amine density calculated relative to the base polymer structures (Table 6-S4) shows that diacrylate B7+S4 polymer repeat units have much lower tertiary amine density than triacrylate B8+S4×2 repeat units, and physical spacing of the tertiary amines in high diacrylate B7 content polymers is greater than that for high triacrylate B8 content polymers. However following end-capping with monomer E6, tertiary amine density is similar among all synthesized polymers, while secondary amine density increased substantially with triacrylate mole fraction from 0.851 to 4.194 mmol per gram polymer for B8-0% and B8-90%, respectively (Table 6-S5).
6.4.1.5. Polymer Series DNA Binding. Assessment of BEAQ/DNA binding strength interactions via Yo-Pro-1 iodide competition binding assays further demonstrated the influence of branching in polymer structure (FIG. 8D, 8F). At pH 5, linear and branched polymers were equally effective at binding plasmid DNA, while in isotonic, neutral buffer at pH 7.4, branched polymers statistically outperformed linear polymers for DNA binding (Table 6-S6). To assess if increases in DNA binding strength of the BEAQs were attributable primarily to branched structure or changes in amine content, we calculated Yo-Pro-1 iodide quenching as a function of secondary, tertiary, and total amine content per base-pair DNA from known structural characteristics of each polymer (FIG. 15). DNA binding normalized to tertiary amine content effectively condensed the binding assay results at pH 5, while normalization of DNA binding in neutral, isotonic buffer to secondary amine content most effectively condensed the results to fit one curve (FIG. 8E, FIG. 8G). Gel electrophoresis DNA retention assays were similarly in agreement with these results, demonstrating that branching improved DNA binding particularly in neutral, isotonic buffer (FIG. 55). These results indicate that BEAQ backbone tertiary amines play an important role in polymer complexation with DNA at low pH, but secondary amines in BEAQ end-cap structures are primarily responsible for binding plasmid DNA following dilution into neutral solutions. Further analysis of the difference between binding at low pH and neutral pH do, however, reveal that the increase in end-cap density of branched polymers was not exclusively responsible for increased binding at neutral pH. Scaling the difference in binding efficacy as a function of total amines per base pair DNA revealed that branched polymers were more effective at maintaining DNA binding in a manner that is attributable to structural changes instead of increases in amine content (FIG. 8H).
6.4.2. Nanoparticle Properties. Dynamic light scattering (DLS) measurements of polymer/DNA polyplex nanoparticles to assess hydrodynamic diameter demonstrated effective independence of nanoparticle properties with regards to branching. DLS measurements of polymeric nanoparticles formed in 25 mM NaAc, pH 5.0 at a 40 w/w ratio to DNA showed that all polymers formed nanoparticles with a hydrodynamic diameter of approximately 50-100 nm that maintained a diameter of approximately 100 nm following a 6-fold dilution into 150 mM PBS (FIG. 9A. All nanoparticle formulations showed similar zeta potential values of approximately +15 mV (FIG. 9B). Select formulations were analyzed via TEM, which showed dried nanoparticle diameters between 30 and 60 nm (FIG. 9C). Notably, the linear 0% triacrylate mole fraction (B8-0%) particles were the smallest when assessed by TEM at 32±3 nm, compared to a mean of 54±6 nm for B8-50% nanoparticles, which may be attributable to slightly stronger intermolecular polymer interactions driven by increased hydrophobic effect for the less branched polymers with higher B7 fraction/lower triacrylate monomer B8 fraction.

### 6.4.3. Cellular Transfection.

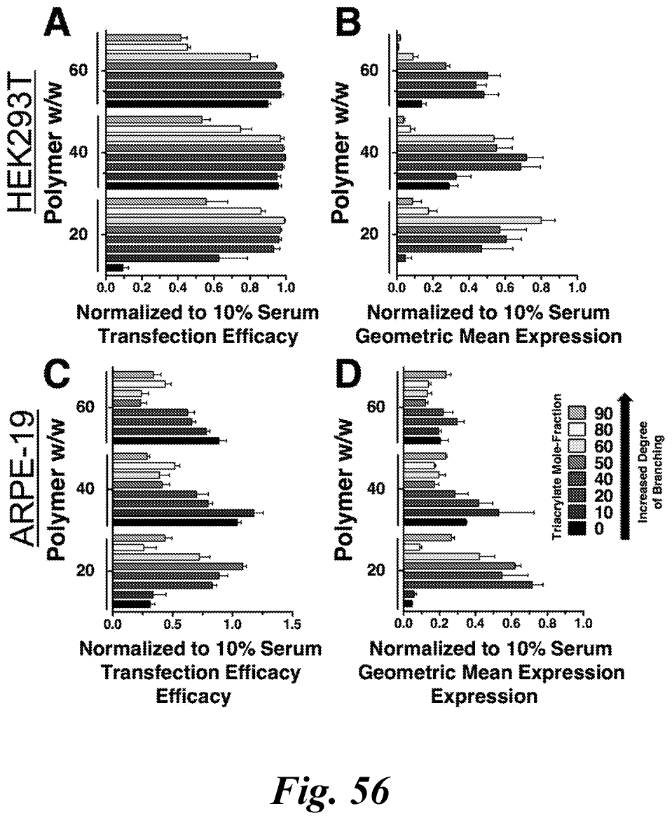

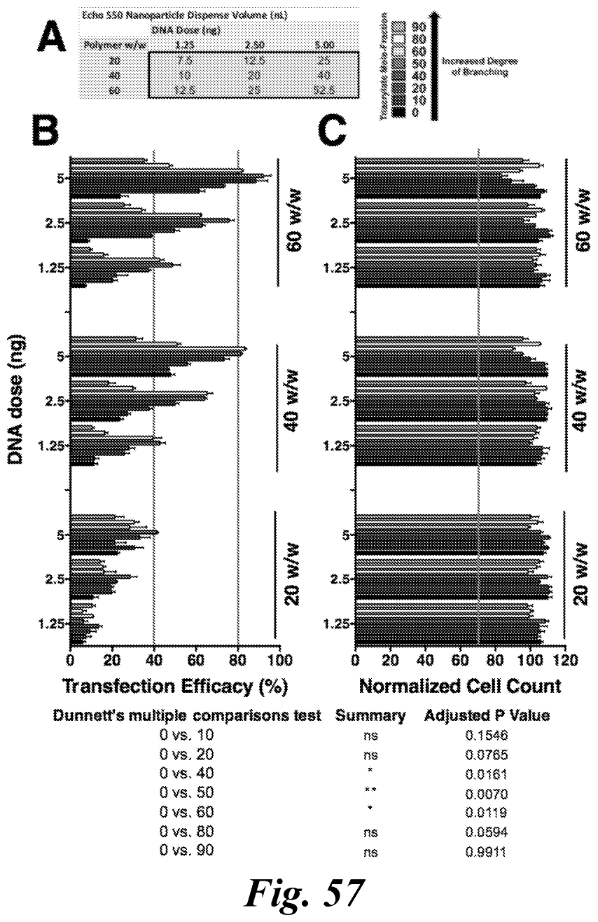

6.4.3.1. Nanoparticle Uptake Was Not Influenced by Branching Structure. We hypothesized that the increased number of end-cap moieties per polymer molecule would result in increased cellular uptake, as end-capping linear PBAEs has been demonstrated to improve cellular uptake compared to acrylate terminated and side-chain monomer terminated linear PBAEs.21 Further, end-cap structures have been shown to convey cell type specificity,21,27 as well as partially contribute buffering capacity of PBAEs in the physiologically relevant pH range.18 To assess whether the increased number of end-cap moieties per polymer molecule for BEAQs would yield greater cell uptake relative to linear PBAEs, we assessed cellular uptake by flow cytometry of nanoparticles formed with Cy5 labeled plasmid DNA in HEK293T and ARPE-19 cells at moderate fluorophore labeling density. All polymers were generally effective for mediating cellular uptake of plasmid DNA, with above 95% of cells testing as positive for DNA uptake gated against the untreated cells (FIG. 16). These branched polymers showed no significant improvement in cellular uptake at equivalent w/w ratios to the linear polymer. Thus, an increased number of 2-β-aminopropylamino) ethanol end-cap moieties per polymer molecule did not mediate higher cellular uptake as hypothesized.
6.4.3.2. BEAQ Nanoparticles Mediate High Transfection Efficacy. To assess the ability of BEAQs to effectively deliver plasmid DNA to both easier-to-transfect and difficult-to-transfect cell types, HEK293T cells and ARPE-19 retinal pigment epithelial cells were chosen for transfection studies with the reporter gene eGFP-N1. In these two cell lines, the BEAQs nanoparticles achieved up to 99% and 77% transfection efficacy, respectively, in complete medium as assessed by flow cytometry, which is greater than any reported transfection efficacy using nonviral methods in either cell line to the best of our knowledge (FIG. 10). Among commercial reagents we fully tested and optimized, including 25 kDa branched polyethlyenimine (BPEI), 4 kDa linear polyethylenimine (LPEI), JetPRIME, and Lipofectamine 2000 (FIG. 22 and FIG. 21). JetPRIME gave the highest level of transfection in ARPE-19 cells at approximately 40% transfection with tolerable viability. Linear PEI gave slightly higher transfection but at the cost of substantial cytotoxicity. The maximum level of transfection achieved in ARPE-19 cells with the reported BEAQ polymers is likewise higher than our previously optimized top linear PBAE 557 formulation, which we found transfected only 40-45% of these cells with keeping cytotoxicity <30%.37 This formulation was previously shown to lead to transfection in vivo following subretinal injection in mice, making it likely for these BEAQ nanoparticles to function in a similar manner in vivo.37
6.4.3.3. Moderate Branching in BEAQs Improves Stability in Physiological Serum Conditions. Effective delivery under physiological serum conditions remains a challenge for cationic nanoparticle based gene delivery, due to the shielding and aggregation effects of serum proteins. To assess nanoparticle performance under these conditions, the BEAQs were evaluated for transfection in HEK293T and ARPE-19 cells incubated in 50% serum medium during a 2 h nanoparticle incubation (FIG. 17). Under these challenging transfection conditions, which more closely model an in vivo systemic administration, BEAQs demonstrated remarkably statistically improved transfection efficacy compared to their linear counterparts, which was particularly pronounced at low w/w ratios in both cell lines (FIG. 11A, FIG. 11B). The optimal BEAQ-50 branched polymer was capable of transfecting 98% and 65% of HEK293T and ARPE-19 cells under 50% serum conditions. After normalizing transfection efficacy results in 50% serum to matched results in 10% serum conditions, BEAQ nanoparticles reported here maintain 80% and 70% geometric mean expression in HEK293T cells and ARPE-19 cells with no reduction in percentage of cells transfected (FIG. 56).
6.4.3.4. Moderate Branching Improves Transfection at Low Plasmid Doses. Transfection at low nanoparticle doses likewise better mimics conditions encountered in vivo following administration and dilution into biological fluids. At very low nanoparticle doses, plasmid concentrations between 16 and 256 pM (0.25-4 pg/cell) in 384-well plates, moderately branched triacrylate mole fraction BEAQs showed statistically higher transfection compared to the optimized corresponding linear PBAE in both cell lines (FIG. 57 and FIG. 18). Overall with statistical assessment at all w/w ratios tested, B8-40% and B8-50% performed the best in both cell lines. Optimal w/w ratio was notably shifted for low DNA dose transfections, such that 60 w/w BEAQ nanoparticles showed better transfection than 20 w/w particles at very low doses (≤5 ng/well). Cell viability was not strongly affected under any of the conditions.

### 6.4.4. Branching Reduces Degree of Lysosomal Accumulation Following Uptake.

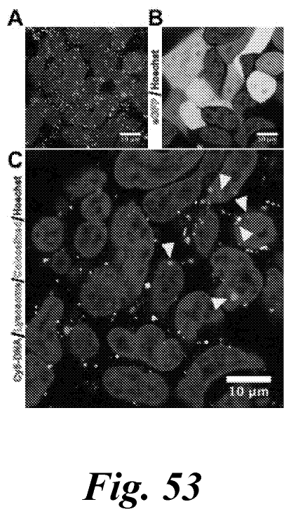

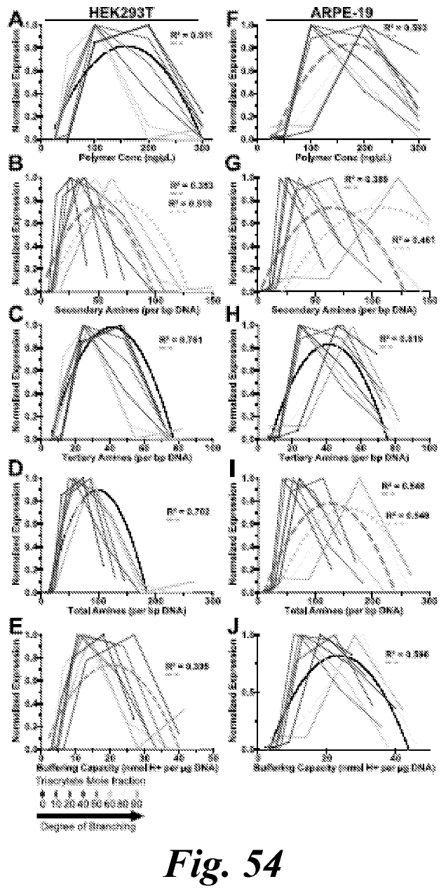

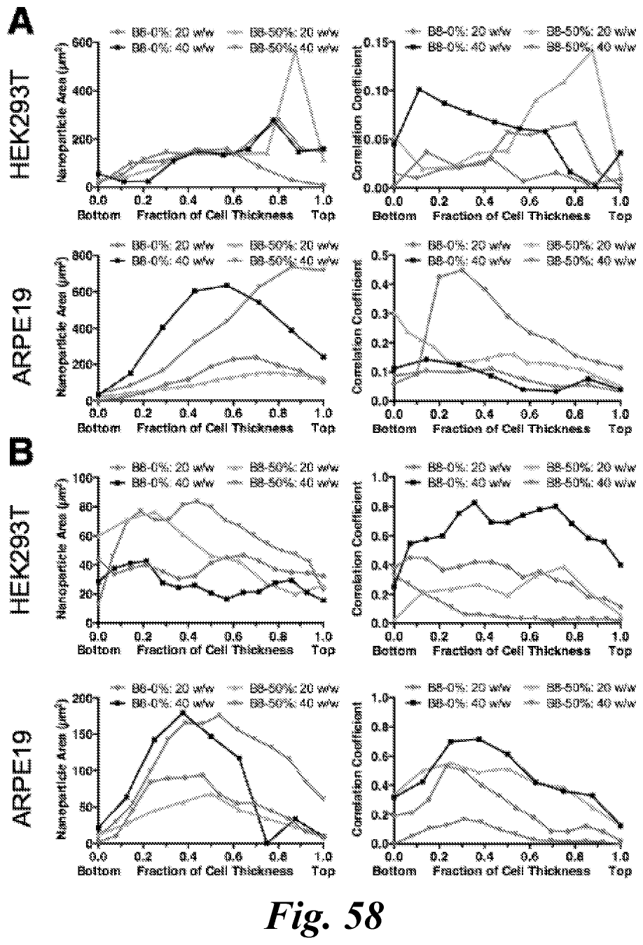

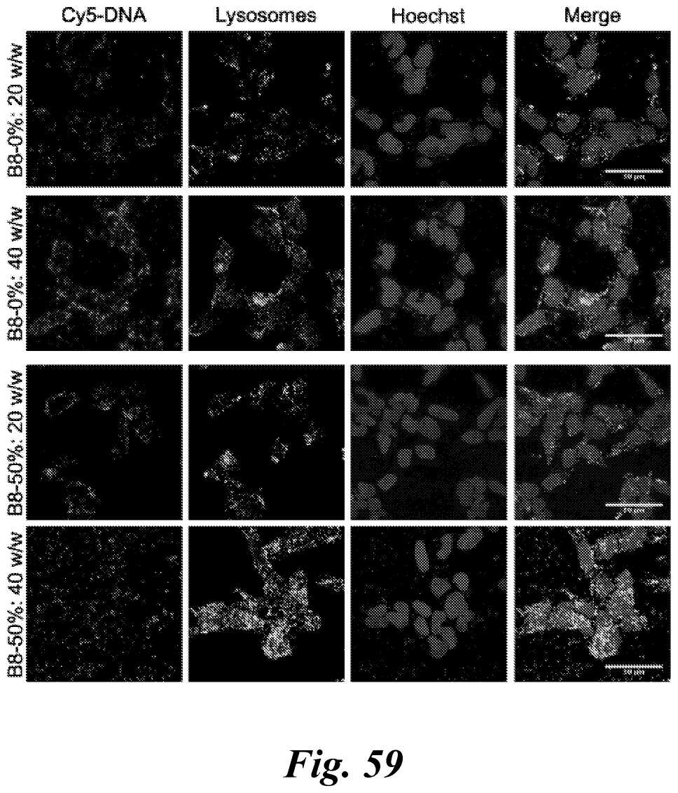

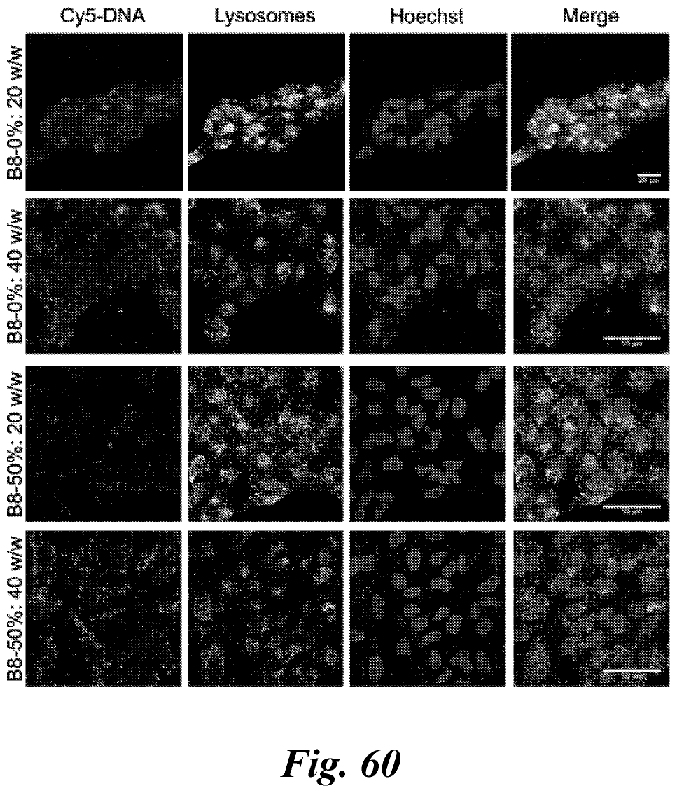

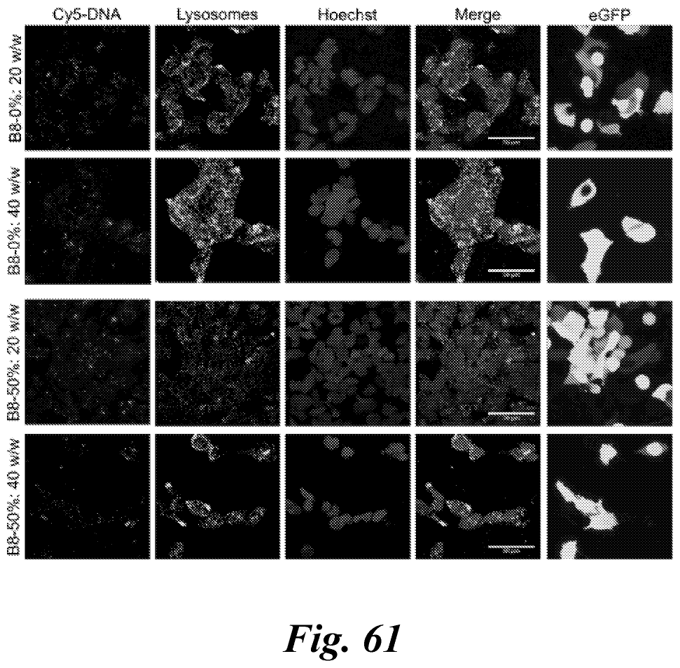

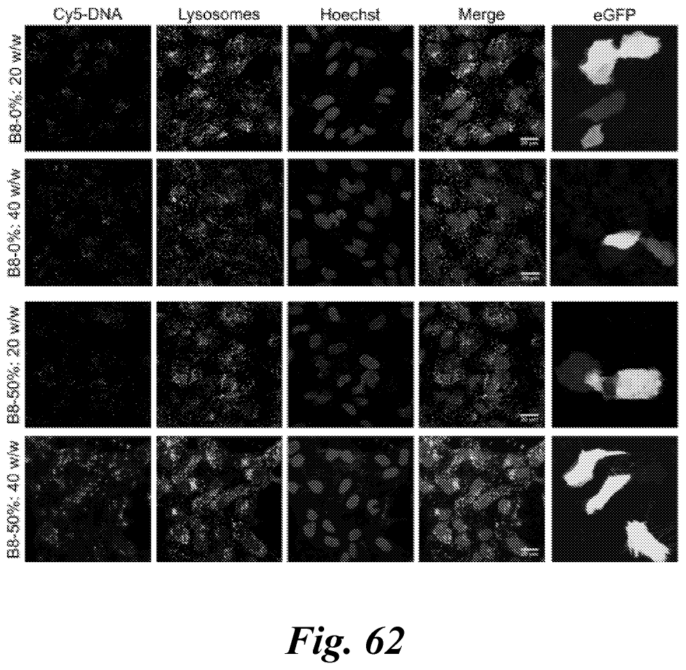

Transfection of HEK293T and ARPE-19 cells with Cy5-labeled plasmid DNA followed by assessment of lysosome colocalization with confocal microscopy at 4 and 24 h following nanoparticle treatment demonstrated that less internalized DNA was colocalized with lysosomes when delivered by B8-50% BEAQs compared to the linear B8-0% polymer (FIG. 52). For accurate quantification of lysosomal colocalization throughout the entire cell volume, Z-stacks were acquired at both time-points, and nanoparticle area per slice was used to scale the respective contribution to calculated z-stack lysosome correlation coefficient (FIG. 58). Representative uncropped maximum intensity projection images of acquired Z-stacks for each condition show a high level of Cy5-DNA uptake with limited lysosome colocalization for all conditions (FIG. 59 and FIG. 60). All nanoparticle formulations tested demonstrated a statistically significant increase in lysosome colocalization between 4 and 24 h following nanoparticle treatment (FIG. 52C); however, the degree of change in lysosome accumulation was lower with the B8-50% BEAQ nanoparticles, specifically for the higher 40 w/w ratio tested, which yielded less than 20% of internalized DNA as detectable in lysosomes at 24 h in either cell type. The degree of lysosome colocalization for the linear B8-0% polymer at 24 h (0.4) was still far below the colocalization we previously measured for PLL (0.78) and BPEI (0.7), despite the ability of BPEI to much more effectively buffer protons on a per-unit basis.39 This result supports the notion that amphiphilic polyesters mediate lysosomal in a different manner than polyethylenimine, as their degree of lysosomal avoidance is not proportional to their buffering capacity. At 24 h following nanoparticle treatment, cells expressing eGFP from the 20% unlabeled fraction of plasmid DNA were visible for all conditions (FIG. 61 and FIG. 62). Cy5-labeled plasmid DNA was also detectable in the nucleus of some cells that typically were also strongly expressing eGFP at the 24 h time point (FIG. 53). Analysis of single slices from Z-stacks did, however, reveal that most plasmid DNA internalized had not localized to the nucleus at 24 h post-treatment, even when it avoided lysosomal degradation.
6.4.5. Trends in Transfection from Differentially Branched BEAQs. We analyzed transfection efficacy of each polymer over the multiple w/w ratios tested as functions of polymer concentration and known specific buffering capacity as well as secondary, tertiary, and total amine content. To account for overall population expression and effects of polymers on viability, we scaled geometric mean expression values by viability and normalized to the maximum geometric mean expression value of each polymer structure to give viability normalized expression. Viability normalized expression was then plotted against each variable of interest (FIG. 54). All BEAQs demonstrated clear biphasic trends in normalized geometric mean expression. Upon fitting a single quadratic curve to the data from all polymers, tertiary amine density as a function of tertiary amines per base pair DNA was revealed to be the most important chemical property for predicting optimal w/w ratio for transfection efficacy. Particularly, a single curve quadratic fit for all polymer data across all structures for HEK293T and ARPE-19 cells gave R2 values of 0.761 and 0.615, respectively. Polyethylenimine did not exhibit the same biphasic trends between amine content and geometric mean expression as BEAQs but did demonstrate optimal amine content of approximately 30 secondary amines, which may be attributable to the greater cytotoxicity encountered with using PEI that limits utilization of high w/w ratios (FIG. 21). Interestingly, highly branched 25 kDa BPEI had a much higher optimal total amines per bp DNA similar to the synthesized BEAQs, which may be attributable to the level of interaction between amines in linear polymers compared to branched polymers. Spatial accessibility of amines in polymer structures and steric hindrance in branched polymers may necessitate greater overall amine content.

### 6.5. Discussion

Branching has been demonstrated to yield enhanced transfection in many cationic polymer systems and studied in PBAEs through the use of monomers with trifunctional amine monomers40 or trifunctional triacrylate monomers for generation of branched polymers.17 Here, we sought to explore the exact nature by which branching can improve transfection efficacy of these polymers through a fair comparison of fully effective linear PBAEs to equivalent branched species. For this purpose, we synthesized a series of polymers with well-defined degrees of branching that was quantified via NMR and GPC. These BEAQs are notable in part due to the manner in which end-capping with the chosen E6 monomer affected amine density, particularly through adding secondary amines to the polymer structure. We hypothesized that the branching structure and high end-cap moiety mass fraction in BEAQs would show improved DNA binding at neutral pH and would be more effective for delivery at lower w/w ratios as compared to linear PBAEs due to their increased secondary amine cationicity. BEAQs were shown via computational and experimental methods to be more water-soluble due to the increased prevalence of hydrophilic end-cap moieties and more effective at buffering in the physiological pH range. We further calculated the effective pKa value of each polymer to demonstrate that branching influenced the pH point of maximal buffering capacity. Given the long-standing hypothesis that titration capability of polycations in the pH 5-7.4 range is responsible for “proton sponge hypothesis” driven endosomal escape,39,41-44 direct variation of the buffering capacity and effective pKa allowed evaluation of the importance of buffering in gene delivery with these polymers. Through quantitative competition DNA binding assays we demonstrated that branching improved DNA binding as a function of both increased secondary amine content via additional end-cap monomers as well as branching structure by normalized binding efficacy to specific amine content of each polymer. Importantly, BEAQs were much more effective at binding nucleic acids compared to the linear polymer following dilution into neutral, isotonic buffer. Using the two well characterized cell lines human embryonic kidney HEK293T and human retinal pigment epithelium ARPE-19, these polymers demonstrated extremely high transfection efficacy (up to 99% and 77%, respectively) with no notable cytotoxicity at utilized doses. BEAQs did not demonstrate greater nanoparticle uptake compared to the linear polymer but did improve transfection efficacy and reduce the necessary w/w ratio, effectively improving polymer efficiency of transfection at a given polymer mass. As the highly branched B8-80% and B8-90% polymers possessed the greatest buffering capacity and the most relevant effective pKa values (nearer to pH 7) but the lowest transfection efficacy, our results further indicate that buffering capability and endosomal escape is likely not the rate-limiting step to mediating successful transfection in this polymer system. These results reinforce findings from other groups in alternative polymer systems that polymer buffering capacity between pH 4-7.4 is a necessary, but not on its own a sufficient property for transfection. 45 Under more challenging transfection conditions of extremely low nanoparticle doses or under physiological serum conditions, moderately branched BEAQs were statistically shown to outperform the equivalent linear PBAE and possess extremely high transfection efficacy for the reported conditions. At ultralow plasmid DNA doses, the efficiency of plasmid DNA delivery was rather remarkable compared to previously reported optimal nanoparticles, including PBAE terpolymers that include alkyl side chains for improved colloidal stability that were shown to require roughly 3× the DNA dose used here to transfect HeLa cells with similar efficacy.46 Further, in physiological serum conditions these BEAQ nanoparticles demonstrated an impressive degree of transfection compared to what has been reported in the literature. Fluorinated PAMAM dendrimers were reported to have their transfection efficacy reduced to 30% of what it was in 10% serum when the nanoparticles were added to cells in 50% serum.47 In contrast, BEAQ nanoparticles maintained >70% geometric mean expression under matched conditions to 10% serum transfections. Other nonviral transfection reagents have similarly been reported to facilitate transfection under physiological serum conditions, but often yield only 30-40% of the mean expression level of the same particles in 10% serum.48 That being said, even at this relatively high level of efficacy of nonviral transfection, much room is left for improvement in nonviral vector efficiency as compared to viral vectors that have evolved for over a billion years for efficient transduction. At the low doses tested of 5-10 ng plasmid DNA/well, there were approximately 200 000-400 000 plasmids available for every cell in the well.

Plasmids per cell were calculated as follows. In 384 well transfection experiments at low nanoparticle doses, moderately branched BEAQs yielded 82% transfection efficacy in HEK293T cells at a dose of 5 ng/well and 42% transfection efficacy in ARPE-19 cells at a dose of 10 ng/well. Cells were seeded at a density of 2500 cells per well and assumed to divide once to yield 5000 cells per well on the day of transfection. The eGFP-N1 plasmid has a size of 4733 bp and molecular weight of approximately 3124 kDa, meaning there were 9.64×10⁸ plasmids/well and 192,800 plasmids per cell available at a 5 ng dose.

Based on recent estimates of approximately 10 plasmids per polyplex nanoparticle.31,49 there could still be over 20,000 nanoparticles added per cell at this dose, which is a high multiplicity of infection (MOI). With an estimated number of 5000 plasmids from polyplex nanoparticles being internalized per cell under higher dose transfection conditions and an estimated ⅕ of those plasmids reaching the nuclear envelope, uptake of nanoparticles appears to be a significant hurdle to effective transfection in vitro.32 In comparison to efficient viruses, the low nanoparticle doses tested here are far above the order of magnitude MOI used for adenovirus (1-1000) and various lentiviruses (1-200) to yield similar levels of expression.50,51 In contrast, naturally occurring AAVs are often used at a much higher MOI of up to 100 000 to achieve similarly detectable reporter gene based levels of transfection in hard-to-transduce cell lines.52,53 Spark Therapeutics recently completed a successful phase III clinical trial using subretinal delivery of AAV for the first FDA approved gene therapy, voretigene neparvovec-rzyl, demonstrating the clinical potential of nonintegrating gene therapy.54 Given the similar level of MOI for BEAQ and AAV and coupled with challenges in scaling production of AAV for clinical utilization1,2 and the limitations of AAV cargo capacity, nonviral delivery of episomal plasmid DNA with this BEAQ system may be a viable strategy for clinical delivery of DNA to RPE cells.

Escape from endosomes and avoidance of lysosomal degradation remains a significant hurdle to nanomaterials aiming to achieve cytosolic delivery. Estimates of endosomal escape of lipid nanoparticles for siRNA has revealed that less than 2% of cargo internalized to endosomes typically reaches the cytosol,55,56 which has been improved by some recent lipid nanoparticle formulations yielding up to 15% escape in HeLa cells.57 Polyplex nanoparticles similarly suffer from low endosomal escape efficacy, with the classic materials such as polyethylenimine and polylysine almost exclusively remaining in acidified vesicles and undergoing lysosomal degradation despite the ability of the former material in particular to buffer hydrogen ions.41 Transport to acidic lysosomes occurs rapidly following internalization, with nanoparticles typically reaching a lysosomal compartment within 1 h following internalization. 58 In contrast to these findings for most other polymeric materials, we demonstrated that BEAQs largely avoid lysosomal degradation with <20% of labeled plasmid DNA being detectable in acidified vesicles at 24 h post-treatment compared to 40-50% DNA delivered with the linear polymer detected in acidified vesicles. These results are promising in that they demonstrate that branching may improve the ability of these polymers to achieve endosomal escape, which remains a primary hurdle to effective gene delivery. Finally, we demonstrated how polymer structure, as a function of hydrophobicity and cationicity, related directly to optimal polymer/DNA mass ratio and to transfection efficacy as these variables have been shown repeatedly to be crucial to yielding robust transfection in other polymer systems. To identify structure—function relationships between these polymers and transfection efficacy, we analyzed viability and geometric mean expression as a function of individual polymer properties including buffering capacity, secondary, tertiary, and total amine content per bp plasmid DNA. This is the first reported analysis of this type reported to our knowledge and yielded insights into the features of polycations that make them effective for transfection. In particular, we demonstrated that the optimal number of tertiary amines per bp plasmid DNA was near constant across the entire range of branching, while optimal numbers of secondary amines increased with degree of branching. With further knowledge of precise desired polymer structures, solid phase synthesis of alternating copolymers is an option that has been utilized in the synthesis of precisely defined polymers for gene delivery.59 Degradation rate of polymers could also play a role in the differences in transfection, as differences in constituent monomers can affect the specific degradation rate. The total possible solution space for BEAQs that may be highly effective for gene delivery is vast, as there are many diacrylate, side-chain amino, and end-capping amino monomers available that have been shown to yield linear polymers effective for transfection of diverse cell types. Synthesis of BEAQs via the guidelines outlined here and in previous publications14 will enable the rapid prototyping of diverse polymers that may yield further gains to efficient nucleic acid delivery as well as insights into polymeric structure/function relationships. The presented method for generating BEAQs can likewise be easily expanded to include utilization of branching monomers with other triacrylate monomer use as well as quaternary or greater functionality such as pentaerythritol tetraacrylate or dipentaerythritol penta-/hexa-acrylate to further increase structural diversity.

### 6.6. Summary

Branched poly(Ester Amine) Quadpolymers (BEAQs) were successfully synthesized and characterized and were demonstrated to have multiple enhancements over leading nonviral gene delivery materials including optimized linear PBAEs, BPEI, JetPRIME, and Lipofectamine 2000. BEAQs with a moderate degree of branching were shown to more tightly bind plasmid DNA, maintain DNA binding following dilution in neutral, isotonic buffer, and possess higher solubility in aqueous media compared to linear analogs. Branched polymers formed from diacrylate (B7) and triacrylate (B8) monomers were highly effective for plasmid DNA delivery, and moderately branched BEAQs best maintained efficacy at physiologically relevant high serum concentrations. Analysis of chemical structure highlighted the importance of the ability to buffer pH at approximately 20 nmol H+/μg DNA as well as the key parameter of tertiary amine content at approximately 40 tertiary amines per base pair of DNA. Through differential control of polymer branching, BEAQs were found to be efficient for nonviral gene delivery to difficult-to-transfect human cells. BEAQs are promising as therapeutic gene delivery vehicles, and these findings have implications for the design, identification, and optimization of next-generation polymeric materials for nucleic acid delivery.

### 6.7 REFERENCES

(1) Kotin, R. M. Large-scale recombinant adeno-associated virus production. Hum. Mol. Genet. 2011, 20 (R1), R2-R6.
(2) Wright, J. F. Manufacturing and characterizing AAV-based vectors for use in clinical studies. Gene Ther. 2008, 15 (11), 840.
(3) Kotterman, M. A.; Schaffer, D. V. Engineering adeno-associated viruses for clinical gene therapy. Nat. Rev. Genet. 2014, 15, 445.
(4) Asokan, A.; Schaffer, D. V.; Samulski, R. J. The AAV vector toolkit: poised at the clinical crossroads. Mol. Ther. 2012, 20 (4), 699-708.
(5) Yin, H.; Kanasty, R. L.; Eltoukhy, A. A.; Vegas, A. J.; Dorkin, J. R.; Anderson, D. G. Non-viral vectors for gene-based therapy. Nat. Rev. Genet. 2014, 15 (8), 541-555.
(6) Mangraviti, A.; Tzeng, S. Y.; Kozielski, K. L.; Wang, Y.; Jin, Y.; Gullotti, D.; Pedone, M.; Buaron, N.; Liu, A.; Wilson, D. R.; Hansen, S. K.; Rodriguez, F. J.; Gao, G.-D.; DiMeco, F.; Brem, H.; Olivi, A.; Tyler, B.; Green, J. J. Polymeric Nanoparticles for Nonviral Gene Therapy Extend Brain Tumor Survival in Vivo. ACS Nano 2015, 9 (2), 1236-1249.
(7) Keeney, M.; Ong, S.-G.; Padilla, A.; Yao, Z.; Goodman, S.; Wu, J. C.; Yang, F. Development of poly(β-amino ester)-based biodegradable nanoparticles for nonviral delivery of minicircle DNA. ACS Nano 2013, 7 (8), 7241-50.
(8) Cho, S.-W.; Yang, F.; Son, S. M.; Park, H.-J.; Green, J. J.; Bogatyrev, S.; Mei, Y.; Park, S.; Langer, R.; Anderson, D. G. Therapeutic angiogenesis using genetically engineered human endothelial cells. J. Controlled Release 2012, 160 (3), 515-24.
(9) Tzeng, S. Y.; Wilson, D. R.; Hansen, S. K.; Quinones-Hinojosa, A.; Green, J. J. Polymeric nanoparticle-based delivery of TRAIL DNA for cancer-specific killing. Bioeng. Transl. Med. 2016, 1 (2), 149-159.
(10) Anderson, D. G.; Lynn, D. M.; Langer, R. Semi-automated synthesis and screening of a large library of degradable cationic polymers for gene delivery. Angew. Chem., Int. Ed. 2003, 42 (27), 3153-8.
(11) Akinc, A.; Anderson, D. G.; Lynn, D. M.; Langer, R. Synthesis of poly (β-amino ester) s optimized for highly effective gene delivery. Bioconjugate Chem. 2003, 14 (5), 979-988.
(12) Green, J. J.; Langer, R.; Anderson, D. G. A combinatorial polymer library approach yields insight into nonviral gene delivery. Acc. Chem. Res. 2008, 41 (6), 749-59.
(13) Intra, J.; Salem, A. K. Characterization of the transgene expression generated by branched and linear polyethylenimine-plasmid DNA nanoparticles in vitro and after intraperitoneal injection in vivo. J. Controlled Release 2008, 130 (2), 129-138.
(14) Cutlar, L.; Zhou, D.; Gao, Y.; Zhao, T.; Greiser, U.; Wang, W.; Wang, W. Highly branched poly (β-amino esters): Synthesis and application in gene delivery. Biomacromolecules 2015, 16 (9), 2609-2617.
(15) Liu, S.; Gao, Y.; Zhou, D.; Greiser, U.; Guo, T.; Guo, R.; Wang, W. Biodegradable Highly Branched Poly ((3-Amino Ester) s for Targeted Cancer Cell Gene Transfection. ACS Biomater. Sci. Eng. 2017, 3, 1283.
(16) Zhou, D.; Gao, Y.; Aied, A.; Cutlar, L.; Igoucheva, O.; Newland, B.; Alexeeve, V.; Greiser, U.; Uitto, J.; Wang, W. Highly branched poly (β-amino ester) s for skin gene therapy. J. Controlled Release 2016, 244, 336-346.
(17) Zhou, D.; Cutlar, L.; Gao, Y.; Wang, W.; O'Keeffe-Ahern, J.; McMahon, S.; Duarte, B.; Larcher, F.; Rodriguez, B. J.; Greiser, U. The transition from linear to highly branched poly (β-amino ester)s: Branching matters for gene delivery. Science advances 2016, 2 (6), No. e1600102.
(18) Sunshine, J. C.; Peng, D. Y.; Green, J. J. Uptake and transfection with polymeric nanoparticles are dependent on polymer end-group structure, but largely independent of nanoparticle physical and chemical properties. Mol. Pharmaceutics 2012, 9 (11), 3375-83.
(19) Bishop, C. J.; Ketola, T.-m.; Tzeng, S. Y.; Sunshine, J. C.; Urtti, A.; Lemmetyinen, H.; Vuorimaa-Laukkanen, E.; Yliperttula, M.; Green, J. J. The effect and role of carbon atoms in poly(β-amino ester)s for DNA binding and gene delivery. J. Am. Chem. Soc. 2013, 135 (18), 6951-7.
(20) Eltoukhy, A. A.; Siegwart, D. J.; Alabi, C. A.; Rajan, J. S.; Langer, R.; Anderson, D. G. Effect of molecular weight of amine endmodified poly(β-amino ester)s on gene delivery efficiency and toxicity. Biomaterials 2012, 33 (13), 3594-603.
(21) Sunshine, J.; Green, J. J.; Mahon, K. P.; Yang, F.; Eltoukhy, A. a.; Nguyen, D. N.; Langer, R.; Anderson, D. G. Small-Molecule End-Groups of Linear Polymer Determine Cell-type Gene-Delivery Efficacy. Adv. Mater. 2009, 21 (48), 4947-4951.
(22) Cho, Y. W.; Kim, J D.; Park, K. Polycation gene delivery systems: escape from endosomes to cytosol. J. Pharm. Pharmacol. 2003, 55 (6), 721-734.
(23) Lim, Y.-b.; Kim, S -m.; Suh, H.; Park, J.-s. Biodegradable, endosome disruptive, and cationic network-type polymer as a highly efficient and nontoxic gene delivery carrier. Bioconjugate Chem. 2002, 13 (5), 952-957.
(24) Hall, A.; Lachelt, U.; Bartek, J.; Wagner, E.; Moghimi, S. M. Polyplex evolution: Understanding biology, optimizing performance. Mol. Ther. 2017, 25, 1476.
(25) Zhou, J.; Liu, J.; Cheng, C. J.; Patel, T. R.; Weller, C. E.; Piepmeier, J. M.; Jiang, Z.; Saltzman, W. M. Biodegradable poly(amine-co-ester) terpolymers for targeted gene delivery. Nat. Mater. 2012, 11 (1), 82-90.
(26) Yan, H.; Zhu, D.; Zhou, Z.; Liu, X.; Piao, Y.; Zhang, Z.; Liu, X.; Tang, J.; Shen, Y. Facile synthesis of semi-library of low charge density cationic polyesters from poly (alkylene maleate) s for efficient local gene delivery. Biomaterials 2018, 178, 559.
(27) Kim, J.; Sunshine, J. C.; Green, J. J. Differential polymer structure tunes mechanism of cellular uptake and transfection routes of poly(β-amino ester) polyplexes in human breast cancer cells. Bioconjugate Chem. 2014, 25 (1), 43-51.
(28) Guerrero-Cazares, H.; Tzeng, S. Y.; Young, N. P.; Abutaleb, A. O.; Quinones-Hinojosa, A.; Green, J. J. Biodegradable polymeric nanoparticles show high efficacy and specificity at DNA delivery to human glioblastoma in vitro and in vivo. ACS Nano 2014, 8 (5), 5141-53.
(29) Tzeng, S. Y.; Higgins, L. J.; Pomper, M. G.; Green, J. J. Student award winner in the Ph.D. category for the 2013 society for biomaterials annual meeting and exposition, Apr. 10-13, 2013, Boston, Mass.: biomaterial-mediated cancer-specific DNA delivery to liver cell cultures using synthetic poly(beta-a. J. Biomed. Mater. Res., Part A 2013, 101 (7), 1837-45.
(30) Zhou, Z.; Liu, X.; Zhu, D.; Wang, Y.; Zhang, Z.; Zhou, X.; Qiu, N.; Chen, X.; Shen, Y. Nonviral cancer gene therapy: Delivery cascade and vector nanoproperty integration. Adv. Drug Delivery Rev. 2017, 115, 115-154.
(31) Wilson, D. R.; Mosenia, A.; Suprenant, M. P.; Upadhya, R.; Routkevitch, D.; Meyer, R. A.; Quinones-Hinojosa, A.; Green, J. J. Continuous microfluidic assembly of biodegradable poly(beta-amino ester)/DNA nanoparticles for enhanced gene delivery. J. Biomed. Mater. Res., Part A 2017, 105 (6), 1813-1825.
(32) Bishop, C. J.; Majewski, R. L.; Guiriba, T.-R. M.; Wilson, D. R.; Bhise, N. S.; Quiñones-Hinojosa, A.; Green, J. J. Quantification of cellular and nuclear uptake rates of polymeric gene delivery nanoparticles and DNA plasmids via flow cytometry. Acta Biomater. 2016, 37, 120-130.
(33) Tzeng, S. Y.; Green, J. J. Subtle changes to polymer structure and degradation mechanism enable highly effective nanoparticles for siRNA and DNA delivery to human brain cancer. Adv. Healthcare Mater. 2013, 2 (3), 468-80.
(34) Sunshine, J. C.; Akanda, M. I.; Li, D.; Kozielski, K. L.; Green, J. J. Effects of base polymer hydrophobicity and end-group modification on polymeric gene delivery. Biomacromolecules 2011, 12 (10), 3592-600.
(35) Bhise, N. S.; Gray, R. S.; Sunshine, J. C.; Htet, S.; Ewald, A. J.; Green, J. J. The relationship between terminal functionalization and molecular weight of a gene delivery polymer and transfection efficacy in mammary epithelial 2-D cultures and 3-D organotypic cultures. Biomaterials 2010, 31 (31), 8088-96.
(36) Voit, B. New developments in hyperbranched polymers. J. Polym. Sci., Part A: Polym. Chem. 2000, 38 (14), 2505-2525.
(37) Sunshine, J. C.; Sunshine, S. B.; Bhutto, I.; Handa, J. T.; Green, J. J. Poly(β-amino ester)-nanoparticle mediated transfection of retinal pigment epithelial cells in vitro and in vivo. PLoS One 2012, 7 (5), e37543-e37543.
(38) Guo, W.; Lee, R. J. Efficient gene delivery via non-covalent complexes of folic acid and polyethylenimine. J. Controlled Release 2001, 77 (1), 131-138.
(39) Wilson, D. R.; Routkevitch, D.; Rui, Y.; Mosenia, A.; Wahlin, K. J.; Quinones-Hinojosa, A.; Zack, D. J.; Green, J. J. A Triple-Fluorophore-Labeled Nucleic Acid pH Nanosensor to Investigate Non-viral Gene Delivery. Mol. Ther. 2017, 25, 1697.
(40) Zhong, Z.; Song, Y.; Engbersen, J. F. J.; Lok, M. C.; Hennink, W. E.; Feijen, J. A versatile family of degradable non-viral gene carriers based on hyperbranched poly (ester amine) s. J. Controlled Release 2005, 109 (1-3), 317-329.
(41) Benjaminsen, R. V.; Mattebjerg, M. A.; Henriksen, J. R.; Moghimi, S. M.; Andresen, T. L. The possible “proton sponge” effect of polyethylenimine (PEI) does not include change in lysosomal pH. Mol. Ther. 2013, 21 (1), 149-157.
(42) Behr, J.-P. The proton sponge: a trick to enter cells the viruses did not exploit. CHIMIA International Journal for Chemistry 1997, 51 (1-2), 34-36.
(43) Boussif, O.; Lezoualc′h, F.; Zanta, M. a.; Mergny, M. D.; Scherman, D.; Demeneix, B.; Behr, J. P. A versatile vector for gene and oligonucleotide transfer into cells in culture and in vivo: polyethylenimine Proc. Natl. Acad. Sci. U.S.A 1995, 92 (16), 7297-301.
(44) Lächelt, U.; Kos, P.; Mickler, F. M.; Herrmann, A.; Salcher, E. E.; Rodl, W.; Badgujar, N.; Brauchle, C.; Wagner, E. Fine-tuning of proton sponges by precise diaminoethanes and histidines in pDNA polyplexes. Nanomedicine 2014, 10 (1), 35-44.
(45) Funhoff, A. M.; van Nostrum, C. F.; Koning, G. A.; Schuurmans-Nieuwenbroek, N. M. E.; Crommelin, D. J. A.; Hennink, W. E. Endosomal Escape of Polymeric Gene Delivery Complexes Is Not Always Enhanced by Polymers Buffering at Low pH. Biomacromolecules 2004, 5 (1), 32-39.
(46) Eltoukhy, A. A.; Chen, D.; Alabi, C. A.; Langer, R.; Anderson, D. G. Degradable terpolymers with alkyl side chains demonstrate enhanced gene delivery potency and nanoparticle stability. Adv. Mater. 2013, 25 (10), 1487-1493.
(47) Liu, H.; Wang, Y.; Wang, M.; Xiao, J.; Cheng, Y. Fluorinated poly (propylenimine) dendrimers as gene vectors. Biomaterials 2014, 35 (20), 5407-5413.
(48) Wen, Y.; Zhang, Z.; Li, J. Highly Efficient Multifunctional Supramolecular Gene Carrier System Self-Assembled from Redox-Sensitive and Zwitterionic Polymer Blocks. Adv. Funct. Mater. 2014, 24 (25), 3874-3884.
(49) Beh, C. W.; Pan, D.; Lee, J.; Jiang, X.; Liu, K. J.; Mao, H.-Q.; Wang, T.-h. Direct interrogation of DNA content distribution in nanoparticles by a novel microfluidics-based single-particle analysis. Nano Lett. 2014, 14 (8), 4729-35.
(50) Green, J. J.; Zugates, G. T.; Tedford, N. C.; Huang, Y. H.; Griffith, L. G.; Lauffenburger, D. a.; Sawicki, J. a.; Langer, R.; Anderson, D. G. Combinatorial Modification of Degradable Polymers Enables Transfection of Human Cells Comparable to Adenovirus. Adv. Mater. 2007, 19 (19), 2836-2842.
(51) Hoyng, S. A.; De Winter, F.; Gnavi, S.; Van Egmond, L.; Attwell, C. L.; Tannemaat, M. R.; Verhaagen, J.; Malessy, M. J. A. Gene delivery to rat and human Schwann cells and nerve segments: a comparison of AAV 1-9 and lentiviral vectors. Gene Ther. 2015, 22 (10), 767.
(52) Ellis, B. L.; Hirsch, M. L.; Barker, J. C.; Connelly, J. P.; Steininger, R. J.; Porteus, M. H. A survey of ex vivo/in vitro transduction efficiency of mammalian primary cells and cell lines with Nine natural adeno-associated virus (AAV1-9) and one engineered adeno-associated virus serotype. Virol. J. 2013, 10 (1), 74.
(53) Gaj, T.; Staahl, B. T.; Rodrigues, G. M. C.; Limsirichai, P.; Ekman, F. K.; Doudna, J. A.; Schaffer, D. V. Targeted gene knock-in by homology-directed genome editing using Cas9 ribonucleoprotein and AAV donor delivery. Nucleic Acids Res. 2017, 45, e98.
(54) Russell, S.; Bennett, J.; Wellman, J. A.; Chung, D. C.; Yu, Z.-F.; Tillman, A.; Wittes, J.; Pappas, J.; Elci, O.; McCague, S. Efficacy and safety of voretigene neparvovec (AAV2-hRPE65v2) in patients with RPE65-mediated inherited retinal dystrophy: a randomised, controlled, open-label, phase 3 trial. Lancet 2017, 390 (10097), 849-860.
(55) Gilleron, J.; Querbes, W.; Zeigerer, A.; Borodovsky, A.; Marsico, G.; Schubert, U.; Manygoats, K.; Seifert, S.; Andree, C.; Stöter, M.; Epstein-Barash, H.; Zhang, L.; Koteliansky, V.; Fitzgerald, K.; Fava, E.; Bickle, M.; Kalaidzidis, Y.; Akinc, A.; Maier, M.; Zerial, M. Image-based analysis of lipid nanoparticle-mediated siRNA delivery, intracellular trafficking and endosomal escape. Nat. Biotechnol. 2013, 31 (7), 638-46.
(56) Wittrup, A.; Ai, A.; Liu, X.; Hamar, P.; Trifonova, R.; Charisse, K.; Manoharan, M.; Kirchhausen, T.; Lieberman, J. Visualizing lipid-formulated siRNA release from endosomes and target gene knockdown. Nat. Biotechnol. 2015, 33 (8), 870-876.
(57) Sabnis, S.; Kumarasinghe, E. S.; Salerno, T.; Mihai, C.; Ketova, T.; Senn, J. J.; Lynn, A.; Bulychev, A.; McFadyen, I.; Chan, J. A Novel Amino Lipid Series for mRNA Delivery: Improved Endosomal Escape and Sustained Pharmacology and Safety in Non-human Primates. Mol. Ther. 2018, 26 (6), 1509-1519.
(58) Stewart, M. P.; Lorenz, A.; Dahlman, J; Sahay, G. Challenges in carrier-mediated intracellular delivery: moving beyond endosomal barriers. Wiley Interdisciplinary Reviews: Nanomedicine and Nanobiotechnology 2016, 8 (3), 465-478.

TABLE 6-S1
Monomer mole ratios for synthesis of BEAQ series

| Theoretical | | | |
|---|---|---|---|
| Triacrylate Mole | | | |
| Fraction (%) | Diacrylate Ratio | Triacrylate Ratio | Amine Ratio |
| 0 | 1.1 | 0.00 | 1 |
| 10 | 0.99 | 0.07 | 1 |
| 20 | 0.88 | 0.15 | 1 |
| 40 | 0.66 | 0.29 | 1 |
| 50 | 0.55 | 0.37 | 1 |
| 60 | 0.44 | 0.44 | 1 |
| 80 | 0.22 | 0.59 | 1 |
| 90 | 0.11 | 0.66 | 1 |

TABLE 6-S2
¹H NMR integrations for all polymers normalized to acrylate peaks
(5.5-6.5 ppm, 3H). To calculate average end-cap moieties per
polymer molecule, first the integrated area for B7 and B8 monomers
was calculated relative to the acrylate peak total area for
each polymer. Next, relative count of B7 and B8 monomers relative
to acrylate groups was calculated by scaling by the specific
number of hydrogen atoms per group to calculate synthesized
triacrylate mole fraction. GPC MN values were then used to calculate
the number of B7 and B8 repeat units per polymer molecule. The
number of theoretical end-cap moieties per polymer molecule
was then calculated as NE6 = 2 + NB8 where NE6 and
NB8 refer to the number average moiety count per polymer molecule
of monomers E6 and E8 respectively.

| Theoretical | | | |
|---|---|---|---|
| Triacrylate | B7 Phenyl | B8 methyl | S4 |
| Mole Fraction | 7.11 & 6.8 ppm | 0.83 ppm | 2.38 ppm |
| (%) | (4H each) | (3H) | (2H) |
| 0 | 9.42 | 0 | 4.62 |
| 10 | 7.16 | 0.91 | 5.05 |
| 20 | 6.41 | 1.25 | 4.62 |
| 40 | 6.27 | 1.87 | 3.70 |
| 50 | 4.08 | 2.58 | 4.63 |
| 60 | 2.19 | 2.02 | 3.40 |
| 80 | 0.951 | 3.10 | 3.19 |
| 90 | 0.611 | 4.16 | 3.57 |

TABLE 6-S3
Number average GPC calculated mass
fraction contributions of monomers

| Theoretical | | | | |
|---|---|---|---|---|
| Triacrylate Mole | | | | |
| Fraction (%) | B7 | B8 | S4 | E6 |
| 0 | 0.815 | 0.000 | 0.128 | 0.057 |
| 10 | 0.686 | 0.090 | 0.133 | 0.091 |
| 20 | 0.619 | 0.135 | 0.132 | 0.115 |
| 40 | 0.516 | 0.204 | 0.130 | 0.150 |
| 50 | 0.418 | 0.275 | 0.136 | 0.171 |
| 60 | 0.320 | 0.334 | 0.127 | 0.220 |
| 80 | 0.127 | 0.468 | 0.131 | 0.275 |
| 90 | 0.063 | 0.516 | 0.138 | 0.282 |

TABLE 6-S4
Backbone polymer amine density calculations. The molecular
weight for polymer repeat units consisting of monomers
B7 + S4, B8 + 2*S4 and ethylenimine were calculated.
Amine density was then determined as the number of amines
per polymer backbone molecular weight in Da. The branching
monomer (B8) gives rise to polymers with the highest tertiary
amine density per unit mass while B7 monomers give rise to
polymers with a lower tertiary amine density.

| | | Tertiary Amine Density |
|---|---|---|
| | Molecular Weight | (mMol Amines per |
| Repeat Unit | (Da) | gram polymer) |
| Diacryate: B7 + S4 | 573 | 1.75 |
| Triacrylate: B8 + 2*S4 | 474 | 4.22 |
| Ethylenimine | 43 | 23.26 |

TABLE 6-S5
Estimated secondary, tertiary and total density as a function
of polymer mass calculated from 1H NMR and GPC MN data.

| Theoretical | | | |
|---|---|---|---|
| Triacrylate Mole | Estimated Amine Density (mMol per gram polymer) | | |
| Fraction (%) | Secondary | Tertiary | Total |
| 0 | 0.8511 | 1.4853 | 2.3363 |
| 10 | 1.2766 | 1.5704 | 2.8470 |
| 20 | 1.6190 | 1.6107 | 3.2297 |
| 40 | 1.8571 | 1.4263 | 3.2835 |
| 50 | 2.3333 | 1.6430 | 3.9764 |
| 60 | 2.1429 | 1.1657 | 3.3086 |
| 80 | 2.2917 | 1.0244 | 3.3161 |
| 90 | 4.1935 | 1.8782 | 6.0718 |

TABLE 6-S6
Yo-Pro-1 iodide competition binding assay. RM one-way ANOVA was
performed with Geisser-Greenhouse corrections and Dunnett test
corrected multiple comparisons to the linear, 0 triacrylate mole
fraction polymer. Results are shown for multiple comparisons
assessment at the concentration of 75 μg/mL BEAQ concentration
tested with an n = 3 well replicates for each polymer.

| Triacrylate mole fraction | pH 5.0, 25 mM | pH 7.4, 150 mM |
|---|---|---|
| 10 | ns | ns |
| 20 | ns | ** |
| 40 | ns | ** |
| 50 | ns | * |
| 60 | ns | ** |
| 80 | ns | *** |
| 90 | ns | * |

### Example 7

### Reducible Branched Ester-Amine Quadpolymers (rBEAQs) Codelivering Plasmid DNA and RNA Oligonucleotides Enable CRISPR/Cas9 Genome Editing

### 7.1 OVERVIEW

Functional codelivery of plasmid DNA and RNA oligonucleotides in the same nanoparticle system is challenging due to differences in their physical properties as well as their intracellular locations of function. In this study, we synthesized a series of reducible branched ester-amine quadpolymers (rBEAQs) and investigated their ability to coencapsulate and deliver DNA plasmids and RNA oligos. The rBEAQs are designed to leverage polymer branching, reducibility, and hydrophobicity to successfully cocomplex DNA and RNA in nanoparticles at low polymer to nucleic acid w/w ratios and enable high delivery efficiency. We validate the synthesis of this new class of biodegradable polymers, characterize the self-assembled nanoparticles that these polymers form with diverse nucleic acids, and demonstrate that the nanoparticles enable safe, effective, and efficient DNA-siRNA codelivery as well as nonviral CRISPR-mediated gene editing utilizing Cas9 DNA and sgRNA codelivery.

### 7.2 BACKGROUND

The introduction of exogenous genetic material into mammalian cells has been widely used in the laboratory to modulate gene expression and induce cellular reprogramming, 1 differentiation,2,3 and programmed cell death.4-6 Recently, these technologies have begun moving into the clinic and mark the beginning of a new paradigm for genetic medicine.7,8 Traditional gene therapies involve the delivery of DNA, often in the form of plasmids or minicircle DNA,9 into target cells. RNA oligonucleotides such as short interfering RNA (siRNA) can enable target-specific gene silencing, 0, and single guide RNAs (sgRNAs) complex with Cas9 endonucleases to achieve site-specific gene editing via the CRISPR/Cas9 system12,13 The biological functionality of these nucleic acids depends heavily on their successful intracellular delivery. 14

Although nonviral vectors delivering either plasmid DNA or siRNA have been widely reported, very few studies have been able to functionally codeliver both in the same nanoparticle system. This can be challenging as DNA and RNA oligonucleotides are vastly different in size (5000 vs 20 bp) and stiffness. 15,16 In this study, we synthesized a series of reducible branched ester-amine quadpolymers (rBEAQs) and investigated their ability to form nanoparticles that could functionally codeliver plasmid DNA and RNA oligonucleotides. The rBEAQs were designed based on recent studies that have demonstrated that hyperbranched cationic polymers are superior to their linear counterparts at DNA17-20 and oligonucleotide21,22 delivery in multiple polymeric vector systems. The branched polymer architecture could increase the charge density of each polymer molecule, allowing for stronger nucleic acid binding affinity.23 Disulfide bonds are another useful functionality as they can enable environmentally triggered cargo release in the reducing cytosolic environment. They can be incorporated into delivery vectors as polymer side chains,34 cross-linking moieties between polymer chains,25 and part of the polymer backbone26 and have been used successfully in several siRNA delivery systems. Finally, increasing polymer hydrophobicity has been shown to improve nanoparticle stability and increase DNA27 as well as siRNA delivery efficacy.28

Using a facile one-pot Michael addition reaction, we were able to tune the reducibility and hydrophobicity of the polymers by simply adjusting the monomer ratios. We found that the nucleic acid binding affinity, release kinetics, nanoparticle uptake, and functional nucleic acid delivery could be modulated in a highly controlled manner Our nanoparticle system enabled up to 77% DNA transfection and 66% siRNA-mediated knockdown. More importantly, delivery of Cas9 DNA and sgRNA enabled 40% gene knockout, further highlighting the robustness of this codelivery system.

### 7.3. MATERIALS AND METHODS

7.3.1. Materials. 2-Hydroxyethyl disulfide (CAS 1892291), triethylamine (CAS 121448), acryloyl chloride (CAS 814686), bisphenol A glycerolate (1 glycerol/phenol) diacrylate (B7; CAS 4687949), trimethylolpropane triacrylate (B8; CAS 15625895), 2-(3-aminopropylamino)ethanol (E6; CAS 4461396), L-buthionine-sulfoximine (CAS 83730534), and solvents were purchased from Sigma Aldrich (St. Louis, Mo.). 4-Amino-1-butanol (S4; CAS 133251005) was purchased from Alfa Aesar (Tewksbury, Mass.). Plasmids pCAGGFPd2 (14760) and piRFP670-N1 (45457) were purchased from Addgene (Cambridge, Mass.). PB-CMV-MCS-EF1a-RFP PiggyBac plasmid (PB512B-1) and PiggyBac transposase expression plasmid (PB200A-1) were purchased from System Biosciences (Palo Alto, Calif.). Negative control siRNA (1027281) was purchased from Qiagen (Germantown, Md.). GFP siRNA targeting the sequence 5′-GCA AGC TGA CCC TGA AGT TC-3′ (SEQ ID NO: 3) (P-002048-01) was purchased from Dharmacon (Lafayette, Colo.). Cy5-labeled siRNA (SIC005) was purchased from Sigma Aldrich.
7.3.2. Polymer Synthesis. Bioreducible monomer 2,2-disulfanediylbis(ethane-2,1-diyl) diacrylate (BR6) was synthesized using a method similar to Kozielski et al.26 Briefly, 2-hydroxyethyl disulfide was acrylated with acryloyl chloride (1:1.1 molar ratio in dichloromethane) in the presence of excess triethylamine After filtering out the precipitate, the product was washed with water, dried with sodium sulfate, and the solvent was removed by rotary evaporation.

For polymer synthesis, monomers BR6, B7, B8, and S4 were dissolved in anhydrous dimethylsulfoxide (DMSO) according to the B monomer molar ratios listed in Table 7-S1 for an overall vinyl/amine ratio of 2.2:1 at a concentration of 150 mg/mL. After overnight reaction at 90° C. with stirring, the polymers were end-capped by reacting with monomer E6 (0.2 M final concentration in DMSO) at room temperature for 1 h. The end-capped polymers were purified by two diethyl ether washes, after which the remaining solvent was removed in a vacuum chamber. Polymers were dissolved in DMSO at 100 mg/mL and stored in aliquots at −20° C. with desiccant.

7.3.3. Yo-Pro-1 Iodide Nucleic Acid Binding Assay. Yo-Pro-1 iodide fluorescent dye (Invitrogen) was mixed with siRNA at a final concentration of 0.5 μM Yo-Pro and 0.5 μM scRNA in 25 mM sodium acetate (NaAc, pH 5.0). Polymers were dissolved in NaAc, and 25 μL of polymer solution was mixed with 75 μL of RNA/Yo-Pro solution per well in 96-well black-bottom plates. The solutions were incubated at 37° C. for 20 min before fluorescence readings were taken on a fluorescence multiplate reader (Biotek Synergy 2). To measure siRNA binding in reducing conditions over time, the polymer concentration was set at the lowest concentration at which each polymer achieved >80% quenching. The polymer/siRNA/Yo-Pro solution was mixed with 10 μL of glutathione solution (final concentration 5 mM) and incubated at 37° C. Fluorescence readings were taken at the indicated time points.
7.3.4. Polymer Characterization: NMR and GPC. The polymer structure was characterized by nuclear magnetic resonance spectroscopy (NMR) via 1H NMR in CDCl3 (Bruker 500 MHz) and analyzed using TopSpin 3.5 software. To measure polymer molecular weight and polydispersity, polymers were dissolved in BHT-stabilized tetrahydrofuran with 5% DMSO and 1% piperidine, filtered through a 0.2 μm PTFE filter, and measured with gel permeation chromatography against linear polystyrene standards (Waters, Milford, Mass.).
7.3.5. Gel Retardation Assay. Nanoparticles were synthesized by dissolving the polymer and siRNA separately in NaAc buffer at the desired concentrations. The solutions were mixed at a 1:1 volume ratio, and nanoparticles were allowed to self-assemble at room temperature for 10 min, after which nanoparticles were incubated in the presence of 5 mM glutathione or 150 mM phosphate-buffered saline (PBS) at 37° C. Samples were taken at various time points and frozen at −80° C. to stop the reaction. For gel retardation assays of R6,7,8_64 nanoparticles coencapsulating plasmid DNA and siRNA, nucleic acids were first premixed at a 1:1 volume ratio and then mixed with polymer to allow for nanoparticle self-assembly. Polymer dosage was varied from 10 to 0 w/w (free nucleic acids). Samples were loaded onto a 1% agarose gel using 30% glycerol as the loading buffer. Gel electrophoresis was performed in TAE buffer at 100 V for 15 min, after which the gel was imaged under UV.
7.3.6. Nanoparticle Characterization. Nanoparticles were prepared as described above and diluted in 150 mM PBS to determine particle size and surface charge in neutral isotonic buffer. Hydrodynamic diameter was measured via nanoparticle tracking analysis at 1:500 dilution in PBS using a NanoSight NS300, whereas ζ-potential was measured at 1:6 dilution in PBS via electrophoretic light scattering on a Malvern Zetasizer NanoZS (Malvern Panalytical). To characterize nanoparticle stability over time in physiological conditions, nanoparticle size was also measured at 1:6 dilution in 10% serum-containing cell culture medium once per hour for 9 h using a Malvern Zetasizer Pro (Malvern Panalytical). Transmission electron microscopy (TEM) images were acquired with a Philips CM120 TEM (Philips Research). Nanoparticles were prepared at a polymer concentration of 1.8 mg/mL in 25 mM NaAc, 30 μL was added to 400-square mesh carbon-coated TEM grids, and the grids were allowed to coat for 20 min Grids were then rinsed with ultrapure water, counterstained with uranyl acetate (0.5% in distilled water), and allowed to fully dry before imaging.
7.3.7. Cell Culture and Cell Line Preparation. HEK-293T human embryonic kidney and Huh7 human hepatocellular carcinoma cells were cultured in Dulbecco's modified Eagle's medium (DMEM; ThermoFisher) supplemented with 10% fetal bovine serum (FBS) and 1% penicillin/streptomycin. A PiggyBac transposon/transposase system was used to generate cell lines constitutively expressing a destabilized form of GFP (GFPd229) with a protein half-life of two hours. The GFPd2 PiggyBac transposon plasmid (PB-CAG-GFPd2 Addgene 115665) was created by inserting the GFPd2 gene into a PiggyBac plasmid through standard restriction enzyme cloning. The transposon plasmid was then cotransfected with the PiggyBac transposase expression plasmid into cells using the method described below. Cells underwent two transfections and were then grown out for five passages to allow the fluorescence signal from transient transfections to fade. Positively expressing cells were isolated via fluorescence-assisted cell sorting (FACS), and colonies grown from single cells were grown out to establish stably expressing cell lines.
7.3.8. Transfection. Cells were seeded onto 96-well tissue culture plates at a density of 15 000 cells per well in 100 μL of complete medium and allowed to adhere overnight. Nanoparticles were formed immediately prior to transfection as described above. For experiments delivering siRNA only, each nanoparticle condition was formulated with a scrambled control RNA (scRNA) or an siRNA targeting GFP (siGFP) with a final RNA concentration of 100 nM per well. For experiments codelivering siRNA and DNA, nanoparticles were formulated with a final dose of 200 ng of DNA per well in addition to 100 nM scRNA or siGFP, respectively, for a final total nucleic acid dose of 400 ng per well. Nanoparticles coencapsulating DNA and siRNA were formed by premixing the nucleic acids at a 1:1 volume ratio in NaAc buffer prior to mixing with the polymer solution. Prior to the addition of nanoparticles, the cell culture medium was replaced with 100 μL of serum-free media. Then, 20 μL of nanoparticles was added per well and incubated with cells for 2 h, at which point the nanoparticle/media mixture was replaced with fresh complete media. Knockdown of GFPd2 fluorescence was assessed via flow cytometry 1 day post-transfection using a BD Accuri C₆ flow cytometer (BD Biosciences). Knockdown was quantified by normalizing the geo metric mean of fluorescence of wells treated with siGFP to that of wells transfected using the same nanoparticle formulation delivering scRNA. For codelivery experiments, DNA transfection was quantified as the percentage of cells positively expressing iRFP when gated against untreated controls (N=4). Transfections in which sodium bicarbonate (NaHCO₃) was used to increase nanoparticle pH were done by forming nanoparticles in acidic NaAc buffer as previously described and then mixing with 50 mg/mL NaHCO₃ buffer (pH 9) at a 1:1 volume ratio before adding to cells. Transfections using commercially available nonviral transfection reagents Lipofectamine 2000, Lipofectamine 3000 (Thermo-Fisher), and jetPrime (Polyplus) were performed according to the manufacturer's instructions. In DNA-siRNA codelivery experiments, 25 kD bPEI was used at 1 w/w.

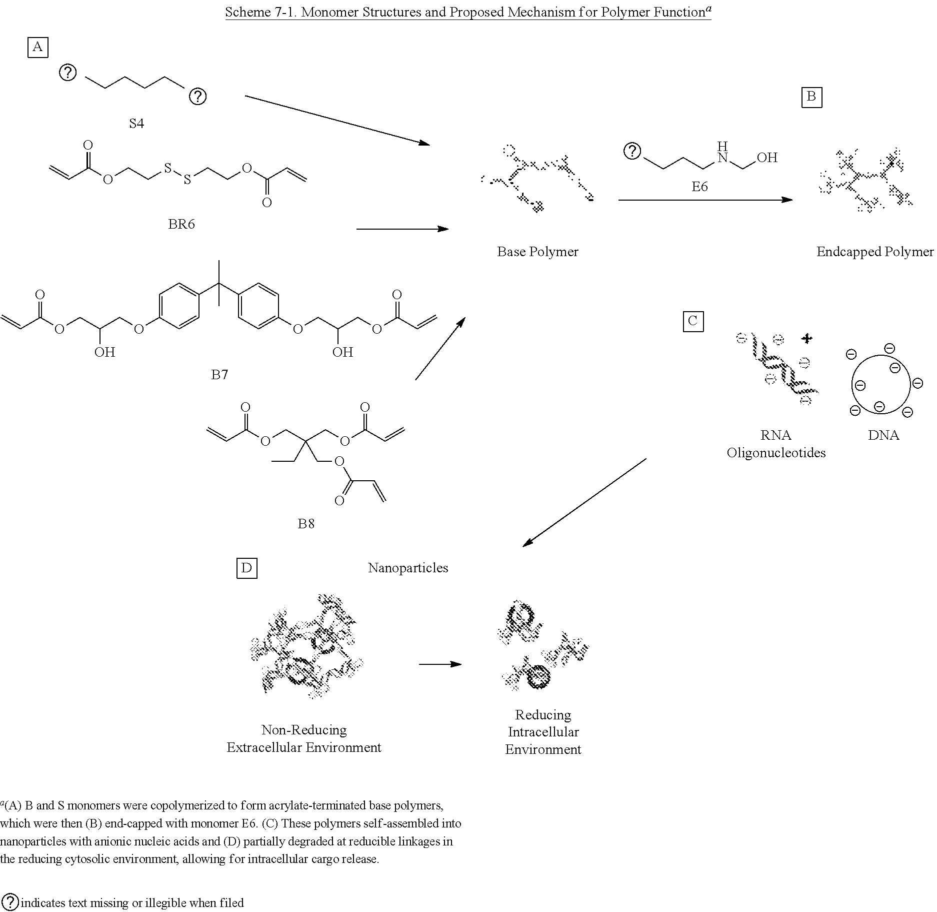

7.3.9 Cellular Uptake and Viability. Cy5-labeled siRNA was diluted 1:5 in unlabeled siRNA and used to formulate nanoparticles as described above. Nanoparticles were added to cells in serum-free media and incubated for 2 h, at which point cells were washed once with PBS and detached via trypsinization. Cells were further washed with heparin (50 μg/mL in PBS) to remove nanoparticles adhering to cells, resuspended in FACS buffer (2% FBS in PBS), and nanoparticle uptake was quantified by flow cytometry. Cell viability was assessed 24 h post-transfection using the MTS CellTiter 96 Aqueous One cell proliferation assay (Promega) following the manufacturer's instructions. Cell viability of treated cells was normalized to that of untreated cells; N=4.
7.3.10 Glutathione Inhibition with L-Buthionine-sulfoximine (BSO). L-Buthionine-sulfoximine (BSO) was dissolved in cell culture media at 2000 μM. Cells were allowed to settle for 3 h after plating, at which time 50 μL of media was replaced by 50 μL of BSO solution for 1000 μM final BSO concentration, which has been shown to effectively inhibit intracellular glutathione levels.30 Cells were incubated for 24 h with complete media containing 1000 μM BSO, which was replaced with serum-free BSO media immediately before transfection. After 2 h incubation with nanoparticles, cells were replenished with fresh BSO-containing complete media and incubated for 24 h, at which point cell viability and flow cytometry assays were performed.
7.3.11 Confocal Microscopy. HEK-293T cells were plated on Nunc Lab-Tek 8 chambered borosilicate cover glass well plates (155411; ThermoFisher) at 30 000 cells/well 1 day prior to transfection in 300 μL of phenol red free DMEM supplemented with 10% FBS and 1% penicillin/streptomycin. R6,7,8_64 nanoparticles nanoparticles (10 w/w) were prepared as described above using premixed Cy3-labeled siRNA and Cy5-labeled plasmid DNA at a 1 w/w ratio of nucleic acids. Cy5-labeled plasmid DNA was prepared as previously described31,32 and mixed at a 4 w/w ratio with unlabeled eGFP-N1 plasmid DNA. Nanoparticles were then diluted into media and added to cells at a total nucleic acid dose of 1000 ng/well and incubated for 2 h. Prior to imaging, cells were stained with Hoechst 33342 at a 1:5000 dilution for nuclei visualization. Images were acquired over an area with sides of 140 μm at Nyquist limit resolution using a Zeiss LSM 780 microscope with Zen Blue software and 63× oil immersion lens. Specific laser channels used were 405 nm diode, 488 nm argon, 561 nm solid-state, and 639 nm diode lasers. Laser intensity and detector gain settings were maintained across all image acquisitions.
7.3.12 CRISPR Gene Editing. The template used for in vitro transcription of sgRNA targeting GFP was synthesized as a gBlock from IDT (sequence listed in Table 7-S2). In vitro transcription was performed using a MEGAshortscript T7 Transcription kit (Invitrogen) according to the manufacturer's instructions, and the sgRNA product was purified using a MEGAclear Transcription Clean-up kit (Invitrogen). Cas9 plasmid DNA (41815)12 was purchased from Addgene and amplified by Aldeveron (Fargo, N. Dak.). For codelivery transfections, DNA and sgRNA were delivered using R6,7,8_64 nanoparticles as described above. Gene knockout was assessed using flow cytometry 5 days post-transfection unless otherwise noted.
2.13. Statistics. Prism 6 (Graphpad, La Jolla, Calif.) was used for all statistical analyses and curve plotting. Statistical tests were performed with a global a value of 0.05. Unless otherwise stated, absence of statistical significance markings where a test was stated to have been performed signified no statistical significance. The statistical test used and the number of experimental replicates are listed in the captions for each figure. Statistical significance was denoted as follows: *p<0.05; **p<0.01, ***p<0.001, ****p<0.0001.

### 7.4 RESULTS AND DISCUSSION

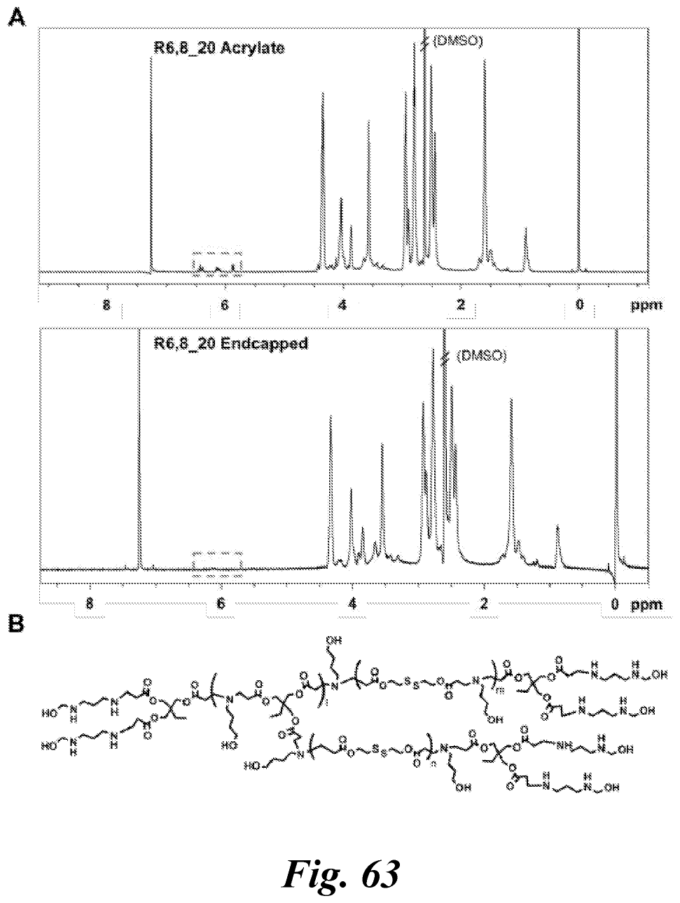

7.4.1. Polymer Synthesis and Characterization. Polymers were synthesized following a facile one-pot Michael addition reaction in which acrylate monomers BR6 and B8 were copolymerized with amine-containing monomer S4 (Scheme 7-1). After end-capping with monomer E6, this class of polymers is referred to as R6,8_N, where N denotes the branching B8 monomer content in the polymer backbone (i.e., R6,8_20 contains 20% B8). In the polymer series containing the additional diacrylate monomer B7, B8 monomer content was kept constant at 20%, and polymers are referred to as R6,7,8_M, where M denotes the B7 monomer content in the polymer backbone. For acrylate-terminated base polymer synthesis, B and S monomers were dissolved in anhydrous DMSO at 150 mg/mL (monomer concentrations >400 mg/mL resulted in complete gelation), and stepwise polymerization reaction was allowed to proceed overnight at 90° C. with stirring. The chemical structures of base polymers were determined via NMR spectroscopy, which verified that the polymers were acrylate terminated by three distinct acrylate peaks at 5.5-6.5 ppm (FIG. 63). Polymer end-capping with monomer E6 was performed at room temperature for 1 h and confirmed by the disappearance of these peaks. Molecular weight data was obtained from GPC analysis, which showed that with increasing B8 content, both Mn and Mw values generally increased (Table 7-1). For R6,7,8-4-6 polymers, for which the B8 content was fixed at 20%, the molecular weight did not change significantly with varying B7 content, suggesting that molecular weight is largely controlled by polymer branching and cross-linking effects contributed by triacrylate monomer B8.

TABLE 7-1
Molecular Weight Data from GPC Characterization and
Monomer Composition Calculated from 1H NMR Spectra

| | | | | | Monomer fraction | | |
|---|---|---|---|---|---|---|---|
| | polymer | | | | in polymer | | |
| | name | Mₙ | M_{w} | PDI | BR6 | B7 | B8 |
| R6,8-4-6 | R6,8_0 | 2224 | 2682 | 1.21 | 1.00 | | |
| | R6,8_20 | 3168 | 4038 | 1.27 | 0.75 | | 0.25 |
| | R6,8_40 | 4050 | 5896 | 1.46 | 0.54 | | 0.46 |
| | R6,8_60 | 4943 | 9949 | 2.01 | 0.36 | | 0.64 |
| | R6,8_80 | 4675 | 8728 | 1.87 | 0.23 | | 0.77 |
| R6,7,8-4-6 | R6,7,8_16 | 4511 | 7616 | 1.69 | 0.62 | 0.17 | 0.21 |
| (20% B8) | R6,7,8_40 | 4570 | 7435 | 1.63 | 0.41 | 0.40 | 0.19 |
| | R6,7-8_64 | 4438 | 7066 | 1.59 | 0.17 | 0.63 | 0.20 |

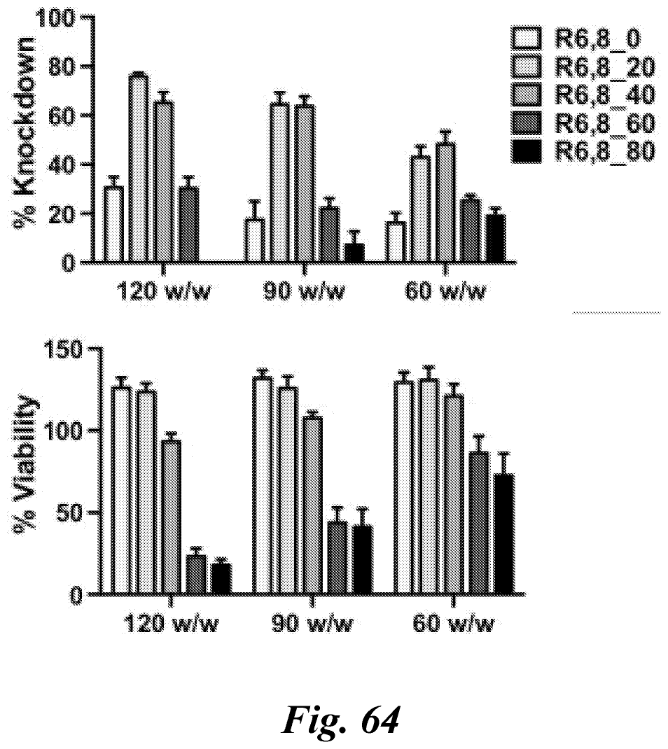

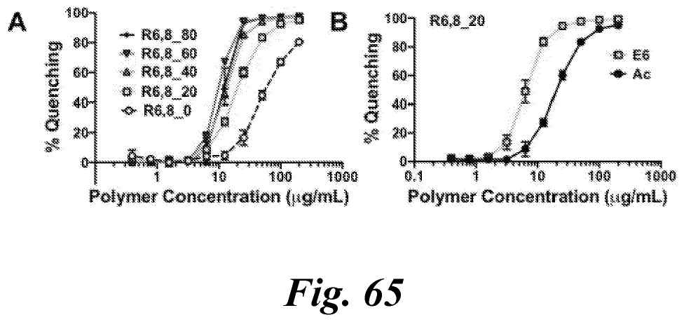

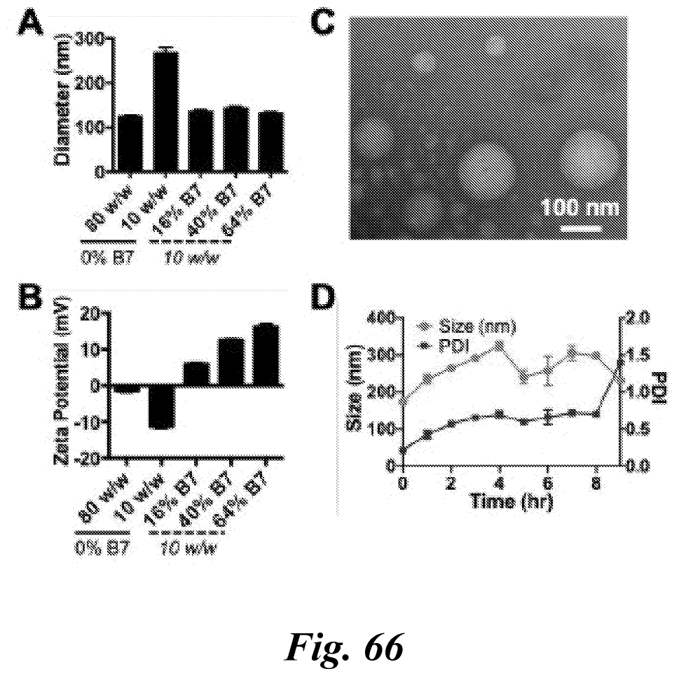

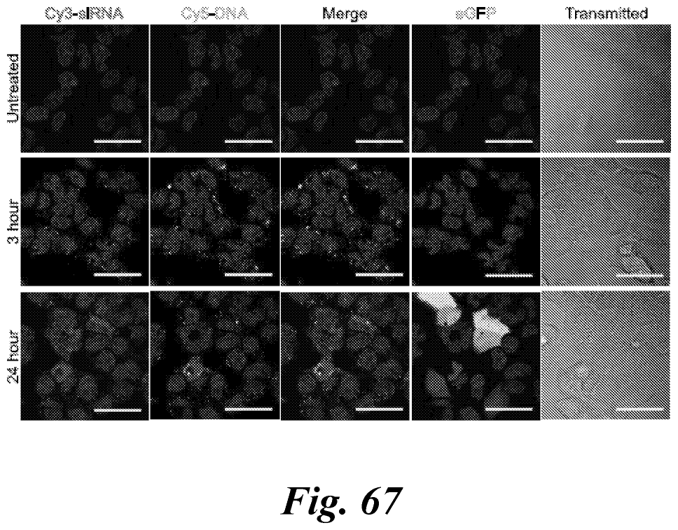

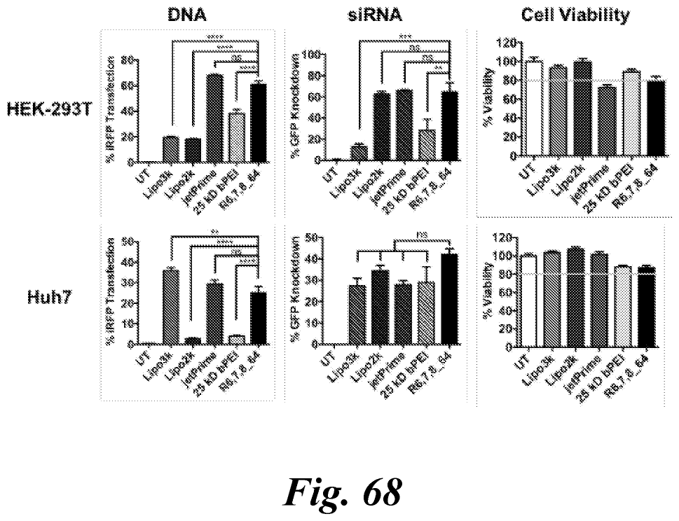

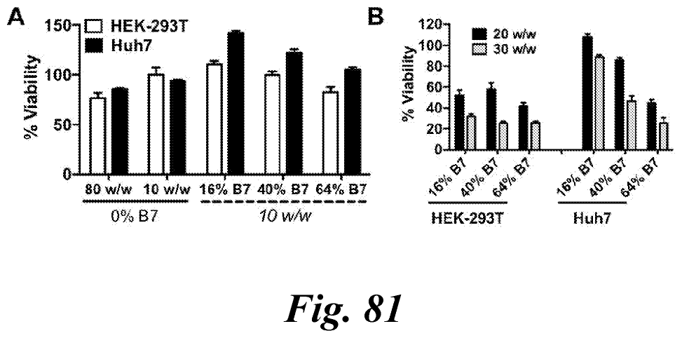

7.4.2. siRNA Delivery: Gene Knockdown, Cellular Uptake, and Cytotoxicity. R6,8-4-6 polymers were used to deliver siRNA targeting GFP (siGFP) in HEK-293T cells stably expressing a destabilized form of GFP with a short halflife (GFPd2).29 At 100 nM siRNA dose and 180 polymersiRNA w/w ratio, R6,8_20 achieved 75% knockdown with negligible cytotoxicity (FIG. 47). All branched polymers in the R6,8_N series with the exception of R6,8_80 achieved significantly higher knockdown than the linear polymer (R6,8_0). Knockdown levels peaked with R6,8_20 and R6,8_40 (FIG. 64), and the same trend was observed for nanoparticle uptake (FIG. 47B). Previous studies have demonstrated that nanoparticle uptake and transfection efficacy increased with increasing polymer molecular weight.33,34 This was not the case in our polymer system as R6,8_60 and R6,8_80 had the highest molecular weight but achieved relatively poor knockdown. This could in part be due to the fact that increasing polymer branching resulted in lower cell viability caused by decreasing reducible BR6 monomer content. Indeed, when cells were pretreated with L-buthioninesulfoximine (BSO) to inhibit cellular production of glutathione, the main intracellular reducing agent,35 nanoparticlemediated cytotoxicity significantly increased (FIG. 47C). This increased toxicity was beyond the additive effects of either nanoparticle or BSO treatment alone, indicating that the cell's inability to reduce disulfide bonds after glutathione blockade induced higher levels of cell death and confirming our hypothesis that polymer reducibility attenuated cytotoxicity by enabling them to rapidly degrade to relatively nontoxic oligomers. Thus, the bioreducibility of the rBEAQ nanoparticles is designed both to enable environmentally triggered cargo release upon entering the cytosol and as a mechanism to limit potential cytotoxicity of the branched polymers by quickly breaking them down into smaller components once they reach their target inside the cell. To elucidate the mechanism by which moderately branched polymers achieved the highest levels of knockdown, we assessed the physical characteristics of the nanoparticles. All polymers in the series formed nanoparticles with hydrodynamic diameters around 100 nm (FIG. 47D). Nanoparticles formed with the linear polymer had negative surface charge, and -potential generally became increasingly positive with increased polymer branching (FIG. 47E). This is likely due to the fact that increased branching resulted in increasing numbers of secondary amine-containing end-groups per polymer molecule, which are positively charged in the pH 5 NaAc buffer. The increased cationic charge of moderately branched polymer nanoparticles likely contributes to nanoparticle uptake and siRNA-mediated knockdown in vitro, which is consistent with many published reports.36-38 This trend does not apply to very highly branched polymers, however, in part due to the high levels of cytotoxicity incurred by these nanoparticle formulations.
7.4.3. siRNA Binding and Environmentally Triggered Release. A competitive binding assay using Yo-Pro-1 iodide (Yo-Pro) was used to assess siRNA binding strength in R6,8-4-6 polymers. Yo-Pro dye fluoresces upon nucleic acid binding, and quenching of fluorescence after the polymer outcompetes the dye for siRNA binding was used as a measure of binding strength. Increasing polymer branching increased the siRNA binding strength, which was seen in both end-capped (FIG. 48A) and acrylate-terminated polymers (FIG. 65A). Plotting knockdown as a function of the polymer EC50 w/w of siRNA binding (where lower EC₅₀ w/w corresponds to tighter siRNA binding and higher degree of polymer branching) revealed a biphasic response (FIG. 48B). Binding affinity and degree of knockdown both increased approximately 4-fold from R6,8_0 to R6,8_20 and decreased steadily when the B8 content exceeded 40%. This suggests that an optimal range for siRNA binding affinity exists, and polymers that bind too tightly cannot release siRNA to achieve efficient knockdown, whereas those that do not bind tightly enough cannot form nanoparticles that effectively promote nanoparticle internalization.39,40 siRNA binding affinity, along with other nanoparticle biophysical and chemical properties such as the size, surface charge, and bioreducibility discussed earlier, all contribute to the differential gene silencing effects seen here. For polymers with the same B8 content, end-capped polymers exhibited stronger binding than their acrylate-terminated counterparts (FIG. 65B). These results suggest that polymer branching increases siRNA binding via two mechanisms. The first is mediated by the increased branching structure in the polymer backbone, which increases the molecular weight of the polymer and drives stronger binding through greater hydrophobic effects. The second is mediated by increased branching endpoints, which increase the number of end-capping molecules. As the secondary amines in the polymer endgroups are positively charged in the pH 5 NaAc buffer, they further increase siRNA binding through electrostatic interactions. We next investigated siRNA release kinetics of R6,8-4-6 nanoparticles in 5 mM glutathione to mimic the reducing intracellular environment.35 Nanoparticles were sampled at specific time points, and standard gel electrophoresis was performed to assess siRNA release (FIG. 48C). The linear polymer released siRNA almost instantaneously, and release was complete by 1 h. Increased polymer branching slowed siRNA release considerably, with R6,8_20 beginning release at 1 h and higher branching polymers at 7 h. The same trend was observed when a Yo-Pro binding assay was performed with nanoparticles incubated over time in reducing buffer conditions (FIG. 48D). These results indicate that siRNA binding and release can be modulated in a highly controlled manner by changing the ratio between branching and reducible monomers and that siRNA release can be designed to occur in an environmentally triggered manner via reduction of disulfide bonds. However, we have also shown that blocking intracellular glutathione levels did not significantly decrease the observed level of siRNA-mediated knockdown (FIG. 47C), which suggests that other polymer degradation mechanisms such as the hydrolysis of ester bonds over a period of 4-6 h41 could also contribute to siRNA release from nanoparticles. Incorporation of disulfide linkages in the rBEAQ polymers helps ensure fragmentation of the polymers into small oligomers, reducing cytotoxicity, and enables higher doses, branching, or w/w formulation ratios of the polymers to be safely utilized.
7.4.4. Codelivery of siRNA and DNA. As moderately branched polymers have been shown to maintain strong nucleic acid binding affinity while effectively releasing siRNA cargo in the reducing cytosolic environment, we hypothesized that they may be suitable for the codelivery of plasmid DNA and siRNA. R6,8_20 (the top polymer for siRNA delivery) was used to encapsulate 200 ng each of siGFP siRNA and a plasmid DNA encoding iRFP670. R6,8_20 nanoparticles enabled efficient codelivery to HEK-293T cells (FIG. 49A), resulting in 66% siRNA-mediated knockdown and 77% DNA transfection with negligible cytotoxicity (FIG. 81A). The same formulation achieved much lower delivery efficiency in harder-to-transfect Huh7 cells (23% knockdown and 5% transfection; FIG. 49B), prompting the need to develop more effective polymers for codelivery. To this end, we investigated the effect of polymer hydrophobicity by incorporating monomer B7 at ratios indicated in Table 7-S1 to synthesize the R6,7,8-4-6 polymer series. B7 was chosen as it contains a bisphenol A group, which has been shown to bind DNA via hydrophobic effects42 and enable high-efficiency DNA transfection.27,43,44 B7-containing polymers effectively complexed nucleic acids at very low w/w, forming nanoparticles around 150 nm in diameter and +6 to +16 mV in ζ-potential (FIG. 66). R6,7,8_64 nanoparticles (10 w/w) were quite stable in complete cell culture media mimicking physiological conditions for several hours with a hydrodynamic diameter doubling time >4 h as assessed by dynamic light scattering (FIG. 66D). In contrast, R6,8-4-6 polymers with 0% B7 content formed much larger nanoparticles (270 nm) with −11 mV ζ-potential at 10 w/w. B7-containing polymers were used at significantly lower w/w formulations compared to R6,8-4-6 polymers used for siRNA complexation earlier because R6,7,8-4-6 polymers incurred significantly higher cytotoxicity than the R6,8-4-6 polymers, limiting their use to very low w/w formulations (FIG. 81B). Nevertheless, B7-containing polymers enabled higher levels of knockdown and transfection at 10 w/w in HEK-293T cells (FIG. 49A, FIG. 49C), though the difference was less notable when R6,8-4-6 polymers were used at higher w/w. More strikingly, R6,7,8-4-6 polymers enabled significantly higher codelivery in Huh7 cells compared to R6,8-4-6 at all w/w formulations, with the best formulation achieving 53% knockdown and 37% transfection (FIG. 49B). A gel retardation assay demonstrated that R6,7,8_64 completely condensed both plasmid DNA and siRNA at 10 w/w, and decreasing the polymer dose resulted in siRNA release at 5 w/w and DNA release at 1 w/w (FIG. 49D). We further explored the intracellular delivery locations of siRNA and DNA using confocal laser scanning microscopy, which demonstrated different fates for internalized siRNA and DNA. At an early 3 h time point following nanoparticle treatment, most endosomes possessed both siRNA and DNA, whereas at 24 h post-treatment, diffuse cytosolic siRNA was detectable in most cells and the occasional z-slice revealed some Cy5-labeled plasmid DNA in the nucleus (FIG. 49E). Using a mix of fluorescently labeled plasmid DNA and unlabeled plasmid DNA encoding a fluorescent reporter protein GFP, we were also able to detect a fraction of the cells expressing GFP at 24 h post-transfection, which was undetectable in cells at 3 h post-treatment (FIG. 67). Studies have shown that polymers optimized for DNA delivery may not be optimal for siRNA and vice versa.45 This may be due to the differences in size and charge density between DNA and siRNA as well as their intracellular sites of action. Bishop et al. approached this problem with a polymercoated gold nanoparticle system where siRNA and DNA were adsorbed onto the nanoparticle using different polymers in a layer-by-layer synthesis scheme; the optimal formulation in this study resulted in 34% knockdown and 14% transfection in human brain cancer cells.46 Another study using poly(L-lysine) polyplexes for codelivery of siRNA and DNA to HEK-293T cells showed >80% knockdown but achieved <10% DNA transfection.47 The delivery system reported herein achieved significantly higher codelivery in both HEK-293T cells and harder-to-transfect Huh7 human liver cancer cells. These polymers are easy to formulate into nanoparticles via selfassembly in a single step and enabled more efficient codelivery of both DNA and siRNA compared to several leading commercially available nonviral transfection reagents (FIG. 68).

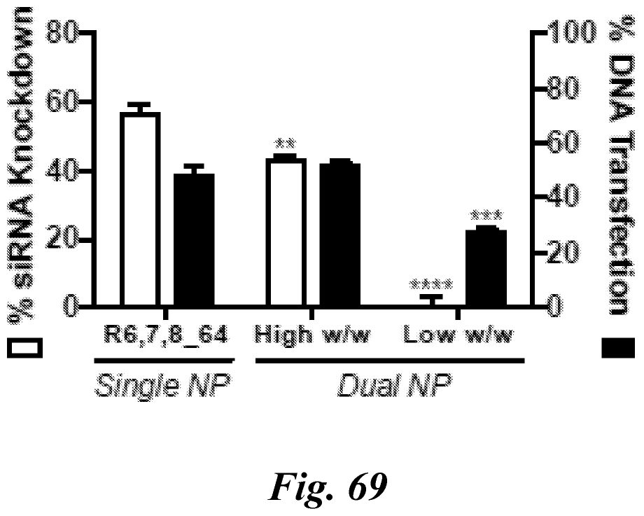

We further compared the DNA-siRNA codelivery efficacy of the system presented herein with that of using nanoparticle formulations previously optimized for the delivery of each nucleic acid separately (FIG. 69). In the latter strategy, plasmid DNA was encapsulated using polymer 446 at 60 w/w (previously optimized for DNA delivery-1) and siRNA was encapsulated using polymer R646 at 120 w/w (previously optimized for siRNA delivery-0). The two nanoparticles were formulated separately and added to cells after nanoparticle formation. In the single nanoparticle strategy, the same amount of nucleic acids was premixed and coencapsulated in R6,7,8_64 nanoparticles (10 w/w). Our results show that using the dual nanoparticle delivery strategy, siRNA knockdown levels were significantly lower than that achieved by the single nanoparticle codelivery strategy while DNA transfection levels were similar. Furthermore, when polymers 446 and R646 were used to formulate nanoparticles at 10 w/w for direct comparison with polymer R6,7,8_64, both siRNA and DNA delivery levels were significantly lower. These results indicate that coencapsulation of multiple nucleic acid cargo types in the same nanoparticle system has the advantages of higher transfection efficiency as well as greater simplicity in formulation; this is especially important for potential clinical translation as it could greatly simplify the synthesis and regulatory approval processes.

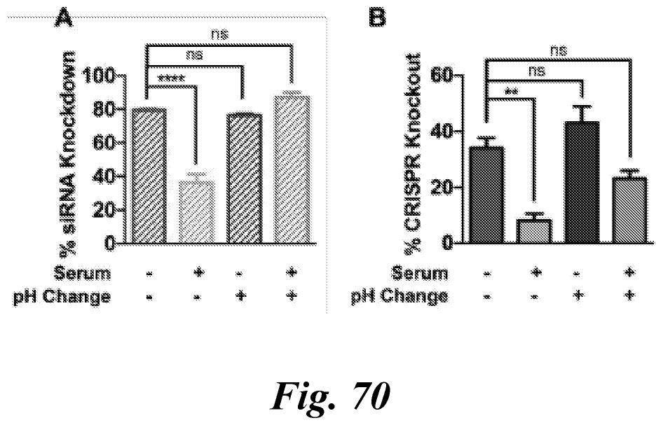

7.4.5. Codelivery of Cas9 DNA and sgRNA for CRISPRMediated Gene Editing. Next, we coencapsulated Cas9 plasmid DNA and sgRNA targeting GFP in our nanoparticles for intracellular delivery of the CRISPR/Cas9 gene editing system within one biodegradable nanoparticle. Gene knockout, which can be assessed by a decrease in GFP fluorescence, is contingent upon codelivery of both components as the Cas9 endonuclease must assemble with sgRNA to form a functional ribonucleoprotein (RNP) complex. This is a rigorous test of codelivery as the two components must be present in the same cell as well as remain bioactive at the same time in order for editing to occur. Our results showed that R6,7,8_64 nanoparticles enabled 40% gene knockout in HEK-293T cells (FIG. 81A). Delivery of either component alone did not result in appreciable levels of knockout, confirming the need for codelivery. The optimal sgRNA-Cas9 plasmid molar ratio was 33. Interestingly, we saw a distinct GFP-negative population (GFP null) in CRISPR-treated cells, which was not observed in cells treated with GFP siRNA (FIG. 81B). siRNA-mediated gene silencing downshifted the GFP fluorescence of the entire population of treated cells, whereas CRISPR-mediated knockout completely turned off GFP in a fraction of cells. Kinetic studies showed that siRNA-mediated gene silencing faded rapidly and fluorescence returned to pretreatment levels after 11 days (FIG. 81C). In contrast, CRISPR-mediated silencing peaked after 5 days and remained constant for the entirety of the period tested. Our results suggest that gene silencing mediated by siRNA knockdown or CRISPR knockout could be suitable for different therapeutic goals. The former has a faster onset and results in significant but transient downregulation in the entire population of treated cells. The latter takes longer to reach peak levels but can produce a sustained and binary downregulation in a smaller fraction of the population. It is important to note that all transfection experiments so far have been performed in serum-free medium. It has been widely reported that the presence of serum may decrease transfection efficacy by inducing polyplex disruption and aggregation.50 On the contrary, some studies have also demonstrated that the presence of serum proteins may prevent disassembly of nanocomplexes.51 To investigate the performance of our nanoparticle system in serum conditions, R6,7,8_64 nanoparticles (10 w/w) were formulated with siRNA or Cas9 DNA and sgRNA and administered to cells in complete medium (10% serum). The presence of serum significantly reduced transfection efficacy in both cases (FIG. 70). However, when NaHCO₃ was added to the nanoparticle formulation to increase nanoparticle pH prior to addition to the cells, transfection in both cases increased back to similar levels as in serum-free conditions. The addition of anionic compounds to nanoparticles to increase transfection in serum conditions has been utilized in other delivery systems52 and is a viable strategy to stabilize polymeric polyplexes.

### 7.5. SUMMARY

We synthesized a new series of reducible branched ester-amine quadpolymers (rBEAQs) that enabled codelivery of plasmid DNA and RNA oligonucleotides in the same biodegradable self-assembled nanoparticle system. Our best formulation achieved 77% DNA transfection and 66% siRNA-mediated knockdown in HEK-293T cells and 37% transfection and 53% knockdown in Huh7 cells. More importantly, codelivery of Cas9 DNA and sgRNA in the same nonviral nanoparticles enabled 40% CRISPR/Cas9-mediated gene knockout. To our knowledge, this is the first time that CRISPR-mediated gene editing has been achieved through the codelivery of Cas9 plasmid and sgRNA. The effective codelivery of plasmid DNA and RNA oligonucleotides reported here, as well as the ability to leverage bioreducibility, hydrophobicity, and polymer branching to enable effective codelivery in different cell types, may prove useful for applications such as novel combinatorial gene therapies and genome editing.

TABLE 7-S1
Backbone B monomer composition for R6,8-
4-6 and R6,7,8-4-6 polymer series.

| | Polymer | Monomer Molar Ratios | | |
|---|---|---|---|---|
| | Name | BR6 | B7 | B8 |
| R6,8-4-6 | R6,8_0 | 100% | 0% | 0% |
| | R6,8_20 | 80% | 0% | 20% |
| | R6,8_40 | 60% | 0% | 40% |
| | R6,8_60 | 40% | 0% | 60% |
| | R6,8_80 | 20% | 0% | 80% |
| R6,7,8-4-6 | R6,7,8_16 | 64% | 16% | 20% |
| (20% B8) | R6,7,8_40 | 40% | 40% | 20% |
| | R6,7-8_64 | 16% | 64% | 20% |

TABLE 7-S2
DNA sequence for sgRNA in vitro
transcription template.

| Sequence | Notes |
|---|---|
| GTTTTTTT [symbol] | T7 promoter |
| ggagcgcaccatcttcttcagttt | sequence |
| tagagctagaaatagcaagttaaa | GFP target |
| aataaaaggctagtccgttatca | sequence |
| acttgaaaaagtggcaccgagtc | sgRNA scaffold |
| ggtgcttttttt | |

### 7.6 REFERENCES

(1) Bhise, N. S.; Wahlin, K. J.; Zack, D. J.; Green, J. J. Evaluating the Potential of Poly(Beta-Amino Ester) Nanoparticles for Reprogramming Human Fibroblasts to Become Induced Pluripotent Stem Cells. Int. J. Nanomed. 2013, 8, 4641-58.
(2) Warren, L.; Manos, P. D.; Ahfeldt, T.; Loh, Y.-H.; Li, H.; Lau, F.; Ebina, W.; Mandal, P. K.; Smith, Z. D.; Meissner, A.; et al. Highly Efficient Reprogramming to Pluripotency and Directed Differentiation of Human Cells with Synthetic Modified Mrna. Cell Stem Cell 2010, 7, 618-630.
(3) Tzeng, S. Y.; Hung, B. P.; Grayson, W. L.; Green, J. J. Cystamine-Terminated Poly(Beta-Amino Ester)S for Sirna Delivery to Human Mesenchymal Stem Cells and Enhancement of Osteogenic Differentiation. Biomaterials 2012, 33, 8142-51.
(4) Powers, M. V.; Clarke, P. A.; Workman, P. Dual Targeting of Hsc70 and Hsp72 Inhibits Hsp90 Function and Induces Tumor-Specific Apoptosis. Cancer Cell 2008, 14, 250-262.
(5) Caldas, H.; Jaynes, F. O.; Boyer, M. W.; Hammond, S.; Altura, R. A. Survivin and Granzyme B-Induced Apoptosis, a Novel Anticancer Therapy. Mol. Cancer Ther. 2006, 5, 693-703.
(6) Mangraviti, A.; Tzeng, S. Y.; Kozielski, K. L.; Wang, Y.; Jin, Y.; Gullotti, D.; Pedone, M.; Buaron, N.; Liu, A.; Wilson, D. R.; Hansen, S. K.; Rodriguez, F. J.; Gao, G.-D.; DiMeco, F.; Brem, H.; Olivi, A.; Tyler, B.; Green, J. J. Polymeric Nanoparticles for Nonviral Gene Therapy Extend Brain Tumor Survival in Vivo. ACS Nano 2015, 9, 1236-1249.
(7) Ledford, H. Engineered Cell Therapy for Cancer Gets Thumbs up from Fda Advisers. Nature 2017, 547, 270.
(8) Fischer, A. FDA Approves Novel Gene Therapy to Treat Patients with a Rare Form of Inherited Vision Loss. https://www.fda.gov/;
(9) Munye, M. M.; Tagalakis, A. D.; Barnes, J. L.; Brown, R. E.; McAnulty, R. J.; Howe, S. J.; Hart, S. L. Minicircle DNA Provides Enhanced and Prolonged Transgene Expression Following Airway Gene Transfer. Sci. Rep. 2016, 6, No. 23125.
(10) Fire, A.; Xu, S. Q.; Montgomery, M. K.; Kostas, S. A.; Driver, S. E.; Mello, C. C. Potent and Specific Genetic Interference by Double-Stranded Rna in Caenorhabditis Elegans. Nature 1998, 391, 806-811.
(11) Hannon, G. J. Rna Interference. Nature 2002, 418, 244-251.
(12) Mali, P.; Yang, L.; Esvelt, K. M.; Aach, J.; Guell, M.; DiCarlo, J. E.; Norville, J. E.; Church, G. M. Rna-Guided Human Genome Engineering Via Cas9. Science 2013, 339, 823-826.
(13) Cong, L.; Ran, F. A.; Cox, D.; Lin, S.; Barretto, R.; Habib, N.; Hsu, P. D.; Wu, X.; Jiang, W.; Marraffini, L. A.; Zhang, F. Multiplex Genome Engineering Using Crispr/Cas Systems. Science 2013, 339, 819-823.
(14) Kozielski, K. L.; Rui, Y.; Green, J. J. Non-Viral Nucleic Acid Containing Nanoparticles as Cancer Therapeutics. Expert Opin. Drug Delivery 2016, 1475-1487.
(15) Kebbekus, P.; Draper, D. E.; Hagerman, P. Persistence Length of Rna. Biochemistry 1995, 34, 4354-4357.
(16) Hagerman, P. J. Flexibility of Rna. Annu. Rev. Biophys. Biomol. Struct. 1997, 26, 139-156.
(17) Tian, H.; Xiong, W.; Wei, J.; Wang, Y.; Chen, X.; Jing, X.; Zhu, Q. Gene Transfection of Hyperbranched Pei Grafted by Hydrophobic Amino Acid Segment Pblg. Biomaterials 2007, 28, 2899-2907.
(18) Kadlecova, Z.; Rajendra, Y.; Matasci, M.; Baldi, L.; Hacker, D. L.; Wurm, F. M.; Klok, H.-A. DNA Delivery with Hyperbranched Polylysine: A Comparative Study with Linear and Dendritic Polylysine. J. Controlled Release 2013, 169, 276-288.
(19) Zhao, T.; Zhang, H.; Newland, B.; Aied, A.; Zhou, D.; Wang, W. Significance of Branching for Transfection: Synthesis of Highly Branched Degradable Functional Poly (Dimethylaminoethyl Meth-acrylate) by Vinyl Oligomer Combination. Angew. Chem., Int. Ed. 2014, 53, 6095-6100.
(20) Wilson, D. R.; Rui, Y.; Siddiq, K.; Routkevitch, D.; Green, J. J. Differentially Branched Ester Amine Quadpolymers with Amphiphilic and Ph Sensitive Properties for Efficient Plasmid DNA Delivery. Mol. Pharmaceutics 2019, 655-668.
(21) Rahbek, U. L.; Nielsen, A. F.; Dong, M.; You, Y.; Chauchereau, A.; Oupicky, D.; Besenbacher, F.; Kjems, J.; Howard, K. A. Bioresponsive Hyperbranched Polymers for Sirna and Mirna Delivery. J. Drug Targeting 2010, 18, 812-820.
(22) Jia, H.-Z.; Zhang, W.; Zhu, J.-Y.; Yang, B.; Chen, S.; Chen, G.; Zhao, Y.-F.; Feng, J.; Zhang, X.-Z. Hyperbranched-Hyperbranched Polymeric Nanoassembly to Mediate Controllable Co-Delivery of Sirna and Drug for Synergistic Tumor Therapy. J. Controlled Release 2015, 216, 9-17.
(23) Wilson, D. R.; Rui, Y.; Siddiq, K.; Routkevitch, D.; Green, J. J. Differentially Branched Ester Amine Quadpolymers with Amphiphilic and Ph-Sensitive Properties for Efficient Plasmid DNA Delivery. Mol. Pharmaceutics 2019, 16, 655-668.
(24) Chen, G.; Wang, Y.; Xie, R.; Gong, S. Tumor-Targeted Ph/Redox Dual-Sensitive Unimolecular Nanoparticles for Efficient Sirna Delivery. J. Controlled Release 2017, 259, 105-114.
(25) Tai, Z.; Wang, X.; Tian, J.; Gao, Y.; Zhang, L.; Yao, C.; Wu, X.; Zhang, W.; Zhu, Q.; Gao, S. Biodegradable Stearylated Peptide with Internal Disulfide Bonds for Efficient Delivery of Sirna in Vitro and in Vivo. Biomacromolecules 2015, 16, 1119-1130.
(26) Kozielski, K. L.; Tzeng, S. Y.; Green, J. J. A Bioreducible Linear Poly(Beta-Amino Ester) for Sirna Delivery. Chem. Commun. 2013, 49, 5319-5321.
(27) Eltoukhy, A. A.; Chen, D.; Alabi, C. A.; Langer, R.; Anderson, D. G. Degradable Terpolymers with Alkyl Side Chains Demonstrate Enhanced Gene Delivery Potency and Nanoparticle Stability. Adv. Mater. 2013, 25, 1487-1493.
(28) Nelson, C. E.; Kintzing, J. R.; Hanna, A.; Shannon, J. M.; Gupta, M. K.; Duvall, C. L. Balancing Cationic and Hydrophobic
Content of Pegylated Sirna Polyplexes Enhances Endosome Escape, Stability, Blood Circulation Time, and Bioactivity in Vivo. ACS Nano 2013, 7, 8870-8880.
(29) Matsuda, T.; Cepko, C. L. Controlled Expression of Transgenes Introduced by in Vivo Electroporation. Proc. Natl. Acad. Sci. U.S.A. 2007, 104, 1027.
(30) Yao, C.; Tai, Z.; Wang, X.; Liu, J.; Zhu, Q.; Wu, X.; Zhang, L.; Zhang, W.; Tian, J.; Gao, Y.; Gao, S. Reduction-Responsive Cross-Linked Stearyl Peptide for Effective Delivery of Plasmid DNA. Int. J. Nanomed. 2015, 10, 3403.
(31) Wilson, D. R.; Mosenia, A.; Suprenant, M. P.; Upadhya, R.; Routkevitch, D.; Meyer, R. A.; Quinones-Hinojosa, A.; Green, J. J. Continuous Microfluidic Assembly of Biodegradable Poly(Beta-Amino Ester)/DNA Nanoparticles for Enhanced Gene Delivery. J. Biomed. Mater. Res. A 2017, 105, 1813-1825.
(32) Wilson, D. R.; Routkevitch, D.; Rui, Y.; Mosenia, A.; Wahlin, K. J.; Quinones-Hinojosa, A.; Zack, D. J.; Green, J. J. A Triple-Fluorophore-Labeled Nucleic Acid Ph Nanosensor to Investigate Non-Viral Gene Delivery. Mol. Ther. 2017, 1697-1709.
(33) Huang, M.; Khor, E.; Lim, L.-Y. Uptake and Cytotoxicity of Chitosan Molecules and Nanoparticles: Effects of Molecular Weight and Degree of Deacetylation. Pharm. Res. 2004, 21, 344-353.
(34) Eltoukhy, A. A.; Siegwart, D. J.; Alabi, C. A.; Rajan, J. S.; Langer, R.; Anderson, D. G. Effect of Molecular Weight of Amine
End-Modified Poly(B-Amino Ester)S on Gene Delivery Efficiency and Toxicity. Biomaterials 2012, 33, 3594-3603.
(35) Griffith, O. W. Biologic and Pharmacologic Regulation of Mammalian Glutathione Synthesis. Free Radicals Biol. Med. 1999, 27, 922-935.
(36) Yu, B.; Zhang, Y.; Zheng, W.; Fan, C.; Chen, T. Positive Surface Charge Enhances Selective Cellular Uptake and Anticancer Efficacy of Selenium Nanoparticles. Inorg. Chem. 2012, 51, 8956-8963.
(37) Harush-Frenkel, O.; Debotton, N.; Benita, S.; Altschuler, Y. Targeting of Nanoparticles to the Clathrin-Mediated Endocytic Pathway. Biochem. Biophys. Res. Commun. 2007, 353, 26-32.
(38) Liu, X.; Howard, K. A.; Dong, M.; Andersen, M. O.; Rahbek, U. L.; Johnsen, M. G.; Hansen, O. C.; Besenbacher, F.; Kjems, J. The Influence of Polymeric Properties on Chitosan/Sirna Nanoparticle Formulation and Gene Silencing. Biomaterials 2007, 28, 1280-1288.
(39) Schaffer, D. V.; Fidelman, N. A.; Dan, N.; Lauffenburger, D. A. Vector Unpacking as a Potential Barrier for Receptor-Mediated Polyplex Gene Delivery. Biotechnol. Bioeng. 2000, 67, 598-606.
(40) Bishop, C. J.; Ketola, T.-m.; Tzeng, S. Y.; Sunshine, J. C.; Urtti, A.; Lemmetyinen, H.; Vuorimaa-Laukkanen, E.; Yliperttula, M.; Green, J. J. The Effect and Role of Carbon Atoms in Poly(B-Amino Ester)S for DNA Binding and Gene Delivery. J. Am. Chem. Soc. 2013, 135, 6951-6957.
(41) Sunshine, J. C.; Peng, D. Y.; Green, J. J. Uptake and Transfection with Polymeric Nanoparticles Are Dependent on
Polymer End-Group Structure, but Largely Independent of Nanoparticle Physical and Chemical Properties. Mol. Pharmaceutics 2012, 9, 3375-3383.
(42) Zhang, Y.-L.; Zhang, X.; Fei, X.-C.; Wang, S.-L.; Gao, H.-W. Binding of Bisphenol a and Acrylamide to Bsa and DNA: Insights into the Comparative Interactions of Harmful Chemicals with Functional Biomacromolecules. J. Hazard. Mater. 2010, 182, 877-885.
(43) Cutlar, L.; Zhou, D.; Gao, Y.; Zhao, T.; Greiser, U.; Wang, W.; Wang, W. Highly Branched Poly (B-Amino Esters): Synthesis and Application in Gene Delivery. Biomacromolecules 2015, 16, 2609-2617.
(44) Gao, Y.; Huang, J.-Y.; O'Keeffe Ahern, J.; Cutlar, L.; Zhou, D.; Lin, F.-H.; Wang, W. Highly Branched Poly (B-Amino Esters) for Non-Viral Gene Delivery: High Transfection Efficiency and Low Toxicity Achieved by Increasing Molecular Weight. Biomacromolecules 2016, 17, 3640-3647.
(45) Tzeng, S. Y.; Green, J. J. Subtle Changes to Polymer Structure and Degradation Mechanism Enable Highly Effective Nanoparticles for Sirna and DNA Delivery to Human Brain Cancer. Adv. Healthcare Mater. 2013, 2, 468-480.
(46) Bishop, C. J.; Tzeng, S. Y.; Green, J. J. Degradable Polymer-Coated Gold Nanoparticles for Co-Delivery of DNA and Sirna. Acta Biomater. 2015, 11, 393-403.
(47) Chang Kang, H.; Bae, Y. H. Co-Delivery of Small Interfering Rna and Plasmid DNA Using a Polymeric Vector Incorporating Endosomolytic Oligomeric Sulfonamide. Biomaterials 2011, 32, 4914-4924.
(48) Wilson, D. R.; Mosenia, A.; Suprenant, M. P.; Upadhya, R.; Routkevitch, D.; Meyer, R. A.; Quinones-Hinojosa, A.; Green, J. J. Continuous Microfluidic Assembly of Biodegradable Poly(Beta-Amino Ester)/DNA Nanoparticles for Enhanced Gene Delivery. J. Biomed. Mater. Res., Part A 2017, 1813-1825.
(49) Kozielski, K. L.; Tzeng, S. Y.; Mendoza, B. A. H. d; Green, J. J. Bioreducible Cationic Polymer-Based Nanoparticles for Efficient and Environmentally Triggered Cytoplasmic Sirna Delivery to Primary Human Brain Cancer Cells. ACS Nano 2014, 8, 3232-3241.
(50) Moore, T. L.; Rodriguez-Lorenzo, L.; Hirsch, V.; Balog, S.; Urban, D.; Jud, C.; Rothen-Rutishauser, B.; Lattuada, M.; Petri-Fink, A. Nanoparticle Colloidal Stability in Cell Culture Media and Impact on Cellular Interactions. Chem. Soc. Rev. 2015, 44, 6287-6305.
(51) Pezzoli, D.; Zanda, M.; Chiesa, R.; Candiiani, G. The Yin of Exofacial Protein Sulfhydryls and the Yang of Intracellular Glutathione in in Vitro Transfection with Ss14 Bioreducible Lipoplexes. J. Controlled Release 2013, 165, 44-53.
(52) Guo, W.; Lee, R. J. Efficient Gene Delivery Via Non-Covalent Complexes of Folic Acid and Polyethylenimine J Controlled Release 2001, 77, 131-138.

### Example 8

### Poly(Beta-Amino Ester) Nanoparticles Enable Non-Viral Delivery of CRISPR/Cas9 Plasmids for Gene Knockout and Gene Deletion

### 8.1 OVERVIEW

The CRISPR/Cas9 system is a powerful gene editing tool with wide-ranging applications, but the safe and efficient intracellular delivery of CRISPR components remains a challenge. In this study, we utilized biodegradable poly(beta-amino ester) nanoparticles to co-deliver plasmid DNA encoding Cas9 and sgRNA, respectively, to enable gene knockout following 1-cut edits as well as gene deletion following 2-cut edits. We designed a reporter system that allows for easy evaluation of both types of edits: gene knockout can be assessed by a decrease in iRFP fluorescence while deletion of an expression stop cassette turns on a red-enhanced nanolantern fluorescence/luminescence dual reporter. Nanoparticles enabled up to 70% gene knockout due to small indels as well as 45% gain-of-function expression after a 600-bp deletion edit. The efficiency of 2-cut edits is more sensitive than 1-cut edits to Cas9 and sgRNA expression level, which is best predicted by the geometric mean fluorescence of a reporter gene when performing nanoparticle transfection screens. Our findings demonstrate promising biodegradable nanoparticle formulations for gene editing as well as provide new insights into the screening and transfection requirements for different types of gene edits and are applicable for designing varied non-viral delivery systems for the CRISPR/Cas9 platform.

### 8.2 BACKGROUND

The CRISPR/Cas9 gene editing system consists of a short guide RNA (sgRNA) conferring target sequence specificity which complexes with the Cas9 endonuclease to enable site-specific DNA cleavage.¹⁻³ This could result in gene knockout following non-homologous end joining (NHEJ) or, in the presence of a repair template, gene knock-in through homology-directed repair (HDR). Targeting sgRNAs to two sites flanking a genomic region of interest can result in the complete removal of the gene segment following NHEJ, which could be important in the silencing of genetic elements with no open reading frames such as microRNAs or long noncoding RNAs.⁴⁻⁵ CRISPR-mediated gene editing is contingent upon nuclear colocalization of both the Cas9 protein and sgRNA, and efficient intracellular delivery of CRISPR components remains a challenge.

Viral vectors have been demonstrated to be effective for delivery but are more challenging to produce for both pre-clinical and clinical studies and restricted in cargo size. This is problematic as the Cas9 gene is over 4 kb long, and delivery using AAVs (packaging capacity ˜4.7 kb) sometimes require that different CRISPR components be packaged in separate viral particles, introducing complexity and potentially reducing efficacy.⁶⁻⁷ Synthetic vectors are largely agnostic to cargo size, and several recent reports have demonstrated strategies for non-viral intracellular delivery of the CRISPR/Cas9 gene editing platform. These include nanoparticle delivery of Cas9 and sgRNA as a ribonucleoprotein (RNP) complex⁸⁻¹² or in the form of Cas9 mRNA and sgRNA.¹³⁻¹⁴ Cas9 and sgRNA encoded in plasmid DNA is another delivery format for CRISPR gene editing. Plasmid DNA can be easily constructed using standard molecular cloning techniques to include different Cas9 structures,¹⁵⁻¹⁶ multiplex sgRNA,¹⁷ and transcriptional targeting elements for cell type-specific editing.¹⁸ Furthermore, large libraries of biomaterials previously used for plasmid DNA delivery can be screened for CRISPR gene editing in a high-throughput manner¹⁹ to yield optimal formulations for gene editing in different applications.

Although several studies have reported strategies for non-viral CRISPR plasmid delivery,^{18, 20-23} most involve gene knockout applications using sgRNA designed to enable cleavage at a single site, and none to our knowledge have investigated the transfection requirements for gene deletion after cleavage at multiple sites. In this study, we designed a novel reporter system for easy detection of gene knockout following CRISPR-mediated cleavage at one genomic site (1-cut edit) as well as gene deletion following DNA cleavage at two sites flanking a region of interest (2-cut edit). We used poly(beta-amino ester)s (PBAEs), a class of biodegradable cationic polymers that has been shown to be effective at plasmid DNA delivery,²⁴ for intracellular delivery of plasmid DNA encoding both the Cas9 endonuclease and sgRNA, respectively, and demonstrate that these polymeric nanoparticles enable efficient 1-cut as well as 2-cut edits. Moreover, we systematically varied transfection parameters to probe the relationship between the expression of CRISPR components and the subsequent efficacy of different types of CRISPR-mediated edits. A non-viral nanoparticle formulation for safe and effective gene-editing containing only a cationic polymer and plasmid DNA is presented, without the need for co-encapsulation of protein or RNA. Further, our results provide important insights on the threshold gene expression levels required for 1- and 2-cut edits in easy-to-transfect as well as hard-to-transfect cell lines.

### 8.3 RESULTS

### 8.3.1 Polymeric Nanoparticles for Gene Delivery

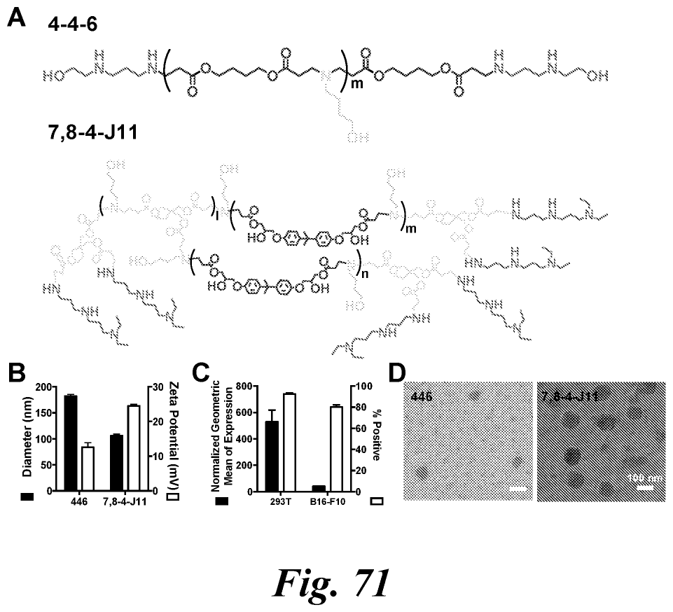

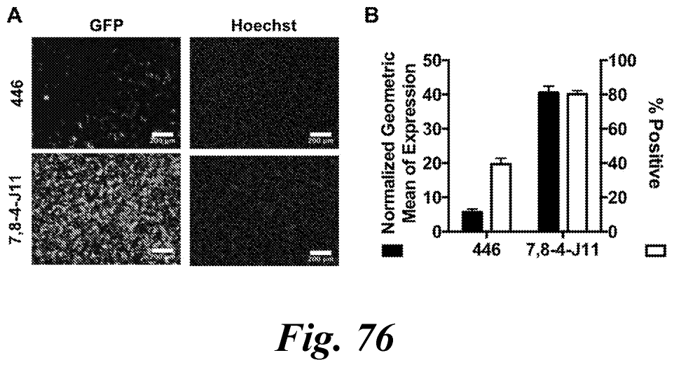

Polymer 446, which has been shown previously to be effective at plasmid DNA delivery to a variety of cells,²⁵⁻²⁶ was used to transfect HEK-293T cells (FIG. 71A). The newly developed branched polymer 7,8-4-J11 enabled higher transfection efficacy in B16-F10 murine melanoma cells²⁷ (FIG. 76) and was used to transfect these cells. Both polymers formed nanoparticles 100-200 nm in diameter with positive zeta potentials (12-25 mV) (FIG. 71B). Transfection efficacy as assessed with a GFP reporter plasmid showed that >80% cells were transfected in both cell lines (FIG. 71C). However, when geometric mean fluorescence was used to quantify expression, 293T cells achieved expression that was nearly 1 order of magnitude higher than B16 cells.

### 8.3.2 Gene Knockout Following 1-Cut Edits

293T cells constitutively expressing a destabilized form of GFP were transfected with nanoparticles encapsulating two plasmids encoding the Cas9 endonuclease and a sgRNA targeting GFP, respectively. Successful gene knockout was assessed by a decrease in GFP fluorescence. Nanoparticles co-delivering both plasmids enabled co-expression, generating 70% gene knockout, while formulations delivering either component alone had negligible effects (FIG. 2A). A kinetic study revealed that gene knockout reached maximal levels on day 3 and was maintained for over 3 weeks (FIG. 2B). The Surveyor® mutation detection assay was performed on cells treated with the combination nanoparticles or each component alone (FIG. 2C) and confirmed that edits occurred only when both CRISPR components were delivered. Sanger sequencing revealed that most edits were single base-pair indels (FIG. 2D), which likely caused frameshift mutations and subsequent gene silencing.

8.3.3 Gain-of-Function Edits after 2-Cut Stop Cassette Deletion

We designed a reporter system based on the Ai9 mouse²⁸ in which an expression stop cassette consisting of two SV40 terminators in series was placed upstream of a red-enhanced nanolantern (ReNL) fluorescence-luminescence dual reporter²⁹ (FIG. 3A). This expression cassette was cloned into a piggyBac transposon plasmid to facilitate genomic integration at high efficiency after co-transfection with a piggyBac transposase plasmid.³% near-infrared fluorescent protein (iRFP670)³¹ was also incorporated into the system as a selection marker for positively-expressing cells during fluorescence-activated cell sorting (FACS). Thus, this system can be easily used to generate stably-expressing reporter cell lines for rapid read-out of knockout as well as deletion mutations.

A sgRNA targeted to remove both SV40 sequences (sg1) via a 630 bp deletion resulted in turning on of ReNL expression while sgRNAs removing either SV40 sequence alone (sg2 or sg3) yielded negligible expression. A plasmid containing both sg2 and sg3 sequences governed by two U6 promoters (sg2+sg3) also resulted in turning on of expression, although at slightly lower levels than sg1 (FIG. 3B). Genomic DNA of cells treated with each sgRNA was PCR amplified for the 800 bp region immediately surrounding the stop cassette. Gel electrophoresis of the PCR products confirmed that turning on expression required the complete removal of both SV40 terminator sequences (>400 bp removed). Interestingly for cells treated with the combination sg2+sg3 plasmid, a faint band around 500 bp was observed, indicating that only one SV40 sequence was deleted in a fraction of edits. This suggests that the lower level of gene deletion achieved by the combination sgRNA plasmid was due to cases of single SV40 removal (FIG. 3C).

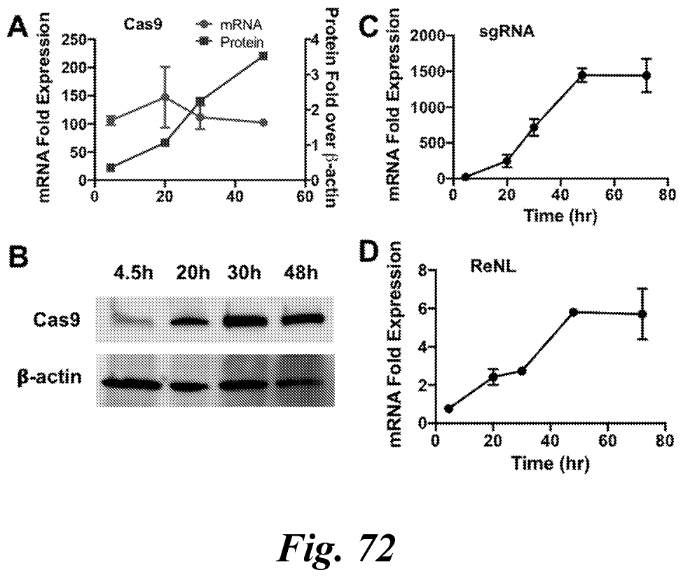

RT-qPCR of cells transfected with combination Cas9 and sg1 plasmids revealed that Cas9 mRNA levels stayed relatively constant throughout all time points evaluated (FIG. 72A). Western blots over the same time course showed that Cas9 protein levels steadily accumulated after transfection (FIG. 72B). sgRNA levels plateaued after 48 hours (FIG. 72C), and the same trend was observed in ReNL mRNA levels after stop cassette removal (FIG. 72D).

### 8.3.4 Expression Thresholds for 1-Cut and 2-Cut Edits

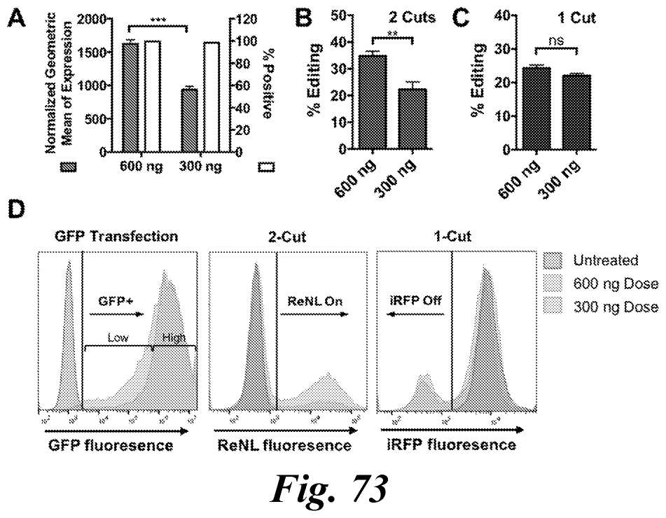

In order to assess the expression levels necessary to achieve 1-cut knockout edits and 2-cut gain-of-function edits, respectively, we varied the dosage of plasmid DNA delivered in nanoparticles. A GFP reporter was used to gauge transfection levels, and results showed that lowering the total DNA dose from 600 to 300 ng did not change the percentage of cells positively expressing GFP, but the geometric mean of fluorescence decreased by nearly 50% (FIG. 73A). This effect can be observed in flow cytometry histograms as the 300 ng treatment yielded a larger population of cells with low GFP fluorescence compared to the 600 ng treatment (FIG. 73D, left panel). Lowering total DNA dose significantly decreased levels of 2-cut deletion edits (FIG. 73B) but did not significantly change the levels of 1-cut knockout edits (FIG. 73C).

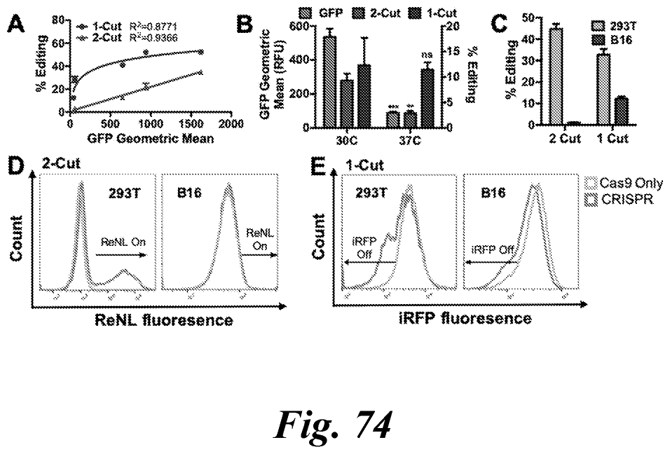

We varied the total DNA dose delivered over a wider range in order to more thoroughly probe the effect of transfection efficacy on gene editing levels (FIG. 74A). Plotting percent editing as a function of GFP geometric mean fluorescence revealed a logarithmic relationship for 1 cut edits (R²=0.8771) and a linear relationship for 2-cut edits (R²=0.9366). Transfection levels were further varied by manipulating cellular metabolic rates through incubation temperature variation (FIG. 74B). Cells were transfected using the same nanoparticle formulation delivering the same DNA dose, after which they were either incubated at standard 37° C. or treated with a transient “cold shock” via incubation at 30° C. Transfection efficacy, as measured by GFP geometric mean fluorescence, increased significantly in cold-shocked cells; the same trend was observed for the level of 2-cut edits. Interestingly, cold shock treatment did not significantly change the level of 1-cut editing efficiency, which is consistent with the results from dose titration experiments.

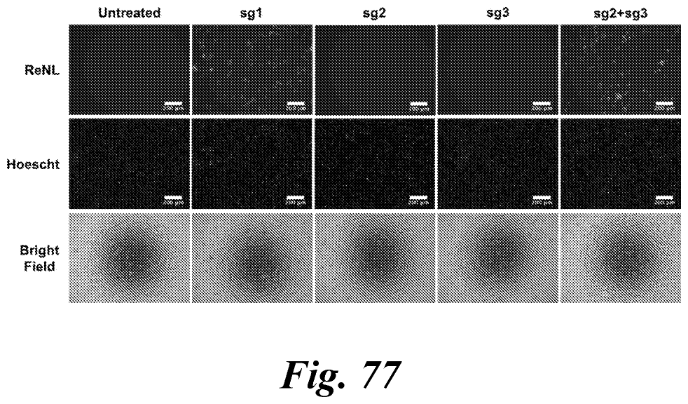

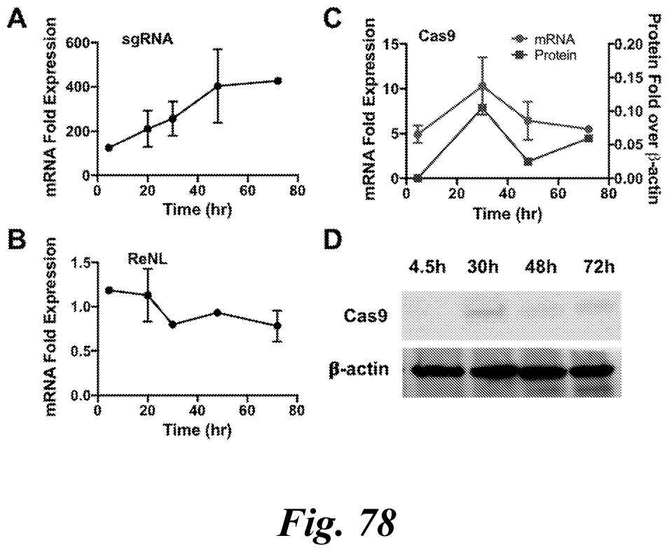

1-cut knockout of iRFP expression and 2-cut gain-of-function edits were also performed on B16-F10 murine melanoma cells, which achieved lower levels of transfection compared to 293T cells (FIG. 71). The lower geometric mean of GFP expression was reflected most notably in the results for 2-cut editing, where the ReNL fluorescence observed in B16 cells was 1 order of magnitude lower than 293T cells (FIG. 74C and FIG. 74D). Interestingly, the effect of lower transfection efficacy was less pronounced for 1-cut iRFP knockout experiments. Although lower knockout levels were observed in B16 compared to 293T cells, the difference was much smaller (12% for B16 and 33% for 293T). This validates results seen earlier with dose titration as well as temperature modulation experiments and confirms our hypothesis that single-cut knockout edits require a lower expression threshold compared to double-cut edits. On the mRNA level, both Cas9 and sgRNA expression levels were an order of magnitude higher in 293T cells compared to B16 cells (FIG. 72 and FIG. 77).

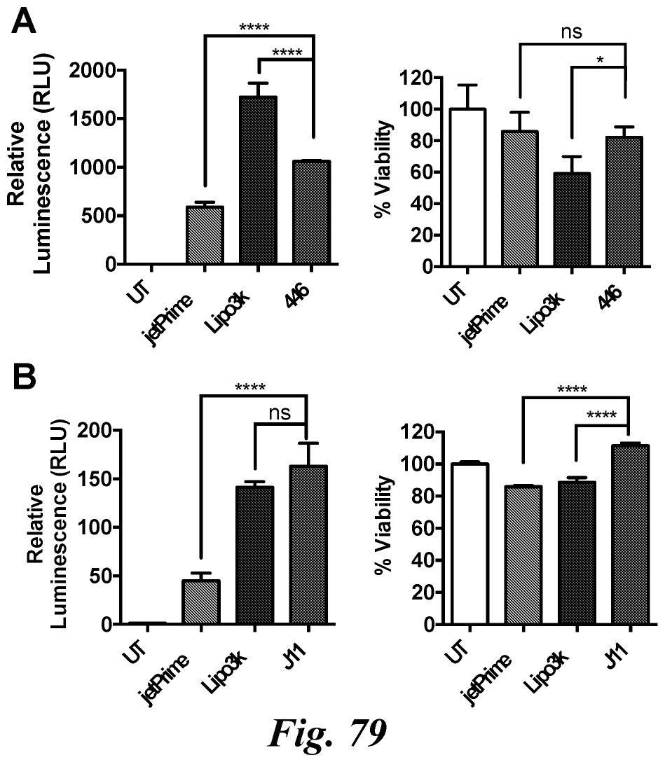

Standard transfection reagents were also used to assess 2-cut editing efficiency. For both cell lines, the commercially available cationic polymer transfection reagent jetPrime® resulted in significantly lower editing levels than PBAE nanoparticles (FIG. 79). The commercially available cationic lipid transfection reagent Lipofectamine™ 3000 enabled significantly higher editing than earlier generation linear PBAE polymer 446 in 293T cells but did not achieve significantly higher levels of editing than the newly developed next-generation branched PBAE polymer 7,8-4-J11 in harder-to-transfect B16 cells. Notably, Lipofectamine™ 3000 caused significantly higher levels of cytotoxicity than both PBAE nanoparticle formulations, further demonstrating the advantage of using a biodegradable gene delivery system.

8.3.5 A Multiplex tRNA-gRNA Expression System

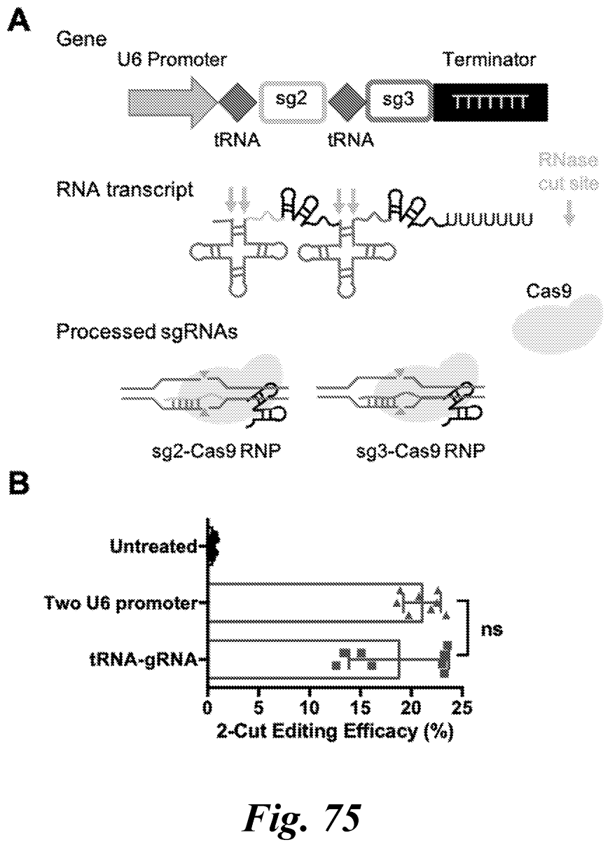

To facilitate a simpler method for multiplex CRISPR editing, we designed a tRNA-gRNA expression system¹⁷ which utilizes the cell's endogenous tRNA processing machinery to generate multiple sgRNAs (FIG. 75A). Using a simple Golden Gate assembly strategy, we created a plasmid in which the targeting sequences and gRNA scaffolds of sg2 and sg3 are arrayed in tandem with pre-tRNA, with all components governed by a single U6 promoter. Mature sgRNA is released after processing of the primary RNA transcript by tRNA-processing RNases. When transfected into cells alongside the Cas9 plasmid, this tRNA-gRNA plasmid enabled similar levels of 2-cut editing as the plasmid in which a U6 promoter governed each sgRNA (FIG. 75B). This demonstrates that the multiplex tRNA-gRNA expression system effectively expressed both sgRNAs required for 2-cut editing.

### 8.4 Discussion

In this work, we demonstrated that both linear and branched PBAE nanoparticles co-delivering two DNA plasmids encoding Cas9 and sgRNA, respectively, can achieve efficient gene editing in both 1-cut knockout as well as 2-cut gene deletion applications. We created a novel reporter system that can be used to assess both types of edits: an iRFP fluorescent reporter can be silenced by indels after 1-cut edits while an expression stop cassette upstream of a ReNL reporter can be deleted using 2-cut edits for gain-of-function ReNL expression. This expression cassette was cloned into a piggyBac transposon system and can be used to generate stably-expressing cell lines to investigate gene editing efficacy in vitro, eliminating the need to culture primary cells from the Ai9 mouse,³² on which our reporter system is based. This system further has the potential to be used as an in vivo reporter for live-animal imaging studies of effective 2-cut gain-of-function ReNL expression using the red-shifted luminescent properties of ReNL. Using two cell lines stably expressing this construct—easy-to-transfect HEK-293T and hard-to-transfect B16-F10—we further investigated the transfection requirements for each type of gene editing.

Several recent studies have demonstrated the feasibility of using polymeric nanoparticles, including a different PBAE formulation,³³ to deliver CRISPR gene editing components in the form of plasmid DNA.^{18, 20-22} All of these systems have exclusively investigated the use of 1-cut editing to achieve gene knockout, and none have presented a systematic study of the expression levels required for 1-cut and 2-cut edits. The removal of a gene segment requires sgRNA to target two sites flanking the region of interest and is significantly more difficult than 1-cut knockout edits.⁵ To date, only 3 studies have reported the use of non-viral delivery vectors for 2-cut gene deletion by delivering Cas9 mRNA and sgRNA¹⁴ or RNP complexes^{11,32}, but plasmid delivery with polymeric nanoparticles to achieve this type of deletion has not been previously reported. The use of DNA plasmids to encode Cas9 overcomes the manufacturing challenges of producing large scales of Cas9 mRNA or Cas9 protein, but the intracellular delivery and expression of exogenous DNA can be more challenging than the delivery of its downstream products.

We evaluated two types of PBAE nanoparticles to encapsulate Cas9 and sgRNA plasmids for intracellular delivery of gene editing complexes. One of these was the well-published linear PBAE polymer 446 that has shown efficacy in multiple cell types and one was a newly developed branched PBAE polymer 7,8-4-J11, and both were found to be useful for developing efficacious biodegradable nanoparticles for gene editing. The cationic polymer and anionic DNA self-assembled into nanoparticles 100-200 nm in diameter with positive zeta potentials (12-25 mV) (FIG. 71). Previous reports have shown that high levels of co-delivery can be achieved by pre-mixing plasmids prior to nanoparticle assembly.³⁴

Using this strategy, we showed successful co-delivery of CRISPR plasmids that enabled robust 1-cut gene knockout (FIG. 2). More importantly, we demonstrated a versatile gene deletion platform in which a single sgRNA targeting sites flanking the region of interest or a combination of sgRNAs targeting sites throughout the region of interest both resulted in successful removal of the entire gene segment (FIG. 3). Successful deletion of up to 630 bp could be easily visualized through the gain-of-function expression of a ReNL fluorescence/luminescence dual reporter.

Evaluation of the expression kinetics of Cas9 and sgRNA revealed that Cas9 mRNA maintained at high levels throughout the time period tested (4.5-48 hr), while sgRNA expression reached peak levels at 48 hr (FIG. 72). Actual Cas9 protein levels reached high levels after 20 hr and accumulated steadily, which is consistent with previous reports using lipid transfection reagents for Cas9 plasmid delivery,³⁵ and the resulting gain of ReNL expression peaked at 48 hr.

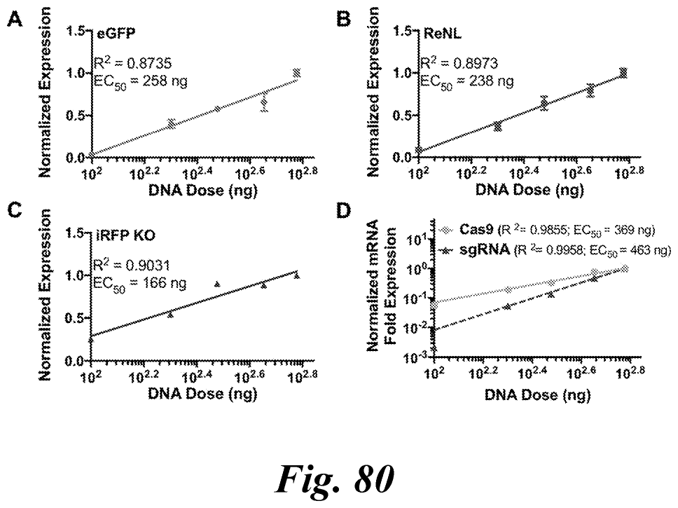

We further explored the transfection requirements for 1-cut and 2-cut edits by titrating the total DNA dose delivered. Interestingly, decreasing total DNA dose from 600 ng to 300 ng significantly decreased the level of 2-cut editing but did not affect the level of 1-cut edits (FIG. 73). In fact, the EC50 DNA dose for 2-cut edits (238 ng) was comparable to that of the eGFP transfection control (258 ng) but significantly higher than that of 1-cut edits (166 ng), suggesting that the efficiency of 2-cut edits depended more heavily on transfection levels (FIG. 80). The same trend was observed when transfection efficiency was varied by treating transfected cells with a minor “cold shock” (FIG. 74). Indeed, a brief cold shock slowed the rate of cellular division, which enhances protein accumulation in expressing cells and decreases the rate of plasmid DNA dilution in the cell population. This increased transfection efficiency and the level of 2-cut edits, which is consistent with previous reports using cold shock treatment to enhance the editing efficiency of ZFN-mediated gene disruption³⁶ or CRISPR-mediated homology-directed repair.³⁷ In contrast, cold shock treatment did not significantly change the efficiency of 1-cut edits. Recent studies on the enzyme kinetics of sgRNA-Cas9 RNPs have reported that while Cas9-sgRNA binding (k=6.1 s⁻¹), target DNA binding (t_{1/2}=4-40 s), and DNA cleavage events (k=25-90 s⁻¹) happen very quickly,³⁸ the release of DNA cleavage products is extremely slow (t_{1/2}=43-91 h),³⁹ causing Cas9 to be virtually a single turnover enzyme. Taken together with these results, our data suggest that 2-cut edits have a much higher expression threshold than 1-cut edits because twice the number of DNA cleavage events, and hence twice the number of RNP complexes, are required for successful edits to occur.

The expression thresholds of single-cut and double-cut edits have important implications on gene editing in different cell types. To demonstrate this, we compared the gene editing efficiency in easier-to-transfect HEK-293T and harder-to-transfect B16-F10 cells. Although the top nanoparticle formulation for each cell line achieved >80% transfection as assessed by percentage of total cells transfected, the level of expression, as assessed by the normalized geometric mean of expression of a GFP reporter, was 1 order of magnitude higher for 293T cells (FIG. 1). This discrepancy was reflected in the level of 2-cut edits as B16 cells showed very minimal levels of ReNL expression after stop cassette deletion (FIG. 74). In contrast, the difference in editing efficiency between the two cell lines was much smaller for 1-cut iRFP knockout (<3-fold difference compared to nearly 44-fold difference for 2-cut edits). These results further validate our hypothesis that the efficiency of 2-cut edits correlates more strongly with the level of DNA expression.

Finally, we designed and implemented a tRNA-gRNA plasmid in which the expression of multiplex sgRNAs is governed under a single U6 promoter. The expression of two sgRNAs required for turning on of ReNL fluorescence in these tRNA-gRNA tandem repeats enabled similar levels of editing compared to that of a plasmid in which each sgRNA is governed by its own U6 promoter (FIG. 75). This expression system has the advantage of ease of synthesis as upwards of 6 sgRNAs can be arranged in tandem using a single Golden Gate assembly reaction.¹⁷ More importantly, the tRNA-gRNA system reduces the need for repeating U6 promoters, enabling the use of a much smaller plasmid construct especially at high numbers of sgRNAs. Originally developed for use in rice plants,¹⁷ this system has also been adapted for use in yeast⁴⁰ and zebrafish.⁴¹ To our knowledge, this is the first time it has been adapted for gene editing in mammalian cells.

In summary, we have demonstrated that PBAE nanoparticles co-delivering plasmids encoding Cas9 and sgRNA can achieve 1-cut knockout as well as 2-cut deletion edits. We designed a novel reporter system whereby both modes of edits can be easily evaluated. 2-cut deletion events required much higher levels of transfection than 1-cut gene knockout edits, which we demonstrated by titrating the DNA dosage delivered, treating transfected cells with a transient cold shock, and comparing editing efficiencies in two cell lines with different transfection efficacy. The PBAE/DNA nanoparticles optimized here are promising for DNA-based non-viral gene editing. Further, the results presented herein have implications on the design and screening of next-generation non-viral delivery vehicles broadly for CRISPR/Cas9 gene editing.

### 8.5 MATERIALS AND METHODS

### 8.5.1 Materials

Small molecules used as monomers for polymer synthesis were obtained as follows: bisphenol A glycerolate (1 glycerol/phenol) diacrylate (B7; 411167), trimethylolpropane triacrylate (B8; 246808), 2-β-aminopropylamino)ethanol (E6; 09293), and N,N-diethyldiethylenetriamine (J11; 518832)⁴² were purchased from Sigma-Aldrich; 1,4-butanediol diacrylate (B4; 32780) and 4-amino-1-butanol (S4; A12680) were purchased from Alfa Aesar. The following plasmids were purchased from Addgene: hCas9 (41815),³ gRNA_GFP-T2 (41820),³ pCAG-GFPd2 (14760),⁴³ PBCAG-eGFP (40973),⁴⁴ piRFP670-N1 (45457),³¹ tubulin-ReNL_pcDNA3 (89530).⁴⁵ PB-CMV-MCS-EF1a-RFP PiggyBac plasmid (PB512B-1) and PiggyBac transposase expression plasmid (PB200A-1) were purchased from System Biosciences. sgRNA gBlock sequences were purchased from IDT and the expression stop cassette was synthesized by SynBio-Tech (Monmouth Junction, N.J.). Restriction enzymes and T4 DNA ligase for molecular cloning were purchased from New England BioLabs.

### 8.5.2 Polymer Synthesis

Polymer 446 was synthesized by reacting monomers B4 and S4 at a molar ratio of 1.1:1 at 90° C. with stirring overnight. The B4-S4 polymer was dissolved in anhydrous THF at 167 mg/mL and added to monomer E6 (0.5 M in THF) at a 3:2 volume ratio and reacted at room temperature for 1 hour. The end-capped polymer was washed in diethyl ether twice to remove unreacted monomers and oligomers. Solvents were removed in a vacuum desiccant chamber and polymer was dissolved in DMSO at 100 mg/mL, then stored at −20° C. with desiccant. Polymer 7,8-4-J11 was synthesized by reacting monomers B7, B8, and S4 at an overall vinyl amine ratio of 2.2:1 and monomer concentration of 200 mg/mL in anhydrous DMSO at 90° C. with stirring overnight; the acrylate monomer composition was 80% B7 and 20% B8 by mole fraction. Polymer end-capping and purification were done following the same procedure as polymer 446 but using monomer J11.

### 8.5.3 Nanoparticle Characterization

Nanoparticle hydrodynamic diameter was measured via dynamic light scattering (DLS) using a Malvern Zetasizer NanoZS (Malvern Instruments). Samples were prepared in 25 mM sodium acetate (NaAc), pH 5.0, and then diluted 1:6 in 150 mM PBS to determine hydrodynamic diameter in neutral, isotonic buffer. Zeta potential was measured by electrophoretic light scattering on the same instrument. Transmission electron microscopy (TEM) images were captured using a Philips CM120 (Philips Research) on 400 square mesh carbon coated TEM grids. Samples were prepared at a polymer concentration of 1.8 mg/mL at 30 w/w in 25 mM NaAc and 30 μL were allowed to coat TEM grids for 20 minutes. Grids were then rinsed with ultrapure water and allowed to fully dry before imaging.

### 8.5.4 Cell Culture and Cell Line Preparation

HEK-293T and B16-F10 cells were cultured in Dulbecco's Modified Eagle Medium (DMEM; ThermoFisher) supplemented with 10% FBS and 1% penicillin/streptomycin. Cells were induced to constitutively express fluorescent protein constructs using the PiggyBac transposon/transposase system. The GFPd2 gene was cloned into the PB-CMV-MCS-EF1a-RFP plasmid using restriction enzyme cloning to create a PiggyBac transposon plasmid containing the GFPd2 gene. A sequence containing iRFP and transcription stop sequences was cloned into the PBCAG-eGFP plasmid backbone, and the ReNL gene was inserted into this plasmid using restriction enzyme cloning to create a PiggyBac transposon plasmid containing the iRFP-STOP-ReNL sequence (plasmid available on Addgene). Each transposon plasmid was co-transfected with the PiggyBac transposase plasmid into HEK-293T and/or B16-F10 cells using nanoparticles as described below. Fluorescent protein signal from DNA not integrated into the cell genome was allowed to fade over 5 passages, after which positive cells were isolated using fluorescence-assisted cell sorting (FACS). Cells were further expanded for 3 more passages and sorted again to generate stably-expressing cell lines.

8.5.6 sgRNA Design and Preparation

Single guide RNAs were designed using the CRISPR.mit.edu platform and ordered as gBlocks containing the U6 promoter, a unique 20 bp targeting sequence, and the duplex optimized sgRNA scaffold from IDT.⁵ The gBlocks were cloned into the pCAG-GFPd2 plasmid backbone using restriction enzyme cloning. sgRNA plasmids were transformed into DH5c competent E. coli (NEB), grown out overnight at 37° C. in 5 mL LB broth liquid cultures, and plasmid DNA was harvested using QIAprep miniprep kit (Qiagen). Plasmid DNA was characterized using NanoDrop spetrophotomoter (ThermoFisher) and sequence confirmed via Sanger sequencing before use in transfections. All sgRNA target sequences are listed in Table 8-S2 and plasmids are available on Addgene.

The gRNA-tRNA plasmid containing multiplex sgRNA constructs under a single U6 promoter was synthesized according to the protocol by Xie et al.¹⁷ Briefly, the pGTR construct containing a sgRNA scaffold sequence fused to a tRNA fragment was synthesized as a gBlock from IDT and cloned into a plasmid via restriction enzyme cloning. This pGTR plasmid was used as the template DNA for PCR reactions which produced amplicons used in a hierarchical Golden Assembly process to generate a DNA fragment containing the tRNA-gRNA tandem arrays. This fragment was then cloned into a backbone plasmid containing a U6 promoter via restriction enzyme cloning. The sequences for the pGTR sequence and PCR primers used are listed in Table 8-S3.

### 8.5.7 Transfection

Cells were plated at 15,000 cells per well (HEK-293T) or 10,000 cells per well (B16-F10) in 100 μL complete medium and allowed to adhere overnight. Polymers and DNA were dissolved separately in 25 mM NaAc at the desired concentrations and then mixed together via pipetting. Nanoparticles were allowed to self-assemble for 10 minutes and then 20 μL of the nanoparticle solution was added per well for a final volume of 120 μL and 600 ng DNA per well unless otherwise noted; for transfection experiments using the CRISPR/Cas9 system, the hCas9 and sgRNA plasmids were used at a 1:1 weight ratio. Nanoparticles were incubated with cells for 2 hours at 37° C., at which point the media and nanoparticles were removed and replaced with fresh complete media. Commercially available transfection reagents jetPrime® (Polyplus) and Lipofectamine™ 3000 (ThermoFisher) were used as instructed by the manufacturer. For cold shock treatment, cells were transfected using standard transfection procedures and allowed to recover at 37° C. after media change for 6 hours before being moved to 30° C. Cells were maintained at 30° C. for 3 days, after which time they were moved back to 37° C.

Transfection and gene editing efficacies were evaluated via flow cytometry using a BD Accuri C₆ flow cytometer (BD Biosciences). CRISPR knockout was quantified by normalizing the geometric mean of fluorescence of treated wells to that of wells transfected with Cas9 plasmid only. Gain of fluorescence was quantified as the percentage of cells positively expressing the fluorescent protein when gated against untreated control. Gene editing in gene deletion experiments was also assessed by luminescence readings using Promega Nano-Glo® Luciferase assay system (Promega) measured with a Synergy2 plate reader (Biotek) with open optics and normalized to untreated control. Cell viability was assessed 24 hours post-transfection using MTS CellTiter 96 Aqueous One cell proliferation assay (Promega). (N=4+/−SEM).

### 8.5.8 Surveyor Assay

Genomic DNA from cells transfected with the combination Cas9-sgRNA plasmids and untransfected control were isolated using GeneJET genomic DNA purification kit (ThermoFisher). A 660 bp region flanking the predicted cut site was PCR amplified, and the PCR products were purified using QIAquick PCR purification kit. 400 ng of PCR amplicons were hybridized, and the Surveyor assay was performed using Surveyor® Mutation Detection Kit (IDT) following manufacturer's instructions. The uncut and cut DNA products were then run on a 2% agarose gel stained with ethidium bromide in tris/borate/EDTA (TBE) buffer and imaged under UV light.

### 8.5.9 Sanger Sequencing to Detect Gene Editing

PCR products for the Surveyor assay were cloned into plasmid vectors using NEB PCR Cloning kit and transformed into DH5c competent E. coli (NEB). 30 colonies were grown out in 5 mL liquid cultures overnight and the plasmid DNA was isolated and characterized by Sanger sequencing.

8.5.10 qRT-PCR

Cells transfected with the combination Cas9-sgRNA plasmids in a 12-well plate were collected, and total RNA including small RNAs (<100 nt) were extracted using miRNeasy Mini kit (Qiagen). RNA was reverse transcribed using iScript cDNA synthesis kit (Bio-Rad), and qRT-PCR was run on a StepOnePlus Real-Time PCR system (ThermoFisher) using SYBR Green PCR Master Mix (ThermoFisher). The qPCR program is as follows: 95° C. for 10 min.; 95° C. 15 sec, 55° C. 30 sec, and 60° C. 30 sec for 40 cycles. Primers used for qRT-PCR are listed in Table 8-S1. Results are shown as fold expression over β-actin.

### 8.5.11 Western Blotting

Transfected cells in 12-well plates were lysed in a solution of 1×RIPA buffer and 1× Protease/Phosphatase Inhibitor Cocktail (ThermoFisher). The lysate was cleared by centrifugation, protein concentration was determined using Pierce Micro BCA assay (ThermoFisher), and samples were denatured in Laemmli sample buffer (Bio-Rad) in the presence of DTT. 50 μg proteins were loaded into 4-15% TGX Precast Protein Gels (Bio-Rad). Proteins were then transferred to a PVDF membrane using a Pierce Power Blotter (ThermoFisher). Membranes were blocked in 5% non-fat milk for 1 hr at RT and probed with primary antibodies against Cas9 (Cell Signaling Technologies 14697; 1:500) or β-actin (Abcam ab8226; 1:10,000) at 4° C. overnight. Secondary antibodies were applied at RT for 1 hr (m-IgGK BP-HRP; Santa-Cruz Sc-516102; 1:1000). The membrane was developed with Amersham ECL Western Blotting Detection Reagent (GE Healthcare) and imaged using an ImageQuant LAS 4000 CCD imager (GE Healthcare).

### 8.6 REFERENCES

1. Jinek, M.; Chylinski, K.; Fonfara, I.; Hauer, M.; Doudna, J. A.; Charpentier, E., A programmable dual-RNA-guided DNA endonuclease in adaptive bacterial immunity. science 2012, 1225829.
2. Jinek, M.; East, A.; Cheng, A.; Lin, S.; Ma, E.; Doudna, J., RNA-programmed genome editing in human cells. elife 2013, 2.
3. Mali, P.; Yang, L.; Esvelt, K. M.; Aach, J.; Guell, M.; DiCarlo, J. E.; Norville, J. E.; Church, G. M., RNA-guided human genome engineering via Cas9. Science 2013, 339 (6121), 823-826.
4. Ho, T.-T.; Zhou, N.; Huang, J.; Koirala, P.; Xu, M.; Fung, R.; Wu, F.; Mo, Y.-Y., Targeting non-coding RNAs with the CRISPR/Cas9 system in human cell lines. Nucleic acids research 2014, 43 (3), e17-e17.
5. Dang, Y.; Jia, G.; Choi, J.; Ma, H.; Anaya, E.; Ye, C.; Shankar, P.; Wu, H., Optimizing sgRNA structure to improve CRISPR-Cas9 knockout efficiency. Genome biology 2015, 16 (1), 280.
6. Yang, Y.; Wang, L.; Bell, P.; McMenamin, D.; He, Z.; White, J.; Yu, H.; Xu, C.; Morizono, H.; Musunuru, K., A dual AAV system enables the Cas9-mediated correction of a metabolic liver disease in newborn mice. Nature biotechnology 2016, 34 (3), 334.
7. Yu, W.; Mookherjee, S.; Chaitankar, V.; Hiriyanna, S.; Kim, J.-W.; Brooks, M.; Ataeijannati, Y.; Sun, X.; Dong, L.; Li, T.; Swaroop, A.; Wu, Z., Nrl knockdown by AAV-delivered CRISPR/Cas9 prevents retinal degeneration in mice. Nature Communications 2017, 8, 14716.
8. Sun, W.; Ji, W.; Hall, J. M.; Hu, Q.; Wang, C.; Beisel, C. L.; Gu, Z., Self-assembled DNA nanoclews for the efficient delivery of CRISPR-Cas9 for genome editing. Angewandte Chemie International Edition 2015, 54 (41), 12029-12033.
9. Zuris, J. A.; Thompson, D. B.; Shu, Y.; Guilinger, J. P.; Bessen, J. L.; Hu, J. H.; Maeder, M. L.; Joung, J. K.; Chen, Z.-Y.; Liu, D. R., Cationic lipid-mediated delivery of proteins enables efficient protein-based genome editing in vitro and in vivo. Nature biotechnology 2015, 33 (1), 73.
10. Wang, M.; Zuris, J. A.; Meng, F.; Rees, H.; Sun, S.; Deng, P.; Han, Y.; Gao, X.; Pouli, D.; Wu, Q.; Georgakoudi, I.; Liu, D. R.; Xu, Q., Efficient delivery of genome-editing proteins using bioreducible lipid nanoparticles. Proceedings of the National Academy of Sciences 2016, 113 (11), 2868.
11. Lee, K.; Conboy, M.; Park, H. M.; Jiang, F.; Kim, H. J.; Dewitt, M. A.; Mackley, V. A.; Chang, K.; Rao, A.; Skinner, C.; Shobha, T.; Mehdipour, M.; Liu, H.; Huang, W.-c.; Lan, F.; Bray, N. L.; Li, S.; Corn, J. E.; Kataoka, K.; Doudna, J. A.; Conboy, I.; Murthy, N., Nanoparticle delivery of Cas9 ribonucleoprotein and donor DNA in vivo induces homology-directed DNA repair. Nature Biomedical Engineering 2017, 1 (11), 889-901.
12. Mout, R.; Ray, M.; Yesilbag Tonga, G.; Lee, Y.-W.; Tay, T.; Sasaki, K.; Rotello, V. M., Direct Cytosolic Delivery of CRISPR/Cas9-Ribonucleoprotein for Efficient Gene Editing. ACS Nano 2017, 11 (3), 2452-2458.
13. Jiang, C.; Mei, M.; Li, B.; Zhu, X.; Zu, W.; Tian, Y.; Wang, Q.; Guo, Y.; Dong, Y.; Tan, X., A non-viral CRISPR/Cas9 delivery system for therapeutically targeting HBV DNA and pcsk9 in vivo. Cell Research 2017, 27, 440.
14. Miller, J. B.; Zhang, S.; Kos, P.; Xiong, H.; Zhou, K.; Perelman, S. S.; Zhu, H.; Siegwart, D. J., Non-Viral CRISPR/Cas Gene Editing In Vitro and In Vivo Enabled by Synthetic Nanoparticle Co-Delivery of Cas9 mRNA and sgRNA. Angewandte Chemie International Edition 2017, 56 (4), 1059-1063.
15. Shen, B.; Zhang, W.; Zhang, J.; Zhou, J.; Wang, J.; Chen, L.; Wang, L.; Hodgkins, A.; Iyer, V.; Huang, X., Efficient genome modification by CRISPR-Cas9 nickase with minimal off-target effects. Nature methods 2014, 11 (4), 399.
16. Konermann, S.; Brigham, M. D.; Trevino, A. E.; Joung, J.; Abudayyeh, O. O.; Barcena, C.; Hsu, P. D.; Habib, N.; Gootenberg, J. S.; Nishimasu, H.; Nureki, O.; Zhang, F., Genome-scale transcriptional activation by an engineered CRISPR-Cas9 complex. Nature 2014, 517, 583.
17. Xie, K.; Minkenberg, B.; Yang, Y., Boosting CRISPR/Cas9 multiplex editing capability with the endogenous tRNA-processing system. Proceedings of the National Academy of Sciences 2015, 112 (11), 3570.
18. Luo, Y.-L.; Xu, C.-F.; Li, H.-J.; Cao, Z.-T.; Liu, J.; Wang, J.-L.; Du, X.-J.; Yang, X.-Z.; Gu, Z.; Wang, J., Macrophage-Specific in Vivo Gene Editing Using Cationic Lipid-Assisted Polymeric Nanoparticles. ACS Nano 2018.
19. Steyer, B.; Carlson-Stevermer, J.; Angenent-Mari, N.; Khalil, A.; Harkness, T.; Saha, K., High content analysis platform for optimization of lipid mediated CRISPR-Cas9 delivery strategies in human cells. Acta biomaterialia 2016, 34, 143-158.
20. Liang, C.; Li, F.; Wang, L.; Zhang, Z.-K.; Wang, C.; He, B.; Li, J.; Chen, Z.; Shaikh, A. B.; Liu, J.; Wu, X.; Peng, S.; Dang, L.; Guo, B.; He, X.; Au, D. W. T.; Lu, C.; Zhu, H.; Zhang, B.-T.; Lu, A.; Zhang, G., Tumor cell-targeted delivery of CRISPR/Cas9 by aptamer-functionalized lipopolymer for therapeutic genome editing of VEGFA in osteosarcoma. Biomaterials 2017, 147, 68-85.
21. Kim, S. M.; Yang, Y.; Oh, S. J.; Hong, Y.; Seo, M.; Jang, M., Cancer-derived exosomes as a delivery platform of CRISPR/Cas9 confer cancer cell tropism-dependent targeting. J Control Release 2017, 266, 8-16.
22. Timin, A. S.; Muslimov, A. R.; Lepik, K. V.; Epifanovskaya, O. S.; Shakirova, A. I.; Mock, U.; Riecken, K.; Okilova, M. V.; Sergeev, V. S.; Afanasyev, B. V.; Fehse, B.; Sukhorukov, G. B., Efficient gene editing via non-viral delivery of CRISPR-Cas9 system using polymeric and hybrid microcarriers. Nanomedicine: Nanotechnology, Biology and Medicine 2018, 14 (1), 97-108.
23. Rui, Y.; Wilson, D. R.; Sanders, K.; Green, J. J., Reducible Branched Ester-Amine Quadpolymers (rBEAQs) Codelivering Plasmid DNA and RNA Oligonucleotides Enable CRISPR/Cas9 Genome Editing. ACS Applied Materials & Interfaces 2019, 11 (11), 10472-10480.
24. Sunshine, J. C.; Peng, D. Y.; Green, J. J., Uptake and transfection with polymeric nanoparticles are dependent on polymer end-group structure, but largely independent of nanoparticle physical and chemical properties. Mol Pharm 2012, 9 (11), 3375-3383.
25. Sunshine, J. C.; Akanda, M. I.; Li, D.; Kozielski, K. L.; Green, J. J., Effects of base polymer hydrophobicity and end-group modification on polymeric gene delivery. Biomacromolecules 2011, 12 (10), 3592-600.
26. Wilson, D. R.; Mosenia, A.; Suprenant, M. P.; Upadhya, R.; Routkevitch, D.; Meyer, R. A.; Quinones-Hinojosa, A.; Green, J. J., Continuous microfluidic assembly of biodegradable poly(beta-amino ester)/DNA nanoparticles for enhanced gene delivery. J Biomed Mater Res A 2017, 105 (6),
1813-1825.
27. Wilson, D. R.; Rui, Y.; Siddiq, K.; Routkevitch, D.; Green, J. J., Differentially Branched Ester Amine Quadpolymers with Amphiphilic and pH-Sensitive Properties for Efficient Plasmid DNA Delivery. Mol Pharm 2019, 16 (2), 655-668.
28. Madisen, L.; Zwingman, T. A.; Sunkin, S. M.; Oh, S. W.; Zariwala, H. A.; Gu, H.; Ng, L. L.; Palmiter, R. D.; Hawrylycz, M. J.; Jones, A. R.; Lein, E. S.; Zeng, H., A robust and high-throughput Cre reporting and characterization system for the whole mouse brain. Nat. Neurosci. 2010, 13 (1),
133-140.
29. Suzuki, K.; Tsunekawa, Y.; Hernandez-Benitez, R.; Wu, J.; Zhu, J.; Kim, E. J.; Hatanaka, F.; Yamamoto, M.; Araoka, T.; Li, Z.; Kurita, M.; Hishida, T.; Li, M.; Aizawa, E.; Guo, S.; Chen, S.; Goebl, A.; Soligalla, R. D.; Qu, J.; Jiang, T.; Fu, X.; Jafari, M.; Esteban, C. R.; Berggren, W. T.; Lajara, J.; Nuñez-Delicado, E.; Guillen, P.; Campistol, J. M.; Matsuzaki, F.; Liu, G.-H.; Magistretti, P.; Zhang, K.; Callaway, E. M.; Zhang, K.; Belmonte, J. C. I., In vivo genome editing via CRISPR/Cas9 mediated homology-independent targeted integration. Nature 2016, 540, 144.
30. Yusa, K.; Zhou, L.; Li, M. A.; Bradley, A.; Craig, N. L., A hyperactive piggyBac transposase for mammalian applications. Proceedings of the National Academy of Sciences 2011, 108 (4), 1531-1536.
31. Shcherbakova, D. M.; Verkhusha, V. V., Near-infrared fluorescent proteins for multicolor in vivo imaging. Nature Methods 2013, 10, 751.
32. Staahl, B. T.; Benekareddy, M.; Coulon-Bainier, C.; Banfal, A. A.; Floor, S. N.; Sabo, J. K.; Urnes, C.; Munares, G. A.; Ghosh, A.; Doudna, J. A., Efficient genome editing in the mouse brain by local delivery of engineered Cas9 ribonucleoprotein complexes. Nature biotechnology 2017, 35 (5), 431.
33. Zhu, D.; Shen, H.; Tan, S.; Hu, Z.; Wang, L.; Yu, L.; Tian, X.; Ding, W.; Ren, C.; Gao, C.; Cheng, J.; Deng, M.; Liu, R.; Hu, J.; Xi, L.; Wu, P.; Zhang, Z.; Ma, D.; Wang, H., Nanoparticles based on poly (β-amino ester) and HPV16 targeting CRISPR/shRNA as potential drugs for HPV16 related cervical malignancy. Molecular Therapy 2018.
34. Bhise, N. S.; Shmueli, R. B.; Gonzalez, J.; Green, J. J., A Novel Assay for Quantifying the Number of Plasmids Encapsulated by Polymer Nanoparticles. Small 2012, 8 (3), 367-373.
35. Liang, X.; Potter, J.; Kumar, S.; Zou, Y.; Quintanilla, R.; Sridharan, M.; Carte, J.; Chen, W.; Roark, N.; Ranganathan, S.; Ravinder, N.; Chesnut, J. D., Rapid and highly efficient mammalian cell engineering via Cas9 protein transfection. J. Biotechnol. 2015, 208, 44-53.
36. Doyon, Y.; Choi, V. M.; Xia, D. F.; Vo, T. D.; Gregory, P. D.; Holmes, M. C., Transient cold shock enhances zinc-finger nuclease-mediated gene disruption. Nature Methods 2010, 7, 459.
37. Guo, Q.; Mintier, G.; Ma-Edmonds, M.; Storton, D.; Wang, X.; Xiao, X.; Kienzle, B; Zhao, D.; Feder, J. N., ‘Cold shock’ increases the frequency of homology directed repair gene editing in induced pluripotent stem cells. Scientific Reports 2018, 8 (1), 2080.
38. Mekler, V.; Minakhin, L.; Semenova, E.; Kuznedelov, K.; Severinov, K., Kinetics of the CRISPR-Cas9 effector complex assembly and the role of 3′-terminal segment of guide RNA. Nucleic acids research 2016, 44 (6), 2837-2845.
39. Raper, A. T.; Stephenson, A. A.; Suo, Z., Functional Insights Revealed by the Kinetic Mechanism of CRISPR/Cas9. J Am Chem Soc 2018, 140 (8), 2971-2984.
40. Zhang, Y.; Wang, J.; Wang, Z.; Zhang, Y.; Shi, S.; Nielsen, J.; Liu, Z., A gRNA-tRNA array for CRISPR-Cas9 based rapid multiplexed genome editing in Saccharomyces cerevisiae. Nature Communications 2019, 10 (1), 1053.
41. Shiraki, T.; Kawakami, K., A tRNA-based multiplex sgRNA expression system in zebrafish and its application to generation of transgenic albino fish. Scientific Reports 2018, 8 (1), 13366.
42. Mishra, B.; Wilson, D. R.; Sripathi, S. R.; Suprenant, M. P.; Rui, Y.; Wahlin, K. J.; Berlinicke, C.; Green, J. J.; Zack, D. J., Combinatorial library of biodegradable polyesters enables delivery of plasmid DNA to polarized human RPE monolayers for retinal gene therapy. bioRxiv 2018, 264390.
43. Matsuda, T.; Cepko, C. L., Controlled expression of transgenes introduced by in vivo electroporation. Proceedings of the National Academy of Sciences 2007, 104 (3), 1027.
44. Chen, F.; LoTurco, J., A method for stable transgenesis of radial glia lineage in rat neocortex by piggyBac mediated transposition. J. Neurosci. Methods 2012, 207 (2), 172-180.
45. Suzuki, K.; Kimura, T.; Shinoda, H.; Bai, G.; Daniels, M. J.; Arai, Y.; Nakano, M.; Nagai, T., Five colour variants of bright luminescent protein for real-time multicolour bioimaging. Nature Communications 2016, 7, 13718.

TABLE 8-S1
PCR primer sequences.

| Target | Sequence | Notes |
|---|---|---|
| GFP | FWD: CTGGTCGAGCTGGACGGCGACG | Amplicon size: |
| | (SEQ ID NO: 4) | 630 bp |
| | REV: CACGAACTCCAGCAGGACCATG | |
| | (SEQ ID NO: 5) | |
| 2X-SV40 | FWD: CGCAAATGGGCGGTAGGCGTG | Amplicon size: |
| Stop | (SEQ ID NO: 6) | 755 bp |
| Cassette | REV: GCCCTTGCTCACCATGAATT | |
| | (SEQ ID NO: 7) | |
| hCas9 | FWD: GGAGTTGACGCCAAAGCAATCC | Amplicon size: |
| | (SEQ ID NO: 8) | 150 bp |
| | REV: AGATTTAAAGTTGGGGGTCAGCC | |
| | (SEQ ID NO: 9) | |
| ReNL | FWD: ATCCCGTATGAAGGTCTGAGCG | Amplicon size: |
| | (SEQ ID NO: 10) | 147 bp |
| | REV: GTCGATCATGTTCGGCGTAACC | |
| | (SEQ ID NO: 11) | |
| sgRNA1 | FWD: ACATTATACGGTTTCAGAGC | Amplicon size: |
| | (SEQ ID NO: 12) | 91 bp |
| | REV: GACTCGGTGCCACTTTTTCA | |
| | (SEQ ID NO: 13) | |
| β-actin | FWD: CATGTACGTTGCTATCCAGGC | Amplicon size: |
| (human) | (SEQ ID NO: 14) | 250 bp |
| | REV: CTCCTTAATGTCACGCACGAT | Primerbank ID: |
| | (SEQ ID NO: 15) | 4501885a1 |
| β-actin | FWD: CTGTCCCTGTATGCCTCTG | |
| (mouse) | (SEQ ID NO: 16) | Amplicon size: |
| | REV: ATGTCACGCACGATTTCC | 218 bp |
| | (SEQ ID NO: 17) | |

TABLE 8-S2
Plasmids deposited with Addgene

| Plasmid Name | Addgene ID | Description |
|---|---|---|
| PB-iRFP- | 113965 | Piggybac transposon plasmid CRISPR gene |
| STOP- | | deletion activatable fluorescence. |
| ReNL | | Constitutive iRFP670 under EF1 A promoter, |
| | | CMV promoter with two SV40 poly A |
| | | followed by red-enhanced |
| | | nanolantern (ReNL) |
| Sg1 | 113966 | Single short guide RNA targeting |
| | | GTATAGCATACATTATACG |
| | | (SEQ ID NO: 18) |
| sg2 | 133967 | Single short guide RNA targeting |
| | | TACCACATTTGTAGAGGTT |
| | | (SEQ ID NO: 19) |
| sg3 | 133968 | Single short guide RNA targeting |
| | | CAATGIATCTTATCATGTC |
| | | (SEQ ID NO: 20) |
| sg1 + sg2 + | 133969 | Triple short guide RNA targeting |
| sg3 | | GTATAGCATACATTATACG (SEQ ID NO: |
| | | 21), TACCACATTTGTAGAGGTT (SEQ ID |
| | | NO: 22) & CAATGTATCTTATCATGTC |
| | | (SEQ ID NO: 23) |
| sg2 + sg3 | 133970 | Double short guide RNA targeting |
| | | TACCACATTTGTAGAGGTT (SEQ ID NO: |
| | | 24) & CAATGTATCTTATCATGTC (SEQ ID |
| | | NO: 25) |
| sgiRFP1 | 133972 | Single short guide RNA targeting |
| | | GATCGAGTTCGAGCCTGCGG (SEQ ID |
| | | NO: 26) in iRFP670 sequence |
| sgiRFP2 | 133973 | Single short guide RNA targeting |
| | | GCGCGTTCTTTGGACGCGA (SEQ ID NO: |
| | | 27) in 1RFP670 sequence |
| sgiRFP3 | 133974 | Single short guide RNA targeting |
| | | CGTGATGTTGTACCGCTTC (SEQ ID NO: |
| | | 28) in iRFP670 sequence |

TABLE 8-S3
DNA and primer sequences used to
generate multiplex tRNA-gRNA
plasmid. The pGTR sequence was cloned into a
backbone plasmid via restriction enzyme cloning
using Spel and HindIII. The pGTR plasmid was
then used as the PCR template for amplifying
gRNA-tRNA sequences for Golden Gate assembly.
To synthesize a multiplex plasmid containing
both sg2 and sg3, PCR amplicons were generated
using the following pairs of primers:
tRNA-start_F + sg2_R (amplicon 1);
sg2_F + sg3_R (amplicon 2); sg3_F +
gRNA-end_R (amplicon 3).
Amplicons 1-3 were then purified,
ligated by Golden Gate assembly, and cloned
into a backbone vector containing a single
U6 promoter using restriction enzyme cloning
with XbaI and HindIII.

| DNA Sequence (SEQ ID NOs: 29-42) | Description and notes |
|---|---|
| ATTATTGACTAGTAGTGGTTTTAGAGCTAGAAATAG | pGTR sequence |
| CAAGTTAAAATAAGGCTAGTCCGTTATCAACTTGAA | SpeI restriction |
| AAAGTGGCACCGAGTCGGTGCAACAAAGCACCAGT | enzyme site |
| GGTCTAGTGGTAGAATAGTACCCTGCCACGGTACAG | HindIII restriction |
| ACCCGGGTTCGATTCCCGGCTGGTGCAGCCAAGCTT | enzyme site |
| GGCGTAA | gRNA scaffold |
| | Pre-tRNA |
| AGTTAGTTtctagaACAAAGCACCAGTGG | tRNA-start_F primer |
| GAACCTCTACAAATGTGGTA | |
| TAGGTCTCCACAAATGTGGTAGTTTTAGAGCTAGAA | sg2_F primer |
| ATGGTCTCATTGTAGAGGTTCTGCACCAGCCGGGAA | sg2_R primer |
| GCAATGTATCTTATCATGTC | sg3 protospacer |
| | sequence; |
| | overlapping base |
| | pairs used in |
| | Golden Gate primers |
| TAGGTCTCCTCTTATCATGTCGTTTTAGAGCTAGAA | sg3_F primer |
| ATGGTCTCAAAGATACATTGCTGCACCAGCCGGGAA | sg3_R primer |
| CAATGTATaagcttAAAAAAAAAAGCACCGACTCG | gRNA-end_R primer |
| | HindIII restriction |
| | enzyme site |

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Bainbridge J W, Tan M H, Ali R R. Gene therapy progress and prospects: the eye. Gene Ther. August 2006; 13(16):1191-1197.
Jia F, Wilson K D, Sun N, et al. A nonviral minicircle vector for deriving human iPS cells. Nat Methods. March 2010; 7(3):197-199.
Nauta A, Seidel C, Deveza L, et al. Adipose-derived stromal cells overexpressing vascular endothelial growth factor accelerate mouse excisional wound healing. Mol Ther. February 2013; 21(2):445-455.
Baum C, Kustikova O, Modlich U, Li Z, Fehse B. Mutagenesis and oncogenesis by chromosomal insertion of gene transfer vectors. Hum Gene Ther. March 2006; 17(3):253-263.
Sasaki A, Kinjo M. Monitoring intracellular degradation of exogenous DNA using diffusion properties. J Control Release. Apr. 2 2010; 143(1):104-111.
Bitner H, Mizrahi-Meissonnier L, Griefner G, Erdinest I, Sharon D, Banin E. A homozygous frameshift mutation in BEST1 causes the classical form of Best disease in an autosomal recessive mode. Invest Ophthalmol Vis Sci. Jul. 18 2011; 52(8):5332-5338.
den Hollander A I, Roepman R, Koenekoop R K, Cremers F P. Leber congenital amaurosis: genes, proteins and disease mechanisms. Prog Retin Eye Res. July 2008; 27(4):391-419.
Liu X, Bulgakov O V, Darrow K N, et al. Usherin is required for maintenance of retinal photoreceptors and normal development of cochlear hair cells. Proc Natl Acad Sci USA. Mar. 13, 2007; 104(11):4413-4418.
Mastrobattista E, Hennink W E. Polymers for gene delivery: Charged for success. Nat Mater. Dec. 15 2011; 11(1):10-12.
Lundstrom K. Latest development in viral vectors for gene therapy. Trends Biotechnol. March 2003; 21(3):117-122.
Boylan N J, Kim A J, Suk J S, et al. Enhancement of airway gene transfer by DNA nanoparticles using a pH-responsive block copolymer of polyethylene glycol and poly-L-lysine. Biomaterials. March 2012; 33(7):2361-2371.
Cheng W, Yang C, Hedrick J L, Williams D F, Yang Y Y, Ashton-Rickardt P G. Delivery of a granzyme B inhibitor gene using carbamate-mannose modified PEI protects against cytotoxic lymphocyte killing. Biomaterials. May 2013; 34(14):3697-3705. de la Fuente M, Ravina M, Paolicelli P, Sanchez A, Seijo B, Alonso M J. Chitosan-based nanostructures: a delivery platform for ocular therapeutics. Adv Drug Deliv Rev. Jan. 31, 2010; 62(1):100-117.
Kim T H, Park I K, Nah J W, Choi Y J, Cho C S. Galactosylated chitosan/DNA nanoparticles prepared using water-soluble chitosan as a gene carrier. Biomaterials. August 2004; 25(17):3783-3792.
Read M L, Singh S, Ahmed Z, et al. A versatile reducible polycation-based system for efficient delivery of a broad range of nucleic acids. Nucleic Acids Res. May 24 2005; 33(9):e86.
Wang H, Shi H B, Yin S K. Polyamidoamine dendrimers as gene delivery carriers in the inner ear: How to improve transfection efficiency. Exp Ther Med. September 2011; 2(5):777-781.
Yu H, Russ V, Wagner E. Influence of the molecular weight of bioreducible oligoethylenimine conjugates on the polyplex transfection properties. AAPS J. September 2009; 11(3):445-455.
Pack D W, Hoffman A S, Pun S, Stayton P S. Design and development of polymers for gene delivery. Nat Rev Drug Discov. July 2005; 4(7):581-593.
Yang F, Green J J, Dinio T, et al. Gene delivery to human adult and embryonic cell-derived stem cells using biodegradable nanoparticulate polymeric vectors. Gene Ther. April 2009; 16(4):533-546.
Sunshine J C, Sunshine S B, Bhutto I, Handa J T, Green J J. Poly(beta-amino ester)-nanoparticle mediated transfection of retinal pigment epithelial cells in vitro and in vivo. PLoS One. 2012; 7(5):e37543.
Shmueli R B, Sunshine J C, Xu Z, Duh E J, Green J J. Gene delivery nanoparticles specific for human microvasculature and macrovasculature. Nanomedicine. October 2012; 8(7):1200-1207.
Sunshine J, Green J J, Mahon K P, et al. Small-Molecule End-Groups of Linear Polymer Determine Cell-type Gene-Delivery Efficacy. Adv Mater. Dec. 28 2009; 21(48):4947-4951.
Strauss O. The retinal pigment epithelium in visual function. Physiol Rev. July 2005; 85(3):845-881.
Kawa M P, Machalinska A, Roginska D, Machalinski B. Complement system in pathogenesis of AMD: dual player in degeneration and protection of retinal tissue J Immunol Res. 2014; 2014:483960.
Wang S, Koster K M, He Y, Zhou Q. miRNAs as potential therapeutic targets for age-related macular degeneration. Future Med Chem. March 2012; 4(3):277-287.
Abul-Hassan K, Walmsley R, Boulton M. Optimization of non-viral gene transfer to human primary retinal pigment epithelial cells. Curr Eye Res. May 2000; 20(5):361-366.
Bejjani R A, BenEzra D, Cohen H, et al. Nanoparticles for gene delivery to retinal pigment epithelial cells. Mol Vis. Feb. 17 2005; 11:124-132.
Chaum E, Hatton M P, Stein G. Polyplex-mediated gene transfer into human retinal pigment epithelial cells in vitro. J Cell Biochem. November 1999; 76(1):153-160.
Jayaraman M S, Bharali D J, Sudha T, Mousa S A. Nano chitosan peptide as a potential therapeutic carrier for retinal delivery to treat age-related macular degeneration. Mol Vis. 2012; 18:2300-2308.
Liu H A, Liu Y L, Ma Z Z, Wang J C, Zhang Q. A lipid nanoparticle system improves siRNA efficacy in RPE cells and a laser-induced murine CNV model. Invest Ophthalmol Vis Sci. Jul. 1, 2011; 52(7):4789-4794.
Mannermaa E, Ronkko S, Ruponen M, Reinisalo M, Urtti A. Long-lasting secretion of transgene product from differentiated and filter-grown retinal pigment epithelial cells after nonviral gene transfer. Curr Eye Res. May 2005; 30(5):345-353.
Mannisto M, Ronkko S, Matto M, et al. The role of cell cycle on polyplex-mediated gene transfer into a retinal pigment epithelial cell line. J Gene Med. April 2005; 7(4):466-476.
Mannisto M, Vanderkerken S, Toncheva V, et al. Structure-activity relationships of poly(L-lysines): effects of pegylation and molecular shape on physicochemical and biological properties in gene delivery. J Control Release. Sep. 18 2002; 83(1):169-182.
Peeters L, Sanders N N, Jones A, Demeester J, De Smedt S C. Post-pegylated lipoplexes are promising vehicles for gene delivery in RPE cells. J Control Release. Aug. 28 2007; 121(3):208-217.
Peng C H, Cherng J Y, Chiou G Y, et al. Delivery of Oct4 and SirT1 with cationic polyurethanes-short branch PEI to aged retinal pigment epithelium. Biomaterials. December 2011; 32(34):9077-9088.
Gan Q, Wang T, Cochrane C, McCarron P. Modulation of surface charge, particle size and morphological properties of chitosan-TPP nanoparticles intended for gene delivery. Colloids Surf B Biointerfaces. August 2005; 44(2-3):65-73.
Vercauteren D, Piest M, van der Aa L J, et al. Flotillin-dependent endocytosis and a phagocytosis-like mechanism for cellular internalization of disulfide based poly(amido amine)/DNA polyplexes. Biomaterials. April 2011; 32(11):3072-3084.
Klimanskaya I, Hipp J, Rezai K A, West M, Atala A, Lanza R. Derivation and comparative assessment of retinal pigment epithelium from human embryonic stem cells using transcriptomics. Cloning Stem Cells. 2004; 6(3):217-245.
Molla M R, Levkin P A. Combinatorial Approach to Nanoarchitectonics for Nonviral Delivery of Nucleic Acids. Adv Mater. Feb. 10 2016; 28(6):1159-1175.
Frohlich E. The role of surface charge in cellular uptake and cytotoxicity of medical nanoparticles. Int J Nanomedicine. 2012; 7:5577-5591.
Tomita Y, Rikimaru-Kaneko A, Hashiguchi K, Shirotake S. Effect of anionic and cationic n-butylcyanoacrylate nanoparticles on NO and cytokine production in Raw264.7 cells. Immunopharmacol Immunotoxicol. December 2011; 33(4):730-737.
Bishop C J, Ketola™, Tzeng S Y, et al. The effect and role of carbon atoms in poly(beta-amino ester)s for DNA binding and gene delivery. J Am Chem Soc. May 8 2013; 135(18):6951-6957.
Tzeng S Y, Wilson D R, Hansen S K, Quinones-Hinojosa A, Green J J. Polymeric nanoparticle-based delivery of TRAIL DNA for cancer-specific killing. Bioeng Transl Med. June 2016; 1(2):149-159.
Maruotti J, Wahlin K, Gorrell D, Bhutto I, Lutty G, Zack D J. A simple and scalable process for the differentiation of retinal pigment epithelium from human pluripotent stem cells. Stem Cells Transl Med. May 2013; 2(5):341-354.
Maruotti J, Sripathi S R, Bharti K, et al. Small-molecule-directed, efficient generation of retinal pigment epithelium from human pluripotent stem cells. Proc Natl Acad Sci USA. Sep. 1, 2015; 112(35):10950-10955.
Gamm D M, Melvan J N, Shearer R L, et al. A novel serum-free method for culturing human prenatal retinal pigment epithelial cells. Invest Ophthalmol Vis Sci. February 2008; 49(2):788-799.
Zhao T et al. (2014) Significance of branching for transfection: Synthesis of highly branched degradable functional poly(dimethylaminoethyl methacrylate) by vinyl oligomer combination. Angew Chemie-Int Ed.
Cutlar L, Zhou D, et al. (2015) Highly Branched Poly(β-Amino Esters): Synthesis and Application in Gene Delivery. Biomacromolecules.

Although the foregoing subject matter has been described in some detail by way of illustration and example for purposes of clarity of understanding, it will be understood by those skilled in the art that certain changes and modifications can be practiced within the scope of the appended claims.

1. A composition comprising a poly(beta-amino ester) (PBAE) of formula (I) or formula (II): 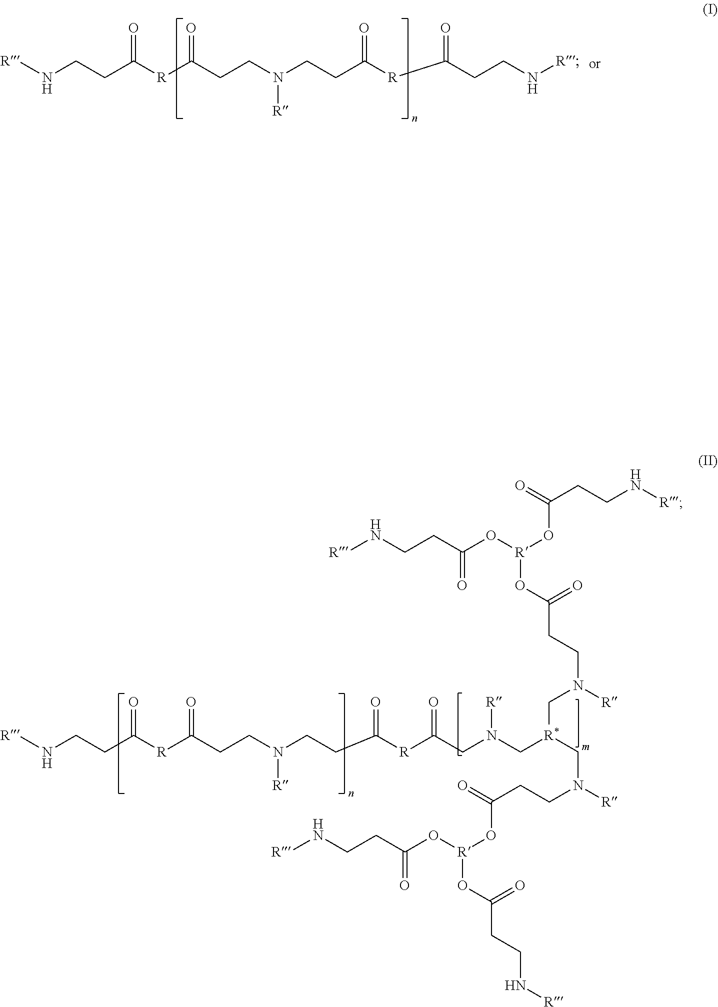
and at least one DNA or RNA molecule comprising a nucleic acid sequence encoding a gene-editing protein or therapeutic protein;
wherein:
n and m are each independently an integer from 1 to 10,000;
each R is independently a diacrylate monomer of the following structure: 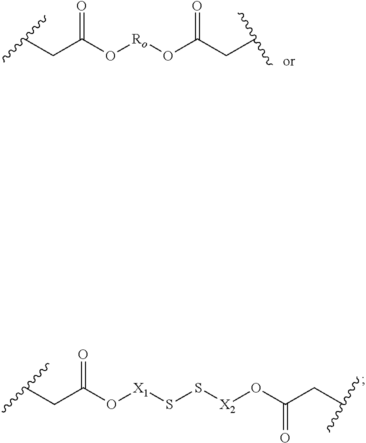
wherein Rₒ comprises a linear or branched C₁-C₃₀ alkylene chain, which may further comprise one or more heteroatoms or one or more carbocyclic, heterocyclic, or aromatic groups and X₁ and X₂ are each independently a linear or branched C₁-C₃₀ alkylene chain;
each R* is a triacrylate, quanternary, or hexafunctional acrylate monomer selected from the group consisting of: 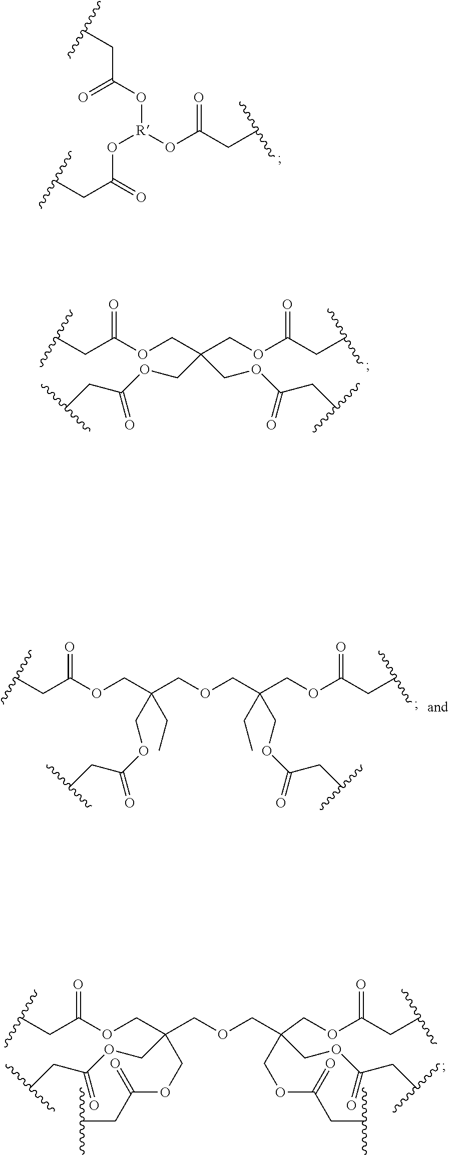
wherein each R′ is independently a trivalent group;
each R″ is independently a side chain monomer comprising a primary, secondary, or tertiary amine; and
each R′″ is independently an end group monomer comprising a primary, secondary, or tertiary amine.

2. The composition of claim 1, wherein the gene-editing protein is selected from the group consisting of CRISPR-associated nuclease, Cre recombinase, Flp recombinase, a meganuclease, a Transcription Activator-Like Effector Nuclease (TALEN), a Zinc-Finger Nuclease (ZFN), or a natural or engineered variant, family-member, orthologue, fragment or fusion construct thereof.

3. The composition of claim 2, wherein the gene-editing protein is a Cas9 endonuclease.

4. The composition of claim 3, wherein the composition further comprises a gRNA or DNA encoding a gRNA, wherein the Cas9 endonuclease and the gRNA are encoded on the same plasmid or are encoded on different plasmids.

5-6. (canceled)

7. The composition of claim 1, wherein the therapeutic protein is selected from the group consisting of CNGA3, CNGB3, GNAT2, sFLT01, Rab Escort Protein (REP-1), RS-1, RPE65, RPGR, MY07A, MERTK, ATP-binding cassette transporter 4 (ABCA4), and SAR-421869.

8. (canceled)

9. The composition of claim 1, wherein R is selected from the group consisting of: 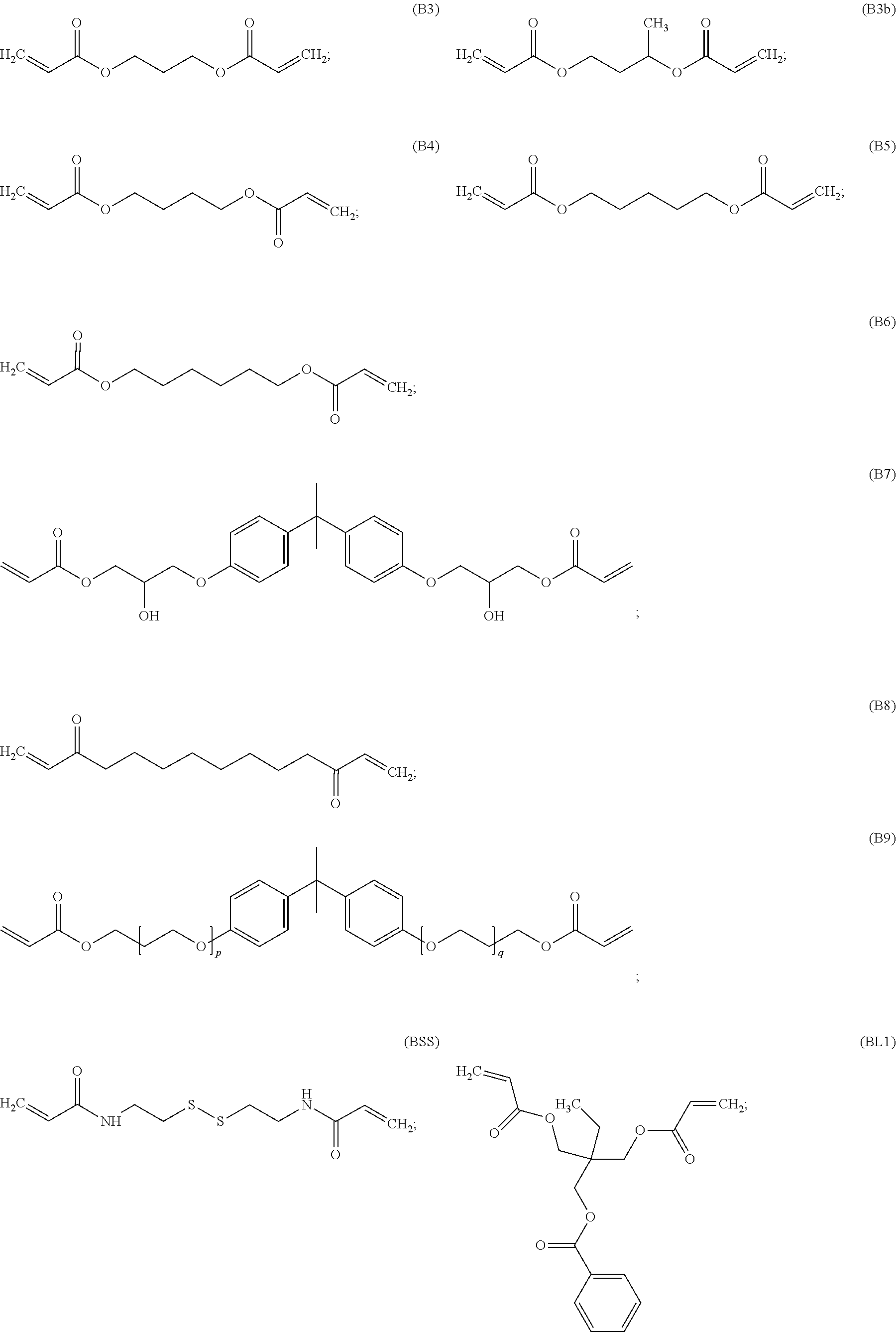 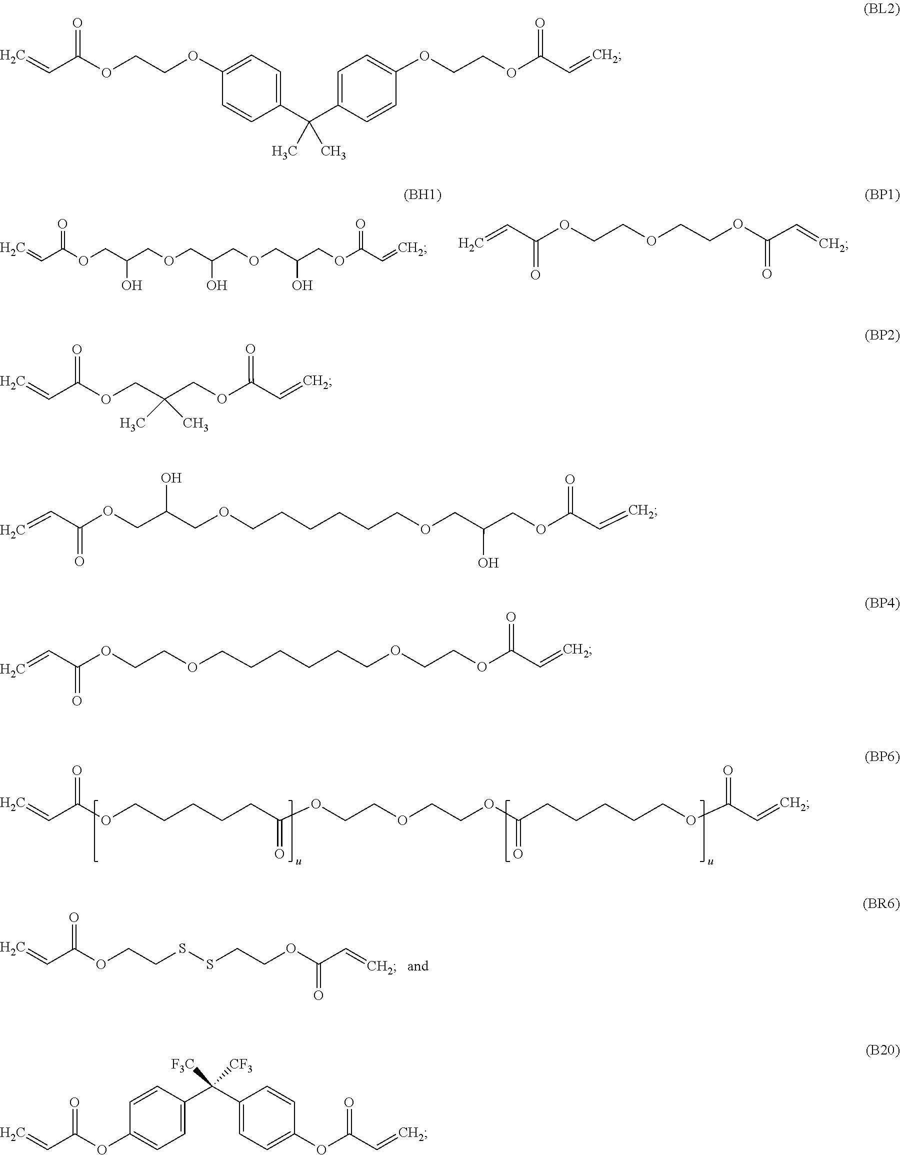
wherein p, q, and u are each independently an integer from 1 to 10,000.

10-11. (canceled)

12. The composition of claim 1, wherein the PBAE of formula (II) is:

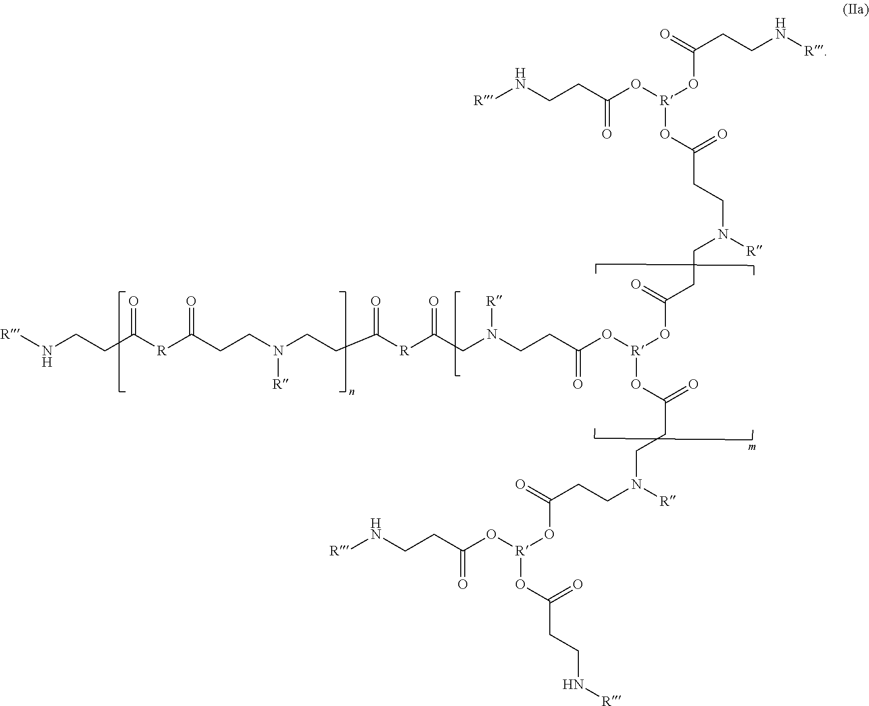

13. The composition of claim 1, wherein the triacrylate monomer is trimethylolpropane triacrylate (TMPTA):

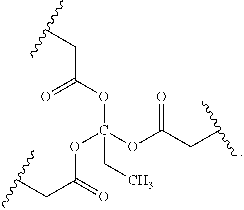 [SMILES: *CC(=O)OC(CC)(OC(=O)C*)OC(=O)C*]

14. The composition of claim 1, wherein R″ is selected from the group consisting of:

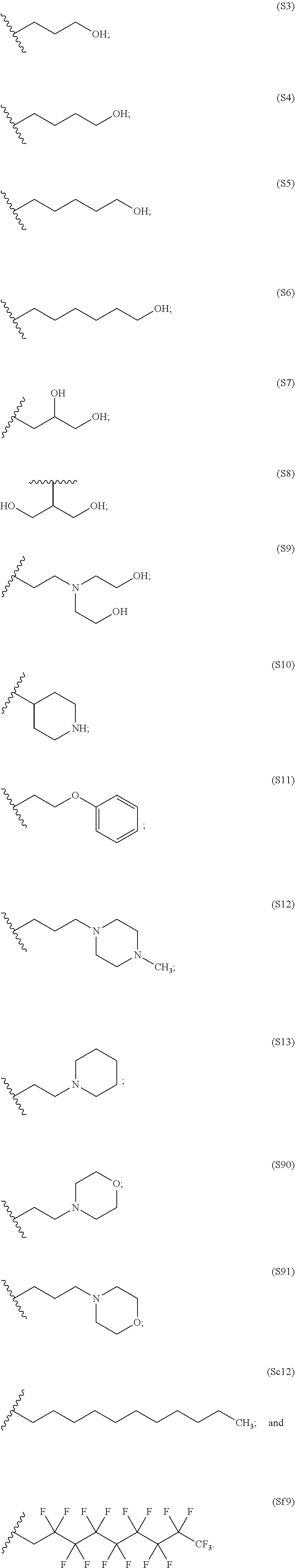

15. (canceled)

16. The composition of claim 1, wherein R′″ is an end group monomer selected from the group consisting of:
Amino Alkanes 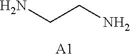 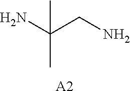 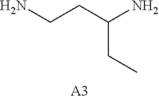 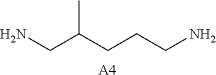 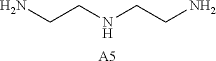 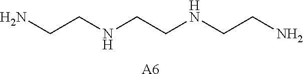 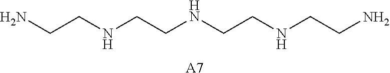 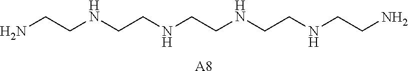 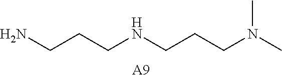 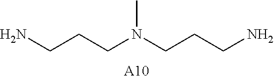 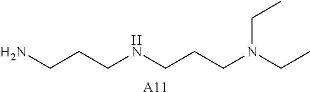 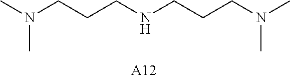 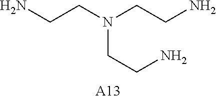 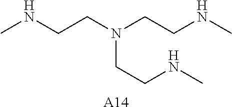
Amino Piperidines 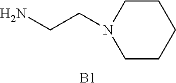 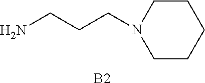 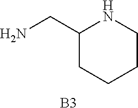 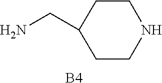
Amino Piperizines 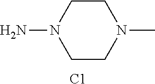 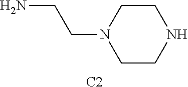 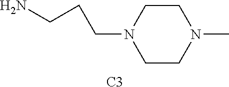 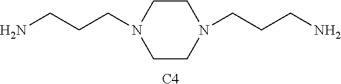
Amino Pyrrolidines 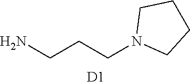 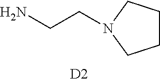
Amino Alcohols 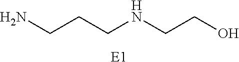 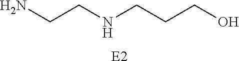 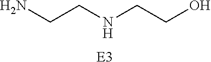 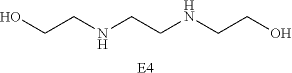 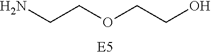 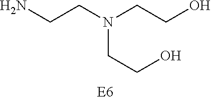 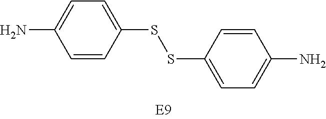 [SMILES: Nc1ccc(SSc2ccc(N)cc2)cc1] 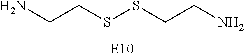 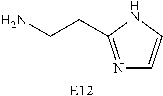 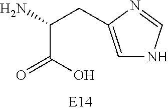 [SMILES: N[C@H](Cc1c[nH]cn1)C(=O)O] 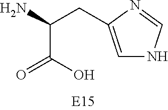 [SMILES: N[C@@H](Cc1c[nH]cn1)C(=O)O] 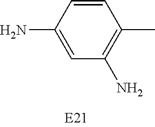 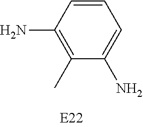 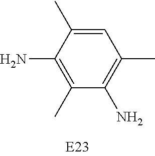 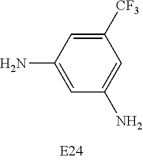 [SMILES: Nc1cc(N)cc(C(F)(F)F)c1] 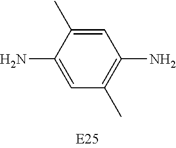 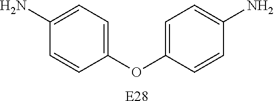 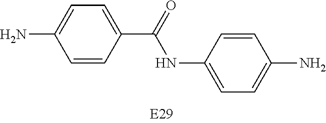 [SMILES: Nc1ccc(NC(=O)c2ccc(N)cc2)cc1] 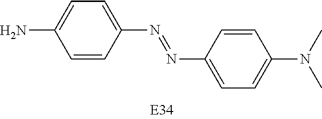 [SMILES: CN(C)c1ccc(/N=N/c2ccc(N)cc2)cc1] 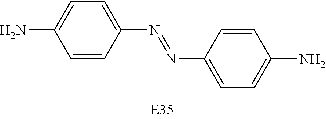 [SMILES: Nc1ccc(/N=N/c2ccc(N)cc2)cc1] 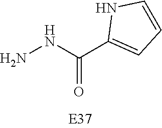 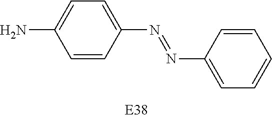 [SMILES: Nc1ccc(/N=N/c2ccccc2)cc1] 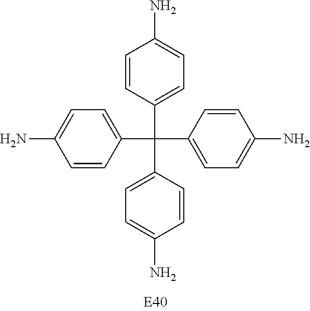 [SMILES: Nc1ccc(C(c2ccc(N)cc2)(c2ccc(N)cc2)c2ccc(N)cc2)cc1] 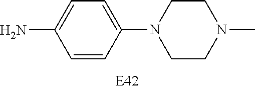 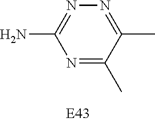 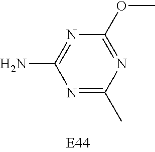 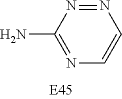 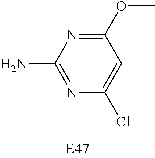 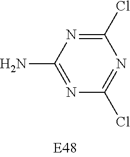
Diamino ethers 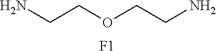 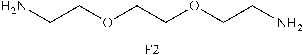 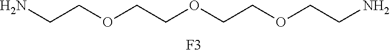 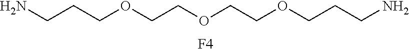
Amino morpholinos

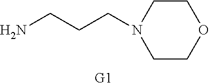

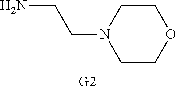

17. (canceled)

18. The compound of claim 1, wherein the PBAE of formula (I) is selected from:

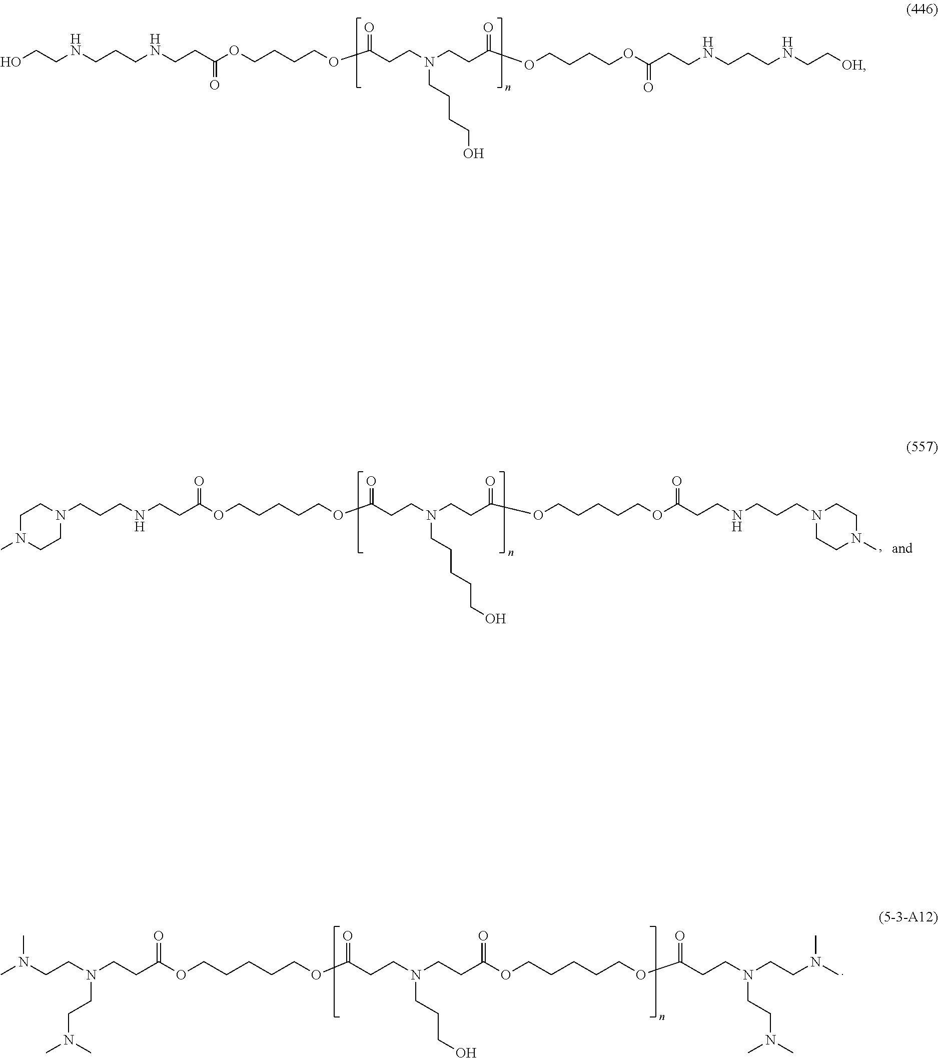

19-21. (canceled)

22. The composition of claim 1, wherein the composition has a PBAE-to-DNA weight-to-weight ratio (w/w) between 5-200 or between 30-90 w/w.

23. The composition of claim 1, wherein the nucleic acid sequence is operably linked to a promoter.

24. (canceled)

25. The of claim 1, further comprising a nanoparticle or microparticle of the PBAE of formula (I) or formula (II).

26. The pharmaceutical formulation of claim 25, wherein the nanoparticle or microparticle of the PBAE of formula (I) or formula (II) is encapsulated in a poly(lactic-co-glycolic acid) (PLGA) nanoparticle or microparticle.

27-28. (canceled)

29. A method for gene editing, the method comprising contacting a cell with the composition of claim 1, wherein the composition comprises at least one DNA plasmid comprising a nucleic acid sequence encoding a gene-editing protein.

30. The method of claim 29, wherein the gene-editing endonuclease directs site-specific target DNA disruption, mutation, deletion, or repair.

31. The method of claim 29, wherein the composition and cell are contacted in vivo or ex vivo.

32. (canceled)

33. The method of claim 29, wherein the cell is selected from an eukaryotic cell, an animal cell, a plant cell, a mammalian cell, a human cell, a stem cell, progenitor cell, multipotent cell, and a pluripotent cell.

34-38. (canceled)

39. A method for treating a retinal eye disease, the method comprising administering to a subject in need of treatment thereof, a composition of claim 1, wherein the composition comprises a therapeutic protein for treating retinal eye disease.

40. The method of claim 39, wherein the retinal eye disease comprises a hereditary retinal eye disease.

41. The method of claim 39, wherein the retinal eye disease is selected from the group consisting of age-related macular degeneration (AMD), including wet macular degeneration and dry macular degeneration, Leber's congenital amaurosis (LCA2) type 2, choroideremia, achromatopsia, retinitis pigmentosa (RP), Stargardt disease (STGD), Usher syndrome, juvenile X-linked retinoschisis (XLRS), and diabetic retinopathy.

42. The method of claim 39, wherein the therapeutic protein is selected from the group consisting of CNGA3, CNGB3, GNAT2, sFLT01, Rab Escort Protein (REP-1), RS-1, RPE65, RPGR, MY07A, MERTK, ATP-binding cassette transporter 4 (ABCA4), and SAR-421869.

43. The method of claim 39, wherein the therapeutic protein is administered via an injection technique selected from the group consisting of intra-cameral injection, sub-conjunctival injection, intravitreal injection, and subretinal injection.

44. The method of claim 39, wherein the composition is delivered to one or more cells of a retinal pigmented epithelium (RPE) of the subject.


SEQUENCE LISTING (45 sequences)

SEQ ID NO: 1 - RNA, 34 nt, Artificial sequence
  Synthetic
  misc_feature (1)..(12): n is a, c, g, or u
nnnnnnnnnnnnguuuaagagcuaugcuguuuug

SEQ ID NO: 2 - protein, 1368 aa, Streptococcus pyogenes [sequence omitted]

SEQ ID NO: 3 - DNA, 20 bp, Artificial sequence
  Synthetic
gcaagctgaccctgaagttc

SEQ ID NO: 4 - DNA, 22 bp, Artificial sequence
  Synthetic
ctggtcgagctggacggcgacg

SEQ ID NO: 5 - DNA, 22 bp, Artificial sequence
  Synthetic
cacgaactccagcaggaccatg

SEQ ID NO: 6 - DNA, 21 bp, Artificial sequence
  Synthetic
cgcaaatgggcggtaggcgtg

SEQ ID NO: 7 - DNA, 20 bp, Artificial sequence
  Synthetic
gcccttgctcaccatgaatt

SEQ ID NO: 8 - DNA, 22 bp, Artificial sequence
  Synthetic
ggagttgacgccaaagcaatcc

SEQ ID NO: 9 - DNA, 23 bp, Artificial sequence
  Synthetic
agatttaaagttgggggtcagcc

SEQ ID NO: 10 - DNA, 22 bp, Artificial sequence
  Synthetic
atcccgtatgaaggtctgagcg

SEQ ID NO: 11 - DNA, 22 bp, Artificial sequence
  Synthetic
gtcgatcatgttcggcgtaacc

SEQ ID NO: 12 - DNA, 20 bp, Artificial sequence
  Synthetic
acattatacggtttcagagc

SEQ ID NO: 13 - DNA, 20 bp, Artificial sequence
  Synthetic
gactcggtgccactttttca

SEQ ID NO: 14 - DNA, 21 bp, Artificial sequence
  Synthetic
catgtacgttgctatccaggc

SEQ ID NO: 15 - DNA, 21 bp, Artificial sequence
  Synthetic
ctccttaatgtcacgcacgat

SEQ ID NO: 16 - DNA, 19 bp, Artificial sequence
  Synthetic
ctgtccctgtatgcctctg

SEQ ID NO: 17 - DNA, 18 bp, Artificial sequence
  Synthetic
atgtcacgcacgatttcc

SEQ ID NO: 18 - DNA, 19 bp, Artificial sequence
  Synthetic
gtatagcatacattatacg

SEQ ID NO: 19 - DNA, 19 bp, Artificial sequence
  Synthetic
taccacatttgtagaggtt

SEQ ID NO: 20 - DNA, 19 bp, Artificial sequence
  Synthetic
caatgtatcttatcatgtc

SEQ ID NO: 21 - DNA, 19 bp, Artificial sequence
  Synthetic
gtatagcatacattatacg

SEQ ID NO: 22 - DNA, 19 bp, Artificial sequence
  Synthetic
taccacatttgtagaggtt

SEQ ID NO: 23 - DNA, 19 bp, Artificial sequence
  Synthetic
caatgtatcttatcatgtc

SEQ ID NO: 24 - DNA, 19 bp, Artificial sequence
  Synthetic
taccacatttgtagaggtt

SEQ ID NO: 25 - DNA, 19 bp, Artificial sequence
  Synthetic
caatgtatcttatcatgtc

SEQ ID NO: 26 - DNA, 20 bp, Artificial sequence
  Synthetic
gatcgagttcgagcctgcgg

SEQ ID NO: 27 - DNA, 19 bp, Artificial sequence
  Synthetic
gcgcgttctttggacgcga

SEQ ID NO: 28 - DNA, 19 bp, Artificial sequence
  Synthetic
cgtgatgttgtaccgcttc

SEQ ID NO: 29 - DNA, 36 bp, Artificial sequence
  Synthetic
attattgactagtagtggttttagagctagaaatag

SEQ ID NO: 30 - DNA, 36 bp, Artificial sequence
  Synthetic
caagttaaaataaggctagtccgttatcaacttgaa

SEQ ID NO: 31 - DNA, 35 bp, Artificial sequence
  Synthetic
aaagtggcaccgagtcggtgcaacaaagcaccagt

SEQ ID NO: 32 - DNA, 36 bp, Artificial sequence
  Synthetic
ggtctagtggtagaatagtaccctgccacggtacag

SEQ ID NO: 33 - DNA, 36 bp, Artificial sequence
  Synthetic
acccgggttcgattcccggctggtgcagccaagctt

SEQ ID NO: 34 - DNA, 7 bp, Artificial sequence
  Synthetic
ggcgtaa

SEQ ID NO: 35 - DNA, 29 bp, Artificial sequence
  Synthetic
agttagtttctagaacaaagcaccagtgg

SEQ ID NO: 36 - DNA, 20 bp, Artificial sequence
  Synthetic
gaacctctacaaatgtggta

SEQ ID NO: 37 - DNA, 36 bp, Artificial sequence
  Synthetic
taggtctccacaaatgtggtagttttagagctagaa

SEQ ID NO: 38 - DNA, 36 bp, Artificial sequence
  Synthetic
atggtctcattgtagaggttctgcaccagccgggaa

SEQ ID NO: 39 - DNA, 20 bp, Artificial sequence
  Synthetic
gcaatgtatcttatcatgtc

SEQ ID NO: 40 - DNA, 36 bp, Artificial sequence
  Synthetic
taggtctcctcttatcatgtcgttttagagctagaa

SEQ ID NO: 41 - DNA, 36 bp, Artificial sequence
  Synthetic
atggtctcaaagatacattgctgcaccagccgggaa

SEQ ID NO: 42 - DNA, 35 bp, Artificial sequence
  Synthetic
caatgtataagcttaaaaaaaaaagcaccgactcg

SEQ ID NO: 43 - DNA, 29 bp, Artificial sequence
  Synthetic
gtccaggagcgcaccatcttctttcaagg

SEQ ID NO: 44 - DNA, 27 bp, Artificial sequence
  Synthetic
gtccaggagcgcaccatcttctcaagg

SEQ ID NO: 45 - DNA, 27 bp, Artificial sequence
  Synthetic
gtccaggagcgcaccatcttcttcagg